SEQUENCE LISTING (9 sequences)

SEQ ID NO: 1 - protein, 8 aa, Artificial Sequence
  Designed Peptide
  DISULFID (2)..(7)
FCFFKTCY

SEQ ID NO: 2 - protein, 8 aa, Artificial Sequence
  Designed Peptide
  DISULFID (2)..(7)
FCYFKTCY

SEQ ID NO: 3 - protein, 8 aa, Artificial Sequence
  Designed peptide
  DISULFID (2)..(7): 3 = Pyridylalanine
FCXFKTCY

SEQ ID NO: 4 - protein, 8 aa, Artificial Sequence
  Designed Peptide
  DISULFID (2)..(7)
FCFWKTCT

SEQ ID NO: 5 - protein, 8 aa, Artificial Sequence
  Designed Peptide
  DISULFID (2)..(7)
FCYWKTCT

SEQ ID NO: 6 - protein, 8 aa, Artificial Sequence
  Designed Peptide
  DISULFID (2)..(7)
FCYWKTCT

SEQ ID NO: 7 - protein, 8 aa, Artificial Sequence
  Designed Peptide
  DISULFID (2)..(7): 3 = L-1-Naphthylalanine
FCXWKTCT

SEQ ID NO: 8 - protein, 19 aa, Artificial Sequence
  Designed Peptide
TFFYGGSRGKRNNFKTEEY

SEQ ID NO: 9 - protein, 7 aa, Polaribacter, P. haliotis
VNTANST

(19) United States
(12) Patent Application Publication
(10) Pub. No.: US 2025/0235566 A1
(43) Pub. Date: Jul. 24, 2025

(54) TRISLINKER-CONJUGATED DIMERIC LABELING PRECURSORS AND RADIOTRACERS DERIVED THEREFROM

(71) Applicant: Medianezia GmbH, Wiesbaden (DE)

(72) Inventors: Frank RÖSCH, Berlin (DE); Marcel MARTIN, Mainz (DE); Tilmann GRUS, Mainz (DE); Euy Sung MOON, Mainz (DE); Chandra Sekhar BAL, Bhubaneswar (IN)

(73) Assignee: Medianezia GmbH, Wiesbaden (DE)

(21) Appl. No.: 18/567,891

(22) PCT Filed: Jun. 7, 2022
(86) PCT No.: PCT/EP2022/065440

§ 371 Date: Dec. 7, 2023

(30) Foreign Application Priority Data
Jun. 8, 2021 (DE) 10 2021 114 711.5

(51) Int. Cl. A61K 51/08; A61K 51/04

(52) U.S. Cl. CPC A61K 51/088; A61K 51/0455; A61K 51/0491; A61K 51/0497; A61K 2121/00; A61K 2123/00

(57) ABSTRACT

The invention relates to a radiotracer labeling precursor includes a first target vector (TV1), a second target vector (TV2), a labeling group (MG) for complexation or covalent binding of a radioisotope, a first spacer (S1), a second spacer (S2), a third spacer (S3) and a trislinker (TL), as illustrated in the structure below:

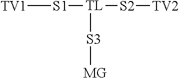

### SEQUENCE LISTING

This application contains a sequence listing whose file name is AFC21008SequenceListing.txt, which was created on Jul. 31, 2024, has a size of 2436 bites and which is hereby incorporated by reference herein in its entirety.

### CROSS-REFERENCE TO RELATED APPLICATIONS

This application is being filed under 35 U.S.C. § 371 as a National Stage Application of pending International Application No. PCT/EP2022/065440 filed Jun. 7, 2022, which claims priority to German Patent Application No. 10 2021 114 711.5, filed Jun. 8, 2021. Both International Application No. PCT/EP2022/065440 and German Patent Application No. 10 2021 114 711.5 are hereby incorporated by reference herein in their entirety.

### FIELD OF THE INVENTION

The present invention relates to dimeric labeling precursors and to radiotracers derived therefrom by complexation with a radioisotope for the diagnosis and treatment of cancer. The labeling precursor has the structure

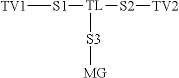

in which TV1 is a first targeting vector, TV2 is a second targeting vector, MG is a labeling group for complexation or the covalent bond of a radioisotope, S1 is a first spacer, S2 is a second spacer, S3 is a third spacer and TL is a tris linker.

The labeling precursors and radiotracers of the invention are intended for imaging nuclear-medical diagnostics, especially positron emission tomography (PET) and single-photon emission computed tomography (SPECT), and also radionuclide therapy/endotherapy of carcinomas and metastases of various cancer types.

### BACKGROUND OF THE INVENTION

In nuclear-medical diagnostics, tumor cells or metastases are labeled and imaged with the aid of a radioactive isotope, for example gallium-68 (⁶⁸Ga), technetium-99m (^{99m}Tc) or scandium-44 (⁴⁴Sc). For metallic radionuclides of the above type, complex-forming chelators are used.

Nonmetallic radioisotopes, such as fluorine-18 (¹⁸F), iodine-123 (¹²³I), iodine-131 (¹³¹I) and astatine-211 (²¹¹At), are bound covalently, i.e. no chelator is required.

By comparison with diagnostics, higher radiation doses are used in nuclear-medical therapy in order to destroy tumor tissue. For this purpose, for example, beta-minus-emitting radioisotopes such as lutetium-177 (¹⁷⁷Lu), yttrium-90 (⁹⁰Y) and iodine-131 (¹³¹I) or alpha emitters such as actinium-225 (²²⁵Ac) are used. Alpha and beta-minus rays have a short range in tissue. The short range enables localized irradiation of tumors and metastases with low radiation dose and damage to the surrounding healthy tissue.

In the last few years, the combination of diagnosis and therapy—referred to as theranostics among specialists—has gained increasing importance. In this context, the same labeling precursor can be used both for diagnostics and for therapy. The labeling precursor is merely labeled here with different radioisotopes, for example with ⁶⁸Ga and ¹⁷⁷Lu, such that PET diagnostics and radiotherapy are performable with chemically essentially identical compounds. This permits translation of the results of imaging nuclear-medical diagnosis to nuclear-medical treatment (theranostics) with improved adjustment of dose.

The labeling group—especially chelators—modifies the configuration and chemical properties of a targeting vector conjugated to the labeling group and generally affects the affinity thereof for tumor cells. Accordingly, the labeling precursor has to be reevaluated with regard to complexation with radioisotopes, and in particular with regard to its biochemical and pharmacological in vitro and in vivo properties. The labeling group and the chemical coupling thereof to the targeting vector are crucial to the biological and nuclear-medical potency of the corresponding radiotracer.

After intravenous injection into the bloodstream, the labeling precursor labeled with the radioisotope—also referred to hereinafter as radiotracer—accumulates at or in tumor cells or metastases. In order to minimize the radiation dose in healthy tissue, radioisotopes with a short half-life of a few hours to a few days are used.

In summary, it can be stated that the labeling precursor and radiotracers derived therefrom must meet the following requirements:
1. rapid and effective complexation or binding of the respective radioisotope;
2. high selectivity for tumor cells and metastases relative to healthy tissue;
3. in vivo stability, i.e. biochemical stability in blood serum under physiological conditions;
4. high enrichment in the tumor and any metastases, which enables precise diagnostics and effective therapy;
5. low retention and rapid excretion from healthy tissue and the blood in order to minimize the dose and toxicity for these organs.

### Prostate Cancer

For men in industrial countries, prostate cancer is the most common type of cancer and the third most deadly cancer. Tumor growth advances only slowly with this disorder, and the 5-year survival rate in the case of diagnosis at an early stage is nearly 100%. But if the disorder is discovered only after the tumor has metastasized, the survival rate drops significantly. On the other hand, excessively early and excessively aggressive action against the tumor can unnecessarily significantly impair the patient's quality of life. For example, the operative removal of the prostate can lead to incontinence and impotence. Reliable diagnosis and information as to the stage of the disease are essential for successful treatment with a high quality of life for the patient. A widespread means of diagnosis alongside the palpation of the prostate by a doctor is the determination of tumor markers in the patient's blood. The most prominent marker for prostate carcinoma is the concentration of the prostate-specific antigen (PSA) in the blood. However, the meaningfulness of the PSA concentration is disputed since patients having slightly elevated values often do not have prostate carcinoma, but 15% of patients having prostate carcinoma do not show an elevated PSA concentration in the blood. A further target structure for the diagnosis of prostate tumors is the prostate-specific membrane antigen (PSMA). By contrast with PSA, PSMA cannot be detected in the blood. It is a membrane-bound glycoprotein having enzymatic activity. Its function is the elimination of C-terminal glutamate from N-acetyl-aspartyl-glutamate (NAAG) and folic acid-(poly)-γ-glutamate. PSMA barely occurs in normal tissue, but is greatly overexpressed by prostate carcinoma cells, with a close correlation of expression with the stage of the tumor disorder. Lymph node metastases and bone metastases of prostate carcinoma also show expression of PSMA to an extent of 40%.

A strategy in the molecular targeting of PSMA is to bind to the protein structure of the PSMA with antibodies. Moreover, ligands that address the enzymatic binding pockets of PSMA are used. The central enzymatic binding pocket of PSMA contains two Zn²⁺ ions that bind glutamate. In front of the central binding pocket is an aromatic binding pocket. The PSMA protein is capable of expanding and of an induced fit to various ligands, such as inhibitors or enzymatically cleavable. Thus, PSMA, as well as NAAG, also binds folic acid, where the pteroic acid group docks in the aromatic binding pocket. The addressing of the PSMA binding pocket with an inhibitor or substrate generally induces cellular incorporation (endocytosis).

PSMA inhibitors are especially suitable as targeting vectors for imaging diagnostic and theranostic radiopharmaceuticals or radiotracers. The radiolabeled inhibitors dock onto the central PSMA binding pocket, where they are not enzymatically converted or cleaved, and the inhibitor/targeting vector is not detached from the radioactive label. Promoted by endocytosis, the inhibitor with the radioactive label is incorporated into the tumor cell and enriched therein.

Inhibitors having high affinity for PSMA (scheme 1) generally contain a glutamate motif and an enzymatically non-cleavable structure. A highly effective PSMA inhibitor is 2-phosphonomethylglutaric acid or 2-phosphonomethylpentanedioic acid (2-PMPA), in which the glutamate motif is bound to a phosphonate group which is not cleavable by PSMA. Moreover, urea-based PSMA inhibitors are used, for example in clinically relevant radiotracers of the PSMA-11 type (scheme 2) and PSMA-617 type (scheme 3).

It has been found to be advantageous, in addition to the central binding pocket, to address the aromatic binding pocket of PSMA. For example, in highly active radiotracers of the PSMA-11 type, the L-lysine-urea-L-glutamate binding motif (KuE) is bound via hexyl (hexyl spacer) to an aromatic HBED chelator (N,N′-bis[2-hydroxy-5-carboxyethyl]benzyl)ethylene-diamine-N,N′-diacetate).

If L-lysine-urea-L-glutamate (KuE), by contrast, is bound to the non-aromatic DOTA chelator (1,4,7,10-tetraazacyclododecane-1,4,7,10-tetraacetate), reduced affinity and enrichment in tumor tissue are established. In order nevertheless to be able to use the DOTA chelator for a radiopharmaceutical having PSMA affinity with therapeutic radionuclides, such as ¹⁷⁷Lu or ²²⁵Ac, the spacer has to be adapted. By means of controlled replacement of the hexyl spacer with various aromatic structures, the PSMA-617 labeling precursor and the highly active ¹⁷⁷Lu-PSMA-617 radiotracer derived therefrom, the current gold standard, were found.

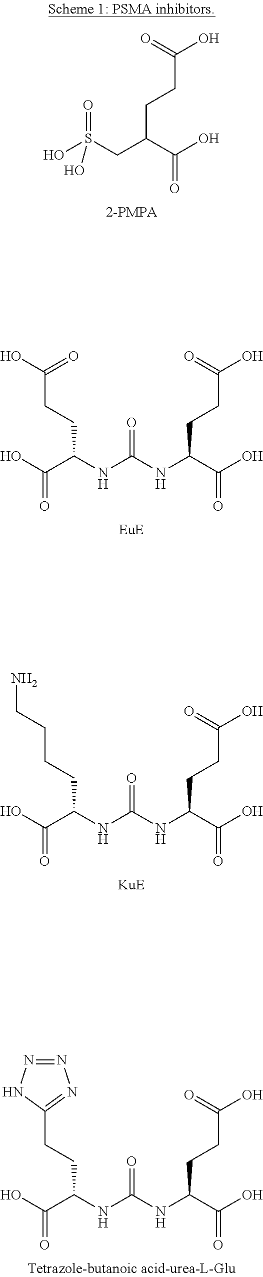

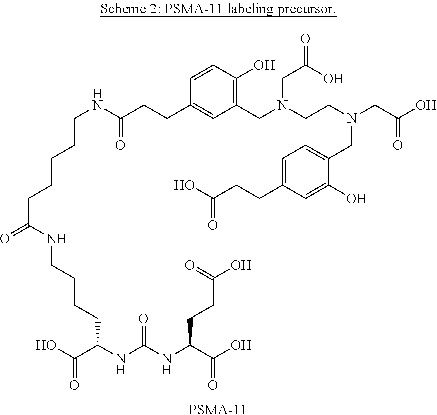

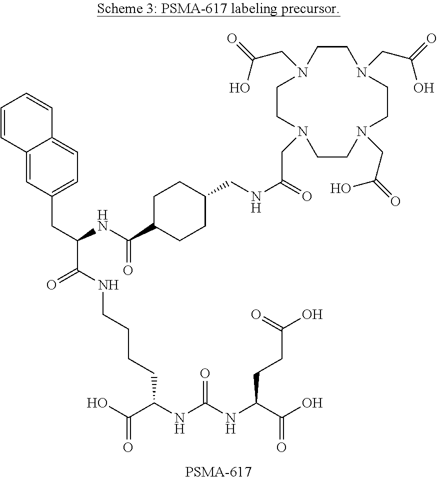

### Tumor Stroma

Malignant epithelial cells are a constituent of many tumors and tumor types and form a tumor stroma surrounding the tumor at the latest from a size of 1-2 mm.

The tumor stroma (tumor microenvironment, TME) comprises various non-malignant types of cells and may account for up to 90% of the total tumor mass. It plays an important role in tumor progression, or tumor growth and metastasis.

The most important cellular components of the tumor stroma are the extracellular matrix including various cytokines, endothelial cells, pericytes, macrophages, immune regulatory cells and activated fibroblasts. The activated fibroblasts surrounding the tumor are referred to as cancer-associated fibroblasts (CAF).

In the course of tumor evolution, CAFs change morphology and biological function. These changes are induced by intercellular communication between cancer cells and CAFs. In this context, CAFs form an environment that promotes the growth of the cancer cells. It has been shown that therapies targeting solely cancer cells are inadequate. Effective therapies must also include the tumor microenvironment and hence also the CAFs.

For more than 90% of all human epithelial carcinomas, CAFs overexpress the fibroblast activation protein (FAP). Therefore, FAP represents a promising point of attack for nuclear-medical diagnosis and therapy. Analogously to PSMA, FAP inhibitors (FAPI or FAPi) in particular are suitable as targeting vectors for FAP labeling precursors and radiotracers derived therefrom. The role of FAP in vivo is not yet fully understood, but it is known that it is an enzyme having specific catalytic activity. It has both dipeptidylpeptidase (DPP) activity and prolyloligopeptidase (PREP) activity. Accordingly, useful inhibitors are those that inhibit the DPP activity and/or the PREP activity of FAP. What is crucial is the selectivity of the inhibitor with respect to other similar enzymes such as the dipeptidylpeptidases DPPII, DPPIV, DPP8 and DPP9, and with respect to prolyloligopeptidase (PREP). In the case of cancer types where both FAP and PREP are overexpressed, however, it is also possible to use inhibitors that do not have high selectivity between PREP and FAP, but inhibit both enzymes.

In 2013, a high-affinity and high-selectivity inhibitor structure was developed and published, the basis of which is a modified glycine-proline unit coupled to a quinoline (JANSEN et al. ACS Med. Chem. Lett. 2013, 4, 491-496). The compound in question, (S)—N-(2-(2-cyanopyrrolidin-1-yl)-2-oxoethyl)quinoline-4-carboxamide, is depicted in scheme 4 (on the left). In subsequent structure-activity studies (SAR), compounds having improved affinity and selectivity were found, including the difluorinated derivative (S)—N-(2-(2-cyano-4,4′-difluoropyrrolidin-1-yl)-2-oxoethyl)quinoline-4-carboxamide, UAMC1110 for short, which is depicted in scheme 4 (on the right) (JANSEN et al. J. Med. Chem. 2014, 57 (7), 3053-3074).

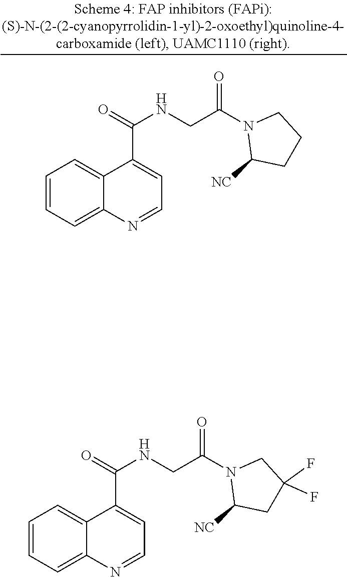 [SMILES: N#C[C@@H]1CCCN1C(=O)CNC(=O)c1ccnc2ccccc12 ; N#C[C@@H]1CC(F)(F)CN1C(=O)CNC(=O)c1ccnc2ccccc12]

UAMC1110 forms the basis for targeting vectors of various FAP labeling precursors and radiotracers for nuclear medical use. Scheme 5 (at the top) shows the FAPI-04 labeling precursor byway of example (LINDNER et al. J. Nucl. Med. 2018, 59 (9), 1415-1422). Scheme 5 (at the bottom) shows a further FAP labeling precursor comprising the DOTA chelator. The DOTA chelator is bonded therein to the quinoline unit of the pharmacophoric FAPi targeting vector via a 4-aminobutoxy group, a squaric acid group and an ethylenediamine group.

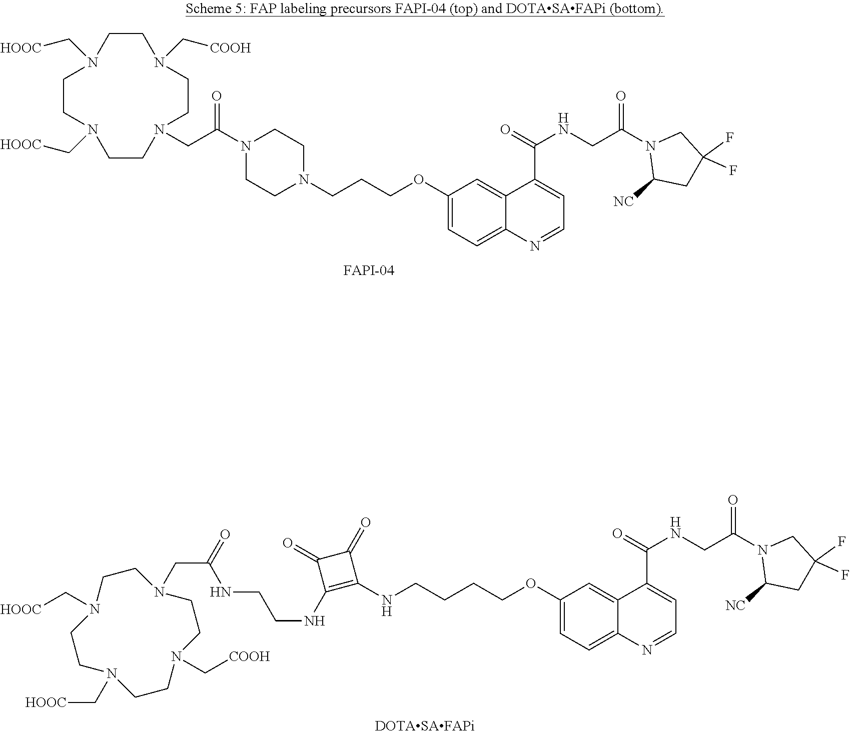 [SMILES: N#C[C@@H]1CC(F)(F)CN1C(=O)CNC(=O)c1ccnc2ccc(OCCCN3CCN(C(=O)CN4CCN(CC(=O)O)CCN(CC(=O)O)CCN(CC(=O)O)CC4)CC3)cc12 ; N#C[C@@H]1CC(F)(F)CN1C(=O)CNC(=O)c1ccnc2ccc(OCCCCNc3c(NCCNC(=O)CN4CCN(CC(=O)O)CCN(CC(=O)O)CCN(CC(=O)O)CC4)c(=O)c3=O)cc12]

### Bone Metastases

Bone metastases express farnesyl pyrophosphate synthase (FPPS), an enzyme in the HMG-CoA reductase (mevalonate) pathway. The inhibition of FPPS suppresses the production of farnesyl, an important molecule for the docking of signal proteins to the cell membrane. As a result, the apoptosis of carcinogenic bone cells is induced. FPPS is inhibited by bisphosphonates, such as alendronate, pamidronate and zoledronate. For example, the BPAMD tracer together with the pamidronate targeting vector is regularly used in the treatment of bone metastases.

A particularly effective tracer for the theranostics of bone metastases has been found to be zoledronate (ZOL), a hydroxy-bisphosphonate with a heteroaromatic imidazole unit. The NODAGA- and DOTA-conjugated zoledronate chelators (scheme 6) are the currently most potent radiotheranostics for bone metastases.

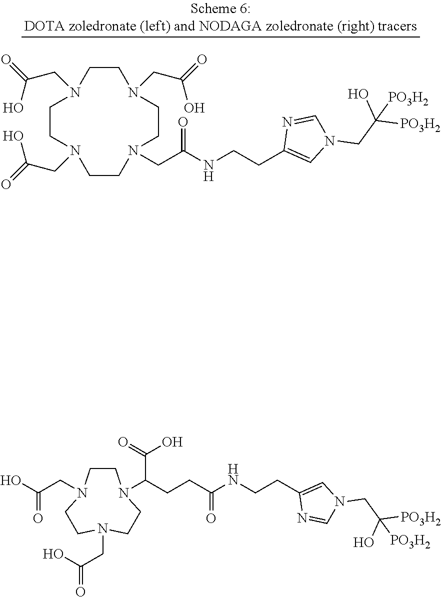

The prior art discloses a multitude of labeling precursors for the diagnosis and theranostics of cancers with radioactive isotopes.

For instance, WO 2015055318 A1 discloses radiotracers for the diagnosis and theranostics of prostate carcinomas or epithelial carcinomas, such as the PSMA-617 labeling precursor shown in scheme 3 inter alia.

Monomeric radiotracers with a targeting vector (TV) play a central role in nuclear medicine and are well deserving of the name “precision oncology”. As of recently, dimeric labeling precursors with two targeting vectors are also being examined. It is assumed here that a radiotracer with two targeting vectors has elevated affinity. The prior art discloses “linear” homodimeric labeling precursors having two identical targeting vectors each coupled to a central chelator, and first studies in this regard support this hypothesis (Zia, N. A. et al. Angw. Chem. Int. Ed. 2019, 58, 14991-14994).

### SUMMARY OF ADVANTAGEOUS EMBODIMENTS OF THE INVENTION

In the present invention, homo- and heterodimeric labeling precursors are provided for the first time, which comprise two identical or two different targeting vectors conjugated via a tris linker (TL) with a labeling group. The tris linker (TL) used is, for example, an amino acid residue, such as, in particular, a lysine residue or glutamic acid residue.

The tris linker (TL) of the invention decouples the chelator and the targeting vectors with regard to steric and electronically induced interactions. The coupling of the tris linker (TL) to the chelator is designed such that it does not impair complexation with radioisotopes of clinical relevance. For this purpose, it is possible to make use of couplings that have been found to be useful for monomeric labeling precursors. The invention enables independent (orthogonal) optimization of radioisotope complexation, of affinity, and of the pharmacokinetics and pharmacodynamics of homo- and heterodimeric radiotracers. By contrast, the known linear, homodimeric labeling precursors entail complex molecular engineering which is often associated with functional impairments.

FAP-addressing labeling precursors and radiotracers of the invention additionally have the following features:

1. A high binding affinity for FAP with IC₅₀ values in the nanomolar and sub-nanomolar range.

2. An exceptional binding specificity with respect to the competing PREP proteases and to the DPPIV family such as, in particular DPP4 (type II integral protein with intracellular and extracellular forms), but also DPP8 and DPP9 (intracellular proteins) (Hamson et al., Proteomics Clin. Appl. 2014, 8, 454-463). The binding affinities of the compounds of the invention are in the micromolar range here, as a result of which the ratio of the binding to the FAP target and the competing proteases usually assumes a value of >1000. The ratio can be illustrated with the aid of a selectivity index (SI) between the IC₅₀ values (see table 2). This significantly reduces the accumulation of the radiolabeled compounds of the invention in tissues outside the tumor micro-environment (healthy tissue) and guarantees exceptionally high contrast in molecular imaging.

3. High hydrophilicity (low log D value), which leads to a short dwell time of the compounds of the invention in the blood. This guarantees exceptionally high contrast in molecular imaging between the tumors and the surrounding perfused healthy tissue.

4. Rapid enrichment and long dwell time of the compounds of the invention in the tumor microenvironment. This ensures that a high radiation dose can be administered in the tumor or its environment even in the case of use of relatively long-lived radioisotopes such as lutetium-177 and actinium-225 in endoradiotherapy.

5. A short dwell time of the compounds of the invention in healthy tissue by rapid elimination via the kidney and bladder. This guarantees not only exceptionally high contrast in molecular imaging between the tumors and the surrounding blood-supplied healthy tissue but also low radiation stress for the patients.

Furthermore, the concept of the invention can readily be applied to compounds having two different targeting vectors. It is possible here, for example, to use a bone metastasis-addressing targeting vector (bisphosphonate) together with a prostate cancer-addressing targeting vector (PSMA inhibitor). This has the advantage that, in prostate cancer patients with bone metastases, these can be addressed better than by radiopharmaceuticals having solely a PSMA targeting vector. The reason for this lies in the high heterogeneity of PSMA expression in the bone metastases of patients, such that these can be addressed only inadequately under some circumstances with PSMA inhibitor structures.

Only in about 90% of patients suffering from prostate carcinoma is there overexpression of PSMA. Accordingly, in the context of the invention, heterodimeric labeling precursors with an FAP targeting vector and a PSMA targeting vector are also envisaged. Such heterodimeric labeling precursors address both PSMA-expressing tumor tissue and tumor-associated FAP-expressing stroma cells. It is thus also possible to detect and visualize prostate carcinomas and metastases that do not overexpress PSMA by means of PET and SPECT.

It is an object of the present invention to provide labeling precursors and radiotracers for improved diagnosis and theranostics of cancer disorders. In particular, labeling precursors and radiotracers are to be provided with elevated selectivity and specificity, effective radioisotope complexation and conjugation, and rapid absorption and systemic excretion.

This object is achieved by a labeling precursor having the structure

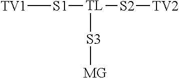

in which TV1 is a first targeting vector, TV2 is a second targeting vector, MG is a chelator or a linker for the complexation or covalent binding of a radioisotope, S1 is a first spacer, S2 is a second spacer, S3 is a third spacer and TL is a tris linker.

### BRIEF DESCRIPTION OF THE FIGURES

FIG. 1 is a graphical illustration of the reaction kinetics of [⁶⁸Ga]Ga-DOTA.Glu.(FAPi)₂;

FIG. 2 is a graphical illustration of a radio-HPLC curve for [⁶⁸Ga]Ga-DOTA.Glu.(FAPi)₂;

FIG. 3 is a graphical illustration of the stability of [⁶⁸Ga]Ga-DOTA.Glu.(FAPi)₂;

FIG. 4 is a graphical illustration of the reaction kinetics of [¹⁷⁷Lu]Lu-DOTA.Glu.(FAPi)₂;

FIG. 5 is a graphical illustration of a radio-HPLC curve for [¹⁷⁷Lu]Lu-DOTA.Glu.(FAPi)₂;

FIG. 6 is a graphical illustration of the stability of [¹⁷⁷Lu]Lu-DOTA.Glu.(FAPi)₂;

FIG. 7 is a graphical illustration of the reaction kinetics of [⁶⁸Ga]Ga-DOTAGA.Glu.(FAPi)₂;

FIG. 8 is a graphical illustration of a radio-HPLC curve for [⁶⁸Ga]Ga-DOTAGA.Glu.(FAPi)₂;

FIG. 9 is a graphical illustration of stability for [⁶⁸Ga]Ga-DOTAGA.Glu.(FAPi)₂;

FIG. 10 is a graphical illustration of the reaction kinetics of [¹⁷⁷Lu]Lu-DOTAGA.Glu.(FAPi)₂;

FIG. 11 is a graphical illustration of a radio-HPLC curve for [¹⁷⁷Lu]Lu-DOTAGA.Glu.(FAPi)₂;

FIG. 12 is a graphical illustration of the stability of [¹⁷⁷Lu]Lu-DOTAGA.Glu.(FAPi)₂;

FIG. 13 is a graphical illustration of the reaction kinetics of [²²⁵Ac]Ac-DOTAGA.Glu.(FAPi)₂;

FIG. 14 is a graphical illustration of the stability of [²²⁵Ac]Ac-DOTAGA.Glu.(FAPi)₂;

FIG. 15 is a graphical illustration of the reaction kinetics of [⁶⁸Ga]Ga-DATA^{5m}.Glu.(FAPi)₂;

FIG. 168 is a graphical illustration of a radio-HPLC curve for [⁶⁸Ga]Ga-DATA^{5m}.Glu.(FAPi)₂; and

FIG. 17 is a graphical illustration of the stability of [⁶⁸Ga]Ga-DATA^{5m}.Glu.(FAPi)₂.

### DETAILED DESCRIPTION OF ADVANTAGEOUS EMBODIMENTS OF THE INVENTION

Appropriate embodiments of the labeling precursor of the invention are characterized by the following features in any combination, to the extent to which the features are not mutually exclusive, and according to which:
TV1 and TV2 are independently chosen from one of the structures [1] to [43]:

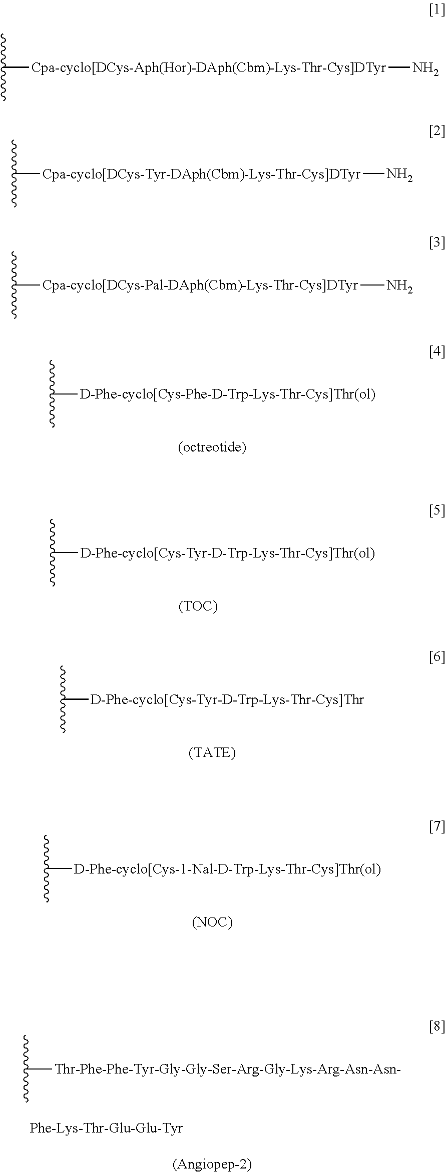

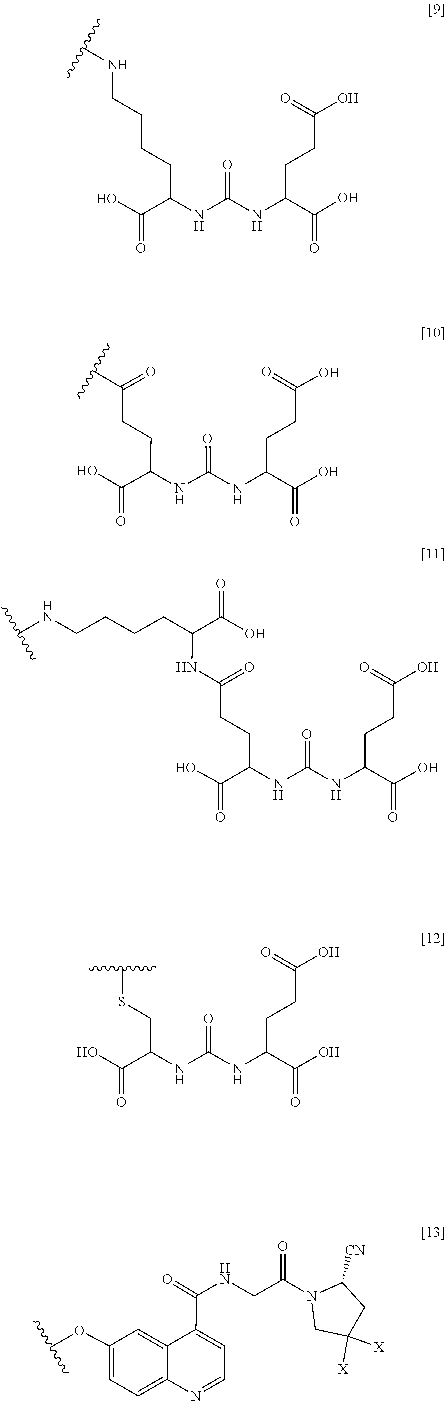

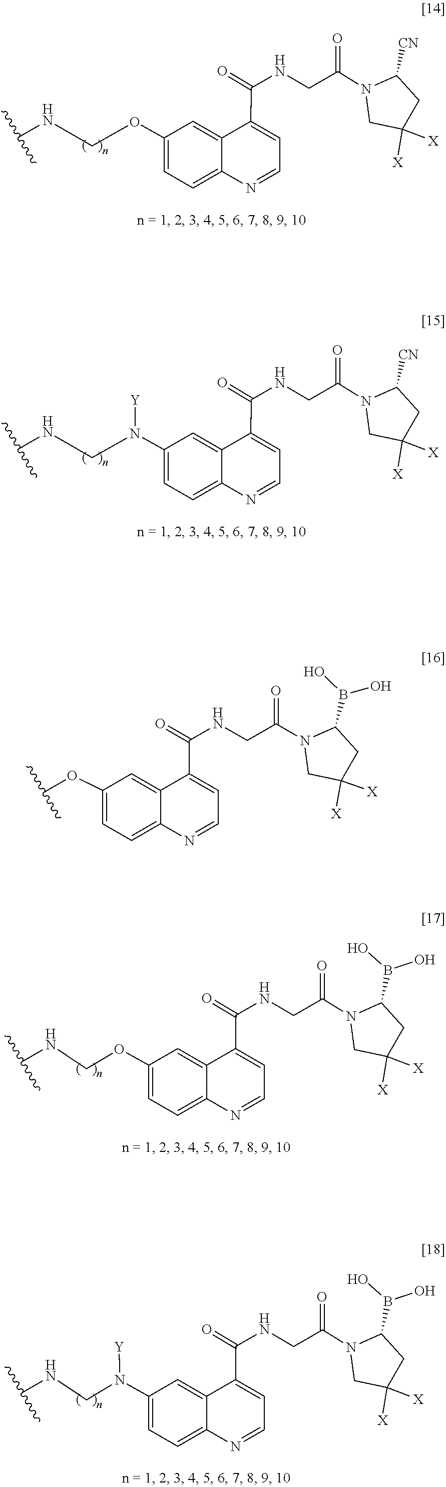

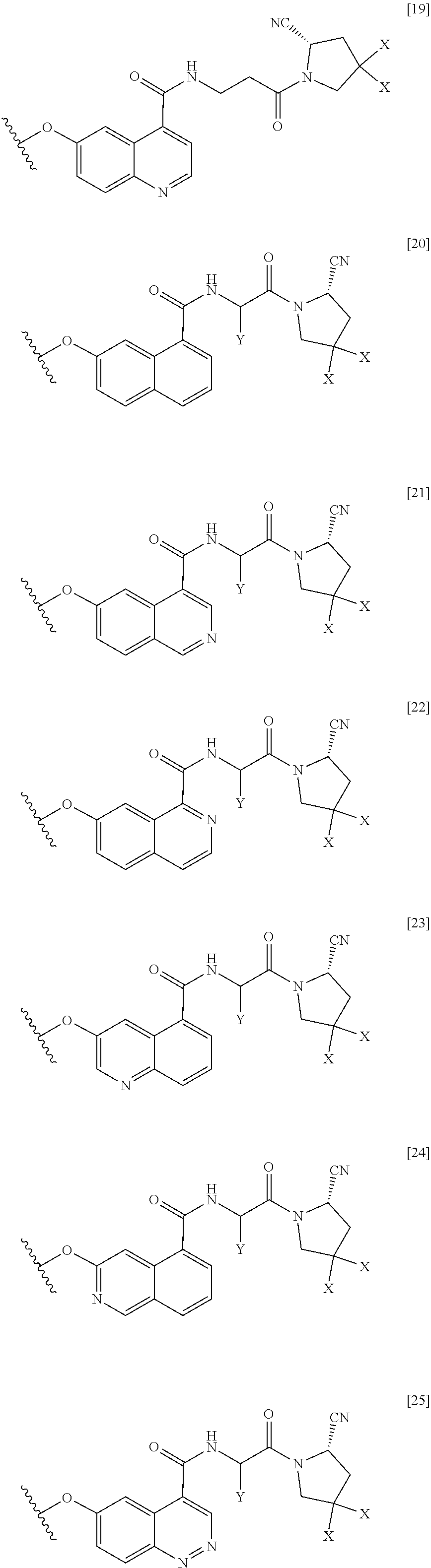

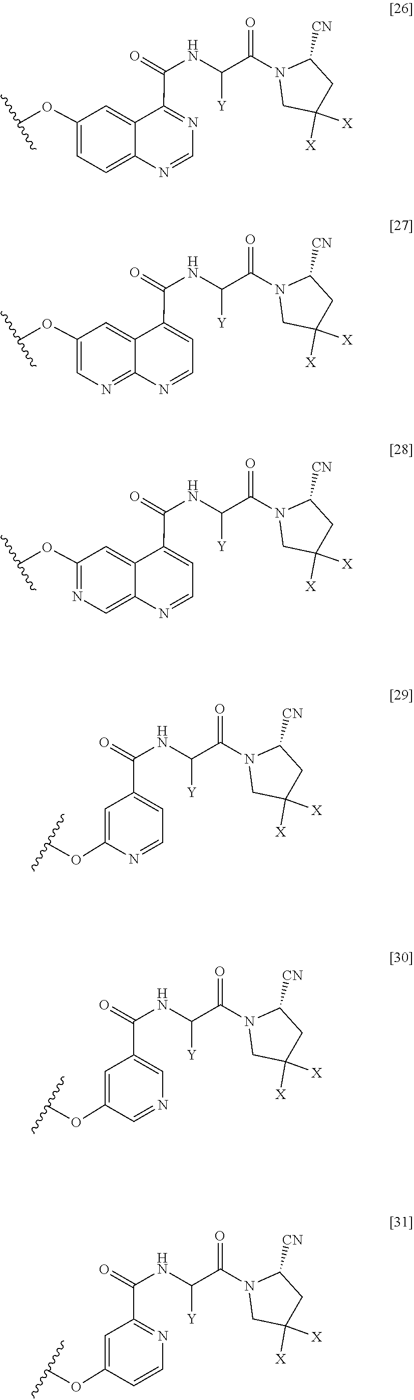

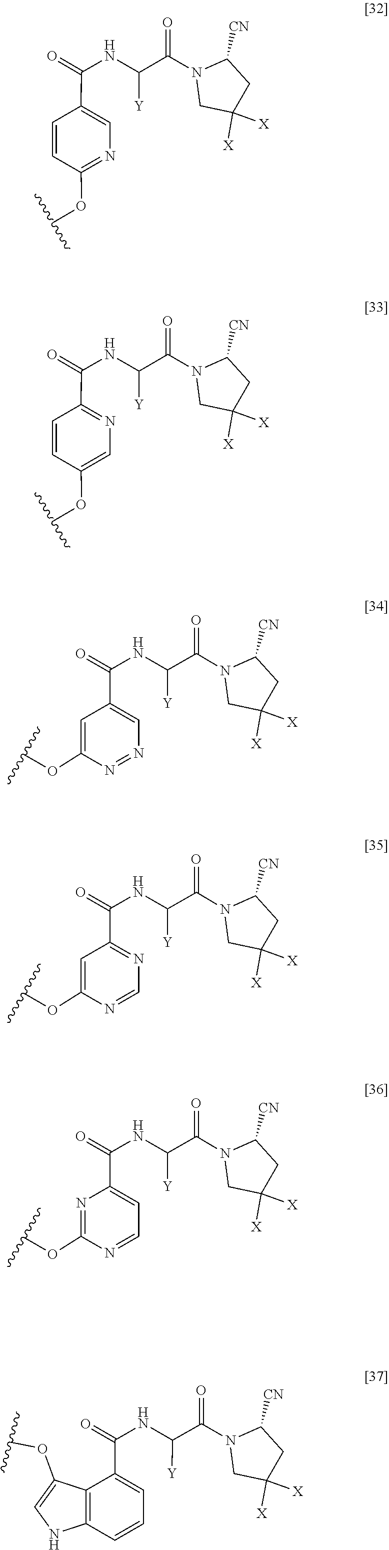

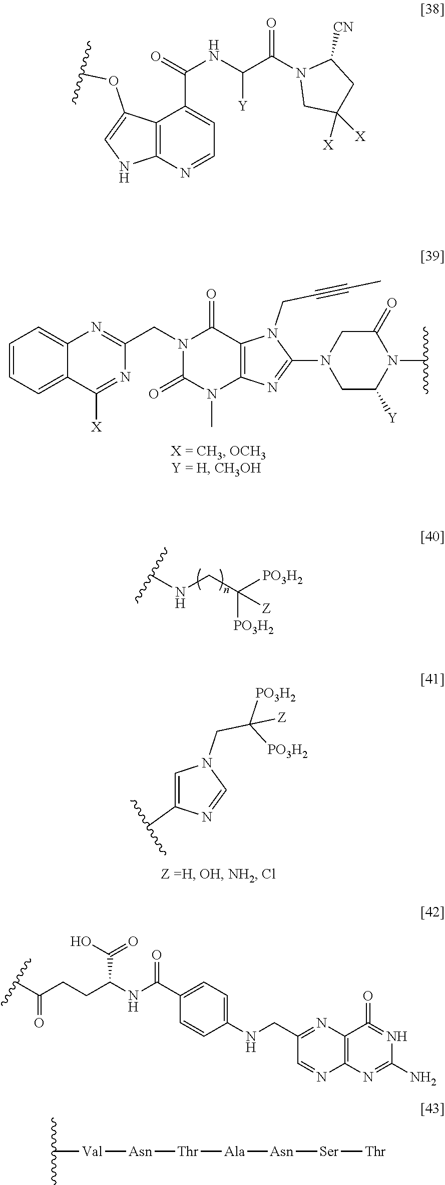

where
structures [1] to [8] and [43] denote peptides;
X═H or F;
Y═H, CH₃, CH(CH₃)₂, C(CH₃)₃ or (CH₂)ₙCH₃ with n=1, 2, 3, 4, 5, 6, 7, 8, 9 or 10;
TV1 is the same as TV2 (TV1=TV2);
TV1 and TV2 are different than one another (TV1≠TV2);
TV1 has the structure [13];
TV1 has the structure [14];
TV2 has the structure [13];
TV2 has the structure [14];
TV1 and TV2 each have the structure [13];
TV1 and TV2 each have the structure [14];
TV1 has one of the structures [9] to [12] and TV2 has one of the structures [13] or [14];
TV1 has one of the structures [9] to [12] and TV2 has one of the structures [40] or [41];
TV2 has one of the structures [9] to [12] and TV1 has one of the structures [13] or [14];
TV2 has one of the structures [9] to [12] and TV1 has one of the structures [40] or [41];
S1, S2 and S3 independently have a structure chosen from

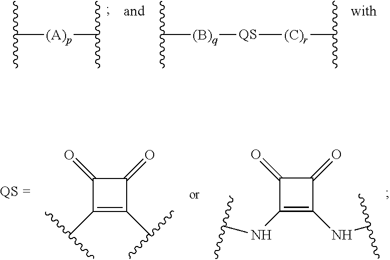

in which A, B, C are independently chosen from the group comprising amide radicals, carboxamide radicals, phosphinate radicals, alkyl radicals, triazole radicals, thiourea radicals, ethylene radicals, maleimide radicals, amino acid residues,

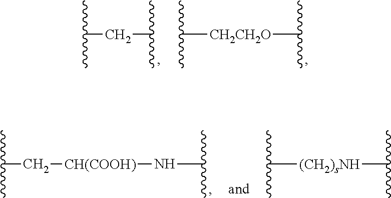

with s=1, 2, 3, 4, 5, 6, 7, 8, 9 or10; and
p, q and r are independently chosen from the set of {0, 1, 2, 3, 4, 5, 6, 7, 8, 9, 10, 11, 12, 13, 14, 15, 16, 17, 18, 19, 20};
S1, S2, S3 independently have the structure

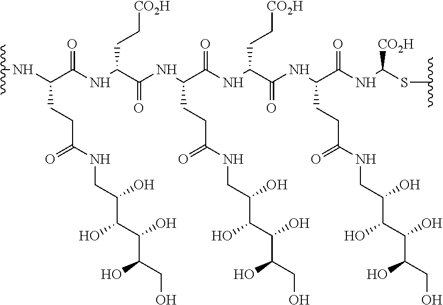 [SMILES: *N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](S*)C(=O)O]

S1, S2, S3 is independently a peptide group having the structure

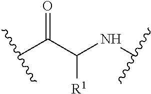

S1, S2, S3 is independently a dipeptide group having the structure

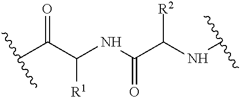

S1, S2, S3 is independently a tripeptide group having the structure

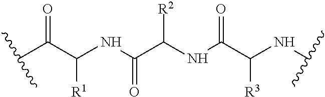

the side chains R¹, R², R³ are peptidic spacers S1, S2, S3 independently chosen from the group comprising —H, —CH₃, —CH(CH₃)₂, —CH₂CH(CH₃)₂, —CH(CH₃)—CH₂CH₃, —CH₂-Phe, —CH₂-Phe—OH, —CH₂SH, —(CH₂)₂—S—CH₃, —CH₂OH, —(CH)(OH)(CH₃), —(CH₂)₄NH₂, —(CH₂)₃NH(C═NH)NH₂, —CH₂COOH, —(CH₂)₂COOH, —CH₂(C═O)NH₂, —(CH₂)₂(C═O)NH₂,

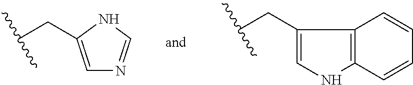 [SMILES: *Cc1cnc[nH]1 ; *Cc1c[nH]c2ccccc12]

MG is a chelator for the complexation of a radioisotope from the group comprising ⁴³Sc, ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹In, ¹³⁵Sm, ¹⁴⁰Pr ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th;
MG is a chelator chosen from the group comprising H₄pypa, EDTA (ethylenediaminetetraacetate), EDTMP (diethylenetriaminepenta(methylenephosphonic acid)), DTPA (diethylenetriaminepentaacetate) and derivatives thereof, NOTA (nona-1,4,7-triamine triacetate) and derivatives thereof, such as NODAGA (1,4,7-triazacyclononane,1-glutaric acid,4,7-acetate), TRAP (triazacyclononanephosphinic acid), NOPO (1,4,7-triazacyclononane-1,4-bis[methylene(hydroxymethyl)phosphinic acid]-7-[methylene(2-carboxyethyl)phosphinic acid]), DOTA (dodeca-1,4,7,10-tetraaminetetraacetate), DOTAGA (2-(1,4,7,10-tetraazacyclododecane-4,7,10)-pentanedioic acid) and other DOTA derivatives, TRITA (trideca-1,4,7,10-tetraaminetetraacetate), TETA (tetradeca-1,4,8,11-tetraaminetetraacetate) and derivatives thereof, PEPA (pentadeca-1,4,7,10,13-pentaaminepentaacetate), HEHA (hexadeca-1,4,7,10,13,16-hexaaminehexaacetate) and derivatives thereof, HBED (N,N′-bis(2-hydroxybenzyl)ethylenediamine-N,N′-diacetate) and derivatives thereof, such as HBED-CC (N,N′-bis[2-hydroxy-5-carboxyethyl]benzyl)ethylenediamine-N,N′-diacetate), DEDPA and derivatives thereof, such as H₂dedpa (1,2-[[6-(carboxyl)pyridin-2-yl]-methylamine]ethane) and H₄octapa (1,2-[[6-(carboxyl)pyridin-2-yl]methylamine]ethane-N,N′-diacetate), DFO (deferoxamine) and derivatives thereof, trishydroxypyridinone (THP) and derivatives thereof, such as H₃THP-Ac and H₃THP-mal (YM103), TEAP (tetraazacyclodecanephosphinic acid) and derivatives thereof, AAZTA (6-amino-6-methylperhydro-1,4-diazepane-N,N,N′,N′-tetraacetate) and derivatives thereof, such as AAZTA⁵(5-[(6-amino)-1,4-diazepane]pentanoic acid-N,N,N′,N′-tetraacetate) DATA^{5m} (5-[[6-(N-methyl)amino]-1,4-diacetate-1,4-diazepane]pentanoic acid-N,N′,N′-triacetate); sarcophagine SAR (1-N-(4-aminobenzyl)-3,6,10,13,16,19-hexaazabicyclo[6.6.6]-eicosane-1,8-diamine) and derivatives thereof, such as (NH₂)₂SAR (1,8-diamino-3,6,10,13,16,19-hexaazabicyclo[6.6.6]eicosane), N4 (3-[(2′-aminoethyl)amino]-2-[(2″-aminoethyl)aminomethyl]propionic acid) and other N₄ derivatives, PnAO (6-(4-isothiocyanatobenzyl)-3,3,9,9-tetramethyl-4,8-diazaundecane-2,10-dione dioxime) and derivatives, such as BMS181321 (3,3′-(1,4-butanediyldiamino)bis(3-methyl-2-butanone) dioxime), MAG2 (mercaptoacetylglycylglycine) and derivatives thereof, MAG3 (mercaptoacetylglycylglycylglycine) and derivatives thereof, such as N₃S-adipate, MAS3 (mercaptoacetylserylserylserine) and derivatives thereof, MAMA (N-(2-mercaptoethyl)-2-[(2-mercaptoethyl)amino]acetamide) and derivatives thereof, EC (ethylenedicysteine) and derivatives thereof, dmsa (dimercaptosuccinic acid) and derivatives thereof, DADT (diaminodithiol), DADS (diaminodisulfide), N₂S₂ chelators and derivatives thereof, aminothiols and derivatives thereof; salts of the aforementioned chelators; hydrazinenicotinamides (HYNIC) and hydrazinenicotinamide derivatives;
the labeling group MG has a structure chosen from the group comprising structures [44], [45], [46] and [47]:

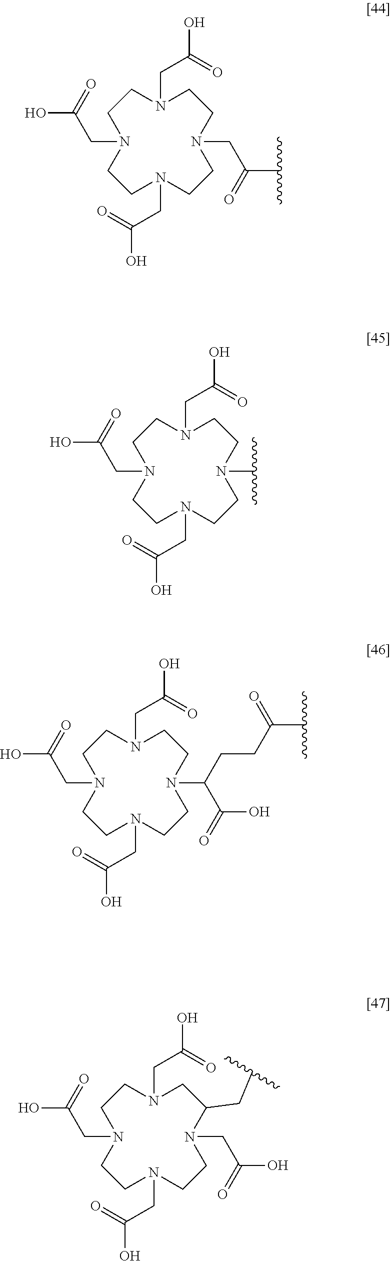 [SMILES: *C(=O)CN1CCN(CC(=O)O)CCN(CC(=O)O)CCN(CC(=O)O)CC1 ; *N1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *CC1CN(CC(=O)O)CCN(CC(=O)O)CCN(CC(=O)O)CCN1CC(=O)O]

the labeling group MG has a structure chosen from the group comprising structures [48], [49], [50] and [51]:

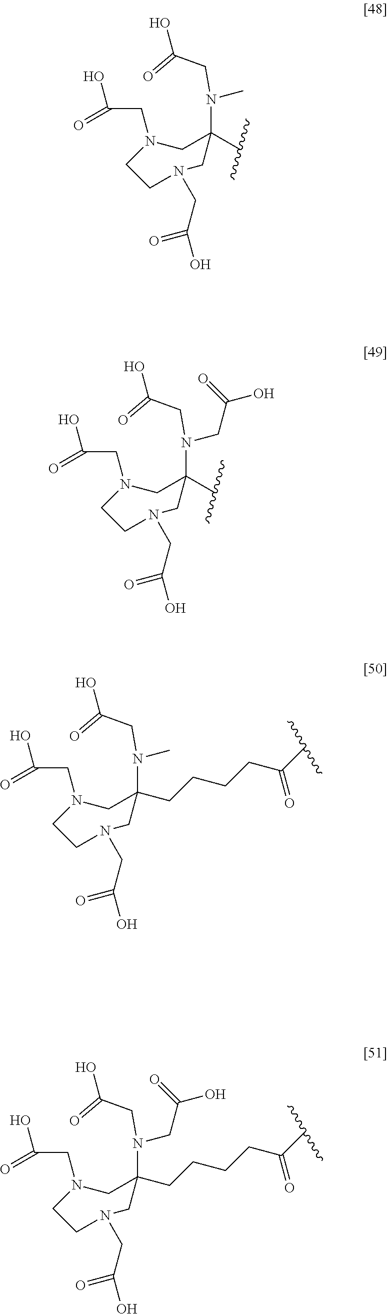 [SMILES: *C1(N(C)CC(=O)O)CN(CC(=O)O)CCN(CC(=O)O)C1 ; *C1(N(CC(=O)O)CC(=O)O)CN(CC(=O)O)CCN(CC(=O)O)C1 ; *C(=O)CCCCC1(N(C)CC(=O)O)CN(CC(=O)O)CCN(CC(=O)O)C1 ; *C(=O)CCCCC1(N(CC(=O)O)CC(=O)O)CN(CC(=O)O)CCN(CC(=O)O)C1]

MG is DOTA (dodeca-1,4,7,10-tetraaminetetraacetate);
MG is DATA^{5m} (1,4-bis(carboxymethyl)-6-[methylcarboxymethylamino]-6-pentanoic acid-1,4-diazepane);
MG is AAZTA (1,4-bis(carboxymethyl)-6-[bis(carboxymethyl)amino]-6-pentanoic acid-1,4-diazepane);
MG is a linker for the covalent binding of ¹⁸F, ¹³¹I or ²¹¹At;
MG is chosen from

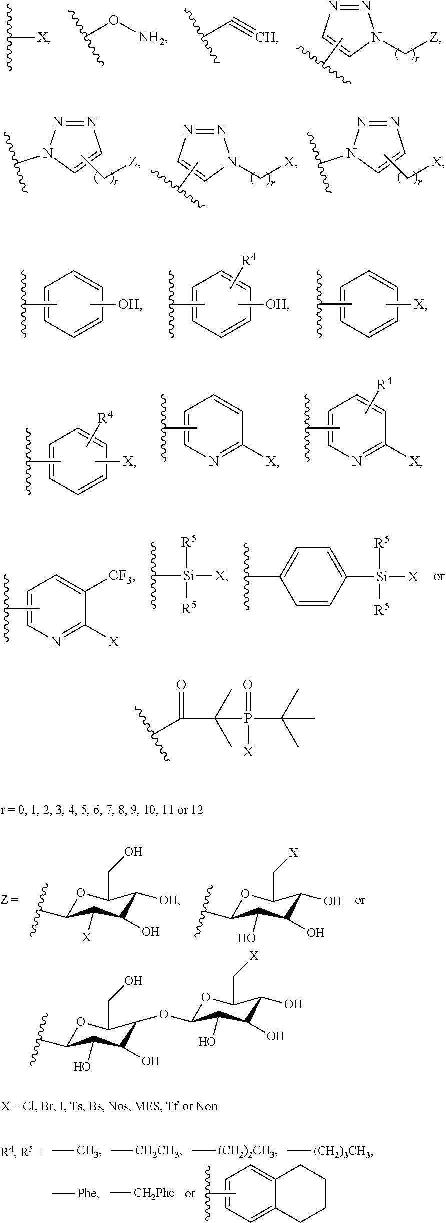

MG is a linker of the

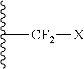

type with a leaving group X for substitution by ¹⁸F, ¹³¹I or ²¹¹At;
MG contains a leaving group X chosen from a radical of bromine (Br), chlorine (CI) or iodine (I), tosyl (Ts), brosylate (Bs), nosylate (Nos), 2-(N-morpholino)ethanesulfonic acid (MES), triflate (Tf) and nonaflate (Non);
the tris linker TL is chosen from one of structures [52] to [64]:

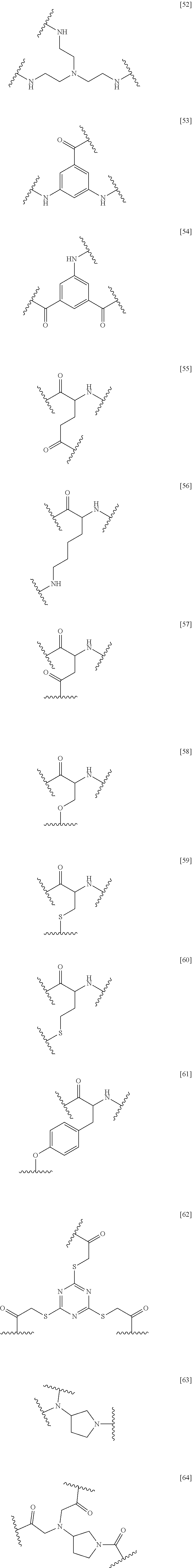

the tris linker TL is chosen from one of structures [65] to [116]:

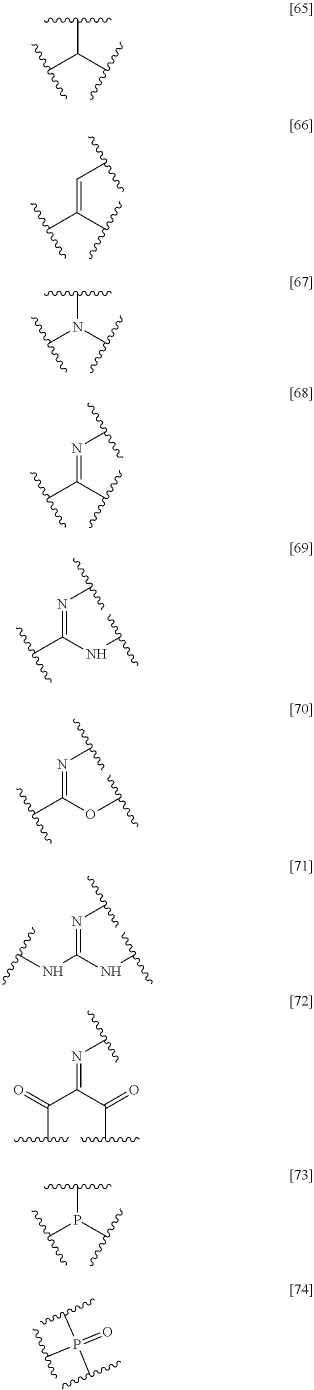

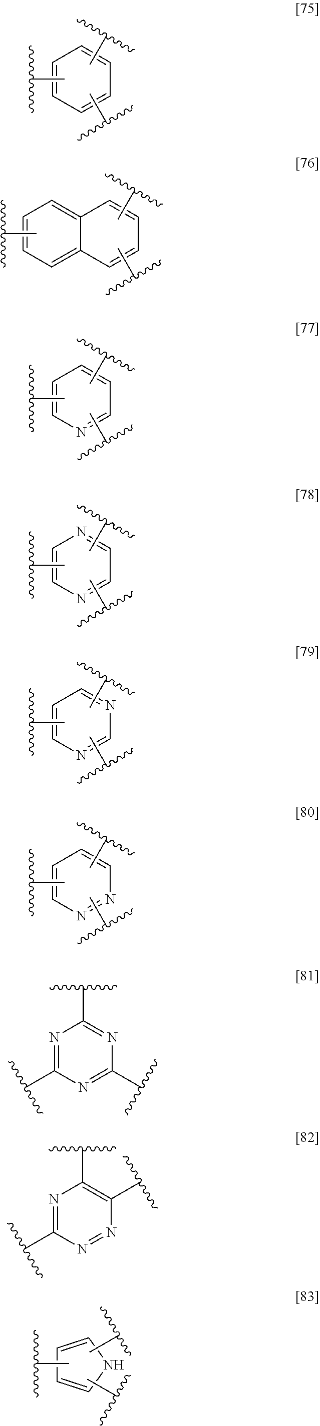

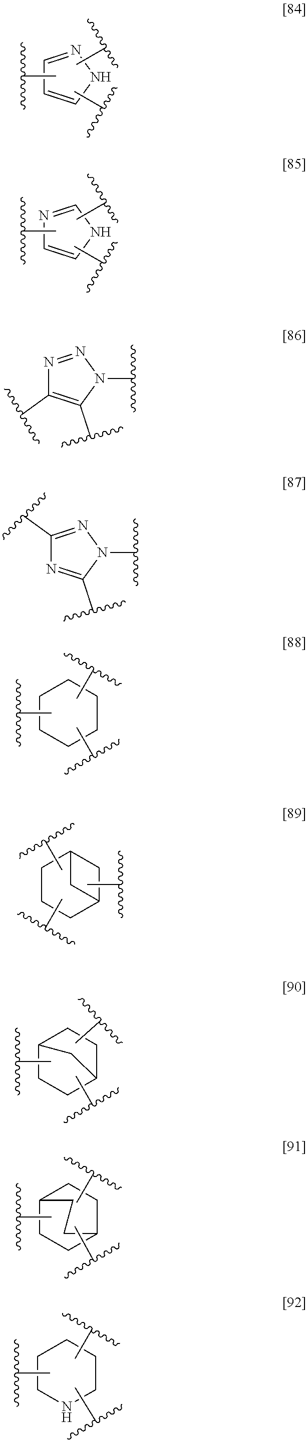

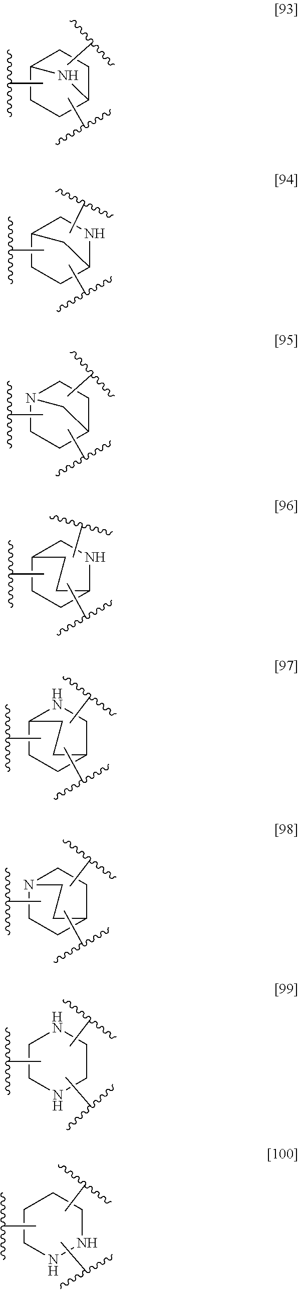

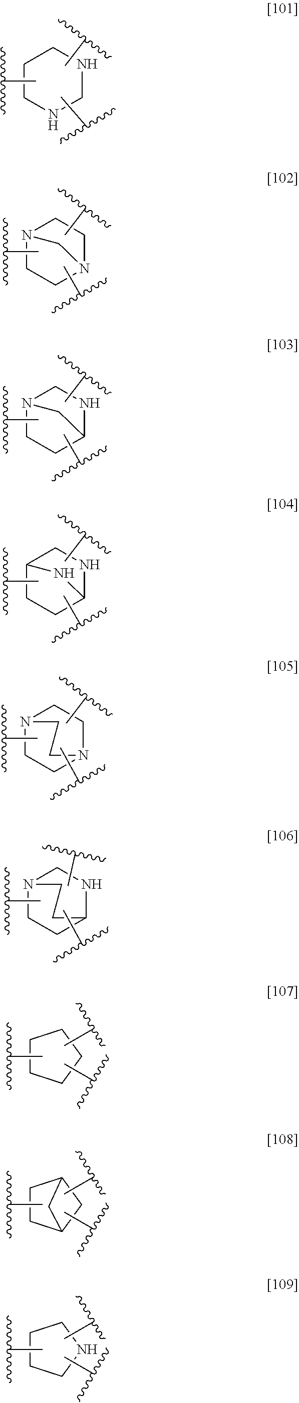

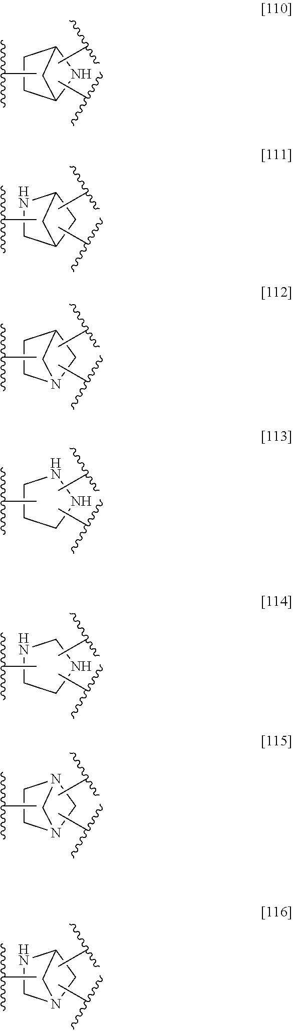

In the peptides or structural formulae [1] to [8], the following terms are used for synthetic amino acids:
Aph(Hor)=4-[2,6-dioxohexahydropyrimidine-4-carbonylamino]-L-phenylalanine
Cpa=4-chlorophenylalanine
D-Aph(Cbm)=D-4-aminocarbamoylphenylalanine
Pal=2-, 3- or 4-pyridylalanine

A labeling group MG for the covalent binding of the radioisotopes ¹⁸F, ¹³¹I or ²¹¹At especially comprises a leaving group X chosen from a radical of bromine (Br), chlorine (CI), iodine (I), tosyl (—SO₂—C₆H₄—CH₃; abbreviated to “Ts”), brosylate (—SO₂—C₆H₄—Br; abbreviated to “Bs”), nosylate or nitrobenzenesulfonate (—OSO₂—C₆H₄—NO₂; abbreviated to “Nos”), 2-(N-morpholino)ethanesulfonic acid (—SO₃—(CH₂)₂—N(CH₂)₄₀; abbreviated to “MES”), triflate or trifluoromethanesulfonyl (—SO₂CF₃; abbreviated to “Tf”) or nonaflate (—OSO₂—C₄F₉; abbreviated to “Non”).

The inventors have found that, surprisingly, the above-described dimeric labeling precursors or the radiotracers derived therefrom that have two targeting vectors TV1 and TV2, by comparison with monomeric radiotracers having one targeting vector, at the same systemic dose and with non-specific enrichment (off-target exposure), have much higher enrichment in tumor tissue (target exposure). It is suspected that this advantageous property is attributable to elevated docking probability and/or selectivity.

The targeting vectors TV1 and TV2 used in accordance with the invention have high binding affinity for tumor markers on the membrane, such as, in particular, PSMA (prostate-specific membrane antigen), FAP (fibroblast activation protein) and FPPS (farnesyl pyrophosphate synthase).

The heterodimeric labeling precursors and radiotracers of the invention can be used to address various tumor tissues and metastases. This is advantageous for the treatment of bone metastases that are induced by prostate carcinoma. Particularly useful for this purpose are labeling precursors or radiotracers having a first targeting vector TV1 for PSMA (PSMA targeting vector) and a second osteotropic targeting vector TV2 for FPPS (FPPS targeting vector).

The labeling precursors and radiotracers of the invention are likewise suitable for the addressing of the tumor stroma. For example, in the case of triple-negative breast cancer (TNBC), there is a lack of specific receptors on the surface of carcinogenic cells that enable direct addressing. One option here is “indirect” addressing of the tumor stroma. In the case of TNBC, the tumor stroma comprises cancer-associated fibroblasts (CAFs) and modified endothelial cells (ECs) that respectively overexpress FAP and PSMA. Accordingly, both homodimeric precursors with PSMAi, FAPi or bisphosphonate vectors and heterodimeric labeling precursors with a first PSMA targeting vector and a second FAP targeting vector are suitable for the diagnosis and treatment of TNBC.

The situation is similar for PSMA-negative prostate carcinomas, i.e. those that do not overexpress PSMA, which is the case for about 10% of prostate cancers. However, PSMA-negative tumors and metastases can be diagnosed and treated by addressing the tumor stroma with the aid of FAP targeting vectors. Accordingly, a heterodimeric labeling precursor with a first PSMA targeting vector and a second FAP targeting vector is suitable for comprehensive diagnosis and treatment of PSMA-positive and PSMA-negative prostate cancers.

The theranostic addressing of the tumor stroma with radioisotopes such as ¹⁷⁷Lu and ²²⁵Ac directly damages the tumor microenvironment which is essential for progression and causes “indirect” radiation damage (radiation induced bystander effect, RIBE) in adjacent cancer cells.

The spacers S1, S2 and S3 function as steric spacers and pharmacokinetic modulators that optimize the biochemical function of the targeting vectors (binding affinity for the target), radiochemical function of the labeling group (stable complexation or conjugation of the radioisotope) and the half-life in the blood serum (hydrophilicity). The spacers S1, S2, S3 preferably contain structural elements, for example squaramides or other aromatic units, that improve affinity for PSMA.

The tris linker TL creates the prerequisite for the orthogonal, sterically and pharmacokinetically optimized coupling of the labeling group MG and the two targeting vectors TV1 and TV2 in analogy with established monomeric radiopharmaceuticals having just one targeting vector. The invention thus enables the synthesis of effective labeling precursors and radiotracers with high theranostic potency.

The invention encompasses radiotracers consisting of one of the above-described labeling precursors and a
radioisotope complexed with the labeling precursor, chosen from the group comprising ⁴³Sc, ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹In, ¹³⁵Sm, ¹⁴⁰Pr ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th; or
radioisotope covalently bonded to the labeling precursor, chosen from the group comprising ¹⁸F, ¹³¹I and ²At.

In an appropriate embodiment of the invention, the radiotracer consists of one of the above-described labeling precursors having
a labeling group MG chosen from the group comprising NOTA (nona-1,4,7-triamine triacetate), DATA^{5m} (5-[[6-(N-methyl)amino]-1,4-diacetate-1,4-diazepane]pentanoic acid-N,N′,N′-triacetate) and NODAGA (1,4,7-triazacyclononane,1-glutaric acid,4,7-acetate); and
the radioactive compound aluminum [¹⁸F]fluoride (i.e. [¹⁸F]AIF) complexed to the labeling precursor.

In the case of a labeling group MG in the form of a chelator, the chelator serves for labeling with a radioisotope chosen from the group comprising ⁴³Sc, ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y, ⁸⁹Zr, ⁹⁰Nb, ^{99m}Tc, ¹¹¹1n ¹³⁵Sm ¹⁴⁰Pr, ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th.

Accordingly, the invention encompasses radiotracers obtainable from the above-described labeling precursors by complexation with a radioisotope, where the radioisotope is chosen from the group comprising ⁴³Sc, ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga, ⁸⁹Zr, ⁸⁶Y, ⁹⁰Y ⁸⁹Zr, ⁹⁰Nb, ^{99m}Tc, ¹¹¹1n ¹³⁵Sm ¹⁴⁰Pr ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac and ²³²Th.

### Chelators

The prior art discloses a multitude of chelators for the complexation of radioisotopes. Scheme 7 shows examples of chelators used in accordance with the invention.

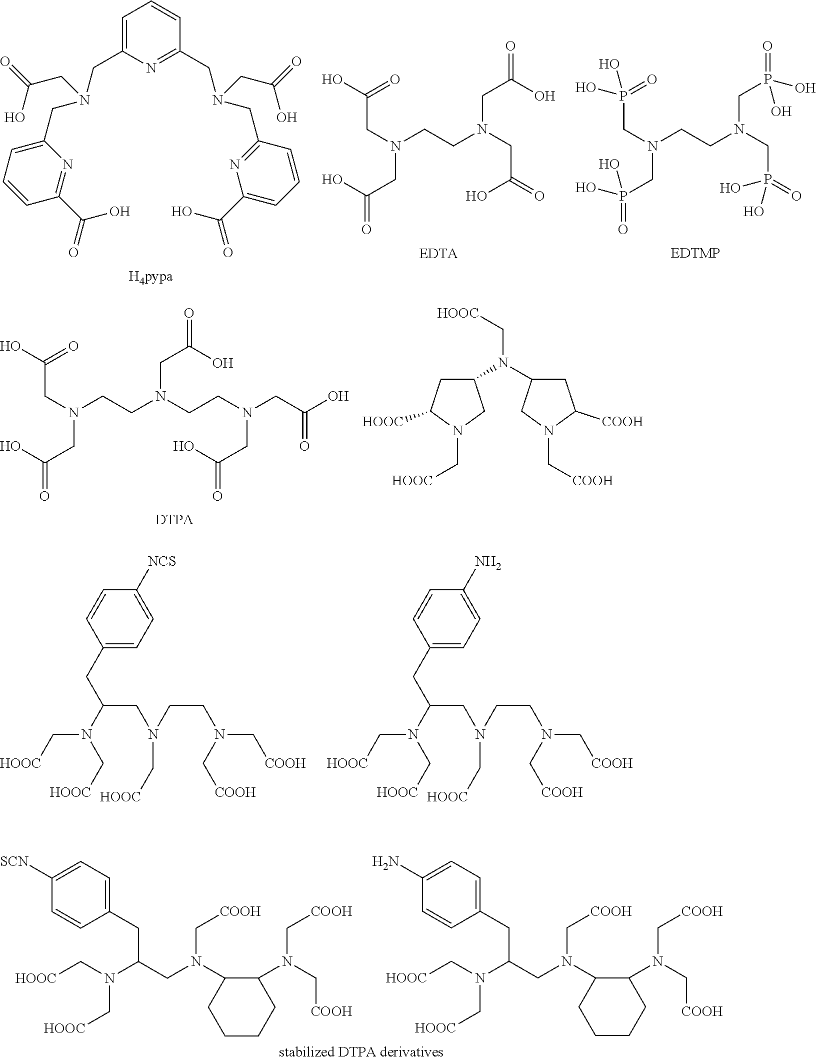

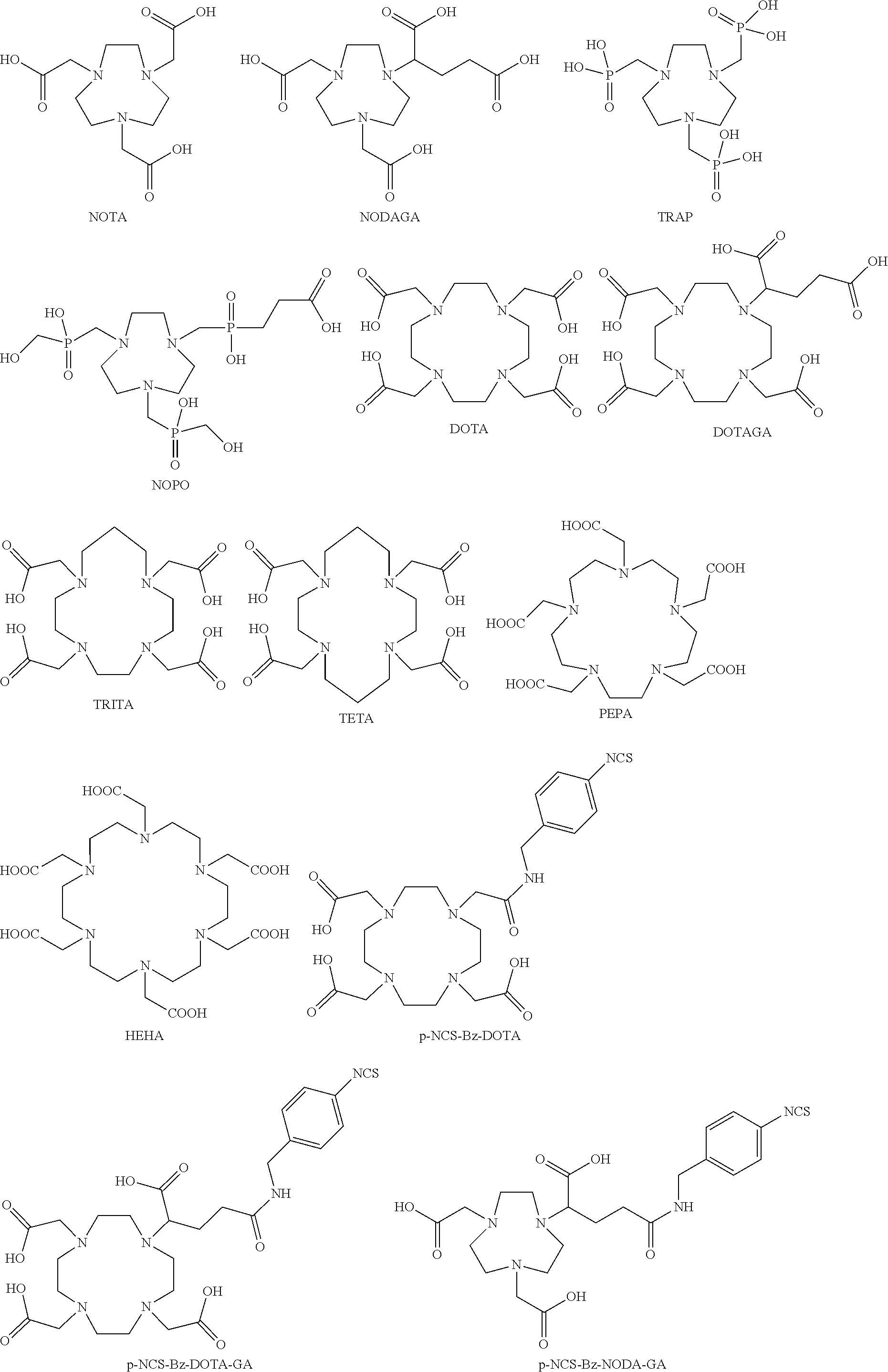

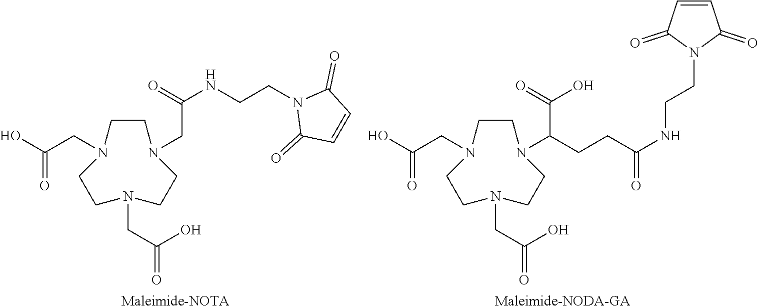

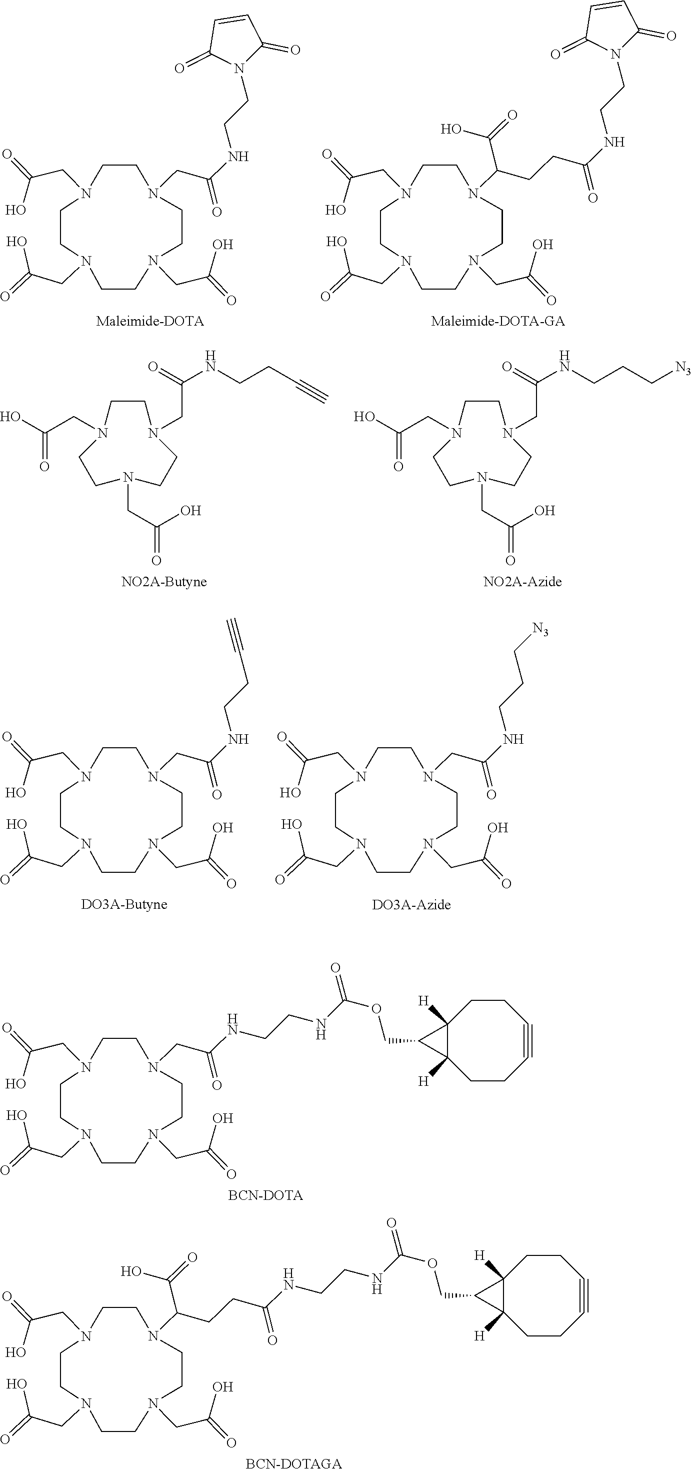

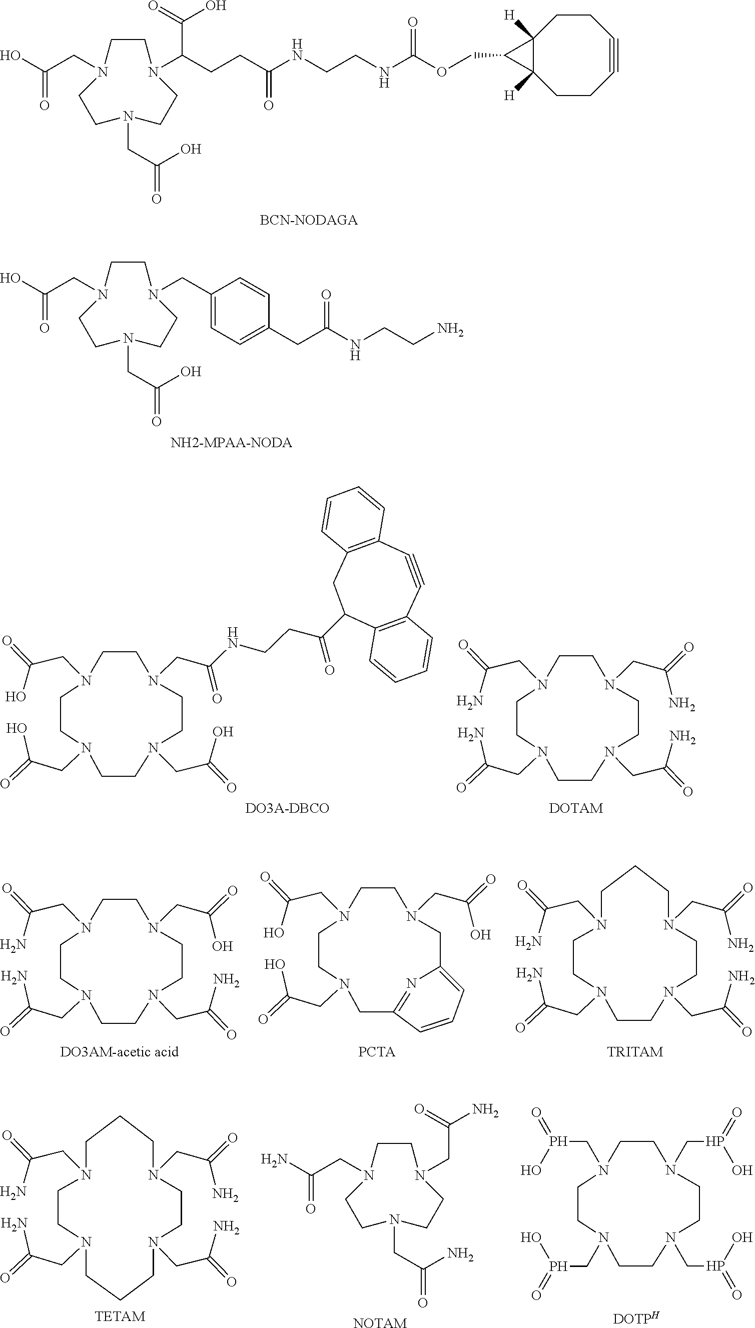

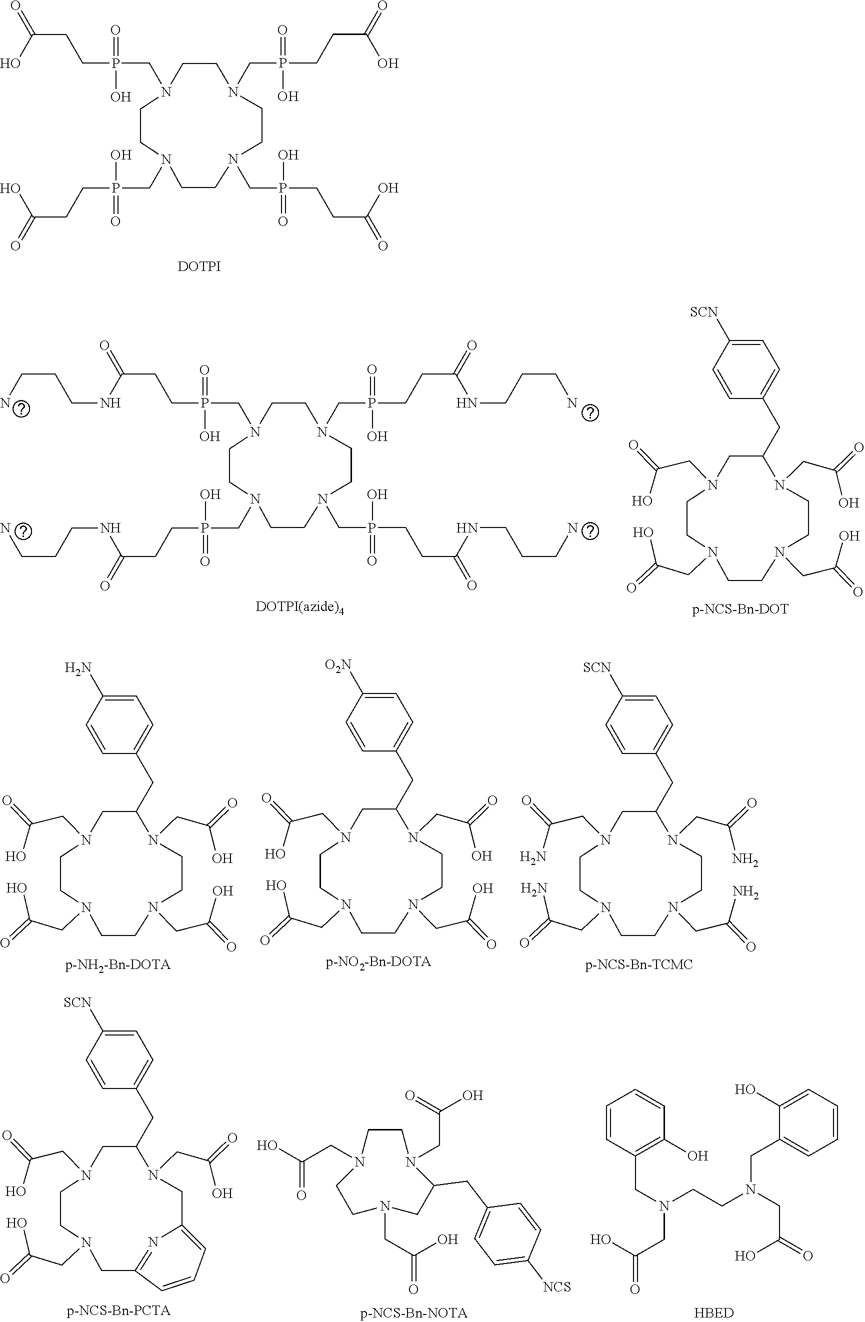

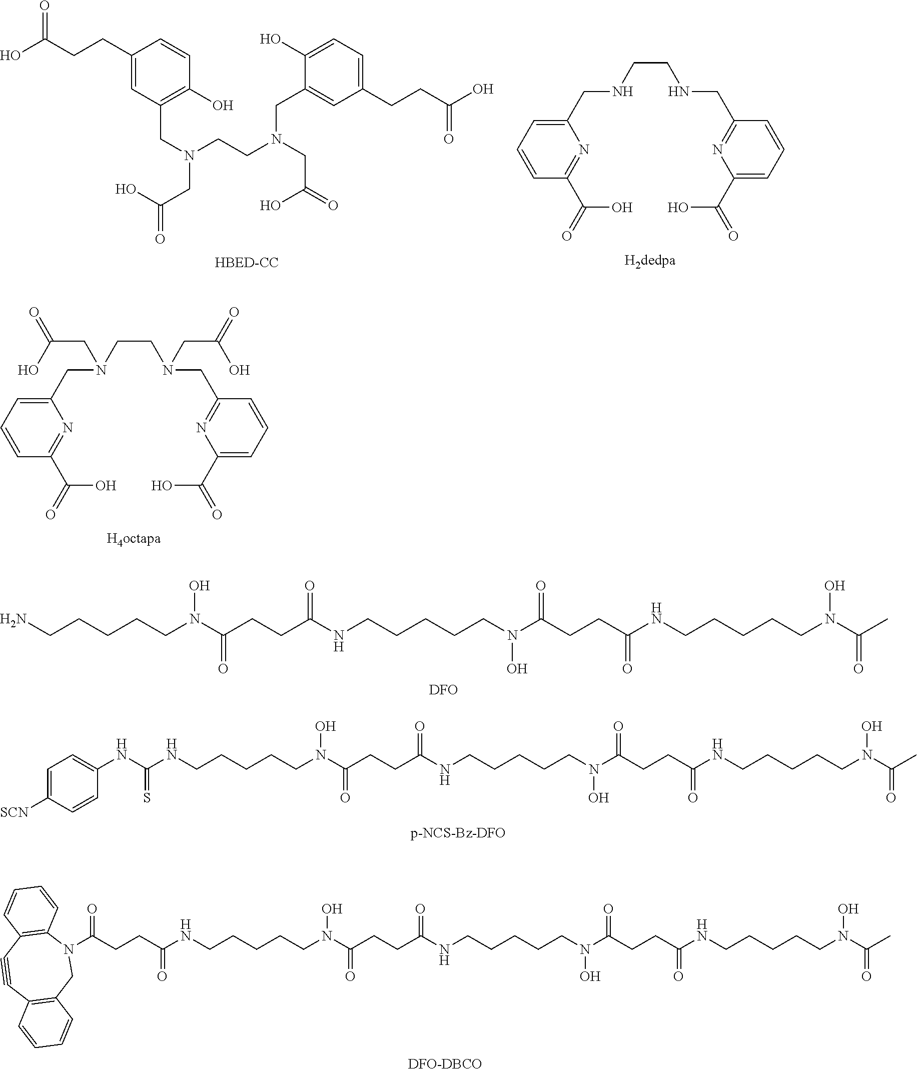

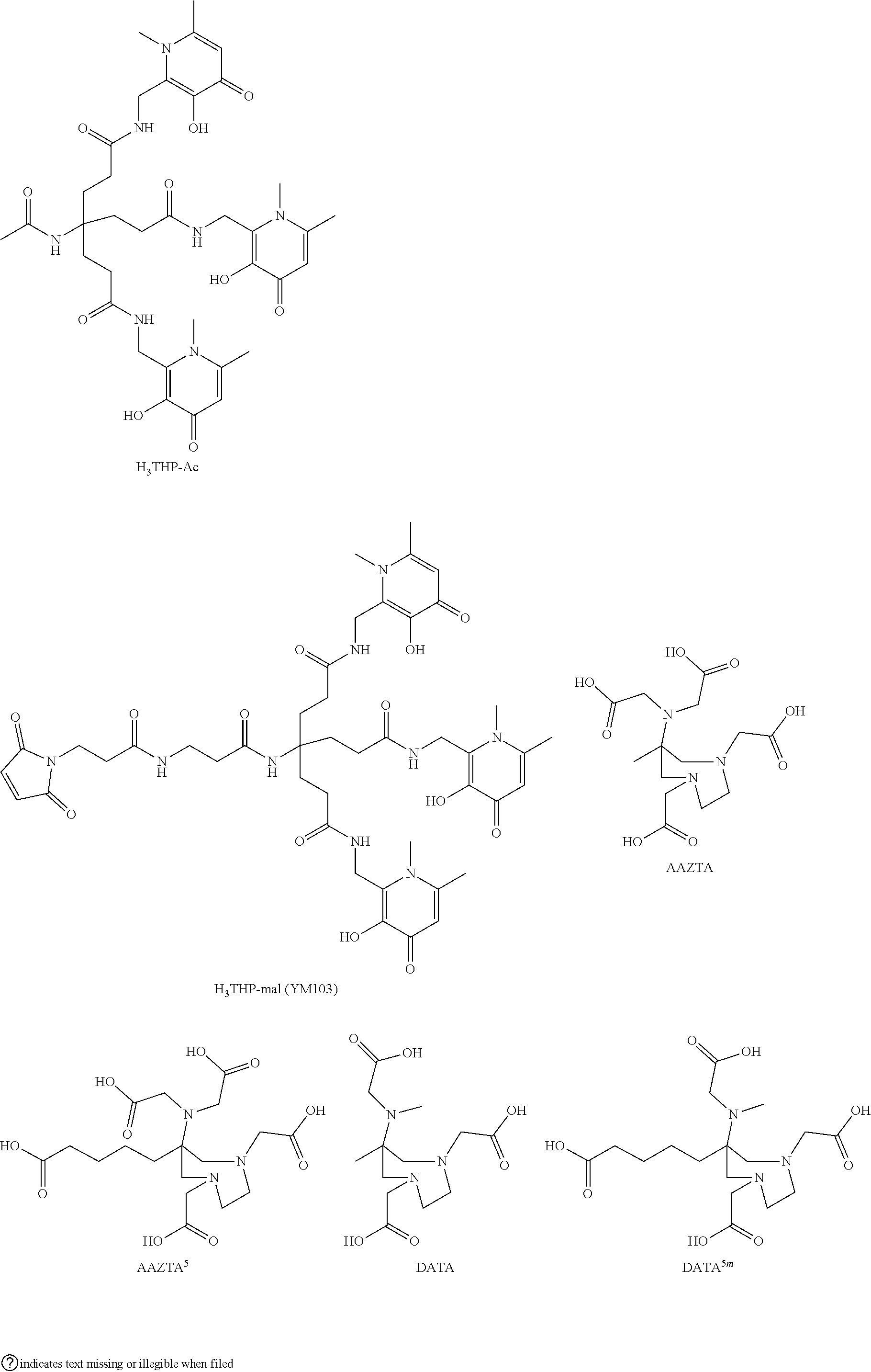

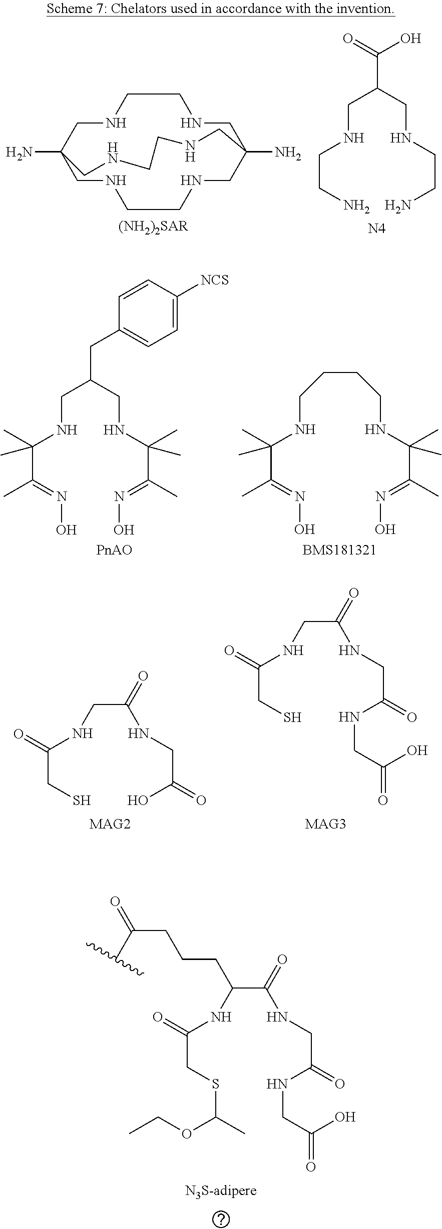

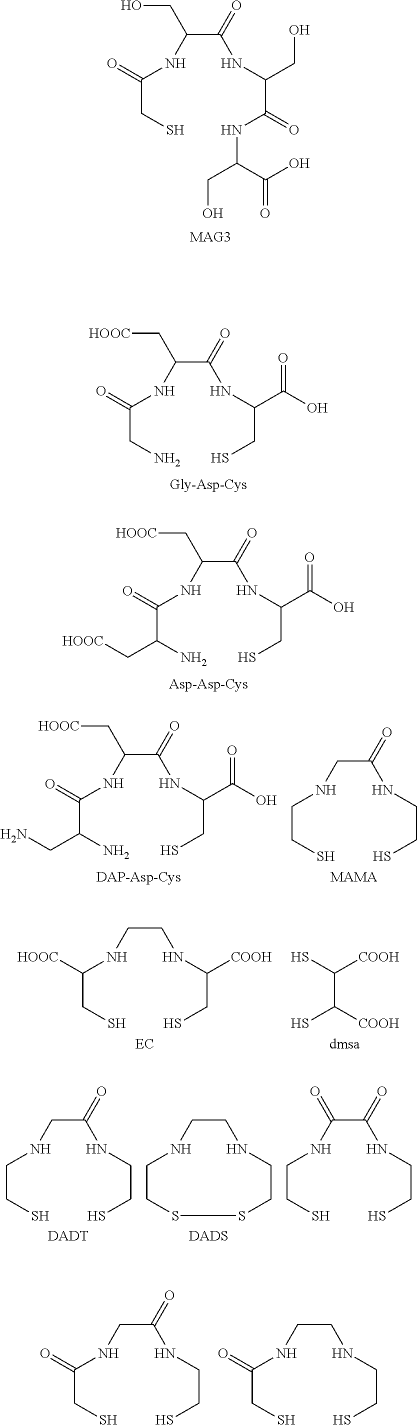

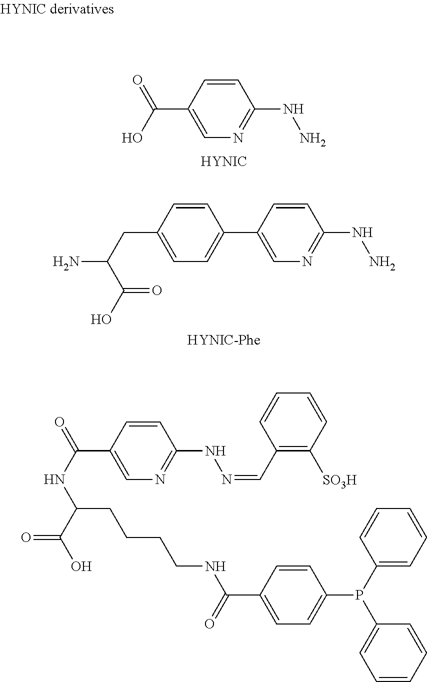

### Amide Coupling

In the invention, functional groups, such as the chelator Chel, the targeting vectors TV1 and TV2, the spacers S1, S2, S3, and the tris linker TL are preferably conjugated by an amide coupling reaction. The amide coupling that forms the backbone of proteins is the most commonly used reaction in medicinal chemistry. A generic example of an amide coupling is shown in scheme 8.

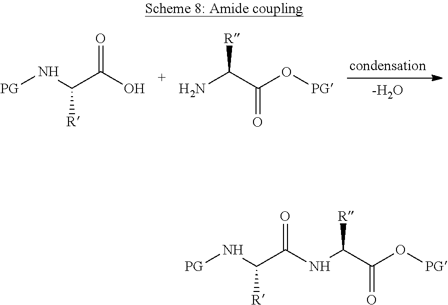

Because of a virtually unlimited set of readily available carboxylic acid and amine derivatives, amide coupling strategies open up a simple route for the synthesis of new compounds. The person skilled in the art is aware of numerous reagents and protocols for amide couplings. The most commonly used amide coupling strategy is based on the condensation of a carboxylic acid with an amine. For this purpose, the carboxylic acid is generally activated. Prior to the activation, remaining functional groups are protected. The reaction is effected in two steps either in one reaction medium (single pot) with direct conversion of the activated carboxylic acid or in two steps with isolation of an activated “trapped” carboxylic acid and reaction with an amine.

The carboxylic acid reacts here with a coupling reagent to form a reactive intermediate that can be isolated or reacted directly with an amine. Numerous reagents are available for carboxylic acid activation, such as acid halides (chloride, fluoride), azides, anhydrides or carbodiimides. In addition, reactive intermediates formed may be esters such as pentafluorophenyl or hydroxysuccinimido esters. Intermediates formed from acyl chlorides or azides are highly reactive. However, harsh reaction conditions and high reactivity are a barrier to use for sensitive substrates or amino acids. Accordingly, amide coupling strategies that use carbodiimides such as DCC (dicyclohexylcarbodiimide) or DIC (diisopropylcarbodiimide) open up a broad spectrum of application. Frequently, especially in the case of solid-phase synthesis, additives are used to improve reaction efficiency. Aminium salts are highly efficient peptide coupling reagents with short reaction times and minimal racemization. With some additives, for example HOBt, it is possible to completely avoid racemization. Aminium reagents are used in an equimolar amount to the carboxylic acid in order to prevent excessive reaction with the free amine of the peptide. Phosphonium salts react with carboxylate, which generally requires two equivalents of a base, for example DIEA. A major advantage of phosphonium salts over iminium reagents is that phosphonium does not react with the free amino group of the amine component. This enables couplings in an equimolar ratio of acid and amine, and helps to avoid the intramolecular cyclization of linear peptides and excessive use of costly amine components.

An extensive collation of reaction strategies and reagents for amide couplings can be found in the review articles:
Analysis of Past and Present Synthetic Methodologies on Medicinal Chemistry: Where Have All the New Reactions Gone?; D. G. Brown, J. Boström; J. Med. Chem. 2016, 59, 4443-4458;
Peptide Coupling Reagents, More than a Letter Soup; A. El-Faham, F. Albericio; Chem. Rev. 2011, 111, 6557-6602;
Rethinking amide bond synthesis; V. R. Pattabiraman, J. W. Bode; Nature, Vol. 480 (2011) 22/29;
Amide bond formation: beyond the myth of coupling reagents; E. Valeur, M. Bradley; Chem. Soc. Rev., 2009, 38, 606-631.

Numerous chelators among those used in accordance with the invention, for example DOTA and derivatives thereof, have one or more carboxy or amine groups. Accordingly, these chelators can be conjugated to the spacer S3 in a simple manner with the aid of one of the amide coupling strategies known in the prior art.

The meaning of some terms used in the context of the present invention is elucidated hereinafter.

Theranostics: Diagnosis and therapy of cancers using nuclear-medical radiotracers with analogous targeting vector.

Labeling precursor: Chemical compound containing a first and second targeting vector, and a chelator or a functional group for labeling with a radioisotope.

Radiotracer: Labeling precursor labeled with a radioisotope for nuclear-medical diagnosis or theranostics, which is used in a low concentration without affecting a patient's metabolism.

Target: Biological target structure, especially (membrane-bound) receptor, protein, enzyme or antibody in the living organism to which a target vector binds.

Targeting vector: Chemical group or radical that functions as ligand, agonist, antagonist or inhibitor for a biological target (e.g. a protein, enzyme or receptor) and has a high binding affinity for that target.

Tris linker: Structural unit having three functional groups for conjugation to a first, second and third spacer for a first and second targeting vector and a labeling group.

Spacer: Structural unit, group or radical that joins a first and second targeting vector and a labeling group to a tris linker and functions as steric and/or pharmacokinetic modulator.

### EXAMPLES

The compound (S)-6-(4-aminobutoxy)-N-(2-(2-cyano-4,4-difluoropyrrolidin-1-yl)-2-oxoethyl)-quinoline-4-carboxamide is abbreviated hereinafter to FAPi-NH₂:

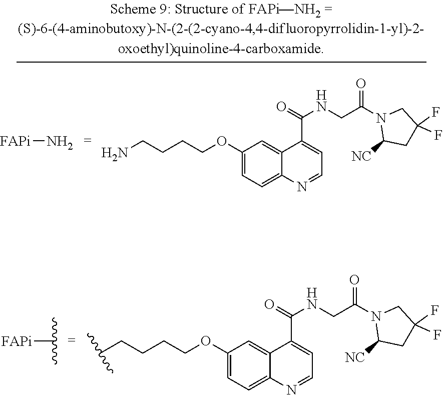

### Materials and Methods

### Nuclear Magnetic Resonance (NMR) Spectroscopy:

NMR spectra were recorded in deuterated solvents on an Avance II 400 (400 MHz) spectrometer with a 5 mm BBFO sample head (z gradient) from Bruker (Rheinstätten, Germany). Chemical shifts δ (in ppm) are based on the proton signal of the deuterated solvent relative to the tetramethylsilane standard (=0.00 ppm). The calculated coupling constants were reported in hertz (Hz). Spin multiplicity was abbreviated as follows: s=singlet, d=doublet, t=triplet, q=quartet and m=multiplets or combinations thereof. The spectra were analyzed using the MestReNova 14.2.0 software from Mestrelab Research (Santiago de Compostela, Spain).

### ESI-LC/MS:

ESI-LC/MS mass spectra were measured with the 1220 Infinity LC from Agilent Technologies, coupled to a 6130B Single Quadruple LC/MS system from Agilent Technologies with an Agilent Zorbax SB-C18 column (21×50 mm, 1.8 μm) with a linear gradient of acetonitrile (ACN)/Milli-Q® water (H₂O)+0.05% formic acid (HFo) and a flow rate of 0.5 mL/min.

### ESI-HPLC/MS:

HPLC-MS measurements were effected with a G6545A Q-ToF from Agilent Technologies with electrospray ionization, coupled to a 1260 Infinity II HPLC system (Agilent Technologies) with a G7111B 1260 quaternary pump, G7129A 1260 vial sampler and G7116A multicolumn thermostat. Separation was effected with an Agilent Poroshell 120 EC-C8 column (2.1×100 mm, 2.7 μm) with H₂O+2% ACN/ACN+2% H₂O+0.05% HFo and a flow rate of 0.1 mL/min.

### RP-HPLC:

Semipreparative reversed-phase high-pressure liquid chromatography (RP-HPLC) was conducted with LaChrom-HPLC (7000 series) from Merck Hitachi with a L-7100 pump, L-7400 UV detector (λ=254 nm), a D-7000 interface and autosampler. Separation was effected with a Phenomenex Synergi Max-RP C18 column (250×10 mm, 4 μm) and with a linear gradient of ACN/H₂O+0.1% trifluoroacetic acid (TFA) and a flow rate of 5 mL/min.

### Radio-TLC:

radio-TLCs were evaluated with a CR-35 Bio Test-Imager and the AIDA software from Raytest.

### Radio-HPLC:

Analytical radio-HPLC was conducted with an identical Merck Hitachi LaChrom-HPLC (7000 series). Separation was effected with a Phenomenex Luna C18 column (250×4.6 mm, 5 μm) and a linear gradient of ACN/H₂O+0.1% TFA and a flow rate of 1 mL/min. The radio-HPLC is additionally equipped with a Ramona radiodetector from Elysia Raytest, the energy window of which for ⁶⁸Ga measurements is set to 100-1200 keV, and for ¹⁷⁷Lu measurements to 100-250 keV.

### Stability Measurements:

The stability of the respective labeled compound in human serum (HS) and phosphate-buffered salt solution (PBS) was examined (n=3 in each case) by incubating about 10 MBq of the labeling solution in 0.5 mL of HS or PBS at 37° C. for about 2 half-lives (⁶⁸Ga: 2 h, ¹⁷⁷Lu: 14 d).

### Determination of log D (Measurement of Lipophilicity):

The log D value of the respective labeled compound was determined by diluting 4× about 10 MBq each time of the labeling solution with PBS to 700 μL. To this was added each time 700 μL of 1-octanol, and the mixture was shaken vigorously for 2 min and then centrifuged for 1 min. The organic and aqueous phases were separated and 400 μL of each was isolated. Samples of 3 μL (PBS) and 6 μL (1-octanol) were dabbed onto a TLC plate. Most of the activity was in the aqueous phase. This was subsequently diluted to 700 μL and extracted twice more with 1-octanol and dabbed on again. The TLC was exposed for about 5 min, and the integral of each spot (octanol phase: lₒ, aqueous PBS phase: l_{w}) was determined. The calculation of the log D value by equation (1) took account of the different volumes Vₒ=6 μL and V_{w}=3 μL:

$$log D = log ( \frac{{I}_{O}}{2 \cdot {I}_{W}} ) \qquad Equation ( 1 )$$

For the evaluation, the values from the 2nd and 3rd extractions of the 4 batches were averaged.

### In Vitro Assays:

The rhFAP (fibroblast activation protein), PREP (prolyl endopeptidase), DPP4 (dipeptidylpeptidase IV), DPP8 (dipeptidylpeptidase VIII) and DPP 9 (dipeptidylpeptidase IX) enzymes were expressed before use in the in vitro assays and then purified.

IC₅₀ measurements were conducted with the Infinite 200 instrument (Tecan Group Ltd.) and evaluated with the Magellan software.

The data were evaluated by GraFit 7 using a non-linear fit according to the following equation:

$$y = \frac{range}{1 + {( \frac{x}{{IC}_{50}} )}^{s}} \qquad Equation ( 2 )$$

where y is the remaining enzyme activity compared to the non-inhibited sample, xis the final inhibitor concentration used in the assay, s is the slope factor and IC₅₀ is the average inhibitory concentration.

### Example 1: FAPi-NH₂

Scheme 10 shows the synthesis of FAPi-NH₂.

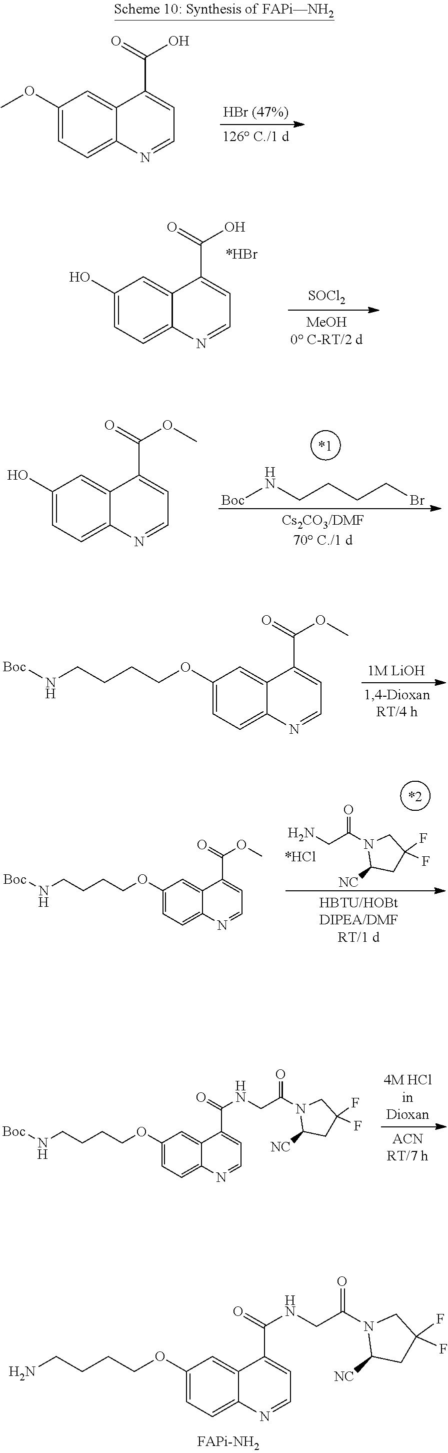

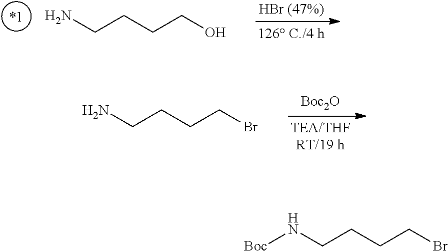 [SMILES: "Boc2O" ; NCCCCO ; NCCCCBr ; CC(C)(C)OC(=O)NCCCCBr]

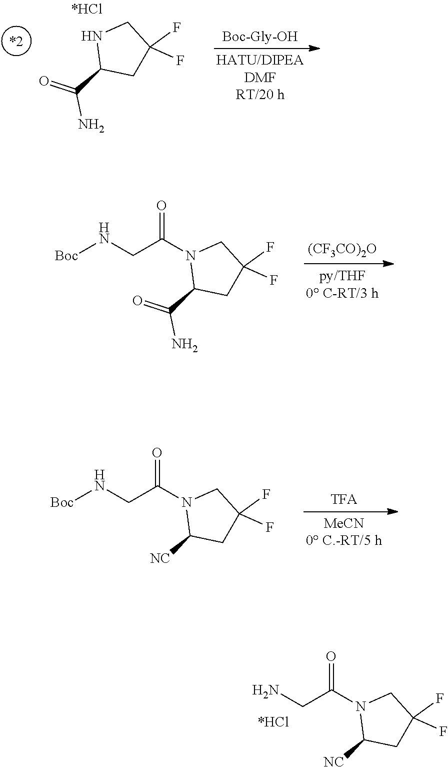

### 4-Bromobutylamine

To 4-aminobutanol (5.39 g, 60.47 mmol, 1.00 eq) was gradually added 70 mL of 47% hydrobromic acid, and then the mixture was heated under reflux for 4 h. The reaction mixture was then concentrated fully under reduced pressure. A colorless solid was obtained (13.521 g, 58.04 mmol, 96%). This was used directly in the next synthesis step without further purification.

MS (ESI-positive): m/z (%)=152.0 (100, [M+H]⁺), 154.0 (98, [M+H]⁺), calculated for C₄H₁₀BrN: 151.00 [M].

¹H NMR (400 MHz, MeOD): δ [ppm]=3.51 (t, J=6.4 Hz, 2H), 2.98 (t, J=7.6 Hz, 2H), 2.00-1.78 (m, 4H).

### tert-Butyl (4-bromobutyl)carbamate

4-Bromobutylamine (7.01 g, 30.09 mmol, 1.0 eq.) was dissolved together with di-tert-butyl dicarbonate (Boc₂O, 7.34 g, 33.63 mmol, 1.12 eq.) in dry THF (34 mL) under argon. Thereafter, TEA (4.6 mL, 36.12 mmol, 1.2 eq.) was added. MeOH (36 mL) was added to the suspension formed until the solution became clear again, and it was then stirred at RT for 19 h. Then the solvent was removed under reduced pressure and dilute HBr was added to the residue, such that a pH=2.5 was attained. The aqueous solution was extracted with Et₂O (5×80 mL) and the combined organic phases were washed once each with a little NaHCO₃ and brine, and then dried over Na₂SO₄. The solvent was removed under reduced pressure. By column chromatography (CH/EA 5:1), a colorless solid (5.08 g, 20.15 mmol, 66%) was obtained.

MS (ESI-positive): m/z (%)=196.0 (100, [M−tBu]⁺), 198.0 (100, [M−tBu]⁺), calculated for C₉H₁₈BrNO₂: 251.05 [M].

¹H NMR (400 MHz, CDCl₃): δ [ppm]=3.36-3.21 (m, 4H), 1.86-1.76 (m, 4H), 1.43 (s, 9H).

### Boc-Gly-Pro-CONH₂ (tert-butyl (S)-(2-(2-carbamoyl-4,4-difluoropyrrolidin-1-yl)-2-oxoethyl)carbamate)

Boc-Gly-OH (1.38 g, 7.88 mmol, 1.05 eq.) and HBTU (3.12 g, 8.20 mmol, 1.1 eq.) were dissolved in dry DCM (8 mL) and DMF (8 mL) under argon. Thereafter, DIPEA (1.53 mL, 8.97 mmol, 1.2 eq.) was added and the mixture was stirred at RT for 1 h. In a further reaction vessel, 4,4-difluoro-L-prolinamide hydrochloride was dissolved in dry DCM (5 mL) and DMF (5 mL), and DIPEA (2.54 mL, 14.90 mmol, 2.0 eq.) was likewise added thereto. The solutions were combined and stirred at RT for 19 h. The precipitated solids were filtered off, and the mother liquor was cooled overnight in order to complete the precipitation. The two precipitates were combined. A colorless solid (1.97 g, 6.41 mmol, 86%) was obtained.

MS (ESI-positive): m/z (%)=207.8 (62, [M−Boc+H]⁺), 251.8 (100, [M−^{t}Bu+H]⁺), 307.9 (39, [M+H]⁺), 329.9 (24, [M+Na]⁺), calculated for C₁₂H₁₉F₂N₃O₄: 307.13 [M]⁺.

¹H NMR (400 MHz, DMSO-d₆): δ [ppm]=7.40 (s, 1H), 7.16 (s, 1H), 6.87 (dt, J=10.4, 5.8 Hz, 1H), 4.45 (dd, J=9.0 Hz, 1H), 4.15-3.85 (m, 2H), 3.86-3.63 (m, 2H), 2.81-2.27 (m, 2H), 1.37 (s, 9H).

### Boc-Gly-Pro-CN (tert-butyl (S)-(2-(2-cyano-4,4-difluoropyrrolidin-1-yl)-2-oxoethyl)carbamate)

Boc-Gly-Pro-CONH₂ (1.97 g, 6.41 mmol, 1.0 eq.) was dissolved in dry THF (50 mL) under argon and cooled to 0° C. Pyridine (4.1 mL, 51.3 mmol, 8.0 eq.) was added. In a further reaction vessel, TFAA (2.7 mL, 19.2 mmol, 3.0 eq.) was dissolved in dry DCM (35 mL) under argon and slowly added dropwise to the reaction solution. The mixture was stirred at RT for 3 h. Thereafter, 1 M HCl (80 mL) was added and the aqueous solution was extracted with DCM (5×80 mL). The combined organic phases were washed once each with a little Na₂CO₃ and brine, and dried over Na₂SO₄. The solvent was removed under reduced pressure and the product was purified via column chromatography (CH/EA=3:2). A colorless solid (1.49 g, 4.81 mmol, 81%) was obtained.

MS (ESI-positive): m/z (%)=190.0 (31, [M−Boc+H]⁺), 233.9 (100, [M−^{t}Bu+H]⁺), calculated for C₁₂H₁₇F₂N₂O₃: 289.12 [M]⁺.

¹H NMR (400 MHz, DMSO-d₆): δ [ppm]=5.34 (s, 1H), 4.97 (t, J=6.5 Hz, 1H), 4.04-3.78 (m, 4H), 2.81-2.69 (m, 2H), 1.45 (s, 9H).

### Gly-Pro-CN ((S)-4,4-difluoroglycylpyrrolidine-2-carbonitrile)

Boc-Gly-Pro-CN (1.15 g, 3.97 mmol, 1.0 eq.) was dissolved in dry MeCN (2 mL) under argon, and TFA (2 mL) was slowly added dropwise. The mixture was stirred at RT for 5 h, and then the solvent was removed under reduced pressure and the residue was co-distilled with MeOH (5×25 mL). A yellowish oil was obtained, which was used in the next stage without further purification.

MS (ESI-positive): m/z (%)=189.9 (100, [M+H]⁺), 231.0 (20, [M+ACN+H]⁺), calculated for CH C₇H₉F₂N₃O: 189.07 [M]⁺.

¹H NMR (400 MHz, MeOD): δ [ppm]=8.25 (s, 2H), 5.22-5.15 (m, 1H), 4.15-3.91 (m, 4H), 3.00-2.81 (m, 2H).

### 6-Hydroxyquinoline-4-carboxylic acid hydrobromide

6-Methoxyquinoline-4-carboxylic acid (2.46 g, 12.1 mmol, 1.0 eq.) was dissolved in 47% HBr (28.18 mL, 242.42 mmol, 20 eq.) and heated under reflux for 1 d. After cooling to RT, the hydrobromic acid was partly removed under reduced pressure, and the precipitate was then filtered and washed first with cold EA (20 mL) and then with a little cold EA/MeOH (90:10). A yellow solid (3.25 g, 12.1 mmol, 100%) was obtained.

MS (ESI-positive): m/z (%)=190.0 (100, [M+H]⁺), 191.0 (12, [M+H]⁺), calculated for C₁₀H₈BrNO₃: 189.04 [M].

¹H NMR (400 MHz, MeOD): δ [ppm]=9.04 (d, J=5.6 Hz, 1H), 8.41 (d, J=5.6 Hz, 1H), 8.34 (d, J=2.6 Hz, 1H), 8.19 (d, J=9.3 Hz, 1H), 7.77 (dd, J=9.3, 2.6 Hz, 1H).

### 6-Hydroxyquinoline-4-carboxylic acid methyl ester

First of all, dry MeOH (20 mL) was cooled to 0° C. under argon, and then SOCl₂ (4.43 mL, 61.09 mmol, 5.0 eq.) was added dropwise. 6-Hydroxyquinoline-4-carboxylic acid hydrobromide was dissolved in dry MeOH (20 mL) and likewise cooled to 0° C. under argon. Thereafter, the SOCl₂-MeOH solution was added dropwise. The reaction solution was warmed to RT and heated under reflux for 1 d. SOCl₂ (2.91 g, 24.44 mmol, 2 eq.) and MeOH (20 mL) were again combined at 0° C. and added to the reaction mixture at RT. The solution was heated under reflux for a further 24 h. The above-described step was repeated once more and, after heating under reflux for a further 4 h, the solvent was removed under reduced pressure. A yellow solid was obtained, which was used in the next stage without further purification.

MS (ESI-positive): m/z (%)=204.0 (100, [M+H]⁺), 205.1 (12, [M+H]⁺), calculated for C₁₁H₉NO₃: 203.06 [M].

¹H NMR (400 MHz, MeOD): δ [ppm]=9.02 (d, J=5.5 Hz, 1H), 8.38 (d, J=5.5 Hz, 1H), 8.24 (d, J=2.6 Hz, 1H), 8.17 (d, J=9.3 Hz, 1H), 7.75 (dd, J=9.3, 2.6 Hz, 1H), 4.09 (s, 3H).

### Boc-Quino-COOMe (6-(4-((tert-butoxycarbonyl)amino)butoxy)quinoline-4-carboxylic acid methyl ester)

Under argon, 6-hydroxyquinoline-4-carboxylic acid methyl ester (2.48 g, 12.1 mmol, 1.0 eq.) and Cs₂CO₃ (4.37 g, 13.4 mmol, 1.25 eq.) was suspended in dry DMF (55 mL). The reaction solution was heated to 70° C. Subsequently, tert-butyl (4-bromobutyl)carbamate (3.76 g, 14.91 mmol, 1.22 eq.) was dissolved in dry DMF (80 mL) and added dropwise to the hot reaction mixture. The solution was stirred at 70° C. for 3 h. After checking the reaction, tert-butyl (4-bromobutyl)carbamate (1.23 g, 4.88 mmol, 0.4 eq.) was again dissolved in dry DMF (20 mL) and added to the reaction mixture. The mixture was stirred at 70° C. overnight. After a further addition (308 mg, 1.22 mmol, 0.1 eq.) and 3 h at 70° C., the solvent was removed under reduced pressure and the residue was taken up in dilute HBr (150 mL, pH=2.6). The mixture was extracted with EA (5×80 mL), and the combined organic phases were washed with brine and dried over Na₂SO₄. The solvent was removed under reduced pressure and the crude product was obtained via column chromatography (CHCl₃/MeOH, 100:1) as a pale yellow solid (2.68 g, 7.17 mmol, 59%).

MS (ESI-positive): m/z (%)=375.2 (100, [M+H]⁺), 376.2 (23, [M+H]⁺), calculated for C₂₀H₂₆N₂O₅: 374.18 [M].

¹H NMR (400 MHz, CDCl₃): δ [ppm]=8.84 (d, J=4.6 Hz, 1H), 8.24 (dd, J=16.7, 2.8 Hz, 1H), 8.11 (d, J=9.2 Hz, 1H), 7.94 (d, J=4.6 Hz, 1H), 7.43 (dd, J=9.2, 2.8 Hz, 1H), 4.74-4.60 (m, 1H), 4.15 (t, J=6.21 Hz, 2H), 4.03 (s, 3H), 3.27-3.16 (m, 2H), 1.95-1.86 (m, 2H), 1.78-1.67 (m, 2H), 1.42 (s, 9H).

Boc-Quino-COOH (6-(4-((tert-Butoxycarbonyl)amino)butoxy)quinoline-4-carboxylic acid) Boc-Quino-COOMe (3.34 g, 8.92 mmol, 1.0 eq.) was dissolved in 1,4-dioxane (40 mL). Subsequently, 1 M LiOH (17.8 mL, 17.84 mmol, 2.0 eq.) was added and the mixture was stirred at RT for 4 h. The organic solvent was removed under reduced pressure and then 1 M HCl was used to set a pH of 3.5. The aqueous solution was extracted with EA (8×80 mL) and the combined organic phases were dried over Na₂SO₄ and the solvent was removed under reduced pressure. A pale yellow solid (1.82 g, 5.05 mmol, 57%) was obtained.

MS (ESI-positive): m/z (%)=261.1 (20, [M−Boc+H]⁺), 361.2 (100, [M+H]⁺), 362.2 (22, [M+H]⁺), calculated for C₁₉H₂₄N₂O₅: 360.17 [M].

¹H NMR (400 MHz, DMSO-d₆): δ [ppm]=8.86 (d, J=4.5 Hz, 1H), 8.15 (d, J=2.8 Hz, 1H), 8.02 (d, J=9.3 Hz, 1H), 7.92 (d, J=4.4 Hz, 1H), 7.49 (dd, J=9.2 Hz, 2.8 Hz, 1H), 6.87 (t, J=5.8 Hz, 1H), 4.10 (t, J=6.3 Hz, 2H), 3.00 (q, J=6.6 Hz, 2H), 1.78 (q, J=11.8, 6.5 Hz, 2H), 1.62-1.51 (m, 2H), 1.37 (s, 9H).

### FAPi-NHBoc (tert-butyl (S)-(4-((4-((2-(2-cyano-4,4-difluoropyrrolidin-1-yl)-2-oxoethyl)carbamoyl)quinolin-6-yl)oxy)butyl)carbamate)

Under argon, Boc-Quino-COOH (1.64 g, 4.55 mmol, 1.0 eq.) and DIPEA (0.93 mL, 5.46 mmol, 1.2 eq.) were dissolved in dry DMF (16 mL). Thereafter, HOBt (0.68 g, 5.01 mmol, 1.1 eq.) and HBTU (1.90 g, 5.01 mmol, 1.1 eq.) were added and the reaction mixture was stirred at RT for 1 h. Subsequently, Gly-Pro-CN, likewise dissolved in dry DMF (10 mL) and with DIPEA (1.93 ml, 11.38 mmol, 2.5 eq.) added thereto, was added and the whole reaction mixture was stirred at RT for a further 1 d. Thereafter, the solvent was removed in vacuo and the residue was taken up in EA. The organic phase was washed with 1 M citric acid, saturated Na₂CO₃ and brine. Then the aqueous phase was extracted with EA (3×100 mL) and the combined organic extracts were dried over Na₂SO₄. The solvent was removed under reduced pressure and the product was obtained via column chromatography (CHCl₃/MeOH, 100:3) as a colorless solid (1.74 g, 3.27 mmol, 72%).

MS (ESI-positive): m/z (%)=432.0 (33, [M−Boc+H]⁺), 476.1 (46, [M−^{t}Bu+H]⁺), 532.4 (100, [M+H]⁺), calculated for C₂₆H₃₁F₂N₅O₅: 531.23 [M]⁺.

¹H NMR (400 MHz, MeOD): δ [ppm]=8.74 (d, J=4.4 Hz, 1H), 7.96 (d, J=9.3 Hz, 1H), 7.93-7.88 (m, 1H), 7.56 (d, J=4.4 Hz, 1H), 7.46 (dd, J=9.3, 2.7 Hz, 1H), 5.15 (dd, J=9.4, 3.1 Hz, 1H), 4.39-3.98 (m, 8H), 3.19-3.09 (m, 2H), 3.02-2.70 (m, 2H), 1.94-1.83 (m, 2H), 1.76-1.65 (m, 2H), 1.43 (s, 9H).

### FAPi-NH₂ ((S)-6-(4-aminobutoxy)-N-(2-(2-cyano-4,4-difluoropyrrolidin-1-yl)-2-oxoethyl)quinoline-4-carboxamide)

FAPi-NHBoc (531.6 mg, 1.0 mmol, 1.0 eq) was dissolved at 0° C. and under argon in dry acetonitrile (10 mL). It was 4 M HCl in 1,4-dioxane (5.0 mL, 5.0 mmol, 5.0 eq) and slowly warmed to RT. After 3 h, 4 M HCl in 1,4-dioxane (2.5 mL, 2.5 mmol, 2.5 eq) was added once again and, after a further 4 h at RT, the mixture was diluted with further acetonitrile (30 mL) and then concentrated fully in vacuo. A colorless solid (467 mg, 1.0 mmol, 100%) was obtained.

MS (ESI-positive): m/z (%)=216.7 (100, [M+H]²⁺), 237.2 (27, [M+ACN+H]²⁺), 432.1 (22, [M+H]⁺), calculated for C₂₁H₂₃O₅F₂N₅O₃: 431.18 [M]⁺.

¹H NMR (400 MHz, MeOD): δ [ppm]=9.10 (d, J=5.5 Hz, 1H), 8.32 (d, J=2.7 Hz, 1H), 8.24 (d, J=9.3 Hz, 1H), 8.08 (d, J=5.5 Hz, 1H), 7.86 (dd, J=9.4, 2.6 Hz, 1H), 5.15 (dd, J=9.4, 3.1 Hz, 1H), 4.48-4.33 (m, 4H), 4.32-4.07 (m, 2H), 3.06 (t, J=6.5 Hz, 2H), 3.02-2.74 (m, 2H), 2.09-1.87 (m, 4H).

### Example 2: DOTA.Glu.(FAPi)₂, DOTAGA.Glu.(FAPi)₂, DATA^{5m}.Glu.(FAPi)₂

There follows a description of the synthesis of the labeling precursors DOTA.Glu.(FAPi)₂, DOTAGA.Glu.(FAPi)₂ and DATA^{5m}.Glu.(FAPi)₂. The first synthesis steps are identical for all 3 compounds, and a representative synthesis is shown in scheme 11.

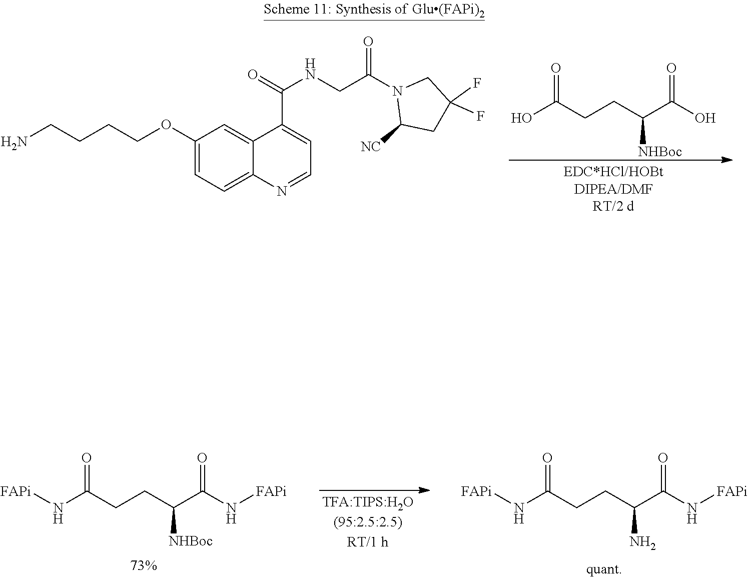

### Boc-Glu.(FAPi)₂ (tert-butyl ((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)carbamate)

tert-Butoxycarbonyl-L-glutamic acid (Boc-Glu-OH, 40 mg, 162 μmol, 1.0 eq), 1-hydroxybenzotriazole (HOBt, 55 mg, 405 μmol, 2.5 eq) and 1-ethyl-3-(3-dimethylaminopropyl)carbodiimide hydrochloride (EDC*HCl, 78 mg, 405 μmol, 2.5 eq) were dissolved in dry N,N-dimethylformamide (DMF, 4 mL), N,N-diisopropylethylamine (DIPEA, 68.9 μL, 405 μmol, 2.5 eq) was added and the mixture was stirred at room temperature (RT) under an argon atmosphere for 90 min. Then a solution of FAPi-NH₂*TFA (265 mg, 486 μmol, 3 eq) and DIPEA (110 μL, 648 μmol, 4 eq) in DMF (4 mL) was added and stirring was continued at RT overnight. Further HOBt (16 mg, 121 μmol, 0.75 eq) and EDC*HCl (23 mg, 121 μmol, 0.75 eq) were added and, after a further 60 min, another solution of FAPi-NH₂*TFA (88 mg, 162 μmol, 1.0 eq) and DIPEA (41.4 μL, 243 μmol, 1.5 eq) in DMF (2 mL). After stirring had been continued overnight at RT, the solvent was removed in vacuo. After column chromatography (CHCl₃/MeOH (100:10-15)), 127 mg (118 μmol, 73%) Boc-Glu.(FAPi)₂ was obtained as a yellow oil.

LC-MS (ESI-positive): m/z (%)=487.8 (100, [M−Boc+H]²⁺), 537.8 (73, [M+H]²⁺), 1074.4 (9, [M+H]⁺), 1075.4 (6, [M+H]⁺), calculated for C₅₂H₅₉F₄N₁₁O₁₀: 1073.44 [M]⁺.

### Glu.(FAPi)₂ ((S)-2-amino-N¹,N⁵-bis(4-((4-((2-((S)-2-cyano-4,4-difluoropyrrolidin-1-yl)-2-oxoethyl)carbamoyl)quinolin-6-yl)oxy)butyl)pentanediamide)

To Boc-Glu.(FAPi)₂ (127 mg, 118 μmol) were added 50 μL of Milli-Q® water, 50 μL of triisopropylsilane (TIPS) and 1.9 mL of TFA (TFA:TIPS:H₂O (95:2.5:2.5)), and the mixture was stirred at RT for 1 h. Subsequently, 5× about 10 mL each of MeOH was added and the solvents were removed again in vacuo, and a yellow oil was obtained. It was used in the next stage without further purification.

LC-MS (ESI-positive): m/z (%)=325.6 (100, [M−Boc+H]³⁺), 487.8 (28, [M+H]²⁺), 974.3 (5, [M+H]*), calculated for C₄₇H₅₁F₄N₁₁O₈: 973.39 [M]⁺.

The synthesis of the labeling precursor DOTA.Glu.(FAPi)₂ is shown below in scheme 12.

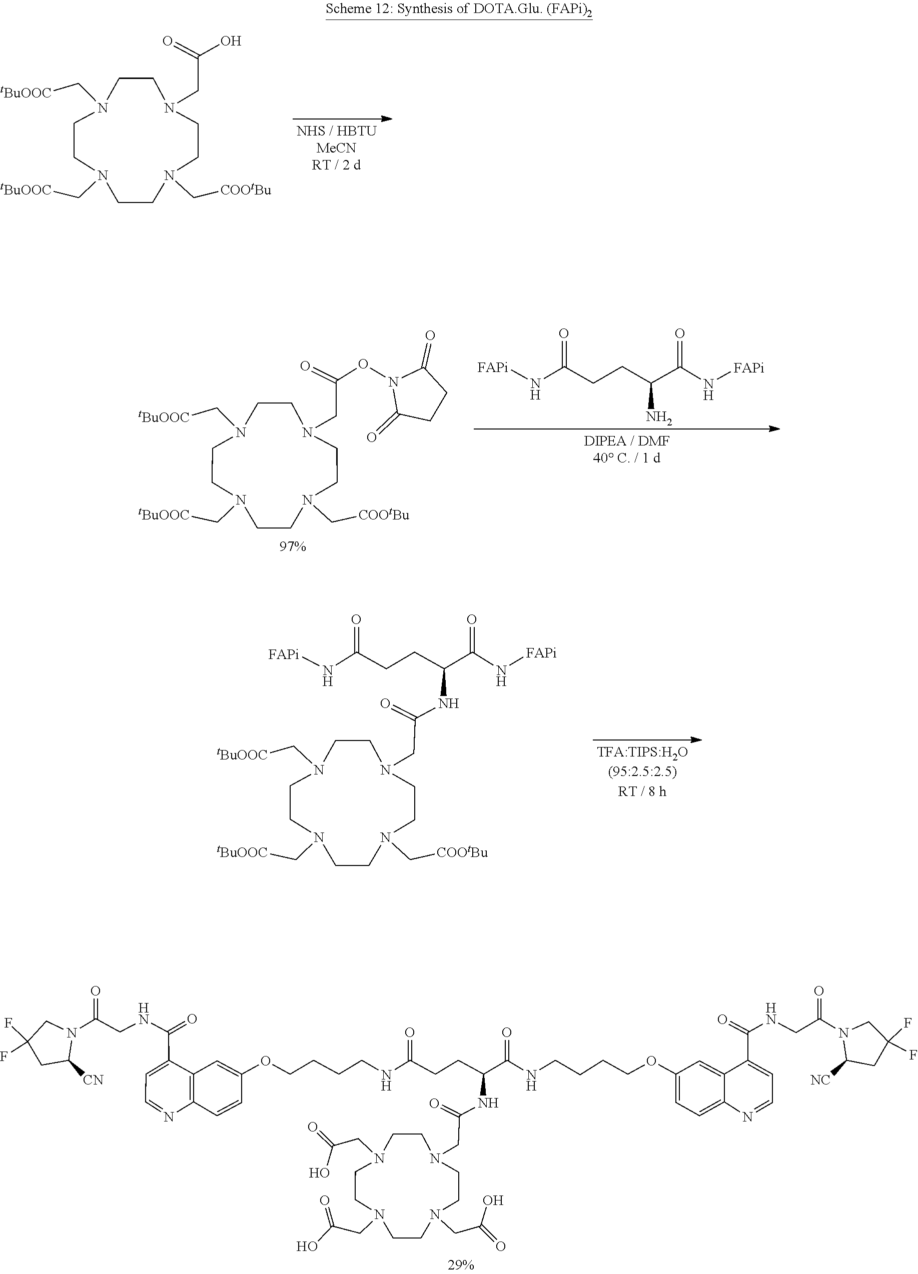

### DOTA(tBu)₃-NHS (2,2′,2″-(10-(2-((2,5-dioxopyrrolidin-1-yl)oxy)-2-oxoethyl)-1,4,7,10-tetraazacyclododecane-1,4,7-triyl)triacetic acid tert-butyl ester)

DOTA-tris(tert-butyl ester) (129 mg, 224 μmol, 1.0 eq) and 2-(1H-benzotriazol-1-yl)-1,1,3,3-tetramethyluronium hexafluorophosphate (HBTU, 87 mg, 229 μmol, 1.0 eq) were dissolved in dry ACN (5 mL). The mixture was stirred at RT under an argon atmosphere for 75 min, and then N-hydroxysuccinimide (NHS, 31 mg, 267 μmol, 1.2 eq) was added. After continued stirring overnight, HBTU (52.2 mg, 138 μmol, 0.6 eq) and, one hour later, NHS (22 mg, 191 μmol, 0.85 eq) were added and the mixture was stirred for a further day. After all the solvents had been removed in vacuo, after column chromatography (DCM:MeOH (100:15)), 145 mg (217 μmol, 97%) DOTA(tBu)₃-NHS was obtained as a colorless solid.

LC-MS (ESI-positive): m/z (%)=335.7 (100, [M+H]²⁺), 670.4 (50, [M+H]⁺), 671.4 (18, [M+H]⁺), calculated for C₃₂H₅₅N₅O₁₀: 669.39 [M]⁺.

### DOTA(tBu)₃.Glu.(FAPi)₂ (2,2′,2″-(10-(2-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-2-oxoethyl)-1,4,7,10-tetraazacyclododecan-1,4,7-triyl)triacetic acid tert-butyl ester)

DOTA(tBu)₃-NHS (40 mg, 60 μmol, 1.2 eq) was dissolved together with Glu.(FAPi)₂ (48.7 mg, 50 μmol, 1.0 eq) in dry DMF (2 mL), and DIPEA (200 μL) was added. The mixture was stirred under an argon atmosphere at 40° C. for 1 d and then all solvents were removed completely in vacuo. A yellow oil was obtained and used directly in the next stage without further purification.

HPLC-MS (ESI-positive): m/z (%)=382.95 (22, [M+H]⁴⁺), 383.20 (19, [M+H]⁴⁺), 491.57 (34, [M−tBu+H]³⁺), 491.90 (28, [M−tBu+H]³⁺), 492.24 (13, [M−tBu+H]³⁺), 510.26 (100, [M+H]³⁺), 510.59 (90, [M+H]³⁺), 510.93 (44, [M+H]³⁺), 511.26 (14, [M+H]³⁺), 764.88 (42, [M+H]²⁺), 765.38 (37, [M+H]²⁺), 765.89 (17, [M+H]²⁺), 1528.76 (25, [M+H]⁺), 1529.76 (22, [M+H]⁺), 1530.77 (10, [M+H]⁺), calculated for: C₇₅H₁₀₁F₄N₁₅O₁₅: 1527.75 [M]⁺.

### DOTA.Glu.(FAPi)₂ (2,2′,2″-(10-(2-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-2-oxoethyl)-1,4,7,10-tetraazacyclododecan-1,4,7-triyl)triacetic acid)

To DOTA(tBu)₃.Glu.(FAPi)₂ were added 50 μL of Milli-Q® water, 50 μL of TIPS and 1.9 mL of TFA (TFA:TIPS:H₂O (95:2.5:2.5)), and the mixture was stirred at RT for 8 h. Subsequently, 4× about 10 mL each time of MeOH was added and the solvents were removed again in vacuo. The crude product was purified by semipreparative RP-HPLC (22-23% ACN in 16 min, t_{R}=14-15 min). 19.9 mg (14.6 μmol, 29%) of a yellow solid was obtained.

LC-MS (ESI-positive): m/z (%)=340.85 (42, [M+H]⁴⁺), 351.00 (57, [M+ACN+H]⁴⁺), 361.35 (13, [M+2ACN+H]⁴⁺), 454.15 (100, [M+H]³⁺), 468.00 (20, [M+ACN+H]³⁺), 680.85 (9, [M+H]²⁺), calculated for C₆₃H₇₇F₄N₁₅O₁₅: 1359.57 [M]⁺.

### [^{nat}Lu]Lu-DOTA.Glu.(FAPi)₂

DOTA.Glu.(FAPi)₂ (2.8 mg, 2.0 μmol, 1.0 eq) was dissolved in 500 μL of 1 M HEPES buffer (pH=5.5), 40 μL of a 0.1 M LuCl₃ solution (4 μmol, 2.0 eq) was added and the mixture was shaken at 90° C. for 4 h. Subsequent semipreparative RP-HPLC (20-25% ACN in 20 min, t_{R}=14-15 min) gave 0.7 mg (0.46 μmol, 23%) [^{nat}Lu]Lu-DOTA.Glu.(FAPi)₂ as a yellow solid.

LC-MS (ESI-positive): m/z (%)=511.55 (100, [M+H]³⁺), 766.75 (14, [M+H]²⁺), calculated for C₆₃H₇₄F₄LuN₁₅O₁₅: 1531.48 [M]⁺.

### [⁶⁸Ga]Ga-DOTA.Glu.(FAPi)₂

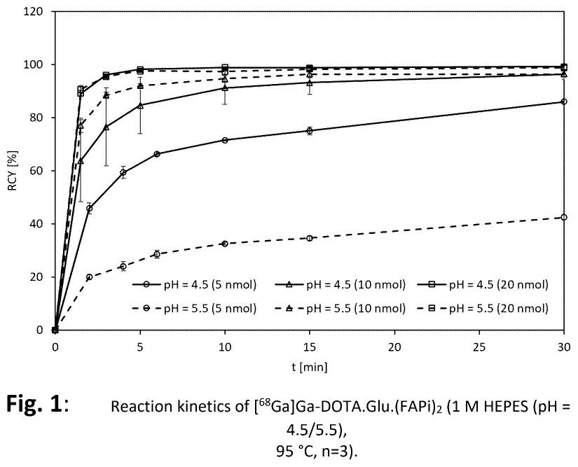

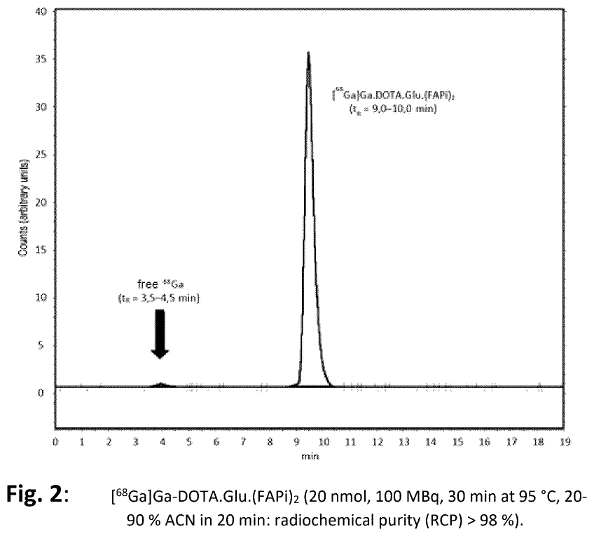

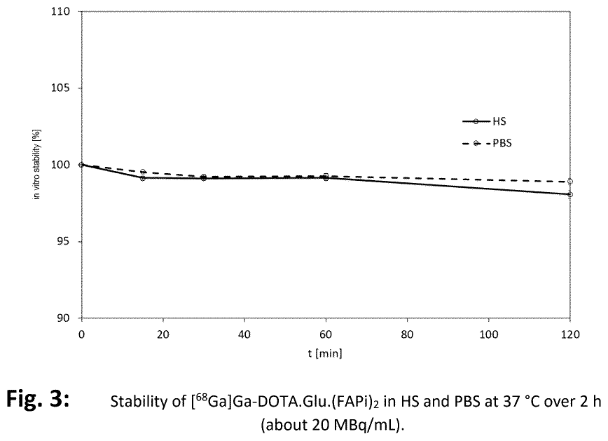

To an initial charge of 100 MBq [⁶⁸Ga]GaCl₃ was added, at 95° C., a solution of 400 μL of 1 M HEPES buffer (pH=4.5 or 5.5) and 5-20 nmol of DOTA.Glu.(FAPi)₂ (5-20 μL of a 1 μmol/mL stock solution with Trace-Select H₂O), and then the mixture was shaken for 30 min. The labeling was conducted at least three times (n=3) for each molar amount (5, 10 and 20 nmol), and was analyzed each time via radio-TLC with 0.1 M Na₃ citrate buffer (pH=4.0) as mobile phase (see FIG. 1). In addition, consistency was examined by comparison with radio-TLCs with 1 M AmOAc (pH=4)/MeOH (1:1) and analytical radio-HPLC (FIG. 2). It was possible to achieve a high radiochemical conversion of >98%. Stability after 2 h in HS and PBS is more than 98% (see FIG. 3). The log D value was determined as −2.08±0.07.

### [¹⁷⁷Lu]Lu-DOTA.Glu.(FAPi)₂

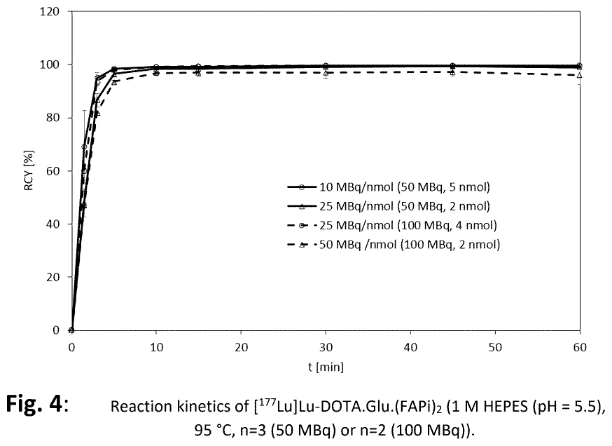

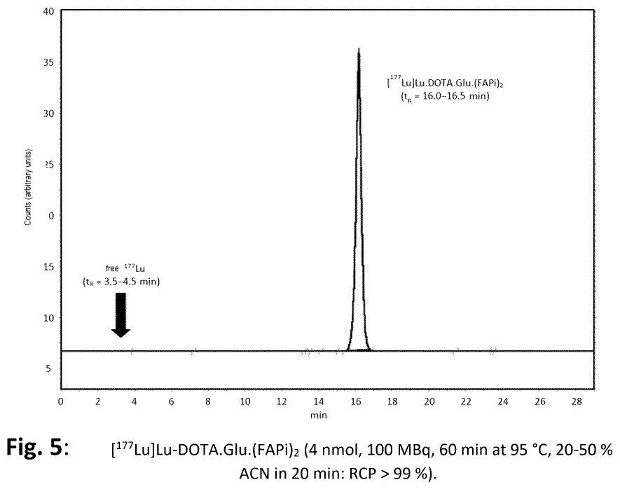

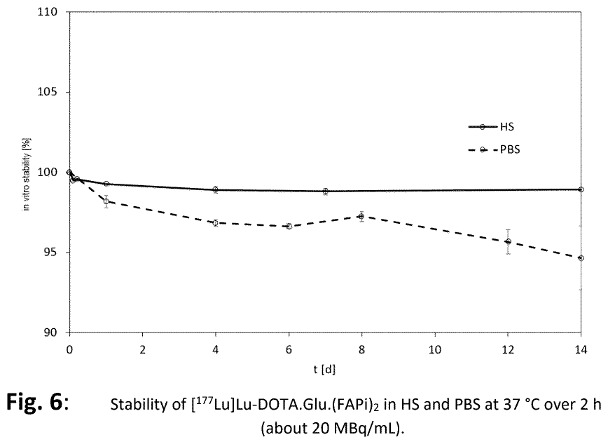

To an initial charge of 50-100 MBq [¹⁷⁷Lu]LuCl₃ in 20-40 μL of 0.04 M HCl were added, at 95° C., a solution of 400 μL of 1 M HEPES buffer (pH=5.5) and 2-5 nmol of DOTA.Glu.(FAPi)₂ (2-5 μL of a 1 μmol/mL stock solution with Trace-Select H₂O), and then the mixture was shaken for 60 min. The labeling was conducted repeatedly (n=3 (50 MBq), n=2 (100 MBq)) and analyzed by developing and evaluating radio-TLCs in each case with 0.1 M Na₃ citrate buffer (pH=4.0) as mobile phase (see FIG. 4). In addition, consistency was examined by comparison with radio-TLCs with 1 M AmOAc (pH=4)/MeOH (1:1) and analytical radio-HPLC (FIG. 5). It was possible to achieve a high radiochemical conversion of >99%. Stability after 14 d is about 99% in HS and 95% in PBS (see FIG. 6). The log D value was determined as −1.77±0.10.

The synthesis of the labeling precursor DOTAGA.Glu.(FAPi)₂ is shown below in scheme 12.

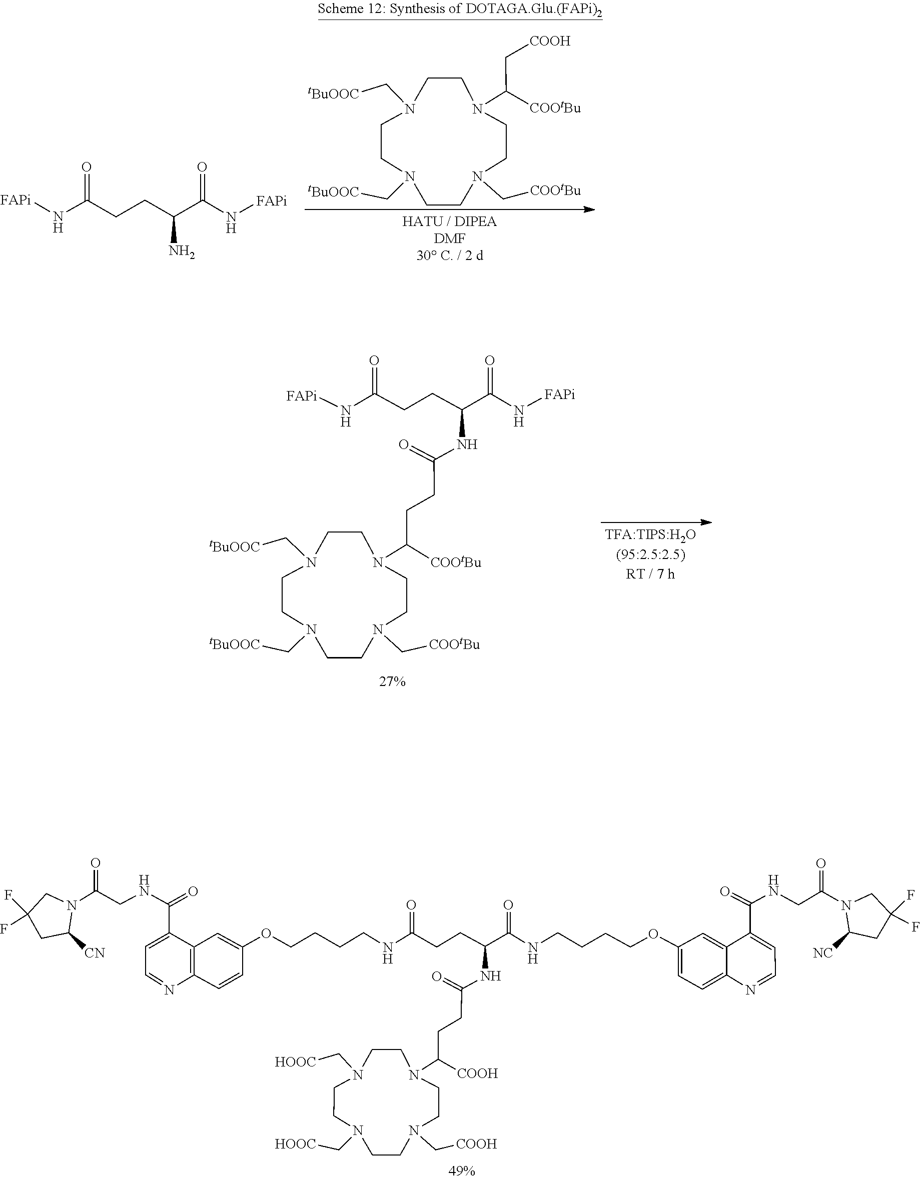

### DOTAGA(tBu)₄.Glu.(FAPi)₂ (2,2,2″-(10-(5-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-1-(tert-butoxy)-1,5-dioxopentan-2-yl)-1,4,7,10-tetraazacyclododecan-1,4,7-triyl)triacetic acid tert-butyl ester)

DOTAGA(tBu)₄ (60 mg, 85.6 μmol, 1.0 eq) and O-(7-azabenzotriazol-1-yl)-N,N,N′,N′-tetramethyluronium hexafluorophosphate (HATU, 36 mg, 94.2 μmol, 1.1 eq) were dissolved under an argon atmosphere in dry DMF (2 mL), and DIPEA (17.5 μL, 103 μmol, 1.2 eq) was added. After 1 h at 30° C., a solution of Glu.(FAPi)₂ (104 mg, 107 μmol, 1.25 eq) and DIPEA (43.7 μL, 257 μmol, 3 eq) in dry DMF (3 ml) was added. The mixture was stirred at 30° C. overnight, and then HATU (16 mg, 42 μmol, 0.5 eq) was added again. After stirring at 30° C. for a further day, the solvent was removed in vacuo. Purification by column chromatography (CHCl₃:MeOH:triethylamine(TEA) (100:10-15:1)) gave 39 mg (23.5 μmol, 27%) DOTAGA(tBu)₄.Glu.(FAPi)₂ as a yellow oil.

HPLC-MS (ESI-positive): m/z (%)=414.97 (13, [M+H]⁴⁺), 415.22 (12, [M+H]⁴⁺), 552.95 (100, [M+H]³⁺), 553.29 (97, [M+H]³⁺), 553.62 (51, [M+H]³⁺), 553.96 (18, [M+H]³⁺), 828.93 (82, [M+H]²⁺), 829.43 (78, [M+H]²⁺), 829.93 (40, [M+H]²⁺), 830.43 (15, [M+H]²⁺), 1656.85 (87, [M+H]⁺), 1657.85 (85, [M+H]⁺), 1658.85 (43, [M+H]⁺), 1659.86 (15, [M+H]⁺), calculated for C₈₂H₁₁₃F₄N₁₅O₁₇: 1655.84 [M]⁺.

### DOTAGA.Glu.(FAPi)₂ (2,2′,2″-(10-(4-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-1-carboxy-4-oxobutyl)-1,4,7,10-tetraazacyclododecan-1,4,7-triyl)triacetic acid)

To DOTAGA(tBu)₄.Glu.(FAPi)₂ were added 50 μL of Milli-Q® water, 50 μL of TIPS and 1.9 mL of TFA (TFA:TIPS:H₂O (95:2.5:2.5)), and the mixture was stirred at RT for 7 h. Subsequently, 5× about 10 mL each time of MeOH was added and the solvents were removed again in vacuo. The crude product was purified by semipreparative RP-HPLC (23% ACN isocratic, t_{R}=10-10.5 min). 16.4 mg (11.5 μmol, 49%) of a yellow solid was obtained.

LC-MS (ESI-positive): m/z (%)=358.85 (65, [M+H]⁴⁺), 369.05 (24, [M+ACN+H]⁴⁺), 478.30 (100, [M+H]³⁺), 717.30 (6, [M+H]²⁺), 1432.40 (1, [M+H]⁺), 1454.70 (1, [M+Na]⁺), calculated for C₆₆H₈₁F₄N₁₅O₁₇: 1431.59 [M]⁺.

### [^{nat}Lu]Lu-DOTAGA.Glu. (FAPi)₂

DOTAGA.Glu.(FAPi)₂ (2.8 mg, 2.0 μmol, 1.0 eq) was dissolved in 550 μL of 1 M HEPES buffer (pH=5.5) and 50 μL of EtOH, 40 μL of a 0.1 M LuCl₃ solution (4 μmol, 2.0 eq) was added and the mixture was shaken at 90° C. for 4 h. Subsequent semipreparative RP-HPLC (23% ACN isocratic, t_{R}=13-14 min) gave 0.5 mg (0.31 μmol, 16%) of [^{nat}Lu]Lu.DOTAGA.Glu.(FAPi)₂ as a yellow solid.

LC-MS (ESI-positive): m/z (%)=535.50 (100, [M+H]³⁺), 802.95 (36, [M+H]²⁺), calculated for C₆₆H₇₈F₄LuN₁₅O₁₇: 1603.50 [M]⁺.

### [⁶⁸Ga]Ga-DOTAGA.Glu.(FAPi)₂

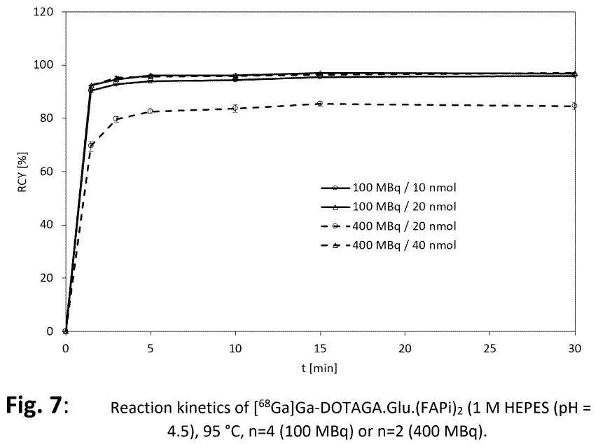

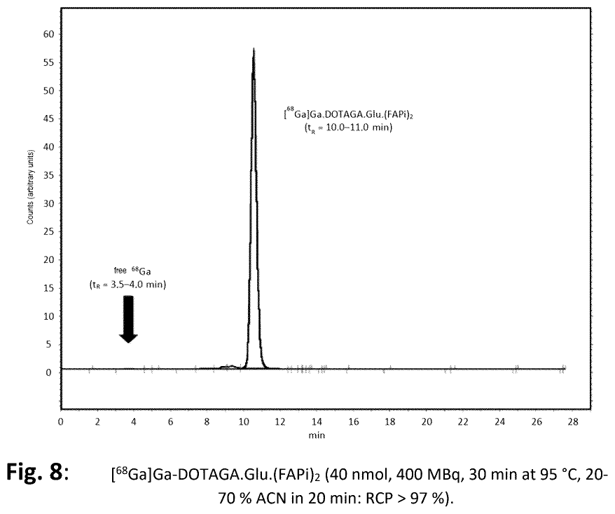

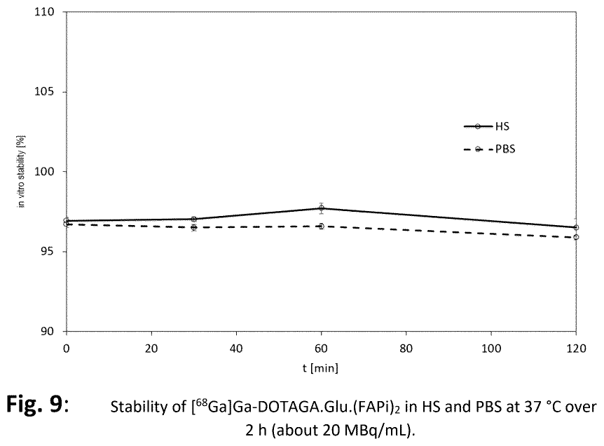

To an initial charge of 100 or 400 MBq [⁶⁸Ga]GaCl₃ in 0.05 M HCl (0.5 or 2 mL) were added, at 95° C., a solution of 0.5 or 2 mL of 1 M HEPES buffer (pH=4.5) and 10-40 nmol of DOTAGA.Glu.(FAPi)₂ (10-40 μL of a 1 μmol/mL stock solution with Trace-Select H₂O), and then the mixture was shaken for 30 min. The labeling was conducted repeatedly (n=4 (100 MBq), n=2 (400 MBq)), and the reaction kinetics were examined in each case via radio-TLC with 0.1 M Na₃ citrate buffer (pH=4.0) as mobile phase (see FIG. 7). In addition, consistency was examined by comparison with radio-TLCs with 1 M AmOAc (pH=4)/MeOH (1:1) and analytical radio-HPLC (FIG. 8). It was possible to achieve a high radiochemical conversion of >97%. Stability after 2 h in HS and PBS is more than 95% (see FIG. 9). The log D value was determined as −2.48±0.05.

### [⁷⁷Lu]Lu-DOTAGA.Glu.(FAPi)₂

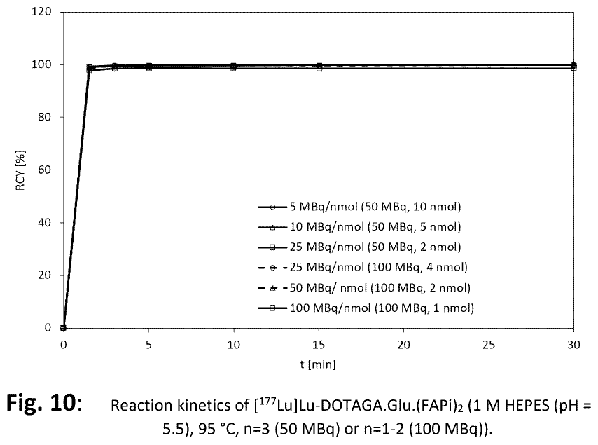

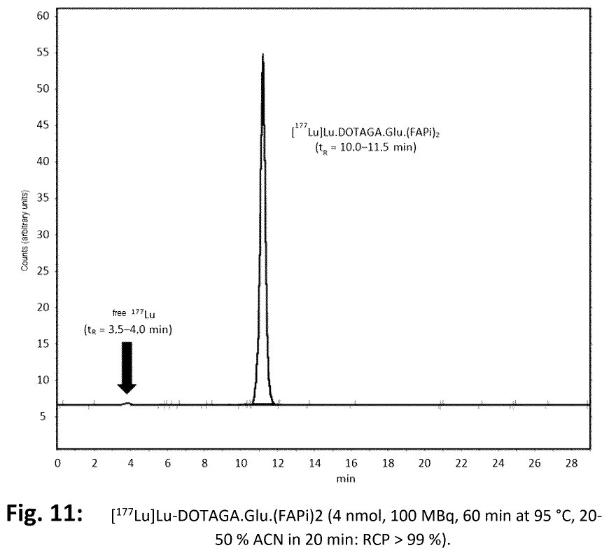

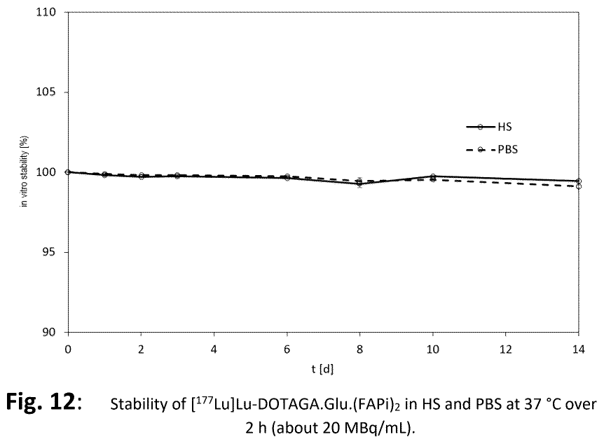

To an initial charge of 50-100 MBq [¹⁷⁷Lu]LuCl₃ in 20-40 μL 0.04 M HCl was added, at 95° C., a solution of 400 μL of 1 M HEPES buffer (pH=5.5) and 1-5 nmol of DOTAGA.Glu.(FAPi)₂ (1-5 μL of a 1 μmol/mL stock solution with Trace-Select H₂O), and then the mixture was shaken for 60 min. The reaction kinetics were examined (number of labelings: n=3 (50 MBq), n=1-2 (100 MBq)) by developing and evaluating radio-TLCs with 0.1 M Na₃ citrate buffer (pH=4.0) as mobile phase (see FIG. 10). In addition, consistency was examined by comparison with radio-TLCs with 1 M AmOAc (pH=4)/MeOH (1:1) and analytical radio-HPLC (FIG. 11). It was possible to achieve a high radiochemical conversion of >99%. Stability after 14 d is >99% in HS and PBS (see FIG. 12). The log D value was determined as −2.77±0.10.

### [²²⁵Ac]Ac-DOTAGA.Glu.(FAPi)₂

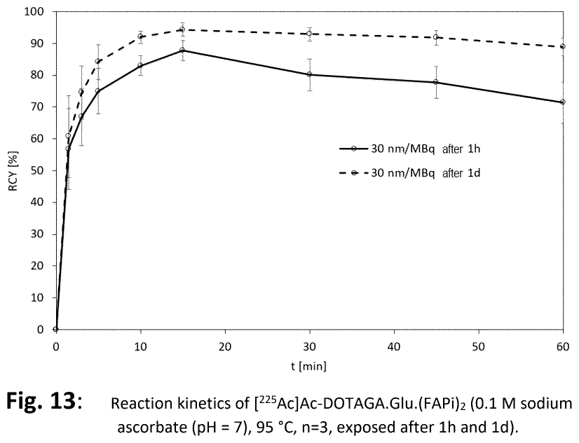

To an initial charge of 1.6-3.2 MBq of [²²⁵Ac]AcCl₃ in 100 μL of 0.04 M HCl was added, at 95° C., a solution of 1 mL of 0.1 M sodium ascorbate (pH=7.0) and 30 nmol/MBq of DOTAGA.Glu.(FAPi)₂ (30 μl/MBq 1 μmol/mL stock solution with Trace-Select H₂O), and then the mixture was shaken for 60 min. The labeling was conducted three times (n=3) and the reaction kinetics were examined. For this purpose, radio-TLCs with 0.1 M Na₃ citrate buffer (pH=4.0) as mobile phase (see FIG. 13) were developed and exposed and evaluated at different times (1 h and 1 d). A high radiochemical conversion of >94.3±2.1% (exposure after 1 d) was observed after 15 min. Subsequent purification by means of a SepPak® Light C18 cartridge ultimately gave the product in high radiochemical purity (>98%, determined via radio-TLC and high-resolution gamma spectroscopy with an HPGe detector).

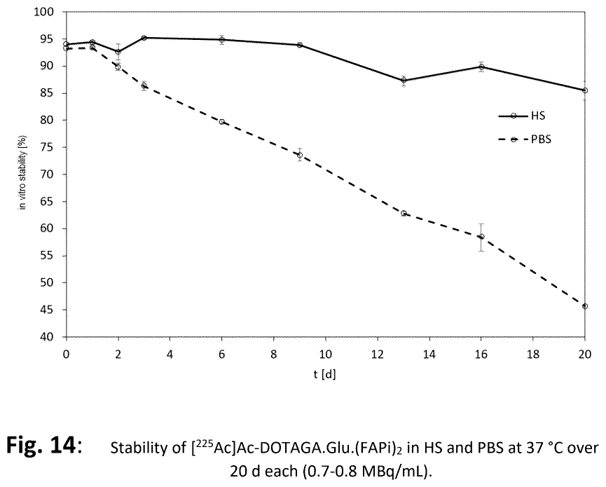

For the measurements of stability of [²²⁵Ac]Ac-DOTAGA.Glu.(FAPi)₂, 350-400 kBq of the labeling solution was added to HS and PBS (n=3 in each case) and incubated at 37° C. for 20 d (see FIG. 14).

The synthesis of the labeling precursor DATA^{5m}.Glu.(FAPi)₂ is shown below in scheme 13:

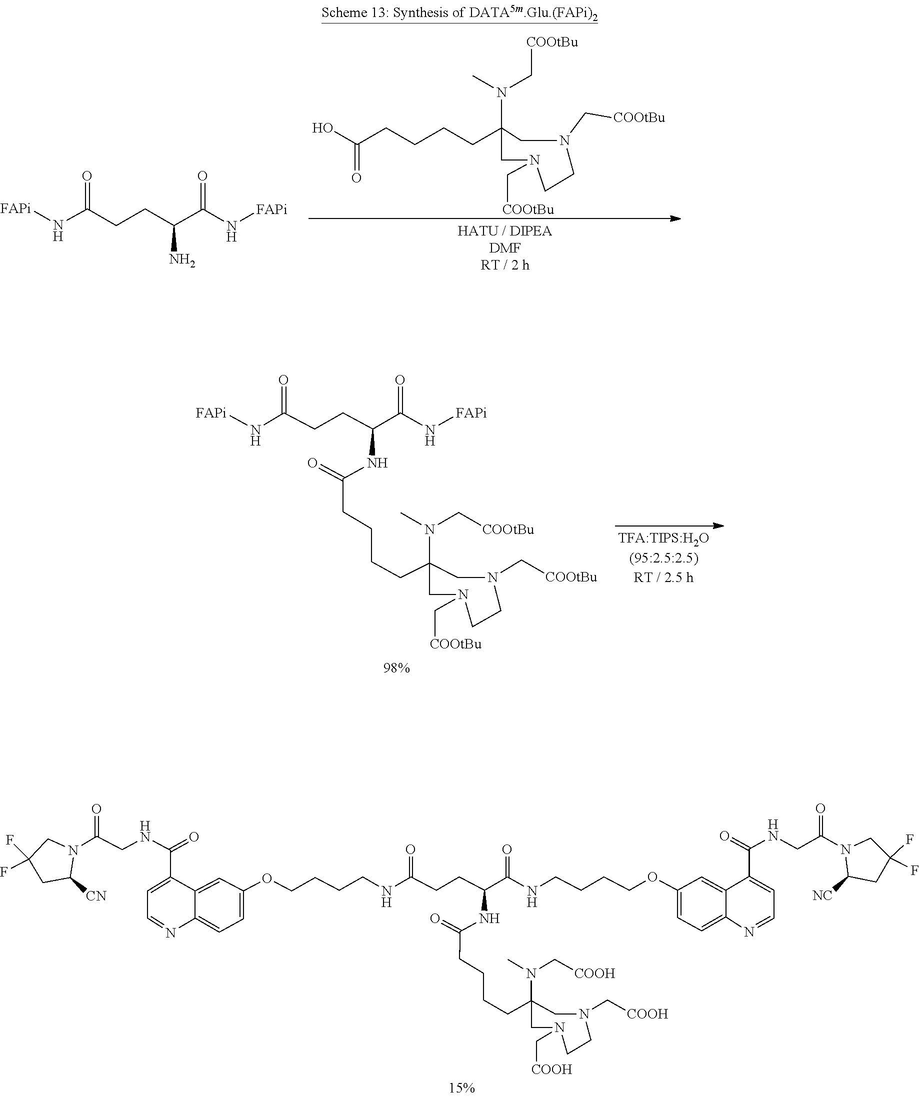

### DATA^{5M}(tBU)₃.Glu.(FAPI)₂ (2,2′-(6-(5-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-5-oxopentyl)-6-((2-(tert-butoxy)-2-oxoethyl)(methyl)amino)-1,4-diazepane-1,4-diyl)diacetic acid tert-butyl ester)

DATA^{5m}(tBu)₃ (22.8 mg, 40 μmol, 1.0 eq) and HATU (17.5 mg, 46 μmol, 1.15 eq) were dissolved in dry DMF (1 mL), and DIPEA (8.5 μL, 50 μmol, 1.25 eq) was added. Under an argon atmosphere, after 1 h at 25° C., a solution of Glu.(FAPi)₂ (39 mg, 40 μmol, 1.0 eq) and DIPEA (17 μL, 100 μmol, 2.5 eq) in dry DMF (2 mL) was added. Stirring was continued at 25° C. for 2 h. The solvent was removed in vacuo, and subsequent purification by column chromatography (CHCl₃:MeOH:triethylamine(TEA) (100:10-15:1)) gave 60 mg (39.2 μmol, 98%) of a yellow oil.

LC-MS (ESI-positive): m/z (%)=510.0 (100, [M+H]³⁺), 764.5 (24, [M+H]²⁺), calculated for C₇₆H₁₀₂F₄N₁₄O₁₅: 1526.76 [M].

### DATA^{5m}.Glu.(FAPi)₂ (2,2′-(6-(5-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-5-oxopentyl)-6-((carboxymethyl)(methyl)amino)-1,4-diazepane-1,4-diyl)diacetic acid)

To DATA^{5m}(tBu)₃.Glu.(FAPi)₂ were added 25 μL of Milli-Q® water, 25 μL of TIPS and 950 μL of TFA (TFA:TIPS:H₂O (95:2.5:2.5)), and the mixture was stirred at RT for 2.5 h. Subsequently, 3× about 10 mL each time of MeOH was added and the solvents were removed again in vacuo. The crude product was purified by semipreparative RP-HPLC (23% ACN isocratic, t_{R}=13-13.5 min). 8.2 mg (6.0 μmol, 15%) of a yellow solid was obtained.

LC-MS (ESI-positive): m/z (%)=340.7 (6, [M+H]⁴⁺), 454.0 (100, [M+H]³⁺), 680.4 (48, [M+H]²⁺), 706.8 (47, [M+Fe]²⁺), 707.3 (35, [M+Fe]²⁺), 1359.5 (6, [M+H]⁺), 1360.5 (5, [M+H]⁺), calculated for C₆₄H₇₈F₄N₁₄O₁₅: 1358.57 [M]⁺.

### [⁶⁸Ga]Ga-DATA^{5m}.Glu.(FAPi)₂

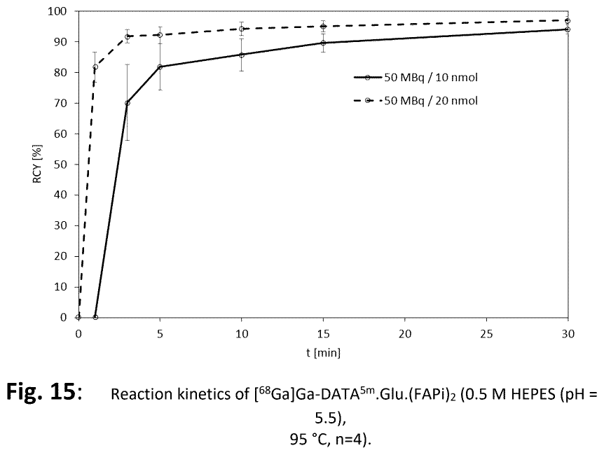

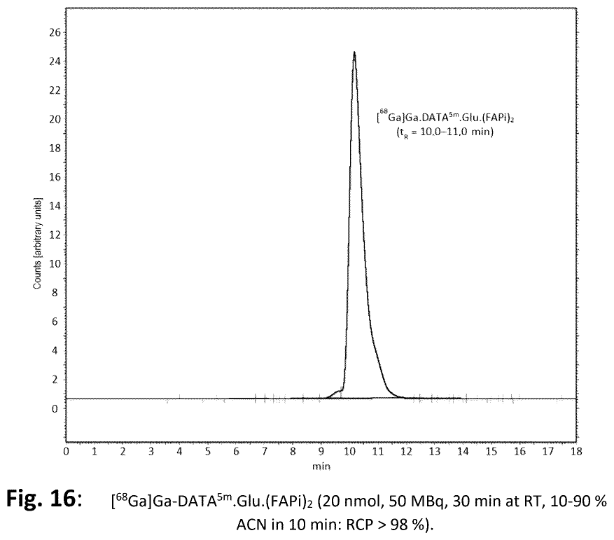

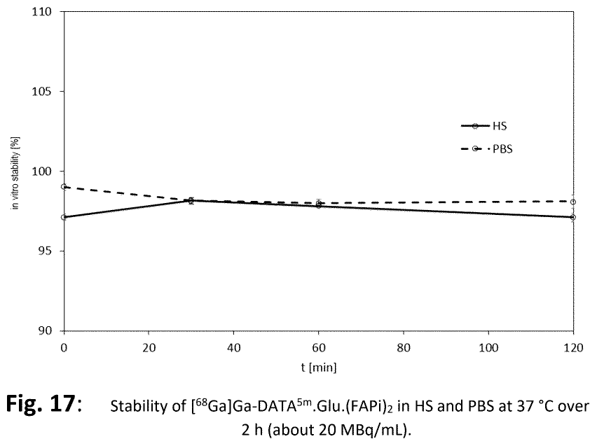

To an initial charge of 50 MBq [⁶⁸Ga]GaCl₃ was added, at room temperature, a solution of 400 μL of 0.5 M HEPES buffer (pH=5.5) and 10-20 nmol of DOTA.Glu.(FAPi)₂ (10-20 μL of a 1 μmol/mL stock solution with Trace-Select H₂O), and then the mixture was shaken for 30 min. The labelings were conducted four times (n=4) for both molar amounts and analyzed via radio-TLC with 0.1 M Na₃ citrate buffer (pH=4.0) as mobile phase (see FIG. 15). In addition, consistency was examined by comparison with radio-TLCs with 1 M AmOAc (pH=4)/MeOH (1:1) and analytical radio-HPLC (FIG. 16). A high radiochemical conversion of >96% was achieved. Stability after 2 h in HS and PBS is >97% (see FIG. 17). The log D value was determined as −2.03±0.05.

Table 1 summarizes the experimentally determined log D values.

TABLE 1
logD measurements of the ⁶⁸Ga- and ¹⁷⁷Lu-
labeled compounds DOTAGA.Glu.(FAPi)₂, DOTA.Glu.(FAPi)₂ and
DATA^{5m}.Glu.(FAPi)₂.

| | DOTAGA.Glu.(FAPi)₂ | DOTA.Glu.(FAPi)₂ | DATA^{5m}.Glu.(FAPi)₂ |
|---|---|---|---|
| ⁶⁸Ga | −2.48 ± 0.05 | −2.08 ± 0.07 | −2.03 ± 0.05 |
| ¹⁷⁷Lu | −2.77 ± 0.10 | −1.77 ± 0.10 | — |

### In Vitro Studies:

### FAP:

IC₅₀ measurements were conducted with Z-Gly-Pro-7-amino-4-methylcoumarin (AMC) as substrate in a concentration of 50 μM at pH=8 (0.05 M Tris-HCl buffer, 1 mg/mL of bovine serum albumin (BSA) and 140 mM NaCl). 8 concentrations of the FAP inhibitors examined were examined, with always the same DMSO concentration. The inhibitors were pre-incubated at 37° C. for 15 min before the Z-Gly-Pro-AMC substrate was added. The release kinetics of AMC were measured at an excitation wavelength λₑₓ=380 nm and emission wavelength Δₑₘ=465 nm for at least 10 min.

### PREP:

IC₅₀ measurements were conducted with N-succinyl-Gly-Pro-AMC as substrate in a concentration of 250 μM at pH=7.4 (0.1 M K phosphate buffer, 1 mM EDTA, 1 mM DTT and 1 mg/mL BSA). 8 concentrations of the FAP inhibitors examined were examined, with always the same DMSO concentration. The inhibitors were pre-incubated at 37° C. for 15 min before the N-succinyl-Gly-Pro-AMC substrate was added. The release kinetics of AMC were measured at an excitation wavelength λₑₓ=380 nm and emission wavelength λₑₘ=465 nm for at least 10 min.

### DPP4, DPP8 and DPP9:

IC₅₀ measurements were conducted with Ala-Pro-p-nitroanilide (pNA) as substrate in a concentration of 25 μM (DPP4), 300 μM (DPP8) or 150 μM (DPP9) at pH=7.4 (0.05 M HEPES-NaOH buffer with 0.1% Tween-20, 1 mg/mL BSA and 150 mM NaCl). At least 8 concentrations of the FAP inhibitors examined were examined, with always the same DMSO concentration. The inhibitors were pre-incubated at 37° C. for 15 min before the Ala-Pro-pNA substrate was added. The release kinetics of pNA were measured at a wavelength of λₑₓ=405 nm for at least 10 min.

Table 2 summarizes the results of the IC₅₀ measurements. The selectivity index (SI) is found from the ratio of the IC₅₀ value of FAP and the respective competing enzyme (PREP, DPP4, DPP8, DPP9).

TABLE 2
IC₅₀ measurements of the compounds DOTAGA.Glu.(FAPi)₂,
DOTA.Glu.(FAPi)₂ and DATA^{5m}.Glu.(FAPi)₂ and of the established
FAP inhibitor UAMC1110 (see scheme 4, on the right).

| | DOTAGA.Glu.(FAPi)₂ | DOTA.Glu.(FAPi)₂ | DATA^{5m}.Glu.(FAPi)₂ | UAMC1110 |
|---|---|---|---|---|
| IC₅₀ (FAP)/ | 0.26 ± 0.04 | 0.60 ± 0.04 | 0.71 ± 0.05 | 0.43 ± 0.02 |
| nM | | | | |
| IC₅₀ (PREP)/ | 0.59 ± 0.10 | 1.00 ± 0.14 | 0.31 ± 0.06 | 1.80 ± 0.01 |
| μM | | | | |
| IC₅₀ (DPP4)/ | 1.19 ± 0.08 | 0.54 ± 0.06 | 1.57 ± 0.06 | >10 |
| μM | | | | |
| IC₅₀ (DPP8)/ | 0.029 ± 0.004 | 1.03 ± 0.18 | 2.22 ± 0.40 | >10 |
| μM | | | | |
| IC₅₀ (DPP9)/ | 0.083 ± 0.0015 | 0.95 ± 0.11 | 0.77 ± 0.11 | 4.70 ± 0.40 |
| μM | | | | |
| SI | 2269 | 1667 | 437 | 4186 |
| (FAP/PREP) | | | | |
| SI | 4577 | 900 | 2211 | 23256 |
| (FAP/DPP4) | | | | |
| SI | 112 | 1717 | 3127 | 23256 |
| (FAP/DPP8) | | | | |
| SI | 319 | 1583 | 1085 | 10930 |
| (FAP/DPP9) | | | | |

### Example 3: DOTA.NPyr.(FAPi)₂, DOTAGA.NPyr.(FAPi)₂

There follows a description of the synthesis of the labeling precursors DOTA.NPyr.(FAPi)₂, DOTAGA.NPyr.(FAPi)₂. The first synthesis steps are identical for both compounds, and a representative synthesis is shown in scheme 14.

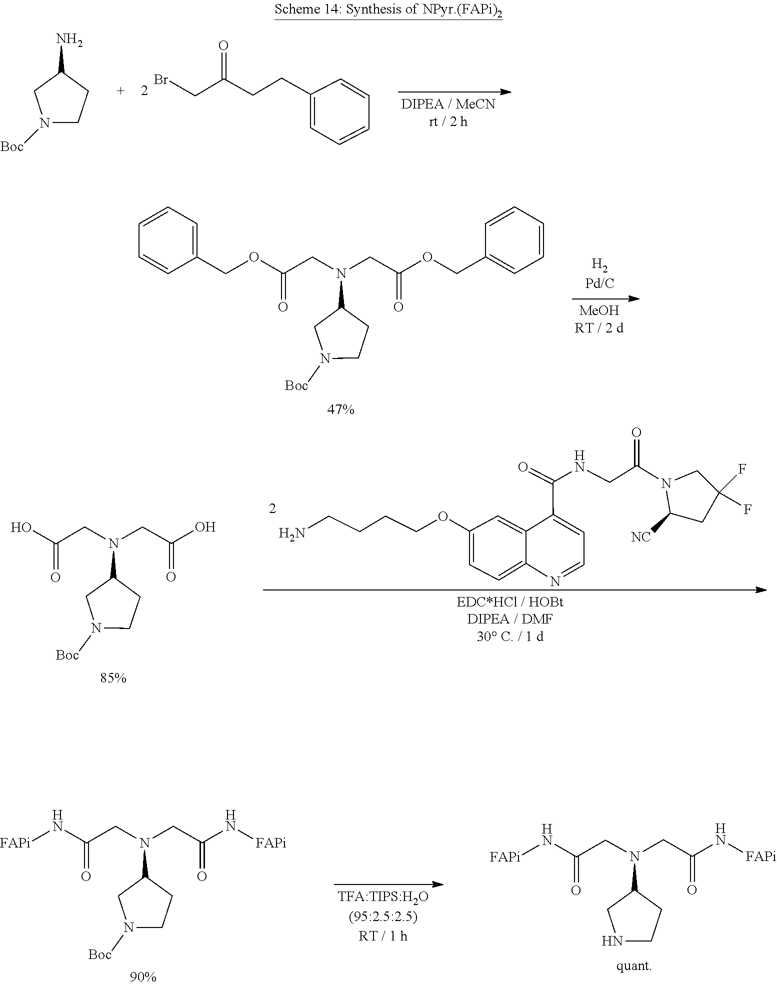

### Boc-NPyr(OBzl)₂ ((S)-2,2′-((1-(tert-butoxycarbonyl)pyrrolidin-3-yl)azanediyl)diacetic acid benzyl ester)

(S)-1-Boc-3-aminopyrrolidine (1.07 g, 5.74 mmol, 1.0 eq) and DIPEA (1.5 mL) were initially charged in acetonitrile (6 mL). After 60 min, a solution of benzyl bromoacetate (1.74 g, 7.55 mmol, 1.3 eq) in acetonitrile (6 mL) was slowly added dropwise and the mixture was stirred at RT for a further 2 h. Acetonitrile was removed under reduced pressure. Subsequent column chromatography (CHCl₃:MeOH (30:1)+1% TEA) gave Boc-NPyr(OBzl)₂ (1.31 g, 2.71 mmol, 47%) as a by-product alongside Boc-NPyr-OBzl (benzyl-(S)—N-(pyrrolidine-3-tert-butoxycarbamate)glycine, 680 mg, 2.03 mmol, 35%).

LC-MS (ESI-positive): m/z (%)=383.2 (45, [M−Boc+H]⁺), 483.2 (100, [M+H]⁺), 484.2 (30, [M+H]⁺), calculated for C₂₇H₃₄N₂O₆: 482.24 [M]⁺.

### Boc-NPyr ((S)-2,2′-((1-(tert-butoxycarbonyl)pyrrolidin-3-yl)azandiyl)diacetic acid)

To Boc-NPyr(OBzl)₂ (1.21 g, 2.51 mmol, 1.0 eq) were added palladium on activated carbon (10 wt % Pd, 53 mg, 50 μmol, 0.02 eq) and dry methanol (8 mL). The mixture was stirred under a hydrogen atmosphere at RT for 2 d. The mixture was filtered through Celite, and then methanol was removed under reduced pressure. A colorless solid was obtained (643 mg, 2.13 mmol, 85%).

LC-MS (ESI-positive): m/z (%)=247.0 (100, [M−^{t}Bu+H]⁺), 303.1 (36, [M+H]⁺), 605.3 (23, [2M+H]⁺), calculated for C₁₃H₂₂N₂O₆: 302.15 [M]⁺.

### Boc-NPyr.(FAPi)₂ (tert-butyl (S)-3-(bis(2-((4-((4-((2-((S)-2-cyano-4,4-difluoropyrrolidin-1-yl)-2-oxoethyl)carbamoyl)quinolin-6-yl)oxy)butyl)amino)-2-oxoethyl)amino)pyrrolidine-1-carboxylate)

Boc-NPyr (30.2 mg, 100 μmol, 1.0 eq), HOBt (36 mg, 266 μmol, 2.7 eq) and EDC*HCl (50 mg, 260 μmol, 2.6 eq) were dissolved in dry DMF (3 mL) and stirred under an argon atmosphere at 30° C. for 60 min. Then a solution of FAPi-NH₂*TFA (110 mg, 202 μmol, 2.0 eq) and DIPEA (51.0 μL, 300 μmol, 3.0 eq) in DMF (2 mL) was added and stirring of the mixture was continued at 30° C. for 3.5 h. Then HOBt (8.5 mg, 63 μmol, 0.63 eq) and EDC*HCl (12 mg, 63 μmol, 0.63 eq) were added and, 30 min later, a solution of FAPi-NH₂*TFA (25 mg, 46 μmol, 0.46 eq) and DIPEA (17.0 μL, 100 μmol, 1.0 eq) in DMF (1 mL). After stirring at 30° C. overnight, the additions were repeated in that HOBt (8.5 mg, 63 μmol, 0.63 eq), EDC*HCl (12 mg, 63 μmol, 0.63 eq) and, after a further 30 min, FAPi-NH₂*TFA (16 mg, 29 μmol, 0.29 eq) and DIPEA (17.0 μL, 100 μmol, 1.0 eq) in DMF (1 mL) were added. The mixture was stirred at 30° C. for a further 5 h, and then the solvent was removed in vacuo. After column chromatography (CHCl₃:MeOH:TEA (100:7.5-10:1)), 102 mg (90.3 μmol, 90%) of Boc-NPyr.(FAPi)₂ was obtained as a yellow oil.

LC-MS (ESI-positive): m/z (%)=358.6 (86, [M−^{t}Bu+H]³⁺), 372.2 (58, [M−^{t}Bu+ACN+H]3+), 377.3 (100, [M+H]³⁺), 390.3 (68, [M+ACN+H]³⁺), 515.3 (36, [M−Boc+H]²⁺), 537.5 (8, [M−^{t}Bu+H]²⁺), 565.5 (84, [M+H]²⁺), 1129.6 (28, [M+H]⁺), 1130.6 (17, [M+H]⁺), calculated for C₅₅H₆₄F₄N₁₂O₁₀: 1128.48 [M]⁺.

### NPyr.(FAPi)₂ (6,6′-((((2,2′-(((S)-Pyrrolidin-3-yl)azanediyl)bis(acetyl))bis(azanediyl))bis(butane-4,1-diyl))bis(oxy))bis(N-(2-((S)-2-cyano-4,4-difluoropyrrolidin-1-yl)-2-oxoethyl)quinoline-4-carboxamide)

To Boc-NPyr.(FAPi)₂ (102 mg, 90 μmol) were added 50 μL of Milli-Q® water, 50 μL of triisopropylsilane (TIPS) and 1.9 mL of TFA (TFA:TIPS:H₂O (95:2.5:2.5)), and the mixture was stirred at RT for 1 h. Subsequently, 5× about 10 mL each time of MeOH were added, and the solvents were removed again in vacuo and a yellow oil was obtained. It was used in the next stage without further purification.

LC-MS (ESI-positive): m/z (%)=344.1 (100, [M+H]³⁺), 357.6 (45, [M+ACN+H]³⁺), 515.5 (18, [M+H]²⁺), 1029.5 (3, [M+H]⁺), calculated for C₅₀H₅₆F₄N₁₂O₈: 1028.43 [M]⁺.

The synthesis of the labeling precursor DOTA.NPyr.(FAPi)₂ is shown below in scheme 15.

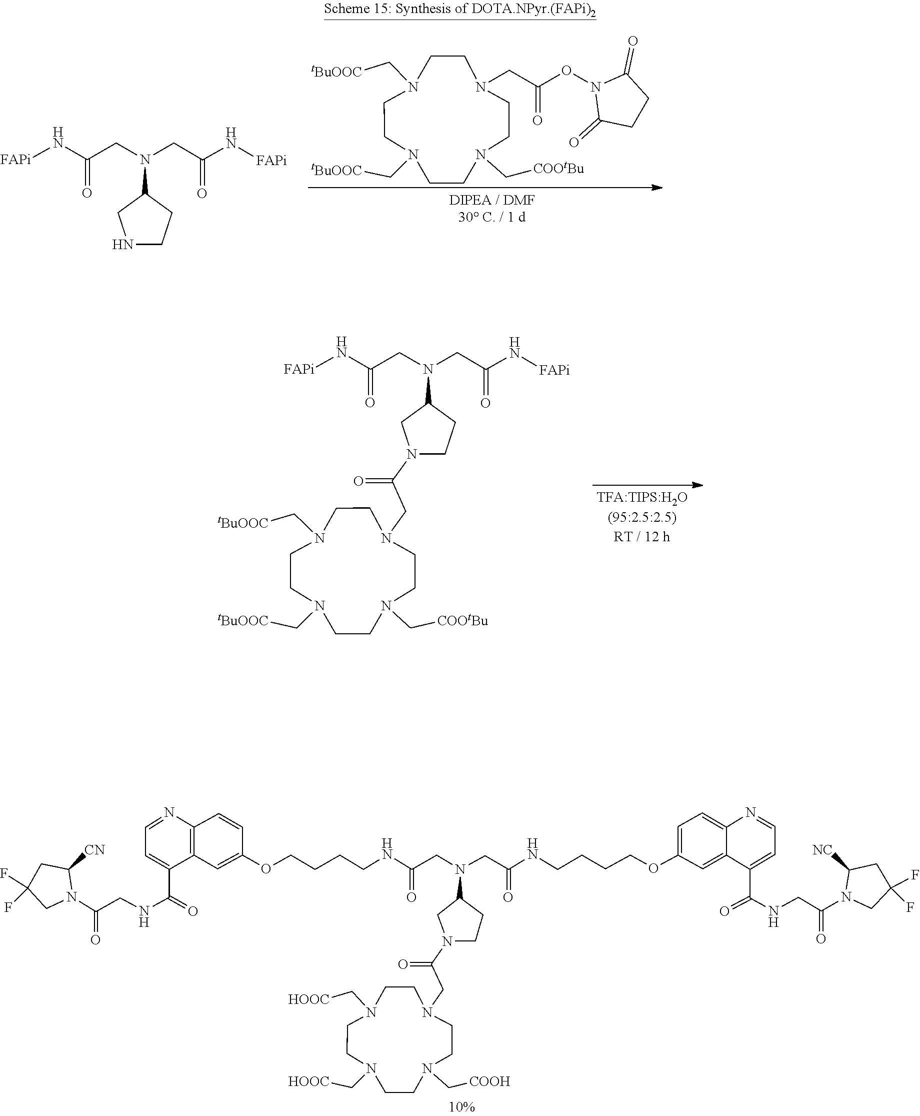

### DOTA(tBu)₃.NPyr.(FAPi)₂ (2,2′,2″-(10-(2-((S)-3-(bis(2-((4-((4-((2-((S)-2-cyano-4,4-difluoropyrrolidin-1-yl)-2-oxoethyl)carbamoyl)quinolin-6-yl)oxy)butyl)amino)-2-oxoethyl)amino)pyrrolidin-1-yl)-2-oxoethy)-1,4,7,10-tetraazacyclododecan-1,4,7-triyl)triacetic acid tert-butyl ester)

DOTA(tBu)₃-NHS (33.5 mg, 50 μmol, 1.25 eq) was dissolved together with NPyr.(FAPi)₂ (41.2 mg, 40 μmol, 1.0 eq) in dry DMF (1 mL), and DIPEA (50 μL) was added. The mixture was stirred at 40° C. under an argon atmosphere for 3 d, and then all solvents were removed completely in vacuo. A yellow oil was obtained and used directly in the next stage without further purification.

HPLC-MS (ESI-positive): m/z (%)=396.71 (35, [M+H]⁴⁺), 396.96 (33, [M+H]⁴⁺), 397.21 (15, [M+H]⁴⁺), 509.92 (48, [M−^{t}Bu+H]³⁺), 510.25 (42, [M−^{t}Bu+H]³⁺), 510.59 (20, [M−^{t}Bu+H]³⁺), 528.61 (100, [M+H]³⁺), 528.94 (95, [M+H]³⁺), 529.27 (50, [M+H]³⁺), 529.61 (17, [M+H]3+), 792.40 (30, [M+H]²⁺), 792.91 (28, [M+H]²⁺), 793.41 (13, [M+H]²⁺), 1583.80 (18, [M+H]⁺), 1584.81 (17, [M+H]⁺), 1585.81 (8, [M+H]⁺), 1605.79 (8, [M+Na]⁺), 1606.79 (8, [M+Na]⁺), calculated for: C₇₈H₁₀₆F₄N₁₆O₁₅: 1582.80 [M]⁺.

### DOTA.NPyr.(FAPi)₂ (2,2′,2″-(10-(2-((S)-3-(bis(2-((4-((4-((2-((S)-2-cyano-4,4-difluoropyrrolidin-1-yl)-2-oxoethyl)carbamoyl)quinolin-6-yl)oxy)butyl)amino)-2-oxoethyl)amino)pyrrolidin-1-yl)-2-oxoethyl)-1,4,7,10-tetraazacyclododecane-1,4,7-triyl)triacetic acid)

To DOTA(tBu)₃.NPyr.(FAPi)₂ were added 50 μL of Milli-Q® water, 50 μL of TIPS and 1.5 mL of TFA (TFA:TIPS:H₂O (94:3:3)), and the mixture was stirred at RT for 12 h. Subsequently, 4× about 10 mL each time of MeOH was added, and the solvents were removed again in vacuo. The crude product was purified by semipreparative RP-HPLC (21-22% ACN in 20 min, t_{R}=18.5-19.5 min). 5.6 mg (4.0 μmol, 10%) of a yellow solid was obtained.

LC-MS (ESI-positive): m/z (%)=354.55 (95, [M+H]⁴⁺), 364.750 (59, [M+ACN+H]⁴⁺), 472.60 (100, [M+H]³⁺), 708.55 (13, [M+H]²⁺), 1415.50 (5, [M+H]⁺), calculated for C₆₆H₈₂F₄N₁₆O₁₅: 1414.61 [M].

The synthesis of the labeling precursor DOTAGA.NPyr.(FAPi)₂ is shown below in scheme 16.

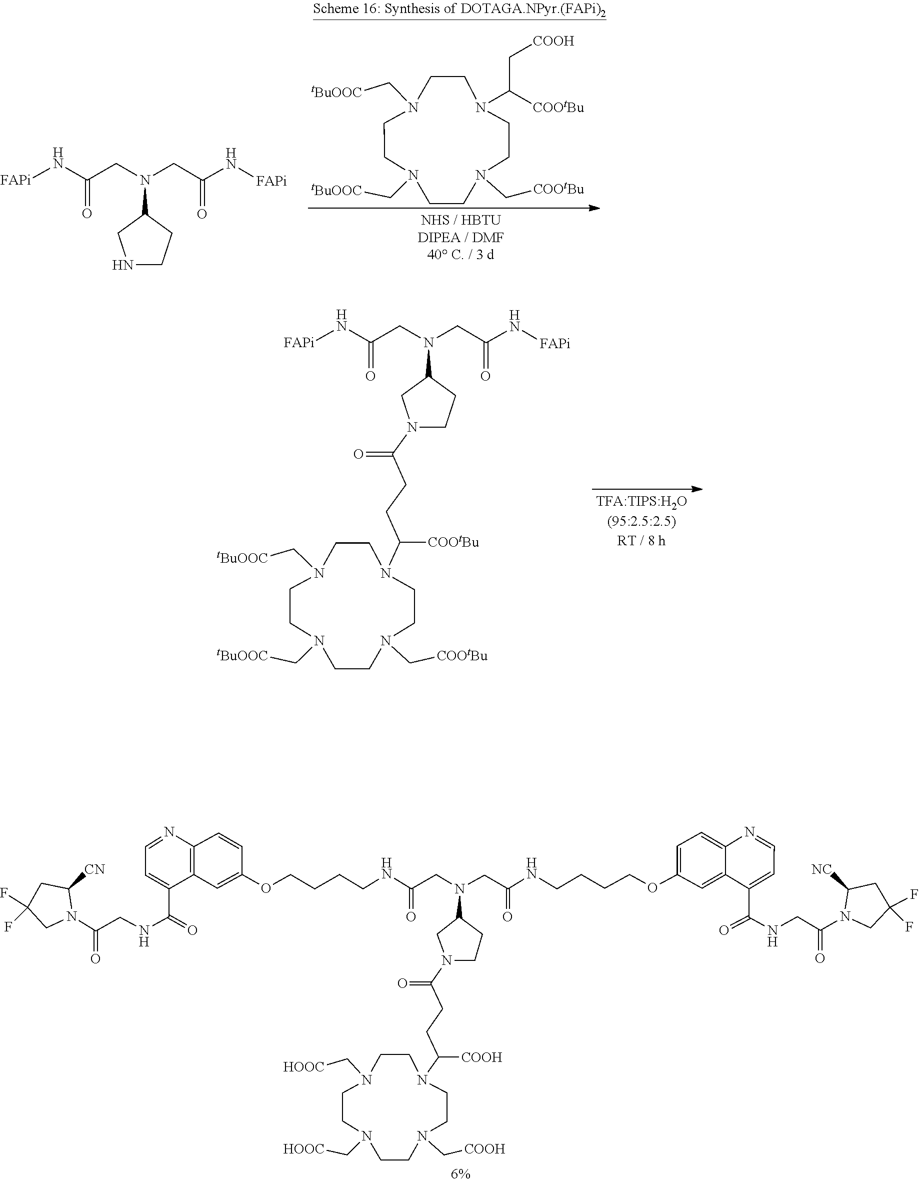

### DOTAGA(tBU)₄.NPyr.(FAPi)₂ (2,2′,2″-(10-(5-((S)-3-(bis(2-((4-((4-((2-((S)-2-cyano-4,4-difluoropyrrolidin-1-yl)-2-oxoethyl)carbamoyl)quinolin-6-yl)oxy)butyl)amino)-2-oxoethyl)amino)pyrrolidin-1-yl)-1-(tert-butoxy)-1,5-dioxopentan-2-yl)-1,4,7,10-tetraazacyclododecane-1,4,7-triyl)triacetic acid tert-butyl ester)

DOTAGA(tBu)₄ (23.5 mg, 33.5 μmol, 1.0 eq), NHS (8.0 mg, 70 μmol, 2.0 eq) and HBTU (26.5 mg, 70 μmol, 2.0 eq) were dissolved in dry DMF (0.5 mL) and shaken at 30° C. overnight. NHS (4.5 mg, 39.0 μmol, 1.26 eq) and HBTU (13.5 mg, 35.6 μmol, 1.06 eq) were added once again. 4 h later, a solution of NPyr.(FAPi)₂ (41.2 mg, 40 μmol, 1.0 eq) and DIPEA (50 μL) in dry DMF (1 mL) was added. The mixture was stirred at 40° C. for 3 d, and then all solvents were removed completely in vacuo. A yellow oil was obtained and used directly in the next stage without further purification.

HPLC-MS (ESI-positive): m/z (%)=428.73 (100, [M+H]⁴⁺), 428.98 (32, [M+H]⁴⁺), 429.23 (25, [M+H]4+), 571.64 (16, [M+H]3+), 571.97 (10, [M+H]3+), 856.45 (5, [M+H]²⁺), 856.95 (5, [M+H]²⁺), 1711.89 (2, [M+H]⁺), 1712.89 (2, [M+H]⁺), 1733.87 (2, [M+Na]⁺), 1734.87 (2, [M+Na]⁺), calculated for: C₈₅H₁₁₈F₄N₁₆O₁₇: 1710.88 [M]⁺.

### DOTAGA.NPyr.(FAPi)₂ (2,2′,2″-(10-(4-((S)-3-(bis(2-((4-((4-((2-((S)-2-cyano-4,4-difluoropyrrolidin-1-yl)-2-oxoethyl)carbamoyl)quinolin-6-yl)oxy)butyl)amino)-2-oxoethyl)amino)pyrrolidin-1-yl)-1-carboxy-4-oxobutyl)-1,4,7,10-tetraazacyclododecane-1,4,7-triyl)triacetic acid)

To DOTA(tBu)₃.NPyr.(FAPi)₂ were added 50 μL of Milli-Q® water, 50 μL of TIPS and 1.9 mL of TFA (TFA:TIPS:H₂O (95:2.5:2.5)), and the mixture was stirred at RT for 8 h. Subsequently, 4× about 10 mL each time of MeOH was added, and the solvents were removed again in vacuo. The crude product was purified by semipreparative RP-HPLC (21% ACN isocratic, t_{R}=23-24 min). 3.0 mg (2.0 μmol, 6%) of a yellow solid was obtained. LC-MS (ESI-positive): m/z (%)=372.55 (100, [M+H]⁴⁺), 382.90 (38, [M+ACN+H]⁴⁺), 496.60 (76, [M+H]³⁺), 744.40 (5, [M+H]²⁺), calculated for C₆₉H₈₆F₄N₁₆O₁₇: 1486.63 [M].

### Example 4: DOTA.PEG2.Glu.(FAPi)₂, DOTAGA.PEG2.Glu.(FAPi)₂

There follows a description of the synthesis of the labeling precursors DOTA.PEG2.Glu.(FAPi)₂, DOTAGA.PEG2.Glu.(FAPi)₂. The first synthesis steps are identical for both compounds, and a representative synthesis is shown in scheme 17.

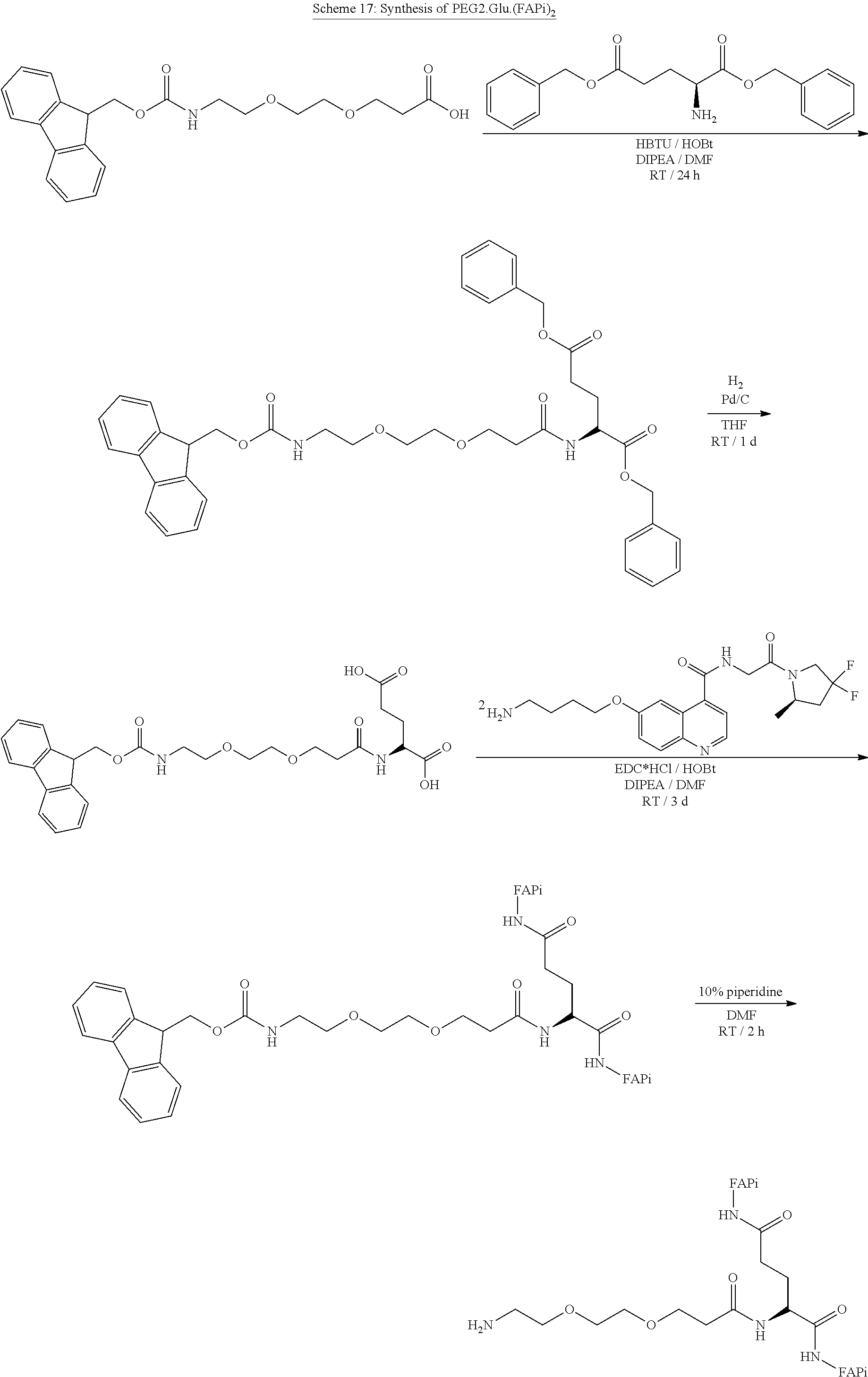

### Fmoc-PEG2.Glu(OBZl)₂ ((1-(9H-fluoren-9-yl)-3-oxo-2,7,10-trioxa-4-azatridecan-13-oyl)-L-glutamic acid dibenzyl ester)

Fmoc-N-amido-dPEG₂ acid (450.0 mg, 1.1 mmol, 1.00 eq.) and DIPEA (182.0 mg, 240 μL, 1.4 mmol, 1.25 eq.) were dissolved in dry DMF (9.0 mL), and HBTU (470.3 mg, 1.2 mmol, 1.10 eq.) and HOBt (167.6 mg, 1.2 mmol, 1.10 eq.) were added. The colorless solution was stirred at 25° C. under an argon atmosphere for 24 h. After one hour, dibenzyl glutamate (460.6 mg, 1.4 mmol, 1.25 eq.) dissolved in dry DMF (3.0 mL) and DIPEA (320.5 mg, 422 μL, 4.5 mmol, 2.20 eq.) were added. After the reaction had ended, the solvent was removed under reduced pressure and the yellowish oil was purified by column chromatography (DCM:MeOH (100:2)). Fmoc-PEG2.Glu(OBzl)₂ (795.1 mg, 1.1 mmol, 99%) was obtained as a colorless oil.

LC-MS (ESI-positive): m/z (%)=709.4 (100, [M+H]⁺), 710.2 (15, [M+H]⁺), calculated for C₄₁H₄₄N₂O₉: 708.30 [M]⁺.

### Fmoc-PEG2.Glu ((1-(9H-fluoren-9-yl)-3-oxo-2,7,10-trioxa-4-azatridecan-13-oyl)-L-glutamic acid)

Fmoc-PEG2.Glu(OBzl)₂ (196.4 mg, 0.3 mmol, 1.00 eq.) was dissolved in dry tetrahydrofuran (THF) (2.0 mL), and palladium on activated carbon (10 wt % Pd, 30.0 mg, 0.3 mmol, 1.00 eq.) was added. The mixture was then stirred under a hydrogen atmosphere for 24 h. The suspension was filtered through Celite, the residue was washed with THF, and the solvent was removed under reduced pressure. Fmoc-PEG2.Glu (122.2 mg, 231.3 μmol, 82%) was obtained as a colorless oil and used in the next stage without further workup.

LC-MS (ESI-positive): m/z (%)=529.25 (100, [M+H]⁺), 530.15 (12, [M+H]⁺), calculated for C₂₇H₃₂N₂O₉: 528.21 [M]⁺.

### Fmoc-PEG2.Glu.(FAPi)₂ ((9H-fluoren-9-yl)methyl ((11S)-19-((4-((2-(2-cyano-4,4-difluoropyrrolidin-1-yl)-2-oxoethyl)carbamoyl)quinolin-6-yl)oxy)-11-((4-((4-((2-(2-cyano-4,4-difluoropyrrolidin-1-yl)-2-oxoethyl)carbamoyl)quinolin-6-yl)oxy)butyl)carbamoyl)-9,14-dioxo-3,6-dioxa-10,15-diazanonadecyl)carbamate)

Fmoc-PEG2.Glu (32.0 mg, 60.0 μmol, 1.00 eq.) was dissolved together with HOBt (20.4 mg, 150.0 μmol, 2.50 eq.) and EDC*HCl (28.8 mg, 150.0 μmol, 2.50 eq.) in dry DMF (1.0 mL) and stirred under an argon atmosphere at room temperature. After 1 h, a colorless solution of FAPi*TFA (65.4 mg, 120.0 μmol, 2.00 eq.), DIPEA (23.3 mg, 30 μL, 180.0 μmol, 3.00 eq.) and dry DMF (0.5 mL) was added. A further 3 h later, HOBt (7.8 mg, 60.0 μmol, 1.00 eq.) and EDC*HCl (11.4 mg, 60.0 μmol, 1.00 eq.) were added again. Shortly thereafter, further FAPi*TFA (16.5 mg, 30.0 μmol, 0.50 eq.), dissolved in DIPEA (7.8 mg, 10 μL, 60.0 μmol, 1.00 eq.) and 0.5 mL of dry DMF, was added. The next day, another half equivalent of HOBt (3.9 mg, 30.0 μmol, 0.50 eq.) and EDC*HCl (5.7 mg, 30.0 μmol, 0.5 eq.) was added, and the reaction was ended after a further 4 h. The DMF was removed under reduced pressure and, after purification by column chromatography (CHCl₃:MeOH (100:10)), Fmoc-PEG2.Glu.(FAPi)₂ (79.1 mg, 58.4 μmol, 97%) was obtained as a pale yellowish solid.

LC-MS (ESI-positive): m/z (%)=452.50 (31, [M+H]³⁺), 678.45 (100, [M+H]²⁺), 679.25 (13, [M+H]²⁺), 1355.85 (9, [M+H]⁺), calculated for C₆₉H₇₄F₄N₁₂O₁₃: 1354.54 [M]⁺.

### PEG2.Glu.(FAPi)₂ ((2S)-2-(3-(2-(2-Aminoethoxy)ethoxy)propanamido)-N¹,N⁵-bis(4-((4-((2-(2-cyano-4,4-difluoropyrrolidin-1-yl)-2-oxoethyl)carbamoyl)quinolin-6-yl)oxy)butyl)-pentanediamide)

Fmoc-PEG2.Glu.(FAPi)₂ (67.0 mg, 50.0 μmol, 1.00 eq.) was dissolved in 1.0 mL of dry DMF, and 10% piperidine (0.1 mL) was added. The pale yellowish solution was stirred at room temperature for 2 h, and then the solvent was removed under reduced pressure. PEG2.Glu.(FAPi)₂ was obtained in quantitative yield, which was used without further purification.

LC-MS (ESI-positive): m/z (%)=378.40 (100, [M+H]³⁺), 567.35 (26, [M+H]²⁺), 1133.35 (3, [M+H]⁺), calculated for C₅₄H₆₄F₄N₁₂O₁₁: 1132.48 [M]⁺.

The synthesis of the labeling precursor DOTA.PEG2.Glu.(FAPi)₂ is shown below in scheme 18.

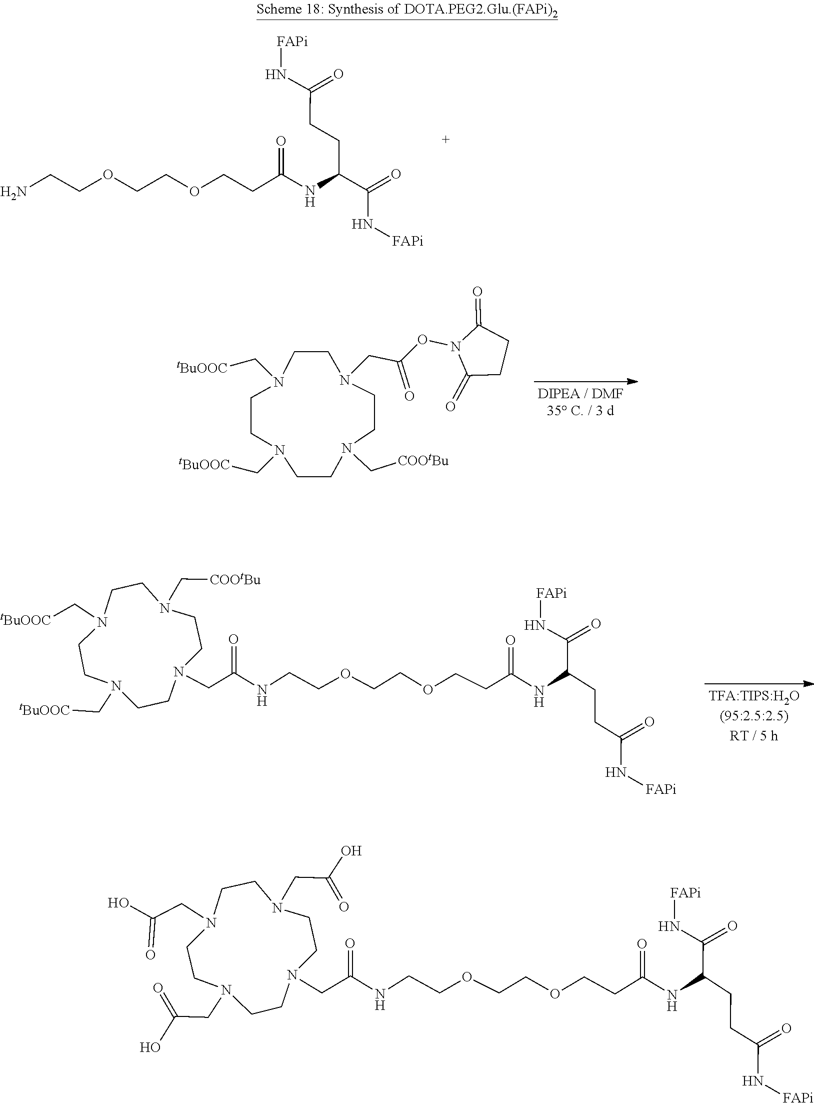

### DOTA(tBu)₃.PEG2.Glu.(FAPi)₂ (2,2′,2″-(10-(2-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1,5-dioxopentan-2-yl)amino)-2-oxoethyl)-1,4,7,10-tetraazacyclododecan-1,4,7-triyl)triacetic acid tert-butyl ester)

PEG2.Glu.(FAPi)₂ (13.4 mg, 20.0 μmol, 1.00 eq.) was dissolved in DMF (0.4 mL) and 1 vol % of DIPEA (10.4 mg, 14 μL, 82.3 μmol), and then DOTA(tBu)₃-NHS (22.7 mg, 20.0 μmol, 1.00 eq.), likewise dissolved in DMF (1.0 mL), was added. The mixture was stirred at 35° C. for three days, and then the DMF was removed under reduced pressure. The yellowish-brown oil was converted further without further workup.

HPLC-MS (ESI-positive): m/z (%)=432.70 (55, [M+H]⁴⁺), 576.60 (26, [M+H]³⁺), 864.90 (18, [M+Na]²⁺), ₁₆₈₇.84 (1, [M+H]⁺), 1709.82 (1, [M+Na]⁺), calculated for C₈₂H₁₁₄F₄N₁₆O₁₈: 1686.84 [M]⁺.

### DOTA.PEG2.Glu.(FAPi)₂ (2,2′,2″-(10-(2-(((S)-1,5-bis((4-((4-((2-((S)-2-cyano-4,4-difluoropyrrolidin-1-yl)-2-oxoethyl)carbamoyl)quinolin-6-yl)oxy)butyl)amino)-1.5-dioxopentan-2-yl)amino)-2-oxoethyl)-1,4,7,10-tetraazacyclododecane-1,4,7-triyl)triacetic acid)

To DOTA(tBu)₃.PEG₂.Glu.(FAPi)₂ were added 50 μL of water, 50 μL of TIPS and 1.5 mL of trifluoroacetic acid (TFA). The brown solution was stirred at room temperature for 5 h, and the solvents were removed under reduced pressure. The resultant dark brown oil was purified by semipreparative RP-HPLC (22-23% ACN in 20 min, t_{R}=16-17 min), and DOTA.PEG₂.Glu.(FAPi)₂ (1.8 mg, 1.2 μmol, 6%) was obtained as a yellowish solid.

LC-MS (ESI-positive): m/z (%)=380.60 (66, [M+H]⁴⁺), 507.30 (100, [M+H]³⁺), 760.30 (12, [M+H]²⁺), 1519.55 (4, [M+H]⁺), 1541.75 (7, [M+Na]⁺), calculated for C₇₀H₉₀F₄N₁₆O₁₈: 1518.66 [M]⁺.

The synthesis of the labeling precursor DOTAGA.PEG2.Glu.(FAPi)₂ is shown below in scheme 19.

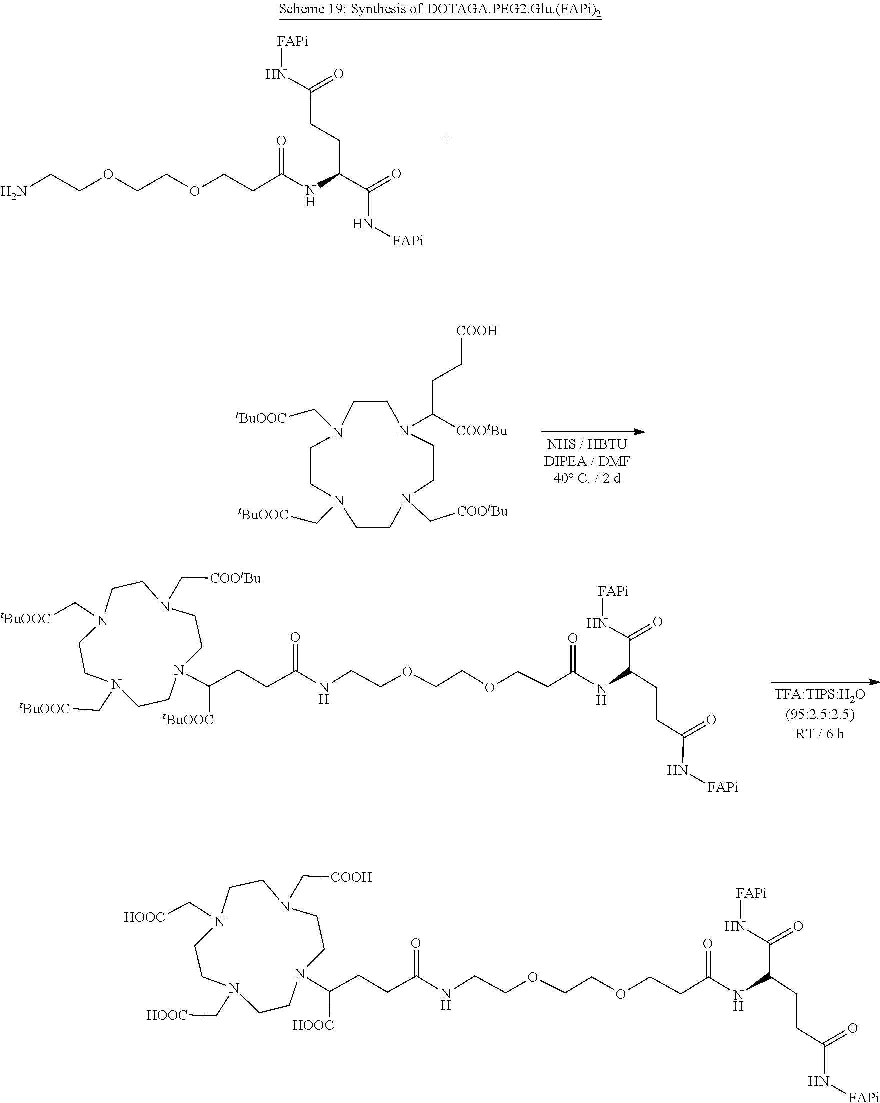

### DOTAGA(tBu)₄.PEG2.Glu.(FAPi)₂ (2,2′,2″-(10-((20S)-28-((4-((2-(2-cyano-4,4-difluoropyrrolidin-1-yl)-2-oxoethyl)carbamoyl)quinolin-6-yl)oxy)-20-((4-((4-((2-(2-cyano-4,4-difluoropyrrolidin-1-yl)-2-oxoethyl)carbamoyl)quinolin-6-yl)oxy)butyl)carbamoyl)-2.2-dimethyl-4,8,18,23-tetraoxo-3,12,15-trioxa-9,19,24-triazaoctacosan-5-yl)-1,4,7,10-tetraazacyclododecane-1,4,7-triyl)triacetic acid tert-butyl ester)

DOTAGA(tBu)₄ (10.0 mg, 14.3 μmol, 1.00 eq.) was dissolved together with HBTU (10.8 mg, 28.6 μmol, 2.00 eq.) in 0.8 mL of dry MeCN, NHS (3.3 g, 28.6 μmol, 2.00 eq.) was added, and the colorless solution was stirred under an argon atmosphere. After 6 h, further HBTU (5.4 mg, 14.3 μmol, 1.00 eq.) and NHS (1.6 mg, 14.3 μmol, 1.00 eq.) were added.

Glu.(FAPi)₂ (8.2 mg, 8.7 μmol, 1.00 eq.) was dissolved in 0.4 mL of dry MeCN and 1.0 mL of dry DMF, 1 vol % of DIPEA (19 mg, 25 μL, 147.0 μmol) was added, and the mixture was added to the red DOTAGA(tBu)₄-NHS solution (11.4 mg, 14.3 μmol, 1.65 eq. in 1.1 mL of MeCN). The reaction was stirred at 40° C. for 24 h and then further PEG2.Glu.(FAPi)₂ (8.2 mg, 8.7 μmol, 1.00 eq.) was added. After a further 24 h, the solvent was removed under reduced pressure and a yellowish oil was obtained, which was used in the next stage without further workup.

HPLC-MS (ESI-positive): m/z (%)=454.99 (100, [M+H]⁴⁺), 606.31 (55, [M+H]³⁺), 908.97 (34, [M+H]²⁺), 1815.93 (4, [M+H]⁺), 1837.91 (2, [M+Na]⁺), calculated for C₈₉H₁₂₆F₄N₁₆O₂₀: 1814.93 [M]⁺.

### DOTAGA.PEG2.Glu.(FAPi)₂ (2,2′,2″-(10-((20S)-28-((4-((2-(2-cyano-4,4-difluoropyrrolidin-1-yl)-2-oxoethyl)carbamoyl)quinolin-6-yl)oxy)-20-((4-((4-((2-(2-cyano-4,4-difluoropyrrolidin-1-yl)-2-oxoethyl)carbamoyl)quinolin-6-yl)oxy)butyl)carbamoyl)-2,2-dimethyl-4,8,18,23-tetraoxo-3,12,15-trioxa-9,19,24-triazaoctacosan-5-yl)-1,4,7,10-tetraazacyclododecane-1,4,7-triyl)triacetic acid)

To DOTAGA(tBu)₄.PEG₂.Glu.(FAPi)₂ were added 50 μL of water, 50 μL of TIPS and 1.5 mL of trifluoroacetic acid (TFA). The dark brown solution was stirred at room temperature for 6 h, and the solvents were removed under reduced pressure. A brown oil was obtained, which was purified by semipreparative RP-HPLC (22% ACN isocratic, t_{R}=17-18 min). DOTAGA.PEG2.Glu.(FAPi)₂ (2.3 mg, 1.5 μmol, 10%) was obtained as a yellowish solid.

LC-MS (ESI-positive): m/z (%)=398.70 (93, [M+H]⁴⁺), 531.30 (100, [M+H]³⁺), 796.20 (8, [M+H]²⁺), 1591.85 (3, [M+H]⁺), calculated for C₇₃H₉₄F₄N₁₆O₂₀: 1590.68 [M]⁺.

### Example 5: DOTA.Glu.Glu.(FAPi)₂, DOTAGA.Glu.Glu.(FAPi)₂

The synthesis of the labeling precursors DOTA.Glu.Glu.(FAPi)₂ and DOTAGA.Glu.Glu.(FAPi)₂ is illustrated below in scheme 20. The first synthesis steps are identical for both compounds.

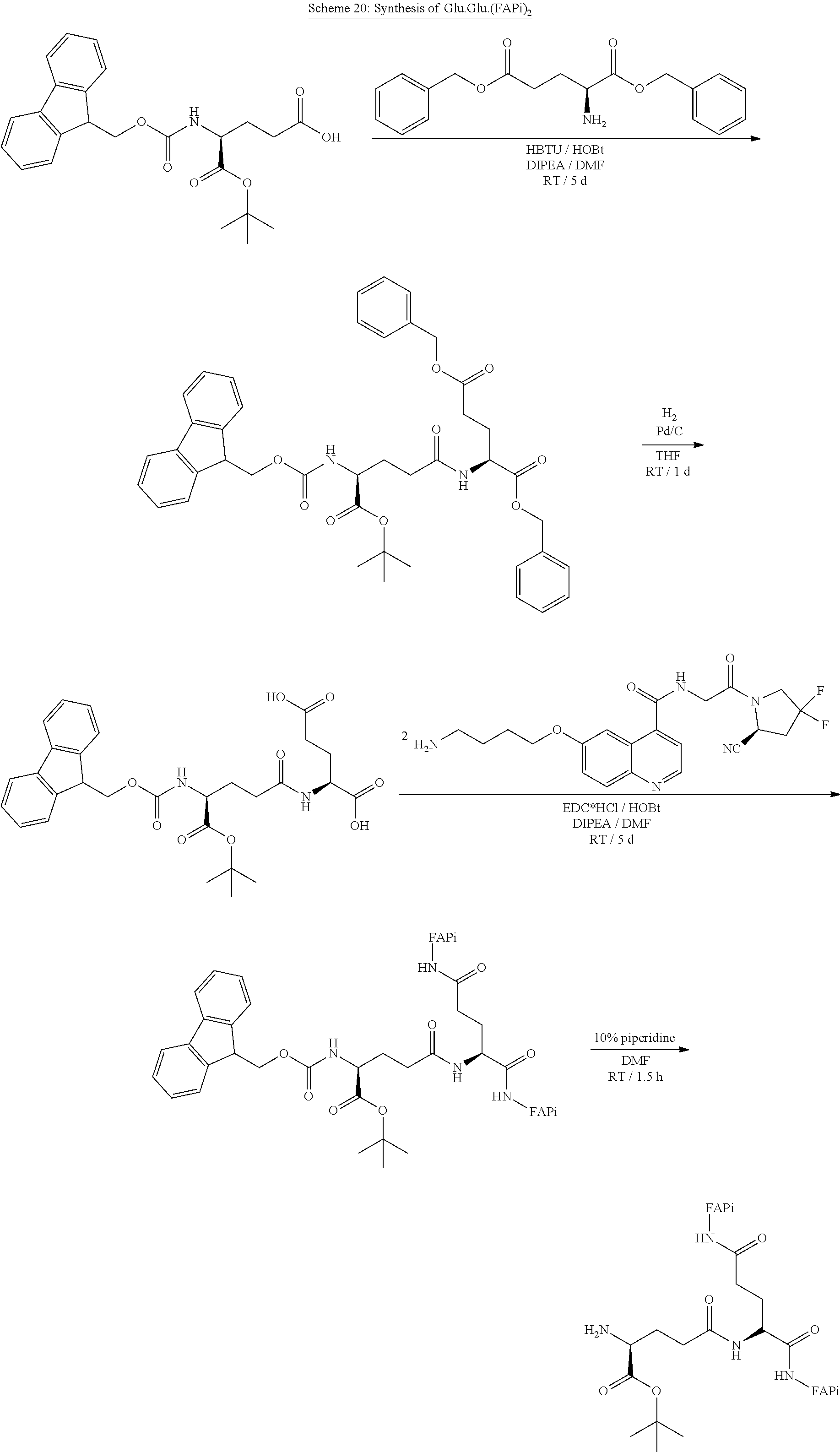

### Fmoc-Glu(OtBu).Glu(OBzl)₂ ((S)-4-((((9H-fluoren-9-yl)methoxy)carbonyl)amino)-5-(tert-butoxy)-5-oxopentanoyl)-L-glutamic acid dibenzyl ester)

Fmoc-Glu-OtBu (400.0 mg, 0.94 mmol, 1.00 eq.) was dissolved in dry DMF (2.0 mL), and DIPEA (151.9 mg, 200 μL, 1.2 mmol, 1.25 eq.) and HATU (393.2 mg, 1.0 mmol, 1.10 eq.) were added. Subsequently, the solution was stirred under an argon atmosphere at 25° C. After one hour, dibenzyl glutamate (384.7 mg, 1.2 mmol, 1.25 eq.) dissolved in dry DMF (1.0 mL) and DIPEA (267.3 mg, 352 μL, 2.1 mmol, 2.20 eq.) were added. The next day, HATU (357.4 mg, 0.9 mmol, 1.00 eq.) and DIPEA (121.5 mg, 156 μL, 0.9 mmol, 1.00 eq.) were added again. Three days later, 1.00 eq. HATU and, one hour later, a solution of dibenzyl glutamate (153.87 mg, 0.5 mmol, 0.50 eq.) and 1.00 eq. of DIPEA in 0.5 mL of DMF were added. After a further day at 25° C., the solvent was removed under reduced pressure and the product was purified by column chromatography (cyclohexane:ethyl acetate (CH:EA, 3:1)). Fmoc-Glu(OtBu).Glu(OBzl)₂ (657.3 mg, 0.89 mmol, 95%) was obtained as a pale yellowish solid.

LC-MS (ESI-positive): m/z (%)=679.20 (27, [M−^{t}Bu+H]⁺), 680.30 (11, [M−^{t}Bu+H]⁺), 735.50 (100, [M+H]⁺), 736.15 (15, [M+H]⁺), calculated for C₄₃H₄₆N₂O₉: 734.32 [M]⁺.

### Fmoc-Glu(OtBu).Glu ((S)-4-((((9H-fluoren-9-yl)methoxy)carbonyl)amino)-5-(tert-butoxy)-5-oxopentanoyl)-L-glutamic acid)

Fmoc-Glu(OtBu).Glu(OBzl)₂ (25.0 mg, 34.0 μmol, 1.00 eq.) was dissolved in 1.0 mL of dry THF, and palladium on activated carbon (10 wt % Pd, 7.25 mg, 78.0 μmol, 2.00 eq.) was added. The suspension was stirred under a hydrogen atmosphere overnight, and the next day was filtered through Celite. The residue was washed with THF, and the latter was then removed under reduced pressure. Fmoc-Glu(OtBu).Glu (17.8 mg, 32.1 μmol, 94%) was obtained as a colorless solid.

LC-MS (ESI-positive): m/z (%)=499.05 (57, [M−^{t}Bu+H]⁺), 500.15 (11, [M−^{t}Bu+H]⁺), 555.25 (100, [M+H]), 556.15 (21, [M+H]), calculated for C₂₉H₃₄N₂O₉: 554.23 [M].

### Fmoc-Glu(OtBu).Glu.(FAPi)₂ (N²-(((9H-fluoren-9-yl)methoxy)carbonyl)-N⁵-((2S)-1,5-bis((4-((4-((2-(2-cyano-4,4-difluoropyrrolidin-1-yl)-2-oxoethyl)carbamoyl)quinolin-6-yl)oxy)butyl)-amino)-1,5-dioxopentan-2-yl)-L-glutamic acid tert-butyl ester)

Fmoc-Glu(OtBu).Glu (33.3 mg, 60.0 μmol, 1.00 eq.) was dissolved together with HOBt (20.4 mg, 15.0 μmol, 2.50 eq.) and EDC*HCl (28.8 mg, 15.0 μmol, 2.50 eq.) in dry DMF (2.5 mL) and stirred under an argon atmosphere at room temperature for 1 h. Then FAPi*TFA (65.4 mg, 12.0 μmol, 2.00 eq.) dissolved in dry DMF (0.5 mL) and DIPEA (23.3 mg, 31 μL, 18.0 μmol, 3.00 eq.) were added. The next day, a further equivalent of HOBt (7.8 mg, 60.0 μmol, 1.00 eq.) and EDC*HCl (11.4 mg, 60.0 μmol, 1.00 eq.) and, 30 min later, a half equivalent of FAPi*TFA (16.5 mg, 30.0 μmol, 0.50 eq.) dissolved in one equivalent of DIPEA (7.8 mg, 10 μL, 60.0 μmol, 1.00 eq.) and 0.5 mL of DMF were added. 24 h later, HOBt (3.9 mg, 30.0 μmol, 0.50 eq.) and EDC*HCl (5.7 mg, 30.0 μmol, 0.50 eq.) were added again and, after one hour, further FAPi*TFA (16.5 mg, 30.0 μmol, 0.50 eq.) and DIPEA (3.9 mg, 5 μL, 30.0 μmol, 0.50 eq.) dissolved in DMF (0.5 mL). This step was repeated once again the next day. The pale yellowish solution was then stirred for a further day, and then the solvent was removed under reduced pressure. By means of column chromatography (CHCl₃:MeOH (100:10)), Fmoc-Glu(OtBu).Glu.(FAPi)₂ (86.7 mg, 62.8 μmol, 79%) was obtained as a yellowish solid.

LC-MS (ESI-positive): m/z (%)=461.25 (32, [M+H]³⁺), 691.45 (100, [M+H]²⁺), 692.25 (12, [M+H]²⁺), 1381.95 (12, [M+H]⁺), calculated for C₇₁H₇₆F₄N₁₂O₁₃: 1380.56 [M]⁺.

### Glu(OtBu).Glu.(FAPi)₂ (N⁵-((2S)-1,5-bis((4-((4-((2-(2-cyano-4,4-difluoropyrrolidin-1-yl)-2-oxoethyl)carbamoyl)quinolin-6-yl)oxy)butyl)amino)-1,5-dioxopentan-2-yl)-L-glutamic acid tert-butyl ester)

Fmoc-Glu(OtBu).Glu.(FAPi)₂ (72.2 mg, 52.2 μmol, 1.00 eq.) was dissolved in dry DMF (1.0 mL), 10% piperidine (0.1 mL) was added and the mixture was stirred at room temperature for 90 min. Subsequently, the solvent was removed under reduced pressure, and a yellowish oil was obtained, which was used directly in the next stage without further purification.

LC-MS (ESI-positive): m/z (%)=387.10 (99, [M+H]³⁺), 580.35 (37, [M+H]²⁺), 1159.30 (4, [M+H]⁺), calculated for C₅₆H₆₆F₄N₁₂O₁₁: 1158.49 [M]⁺.

The synthesis of the labeling precursor DOTA.Glu.Glu.(FAPi)₂ is shown below in scheme 21.

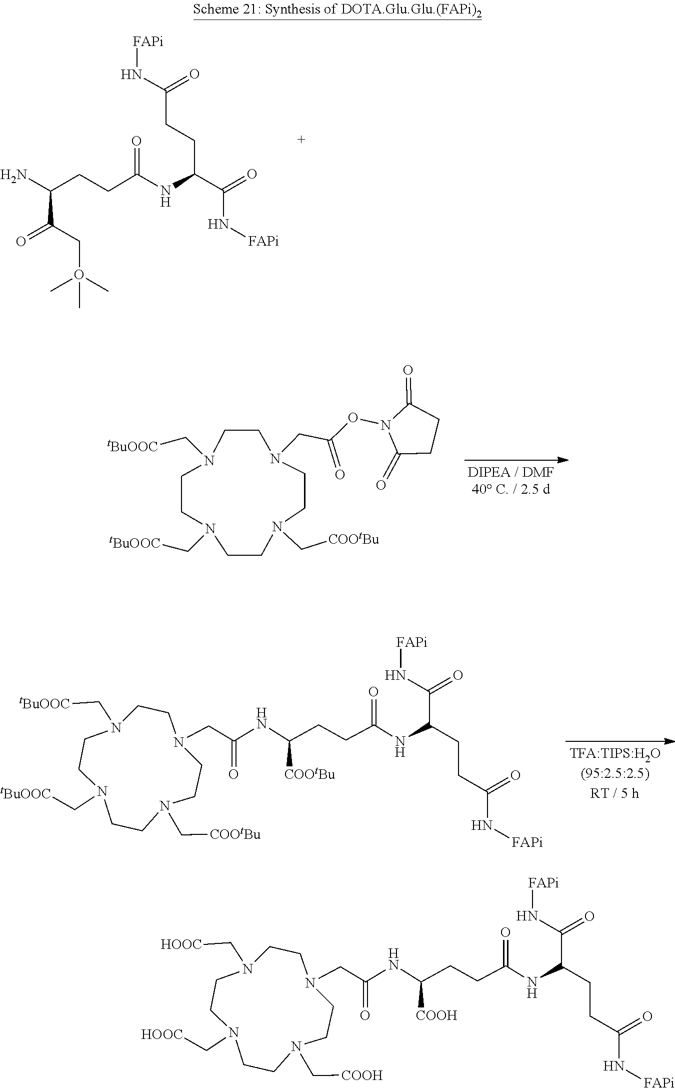

### DOTA(tBu)₃.Glu(OtBu).Glu.(FAPi)₂ (2,2′,2″-(10-(2-(((2S)-5-(((2S)-1,5-bis((4-((4-((2-(2-cyano-4,4-difluoropyrrolidin-1-yl)-2-oxoethyl)carbamoyl)quinolin-6-yl)oxy)butyl)amino)-1,5-dioxopentan-2-yl)amino)-1-(tert-butoxy)-1,5-dioxopentan-2-yl)amino)-2-oxoethyl)-1,4,7,10-tetraazacyclododecan-1,4,7-triyl)triacetic acid tert-butyl ester)

DOTA(tBu)₃-NHS (17.5 mg, 26.1 μmol, 1.00 eq.) was dissolved in 1.0 mL of dry DMF, and Glu(OtBu).Glu.(FAPi)₂ (30.3 mg, 26.1 μmol, 1.00 eq.) dissolved in 0.5 mL of DMF and 1 vol % of DIPEA (11.4 mg, 15 μL, 88.2 μmol) was added. The pale yellowish solution was stirred at 40° C. under an argon atmosphere for 24 h and then the solvent was removed under reduced pressure.

Subsequently, the yellowish oil obtained was dissolved in 0.5 mL of dry DMF, and DIPEA (3.4 mg, 4 μL, 26.1 μmol, 1.00 eq.) was added. DOTA (17.5 mg, 26.1 μmol, 1.00 eq.), HATU (14.9 mg, 39.2 μmol, 1.50 eq.) and DIPEA (6.7 mg, 9 μL, 52.2 μmol, 2.00 eq.) were initially charged in 0.5 mL of dry DMF, and the mixture was stirred for one hour and then added. The yellowish solution was stirred at 30° C. under an argon atmosphere for 24 h and then further HATU (1.50 eq.) and DIPEA (2.00 eq.) were added. After a further 6 h at 40° C., HATU (1.50 eq.) and DIPEA (2.00 eq.) were added once more. The next day, the solvent was removed under reduced pressure and a yellowish oil was obtained, which was converted further without further workup.

HPLC-MS (ESI-positive): m/z (%)=429.47 (9, [M+H]⁴⁺), 571.96 (10, [M+H]³⁺), 857.43 (3, [M+H]²⁺), calculated for C₈₄H₁₁₆F₄N₁₆O₁₈: 1712.94 [M]⁺.

### DOTA.Glu.Glu.(FAPi)₂ (2,2′,2″-(10-(2-(((15)-4-(((25)-1,5-bis((4-((4-((2-(2-cyano-4,4-difluoropyrrolidin-1-yl)-2-oxoethyl)carbamoyl)quinolin-6-yl)oxy)butyl)amino)-1,5-dioxopentan-2-yl)amino)-1-carboxy-4-oxobutyl)amino)-2-oxoethyl)-1,4,7,10-tetraazacyclododecane-1,4,7-triyl)triacetic acid)

To DOTA(tBu)₃.Glu(OtBu).Glu.(FAPi)₂ were added 50 μL of water, 50 μL of TIPS and 1.5 mL of trifluoroacetic acid (TFA). The yellowish solution was stirred at room temperature for 5 h and the solvents were removed under reduced pressure. The crude product was purified by semipreparative RP-HPLC (22-23% ACN in 20 min, t_{R}=13-14 min), and DOTA.Glu.Glu.(FAPi)₂ (6.6 mg, 4.4 μmol, 17%) was obtained as a yellowish solid.

LC-MS (ESI-positive): m/z (%)=373.05 (84, [M+H]⁴⁺), 497.15 (100, [M+H]³⁺), 745.70 (5, [M+H]²⁺), 1511.35 (1, [M+Na]⁺), calculated for C₆₈H₈₄F₄N₁₆O₁₈: 1488.61 [M]⁺.

The synthesis of the labeling precursor DOTAGA.Glu.Glu.(FAPi)₂ is shown below in scheme 22.

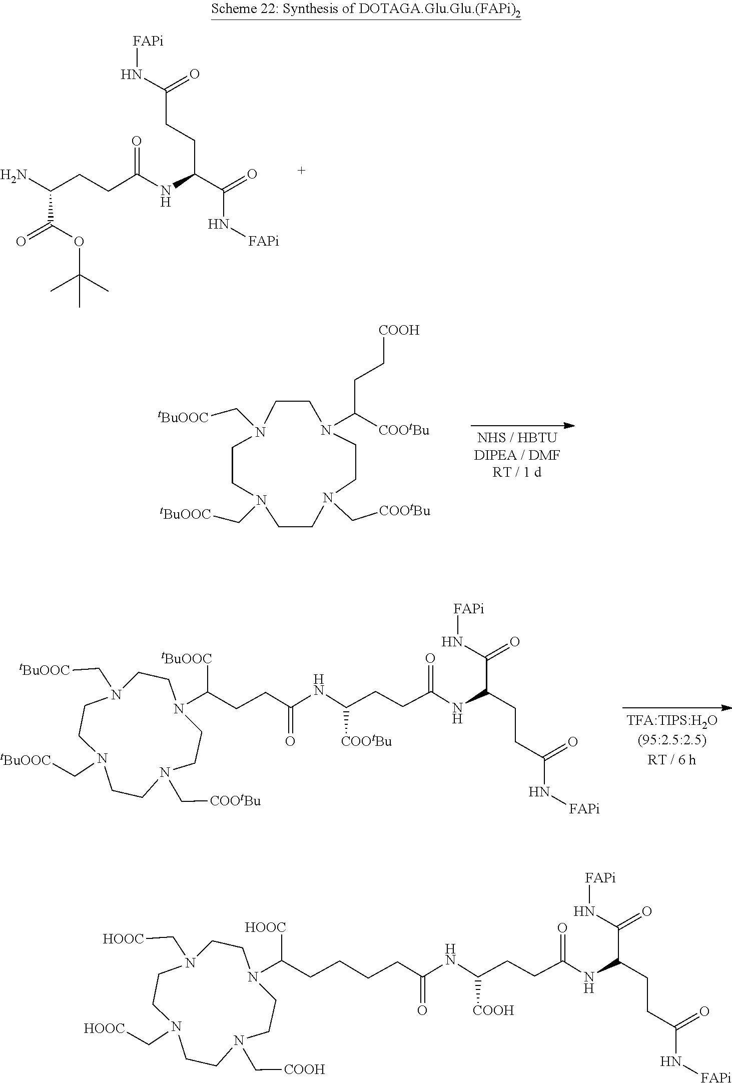

### DOTAGA(tBu)₄.Glu(OtBu).Glu.(FAPi)₂ (2,2′,2″-(10-((10S,15S)-10-(tert-butoxycarbonyl)-23-((4-((2-(2-cyano-4,4-difluoropyrrolidin-1-yl)-2-oxoethyl)carbamoyl)quinolin-6-yl)oxy)-15-((4-((4-((2-(2-cyano-4,4-difluoropyrrolidin-1-yl)-2-oxoethyl)carbamoyl)quinolin-6-yl)oxy)butyl)-carbamoyl)-2,2-dimethyl-4,8,13,18-tetraoxo-3-oxa-9,14,19-triazatricosan-5-yl)-1,4,7,10-tetraazacyclododecane-1,4,7-triyl)triacetic acid tert-butyl ester)

DOTAGA(tBu)₄ (22.4 mg, 32.6 μmol, 1.00 eq.) was dissolved together with HBTU (24.7 mg, 65.3 μmol, 2.00 eq.) in dry MeCN (1.0 mL), and NHS (7.5 mg, 65.3 μmol, 2.00 eq.) was added. The colorless solution was stirred under an argon atmosphere for 4 h, and HBTU (12.4 mg, 32.6 μmol, 1.00 eq.) dissolved in DMF (0.2 mL) and NHS (3.8 mg, 32.6 μmol, 1.00 eq.) were added. Subsequently, Glu(OtBu).Glu.(FAPi)₂ (30.3 mg, 26.1 μmol, 1.00 eq.) dissolved in DMF (1.0 mL) and 1 vol % of DIPEA (19 mg, 25 μL, 147.0 μmol) was added. The colorless solution was stirred at room temperature overnight and, the next day, the solvent was removed under reduced pressure. A yellowish oil was obtained and was converted further without workup.

HPLC-MS (ESI-positive): m/z (%)=461.49 (52, [M+H]⁴⁺), 614.99 (100, [M+H]³⁺), 921.97 (56, [M+H]²⁺), 1841.94 (35, [M+H]⁺), 1863.93 (6, [M+Na]⁺), calculated for C₉₁H₁₂₈F₄N₁₆O₂₀: 1840.94 [M].

### DOTAGA.Glu.Glu.(FAPi)₂ (2,2′,2″-(10-(4-(((1S)-4-(((25)-1,5-bis((4-((4-((2-(2-cyano-4,4-difluoropyrrolidin-1-yl)-2-oxoethyl)carbamoyl)quinolin-6-yl)oxy)butyl)amino)-1,5-dioxopentan-2-yl)amino)-1-carboxy-4-oxobutyl)amino)-1-carboxy-4-oxobutyl)-1,4,7,10-tetraazacyclododecane-1,4,7-triyl)triacetic acid)

To DOTAGA(tBu)₄.Glu(OtBu).Glu.(FAPi)₂ were added 50 μL of water, 50 μL of TIPS and 1.5 mL of trifluoroacetic acid (TFA). The yellowish solution was stirred at room temperature for 6 h, and the solvents were removed under reduced pressure. The crude product was purified by semipreparative RP-HPLC (22% ACN isocratic, t_{R}=14-15 min), and DOTAGA.Glu.Glu.(FAPi)₂ (2.0 mg, 1.3 μmol, 5%) was obtained as a yellowish solid.

LC-MS (ESI-positive): m/z (%)=391.10 (78, [M+H]⁴⁺), 401.15 (19, [M+ACN+H]⁴⁺), 521.30 (100, [M+H]³⁺), 781.75 (6, [M+H]²⁺), 1561.65 (3, [M+H]⁺) calculated for C₇₁H₈₈F₄N₁₆O₂₀: 1560.63 [M]*.

### Example 6

Examples of inventive compounds without spacer units (S1, S2, S3) are shown below.

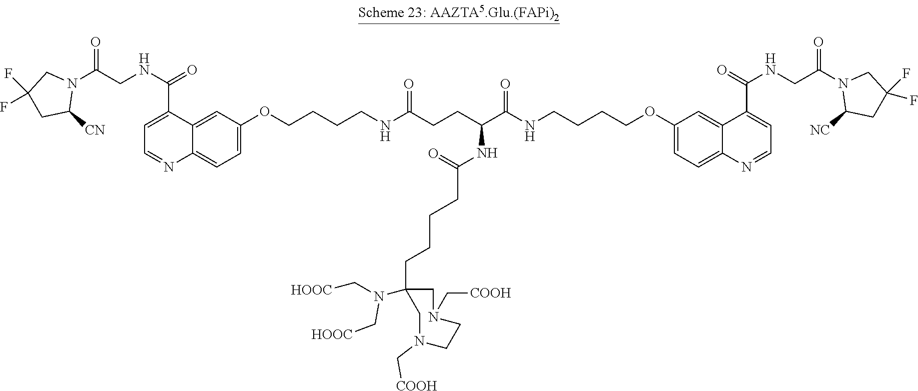 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCCCC3(N(CC(=O)O)CC(=O)O)CN(CC(=O)O)CCN(CC(=O)O)C3)cc12]

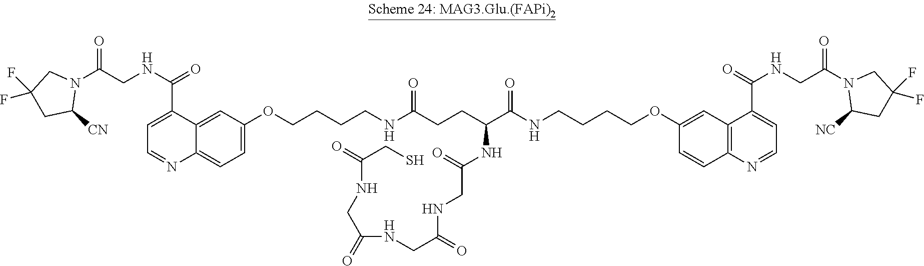 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CNC(=O)CNC(=O)CNC(=O)CS)cc12]

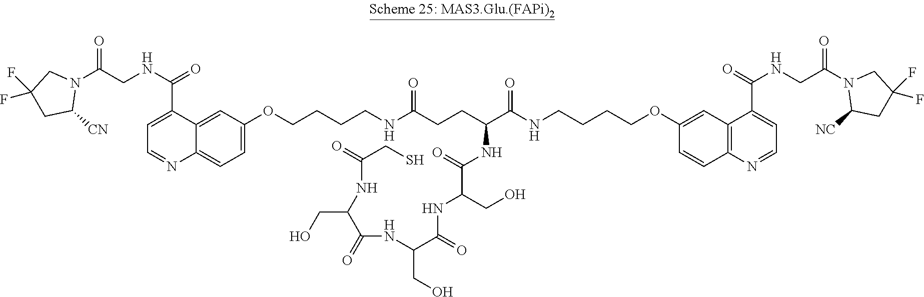 [SMILES: N#C[C@@H]1CC(F)(F)CN1C(=O)CNC(=O)c1ccnc2ccc(OCCCCNC(=O)CC[C@H](NC(=O)C(CO)NC(=O)C(CO)NC(=O)C(CO)NC(=O)CS)C(=O)NCCCCOc3ccc4nccc(C(=O)NCC(=O)N5CC(F)(F)C[C@H]5C#N)c4c3)cc12]

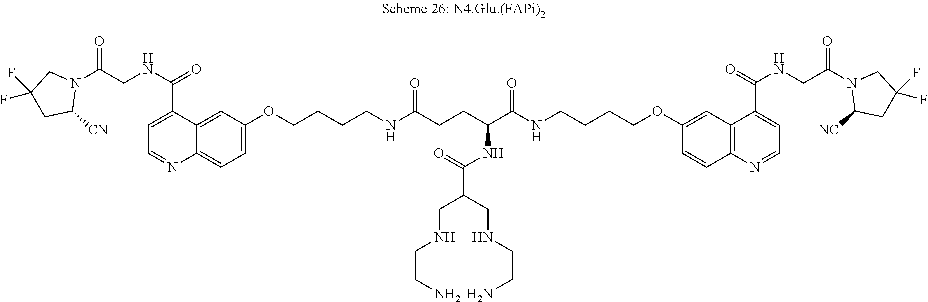 [SMILES: N#C[C@@H]1CC(F)(F)CN1C(=O)CNC(=O)c1ccnc2ccc(OCCCCNC(=O)CC[C@H](NC(=O)C(CNCCN)CNCCN)C(=O)NCCCCOc3ccc4nccc(C(=O)NCC(=O)N5CC(F)(F)C[C@H]5C#N)c4c3)cc12]

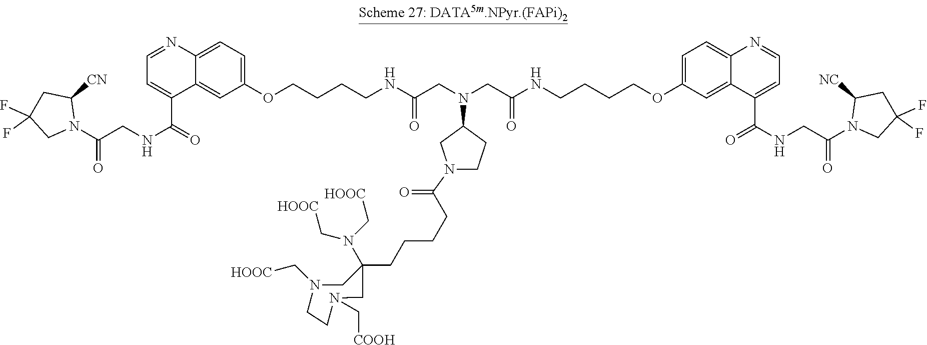 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCCCC4(N(CC(=O)O)CC(=O)O)CN(CC(=O)O)CCN(CC(=O)O)C4)C3)cc12]

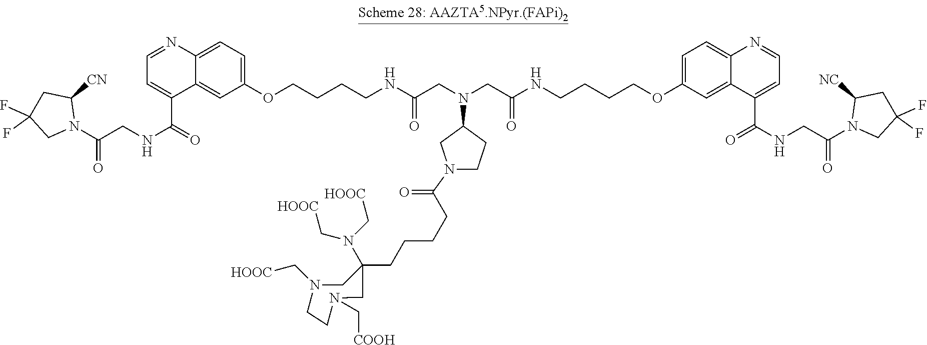 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCCCC4(N(CC(=O)O)CC(=O)O)CN(CC(=O)O)CCN(CC(=O)O)C4)C3)cc12]

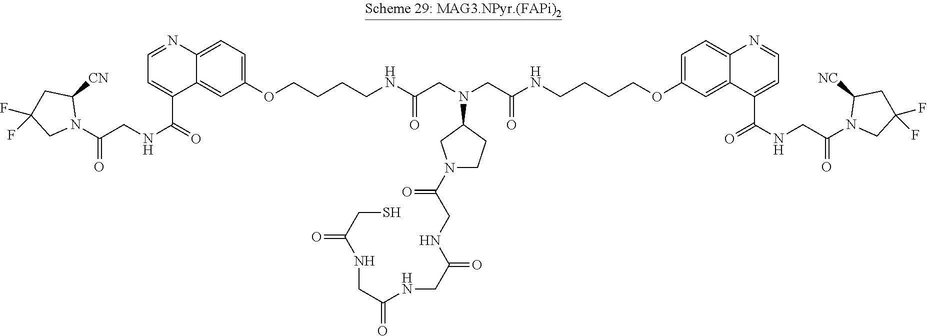 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CNC(=O)CNC(=O)CNC(=O)CS)C3)cc12]

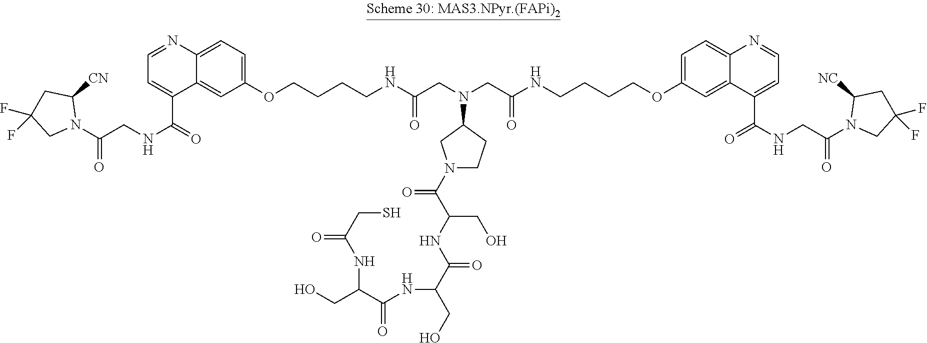 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)C(CO)NC(=O)C(CO)NC(=O)C(CO)NC(=O)CS)C3)cc12]

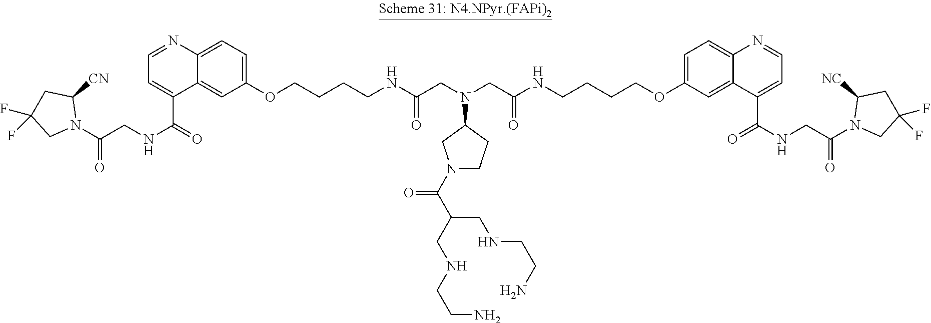 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)C(CNCCN)CNCCN)C3)cc12]

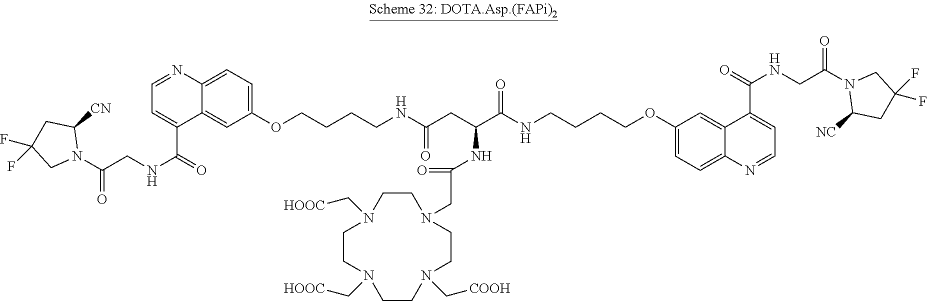 [SMILES: N#C[C@@H]1CC(F)(F)CN1C(=O)CNC(=O)c1ccnc2ccc(OCCCCNC(=O)C[C@H](NC(=O)CN3CCN(CC(=O)O)CCN(CC(=O)O)CCN(CC(=O)O)CC3)C(=O)NCCCCOc3ccc4nccc(C(=O)NCC(=O)N5CC(F)(F)C[C@H]5C#N)c4c3)cc12]

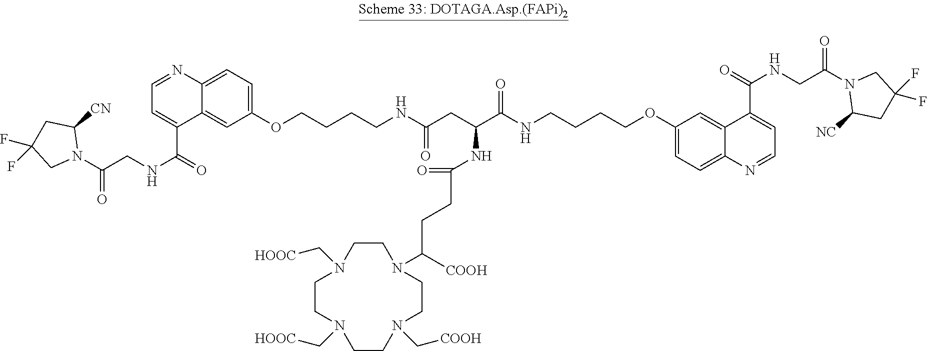 [SMILES: N#C[C@@H]1CC(F)(F)CN1C(=O)CNC(=O)c1ccnc2ccc(OCCCCNC(=O)C[C@H](NC(=O)CCC(C(=O)O)N3CCN(CC(=O)O)CCN(CC(=O)O)CCN(CC(=O)O)CC3)C(=O)NCCCCOc3ccc4nccc(C(=O)NCC(=O)N5CC(F)(F)C[C@H]5C#N)c4c3)cc12]

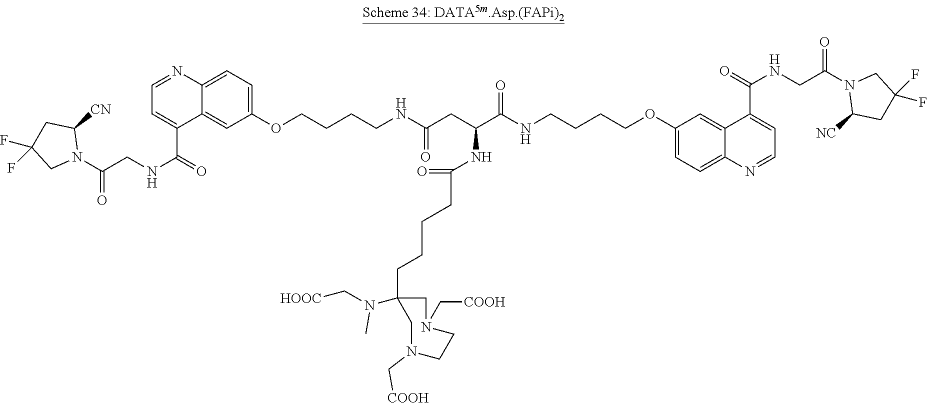 [SMILES: CN(CC(=O)O)C1(CCCCC(=O)N[C@@H](CC(=O)NCCCCOc2ccc3nccc(C(=O)NCC(=O)N4CC(F)(F)C[C@H]4C#N)c3c2)C(=O)NCCCCOc2ccc3nccc(C(=O)NCC(=O)N4CC(F)(F)C[C@H]4C#N)c3c2)CN(CC(=O)O)CCN(CC(=O)O)C1]

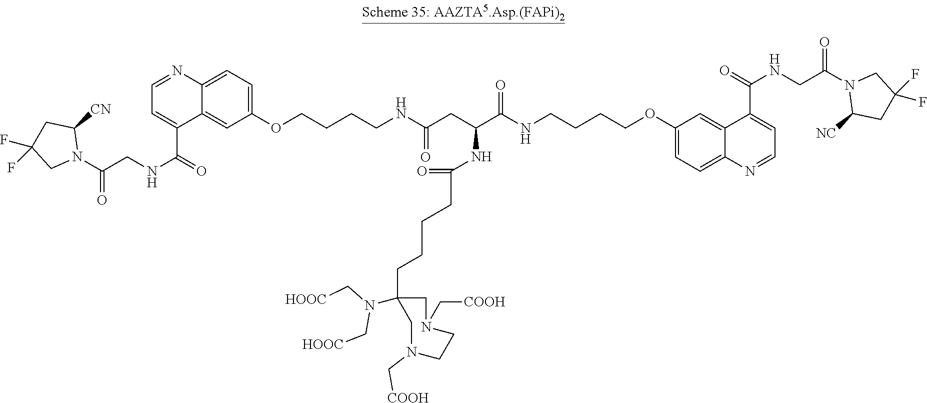 [SMILES: N#C[C@@H]1CC(F)(F)CN1C(=O)CNC(=O)c1ccnc2ccc(OCCCCNC(=O)C[C@H](NC(=O)CCCCC3(N(CC(=O)O)CC(=O)O)CN(CC(=O)O)CCN(CC(=O)O)C3)C(=O)NCCCCOc3ccc4nccc(C(=O)NCC(=O)N5CC(F)(F)C[C@H]5C#N)c4c3)cc12]

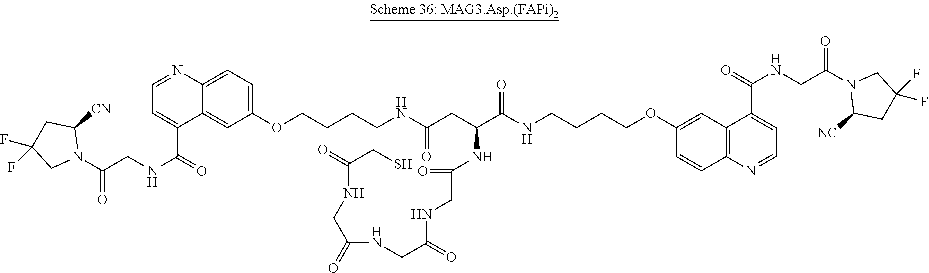 [SMILES: N#C[C@@H]1CC(F)(F)CN1C(=O)CNC(=O)c1ccnc2ccc(OCCCCNC(=O)C[C@H](NC(=O)CNC(=O)CNC(=O)CNC(=O)CS)C(=O)NCCCCOc3ccc4nccc(C(=O)NCC(=O)N5CC(F)(F)C[C@H]5C#N)c4c3)cc12]

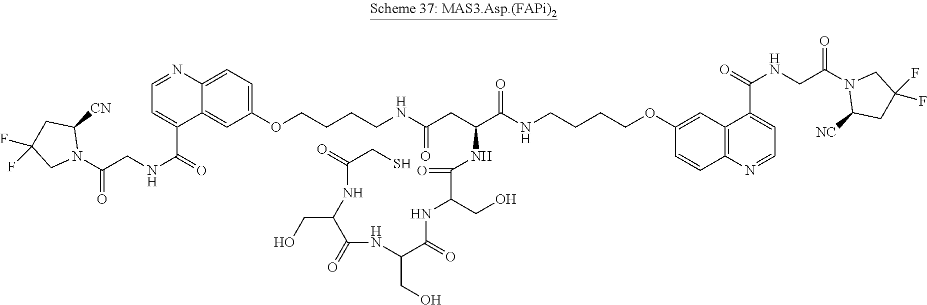 [SMILES: N#C[C@@H]1CC(F)(F)CN1C(=O)CNC(=O)c1ccnc2ccc(OCCCCNC(=O)C[C@H](NC(=O)C(CO)NC(=O)C(CO)NC(=O)C(CO)NC(=O)CS)C(=O)NCCCCOc3ccc4nccc(C(=O)NCC(=O)N5CC(F)(F)C[C@H]5C#N)c4c3)cc12]

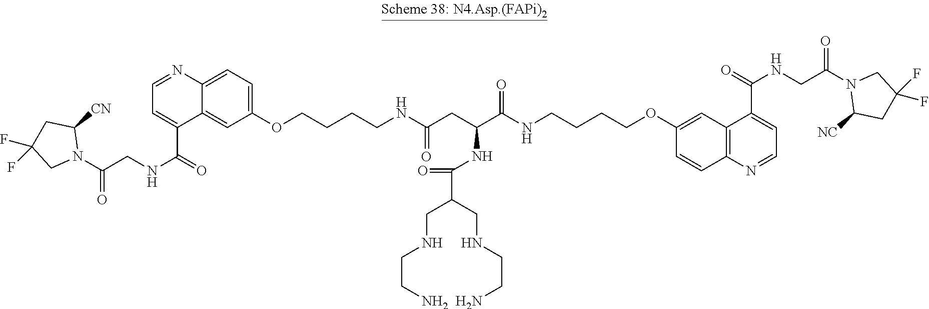 [SMILES: N#C[C@@H]1CC(F)(F)CN1C(=O)CNC(=O)c1ccnc2ccc(OCCCCNC(=O)C[C@H](NC(=O)C(CNCCN)CNCCN)C(=O)NCCCCOc3ccc4nccc(C(=O)NCC(=O)N5CC(F)(F)C[C@H]5C#N)c4c3)cc12]

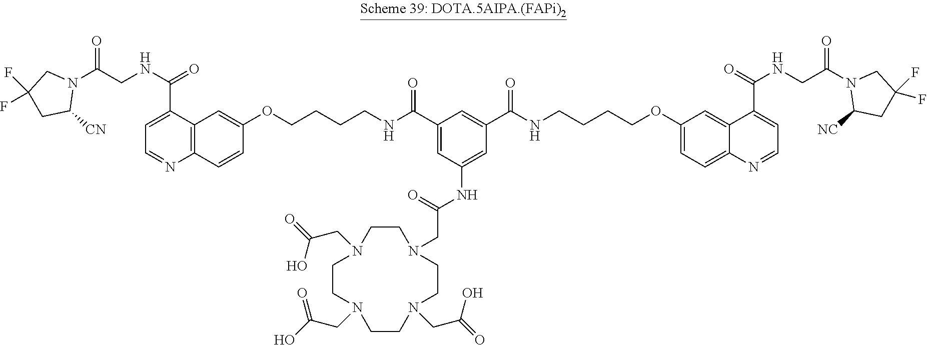 [SMILES: N#C[C@@H]1CC(F)(F)CN1C(=O)CNC(=O)c1ccnc2ccc(OCCCCNC(=O)c3cc(NC(=O)CN4CCN(CC(=O)O)CCN(CC(=O)O)CCN(CC(=O)O)CC4)cc(C(=O)NCCCCOc4ccc5nccc(C(=O)NCC(=O)N6CC(F)(F)C[C@H]6C#N)c5c4)c3)cc12]

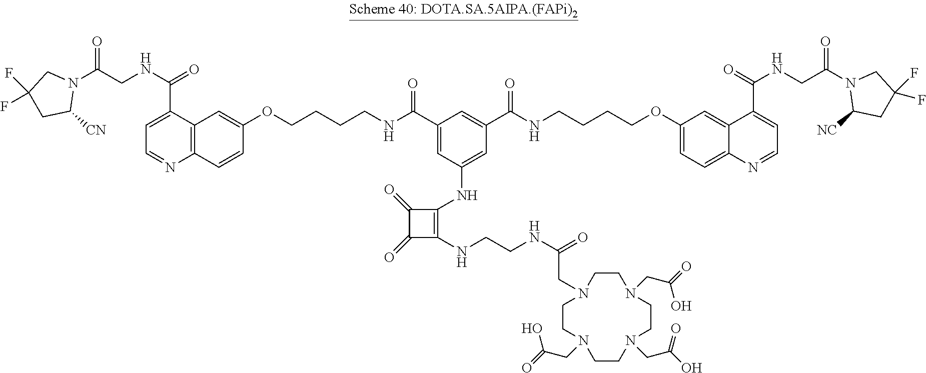 [SMILES: N#C[C@@H]1CC(F)(F)CN1C(=O)CNC(=O)c1ccnc2ccc(OCCCCNC(=O)c3cc(Nc4c(NCCNC(=O)CN5CCN(CC(=O)O)CCN(CC(=O)O)CCN(CC(=O)O)CC5)c(=O)c4=O)cc(C(=O)NCCCCOc4ccc5nccc(C(=O)NCC(=O)N6CC(F)(F)C[C@H]6C#N)c5c4)c3)cc12]

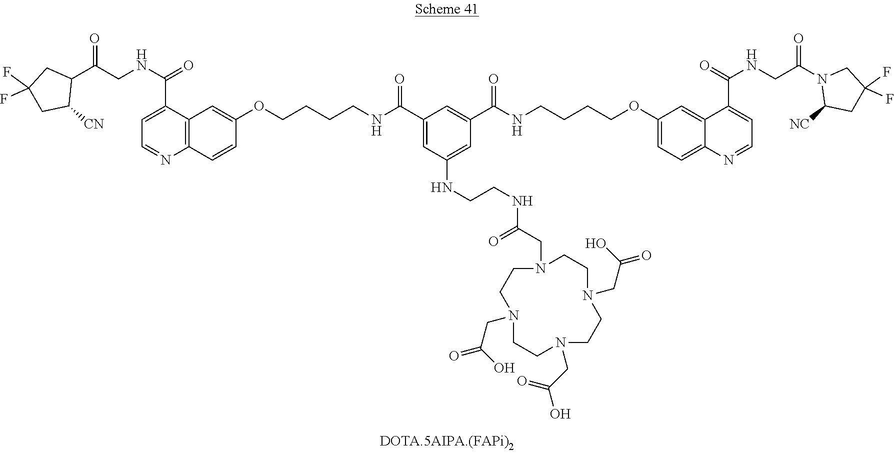 [SMILES: N#C[C@@H]1CC(F)(F)CC1C(=O)CNC(=O)c1ccnc2ccc(OCCCCNC(=O)c3cc(NCCNC(=O)CN4CCN(CC(=O)O)CCN(CC(=O)O)CCN(CC(=O)O)CC4)cc(C(=O)NCCCCOc4ccc5nccc(C(=O)NCC(=O)N6CC(F)(F)C[C@H]6C#N)c5c4)c3)cc12]

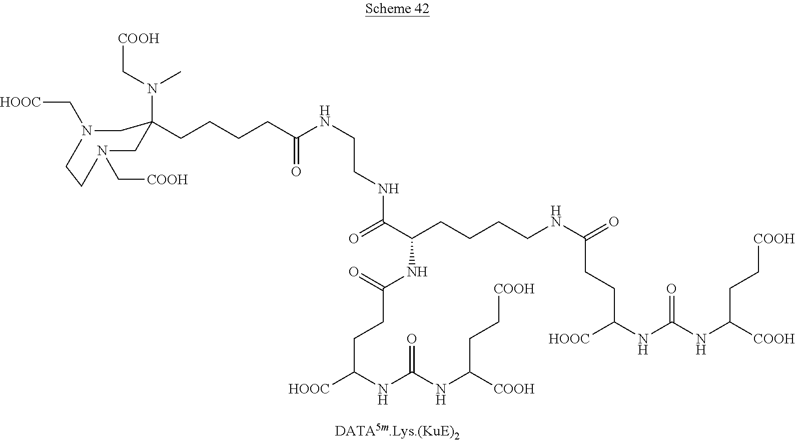 [SMILES: CN(CC(=O)O)C1(CCCCC(=O)NCCNC(=O)[C@H](CCCCNC(=O)CCC(NC(=O)NC(CCC(=O)O)C(=O)O)C(=O)O)NC(=O)CCC(NC(=O)NC(CCC(=O)O)C(=O)O)C(=O)O)CN(CC(=O)O)CCN(CC(=O)O)C1]

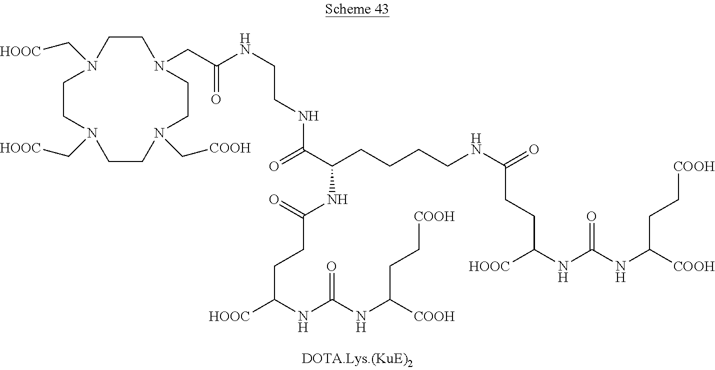 [SMILES: O=C(O)CCC(NC(=O)NC(CCC(=O)NCCCC[C@H](NC(=O)CCC(NC(=O)NC(CCC(=O)O)C(=O)O)C(=O)O)C(=O)NCCNC(=O)CN1CCN(CC(=O)O)CCN(CC(=O)O)CCN(CC(=O)O)CC1)C(=O)O)C(=O)O]

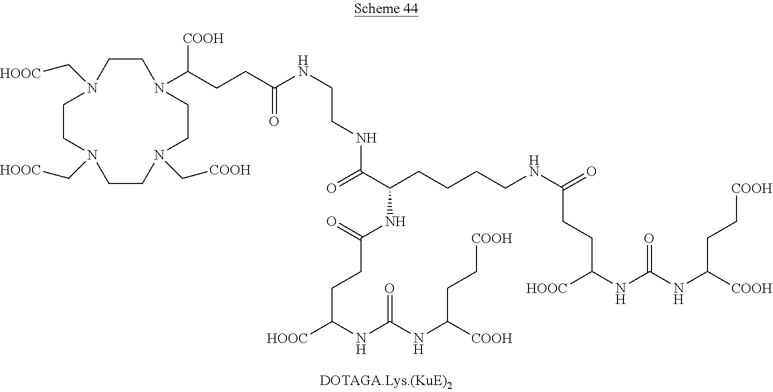 [SMILES: O=C(O)CCC(NC(=O)NC(CCC(=O)NCCCC[C@H](NC(=O)CCC(NC(=O)NC(CCC(=O)O)C(=O)O)C(=O)O)C(=O)NCCNC(=O)CCC(C(=O)O)N1CCN(CC(=O)O)CCN(CC(=O)O)CCN(CC(=O)O)CC1)C(=O)O)C(=O)O]

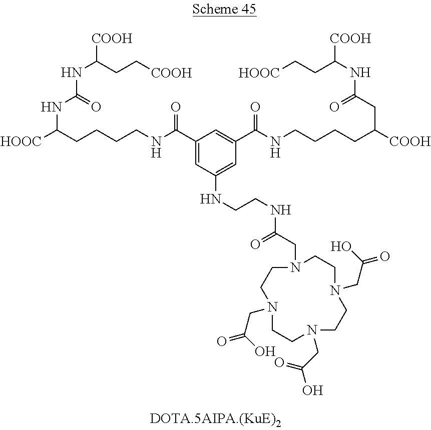 [SMILES: O=C(O)CCC(NC(=O)CC(CCCCNC(=O)c1cc(NCCNC(=O)CN2CCN(CC(=O)O)CCN(CC(=O)O)CCN(CC(=O)O)CC2)cc(C(=O)NCCCCC(NC(=O)NC(CCC(=O)O)C(=O)O)C(=O)O)c1)C(=O)O)C(=O)O]

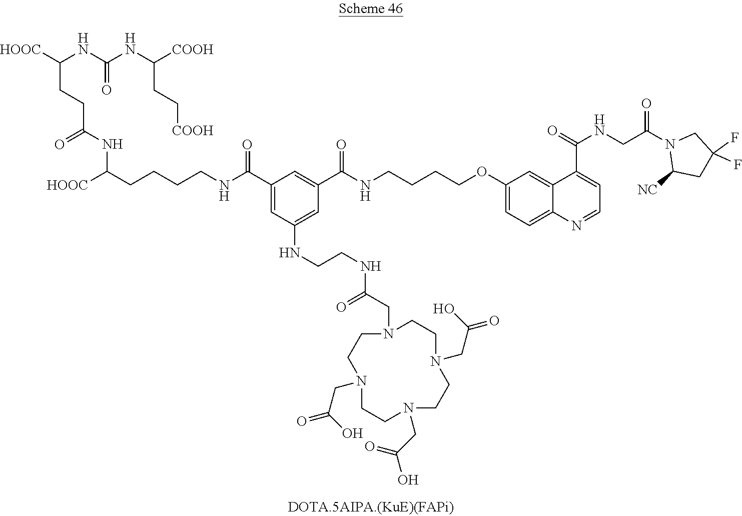 [SMILES: N#C[C@@H]1CC(F)(F)CN1C(=O)CNC(=O)c1ccnc2ccc(OCCCCNC(=O)c3cc(NCCNC(=O)CN4CCN(CC(=O)O)CCN(CC(=O)O)CCN(CC(=O)O)CC4)cc(C(=O)NCCCCC(NC(=O)CCC(NC(=O)NC(CCC(=O)O)C(=O)O)C(=O)O)C(=O)O)c3)cc12]

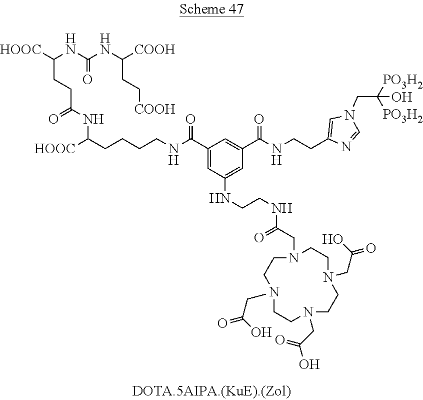

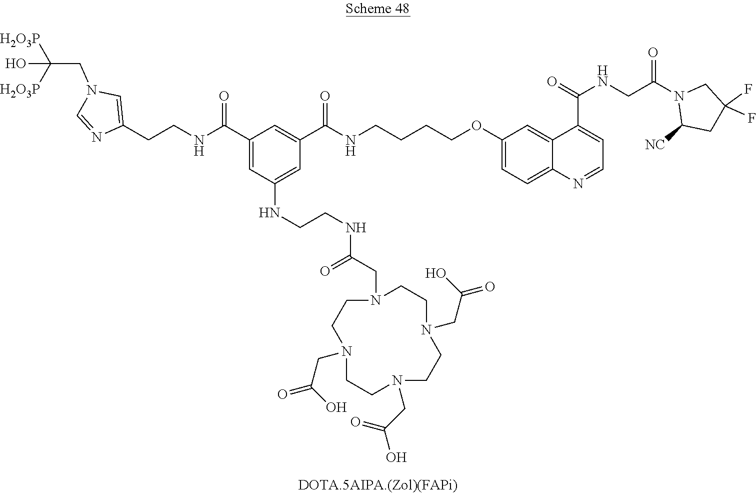

### Example 7

Examples of inventive compounds with a spacer unit (S3) are shown below.

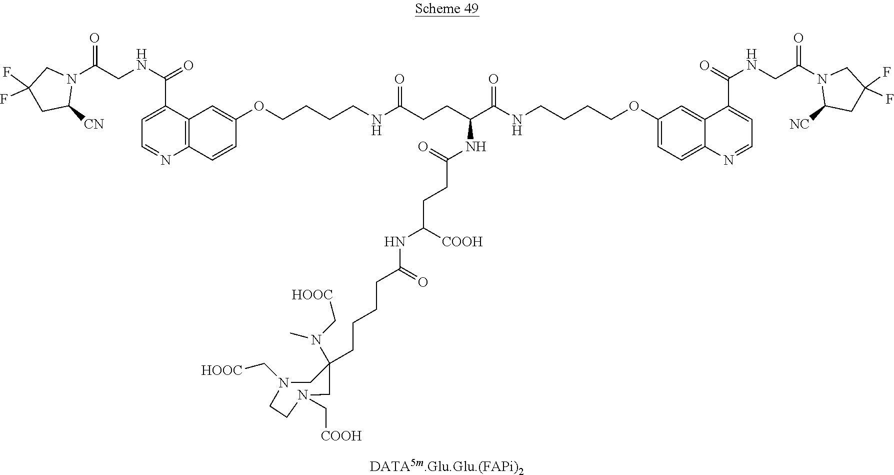 [SMILES: CN(CC(=O)O)C1(CCCCC(=O)NC(CCC(=O)N[C@@H](CCC(=O)NCCCCOc2ccc3nccc(C(=O)NCC(=O)N4CC(F)(F)C[C@@H]4C#N)c3c2)C(=O)NCCCCOc2ccc3nccc(C(=O)NCC(=O)N4CC(F)(F)C[C@H]4C#N)c3c2)C(=O)O)CN(CC(=O)O)CCN(CC(=O)O)C1 ; "DATA5m.Glu.Glu.(FAPi)2"]

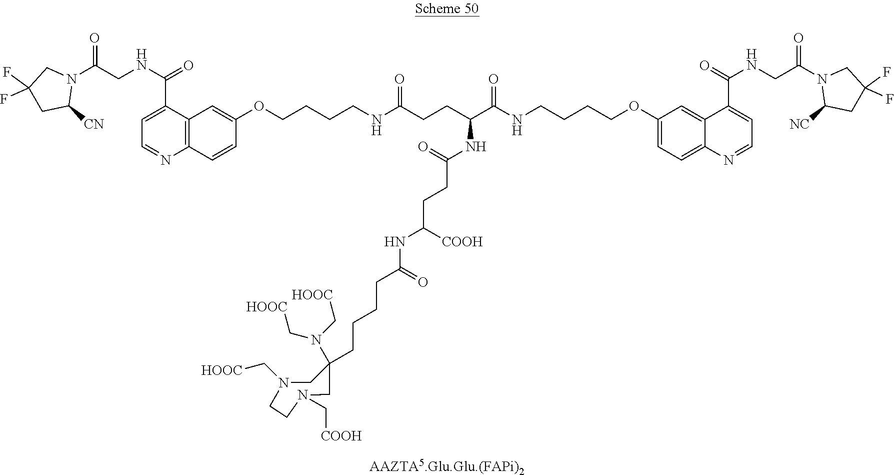 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCC(NC(=O)CCCCC3(N(CC(=O)O)CC(=O)O)CN(CC(=O)O)CCN(CC(=O)O)C3)C(=O)O)cc12 ; "AAZTA5.Glu.Glu.(FAPi)2"]

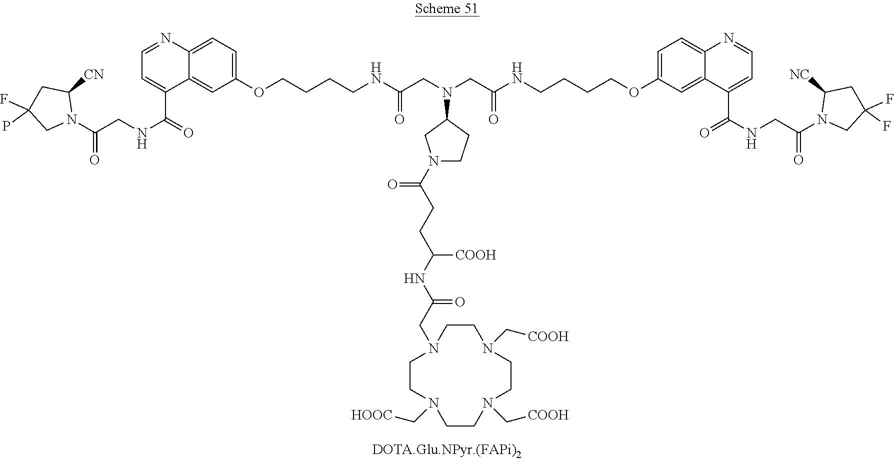

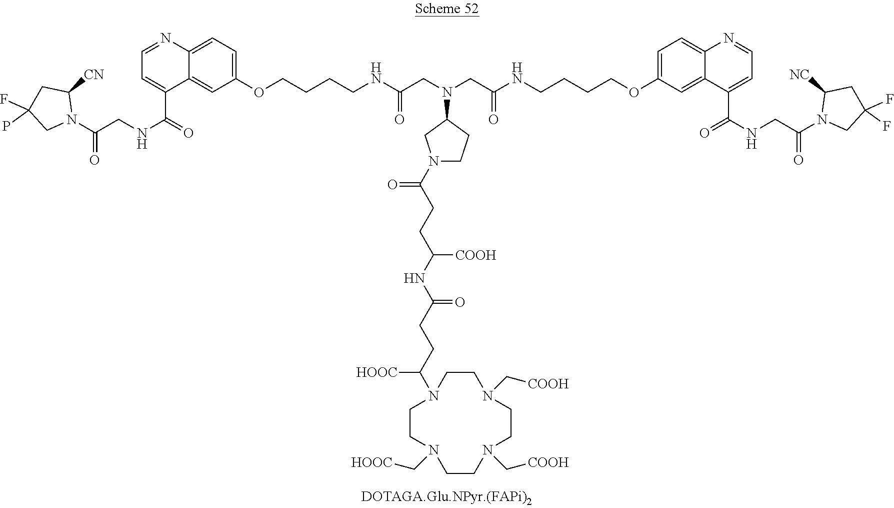

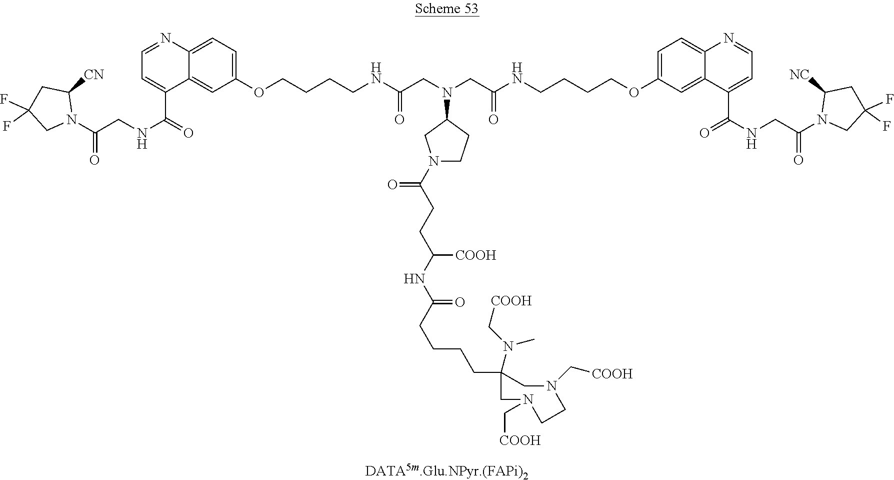 [SMILES: CN(CC(=O)O)C1(CCCCC(=O)NC(CCC(=O)N2CC[C@H](N(CC(=O)NCCCCOc3ccc4nccc(C(=O)NCC(=O)N5CC(F)(F)C[C@H]5C#N)c4c3)CC(=O)NCCCCOc3ccc4nccc(C(=O)NCC(=O)N5CC(F)(F)C[C@@H]5C#N)c4c3)C2)C(=O)O)CN(CC(=O)O)CCN(CC(=O)O)C1]

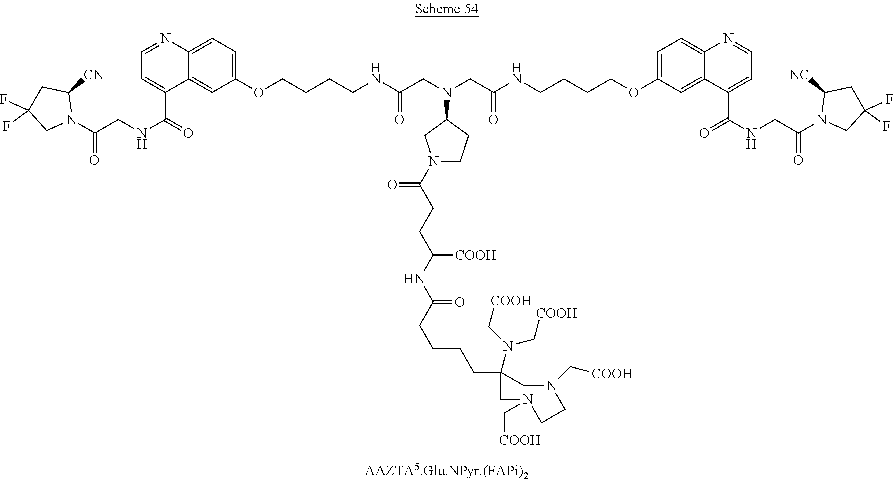 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCC(NC(=O)CCCCC4(N(CC(=O)O)CC(=O)O)CN(CC(=O)O)CCN(CC(=O)O)C4)C(=O)O)C3)cc12]

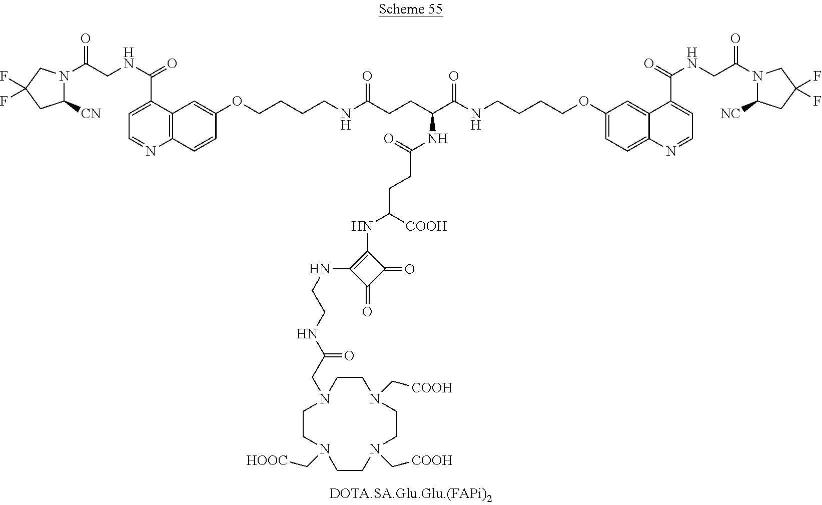 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCC(Nc3c(NCCNC(=O)CN4CCN(CC(=O)O)CCN(CC(=O)O)CCN(CC(=O)O)CC4)c(=O)c3=O)C(=O)O)cc12]

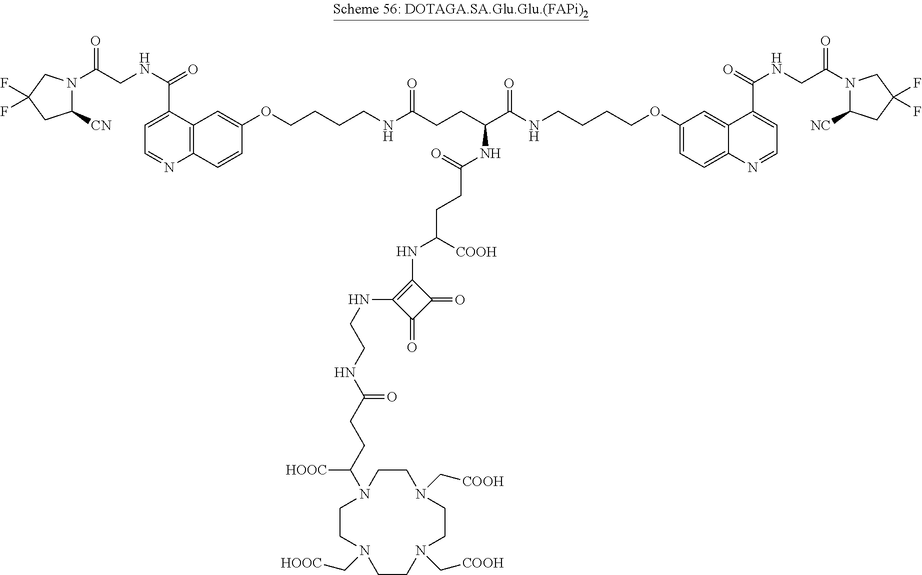 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCC(Nc3c(NCCNC(=O)CCC(C(=O)O)N4CCN(CC(=O)O)CCN(CC(=O)O)CCN(CC(=O)O)CC4)c(=O)c3=O)C(=O)O)cc12]

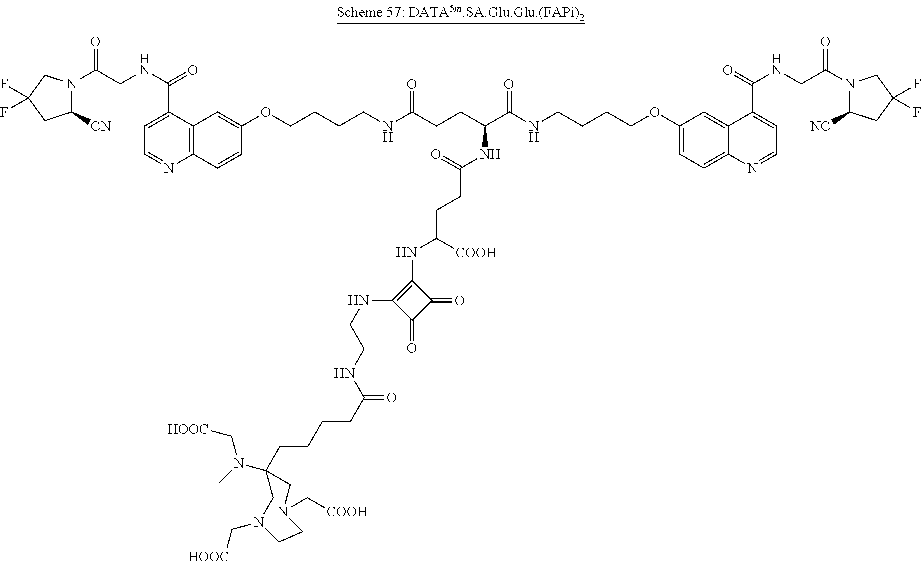 [SMILES: CN(CC(=O)O)C1(CCCCC(=O)NCCNc2c(NC(CCC(=O)N[C@@H](CCC(=O)NCCCCOc3ccc4nccc(C(=O)NCC(=O)N5CC(F)(F)C[C@@H]5C#N)c4c3)C(=O)NCCCCOc3ccc4nccc(C(=O)NCC(=O)N5CC(F)(F)C[C@H]5C#N)c4c3)C(=O)O)c(=O)c2=O)CN(CC(=O)O)CCN(CC(=O)O)C1]

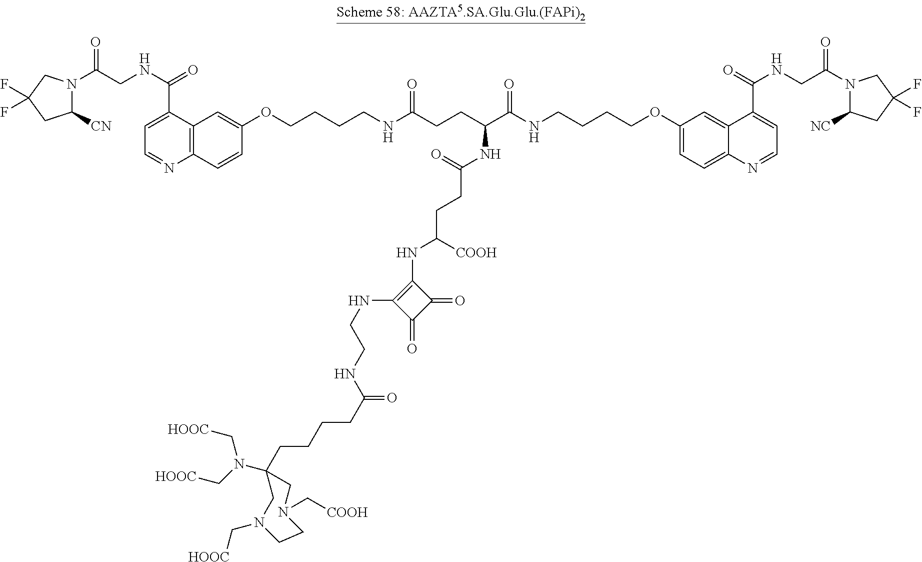 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCC(Nc3c(NCCNC(=O)CCCCC4(N(CC(=O)O)CC(=O)O)CN(CC(=O)O)CCN(CC(=O)O)C4)c(=O)c3=O)C(=O)O)cc12]

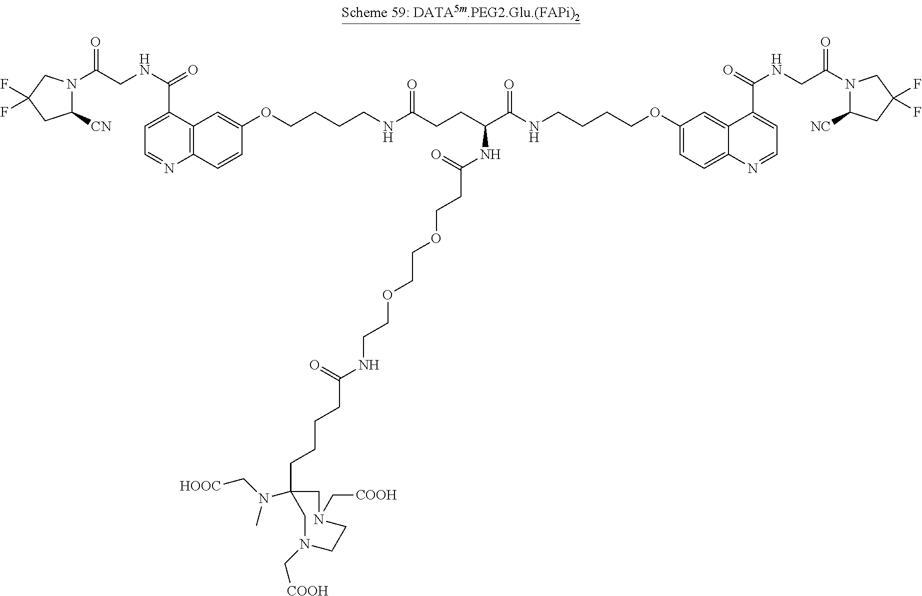 [SMILES: CN(CC(=O)O)C1(CCCCC(=O)NCCOCCOCCC(=O)N[C@@H](CCC(=O)NCCCCOc2ccc3nccc(C(=O)NCC(=O)N4CC(F)(F)C[C@@H]4C#N)c3c2)C(=O)NCCCCOc2ccc3nccc(C(=O)NCC(=O)N4CC(F)(F)C[C@H]4C#N)c3c2)CN(CC(=O)O)CCN(CC(=O)O)C1]

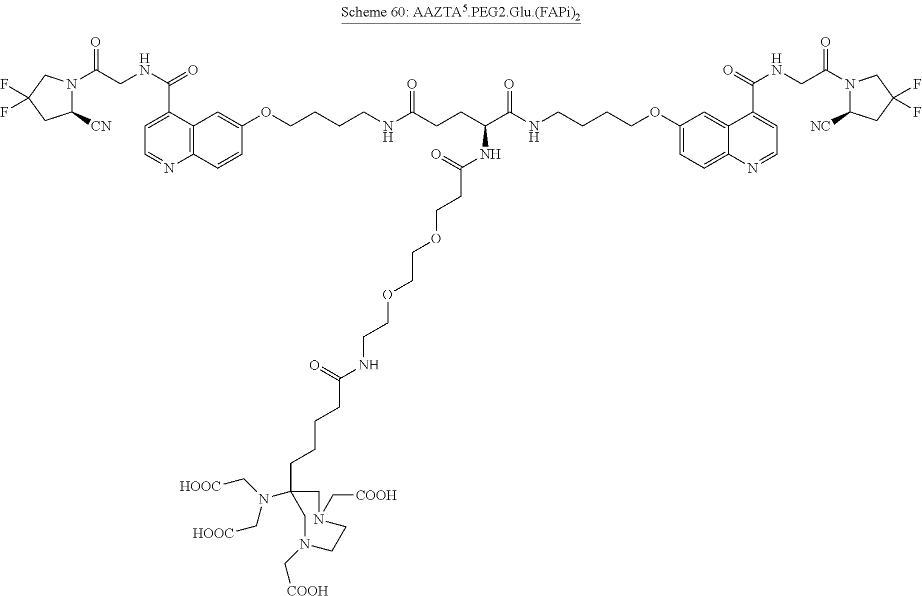 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCNC(=O)CCCCC3(N(CC(=O)O)CC(=O)O)CN(CC(=O)O)CCN(CC(=O)O)C3)cc12]

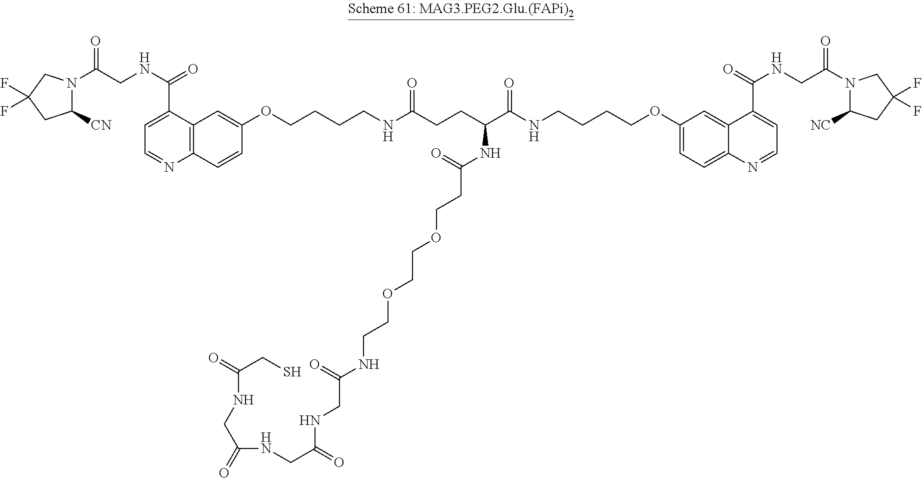 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCNC(=O)CNC(=O)CNC(=O)CNC(=O)CS)cc12]

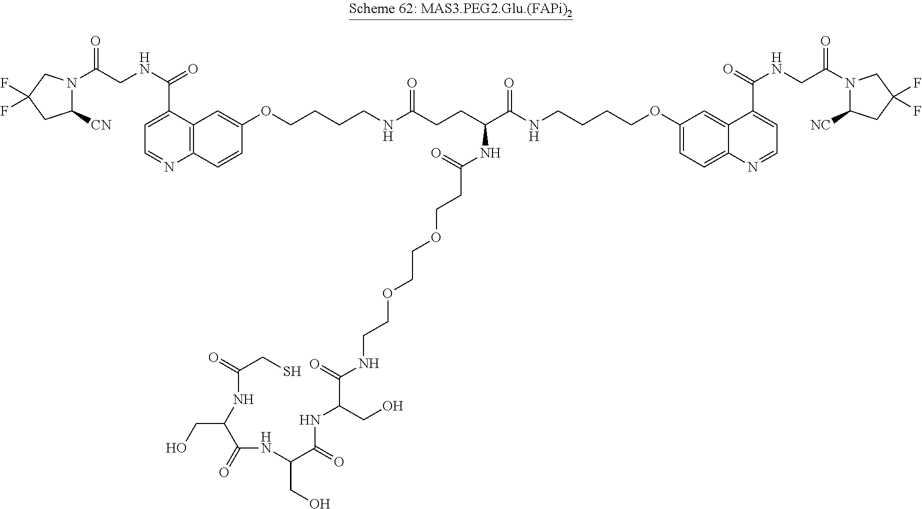 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCNC(=O)C(CO)NC(=O)C(CO)NC(=O)C(CO)NC(=O)CS)cc12]

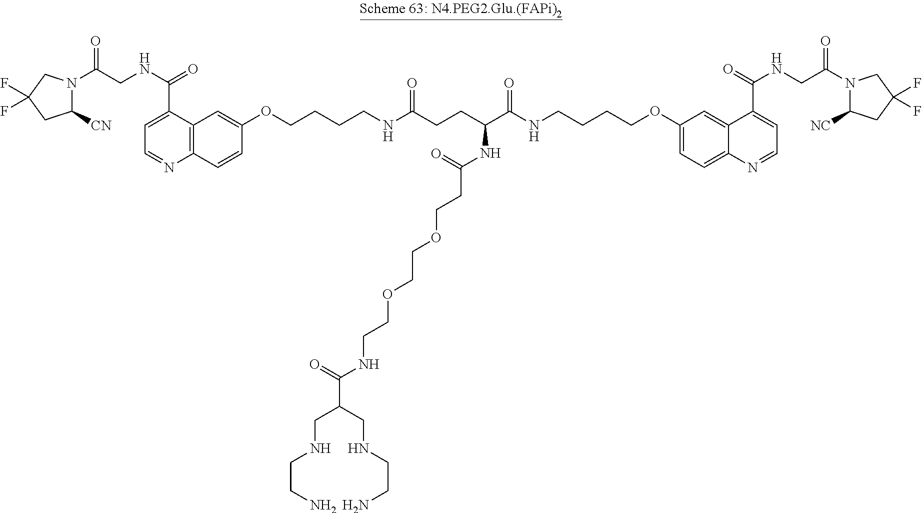 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCNC(=O)C(CNCCN)CNCCN)cc12]

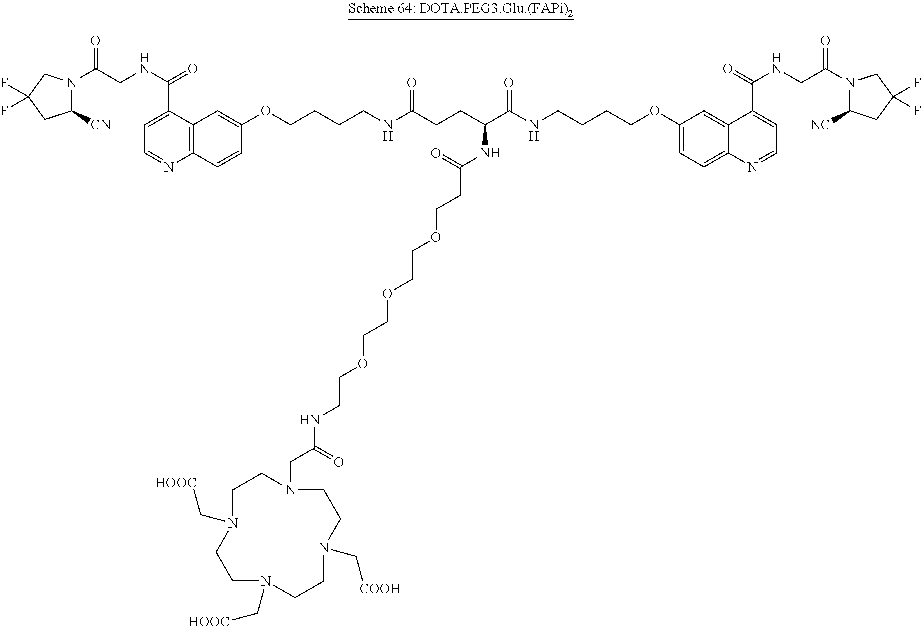 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCOCCNC(=O)CN3CCN(CC(=O)O)CCN(CC(=O)O)CCN(CC(=O)O)CC3)cc12]

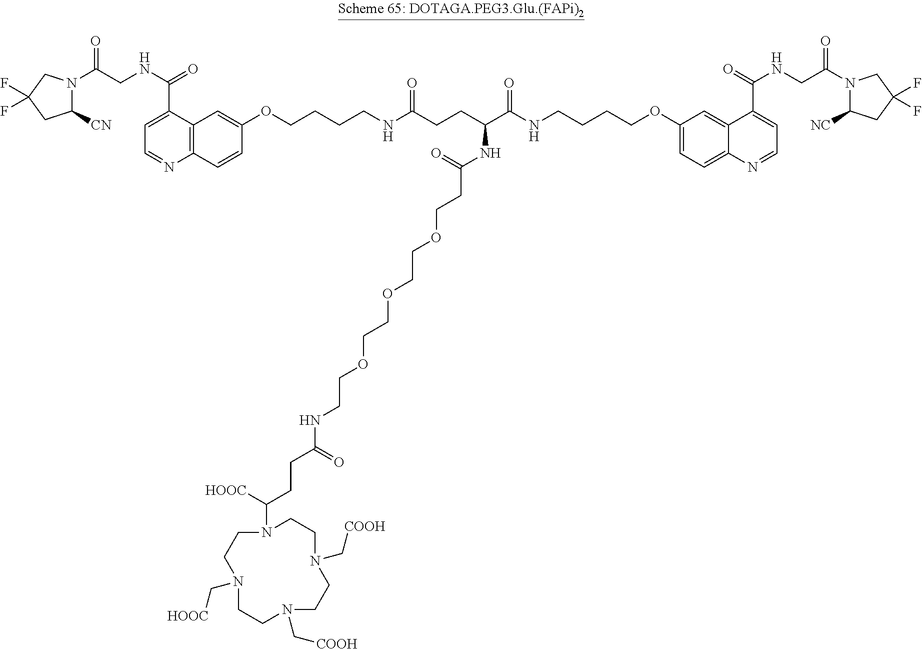 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCOCCNC(=O)CCC(C(=O)O)N3CCN(CC(=O)O)CCN(CC(=O)O)CCN(CC(=O)O)CC3)cc12]

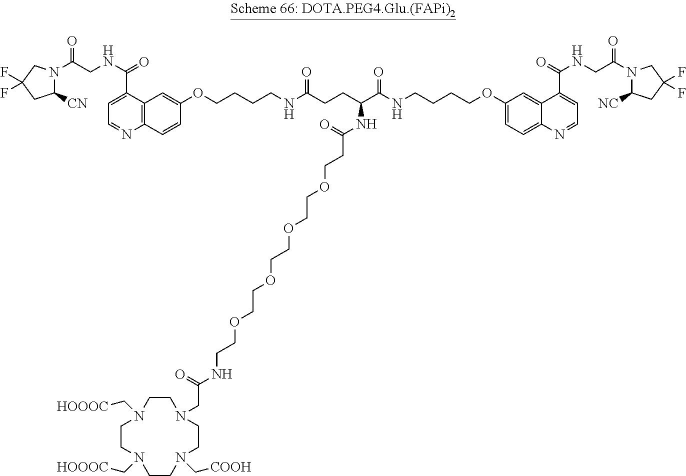

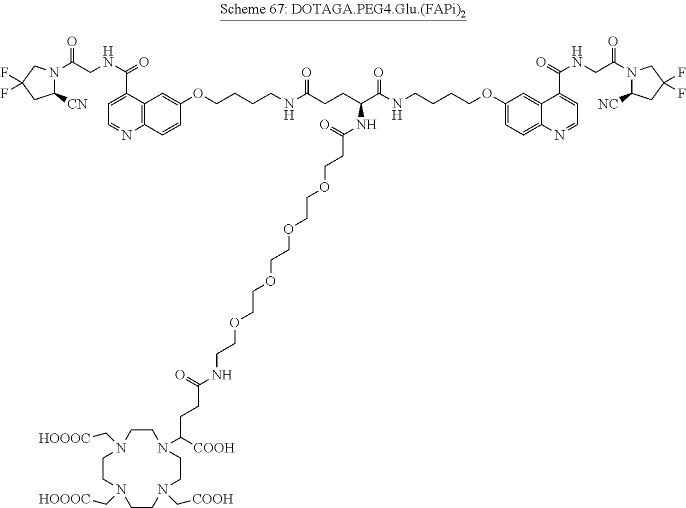

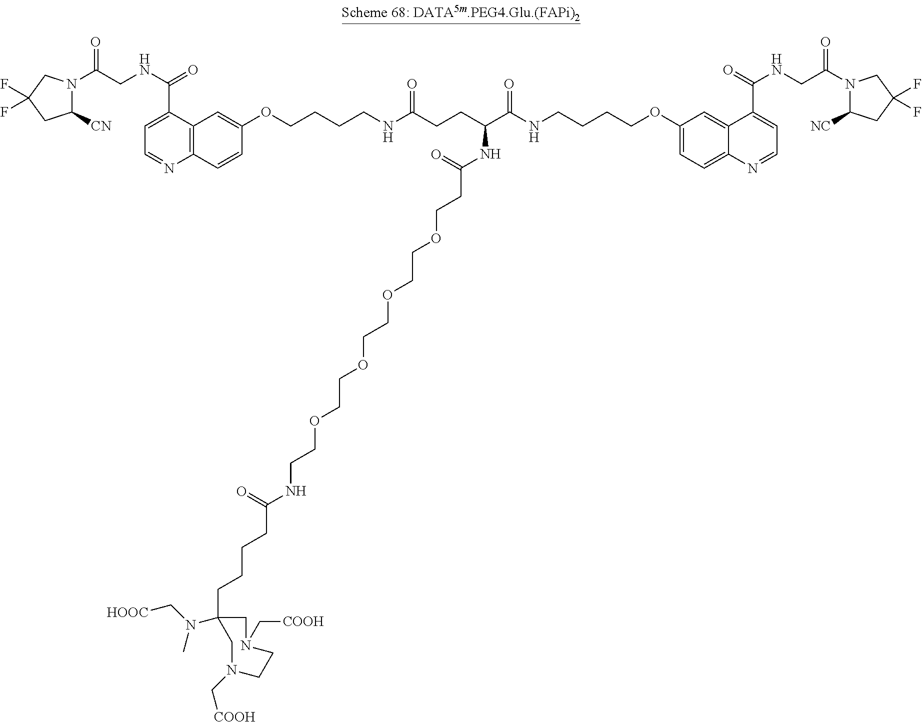 [SMILES: CN(CC(=O)O)C1(CCCCC(=O)NCCOCCOCCOCCOCCC(=O)N[C@@H](CCC(=O)NCCCCOc2ccc3nccc(C(=O)NCC(=O)N4CC(F)(F)C[C@@H]4C#N)c3c2)C(=O)NCCCCOc2ccc3nccc(C(=O)NCC(=O)N4CC(F)(F)C[C@H]4C#N)c3c2)CN(CC(=O)O)CCN(CC(=O)O)C1]

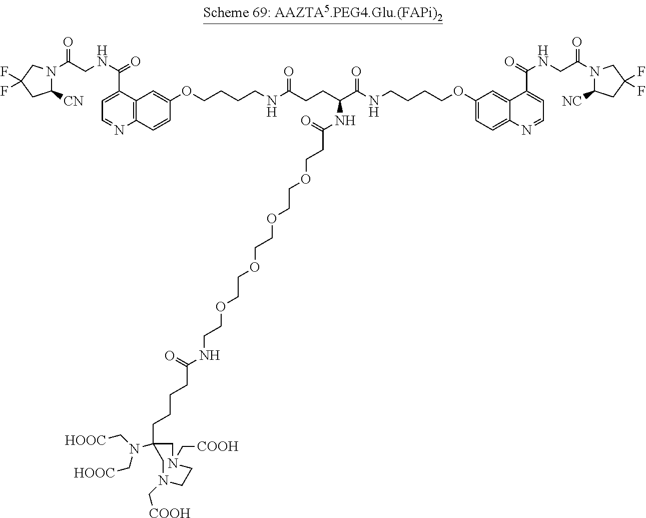 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCOCCOCCNC(=O)CCCCC3(N(CC(=O)O)CC(=O)O)CN(CC(=O)O)CCN(CC(=O)O)C3)cc12]

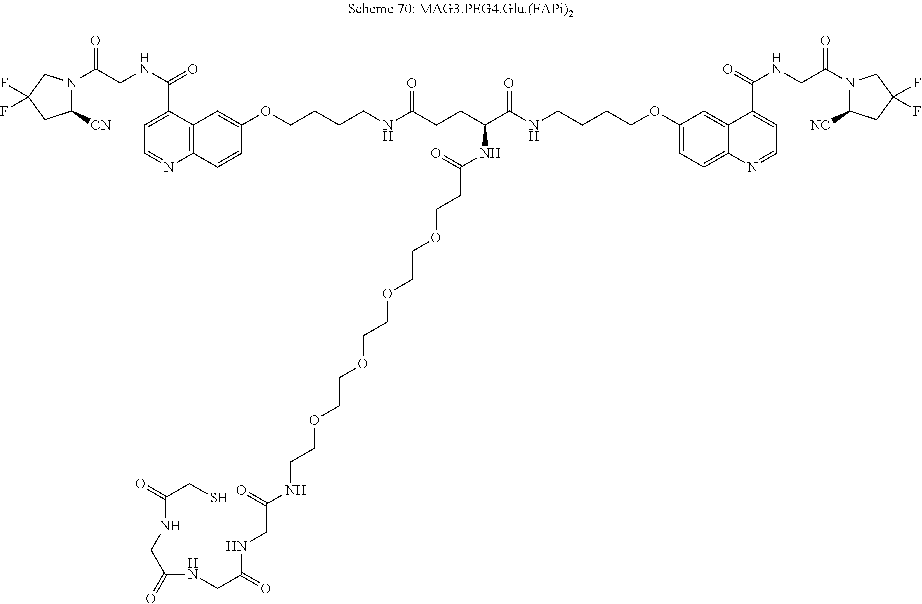 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCOCCOCCNC(=O)CNC(=O)CNC(=O)CNC(=O)CS)cc12]

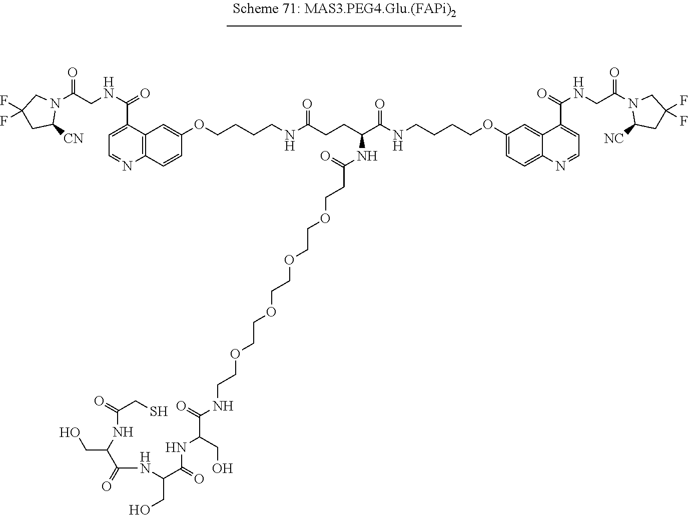 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCOCCOCCNC(=O)C(CO)NC(=O)C(CO)NC(=O)C(CO)NC(=O)CS)cc12]

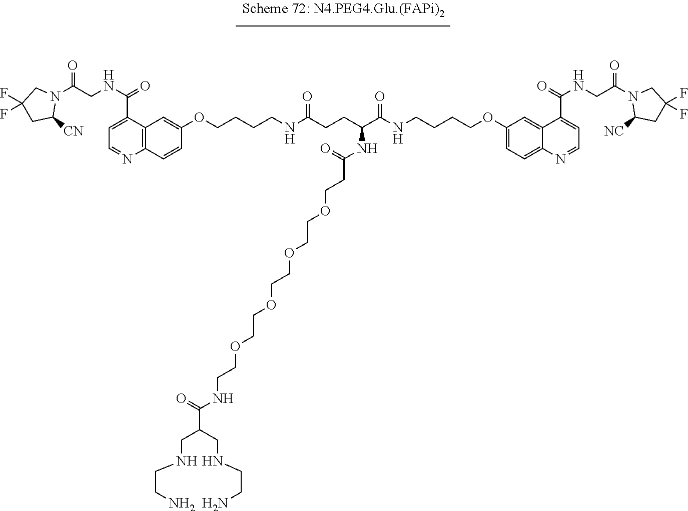 [SMILES: N#C[C@@H]1CC(F)(F)CN1C(=O)CNC(=O)c1ccnc2ccc(OCCCCNC(=O)[C@H](CCC(=O)NCCCCOc3ccc4nccc(C(=O)NCC(=O)N5CC(F)(F)C[C@@H]5C#N)c4c3)NC(=O)CCOCCOCCOCCOCCNC(=O)C(CNCCN)CNCCN)cc12]

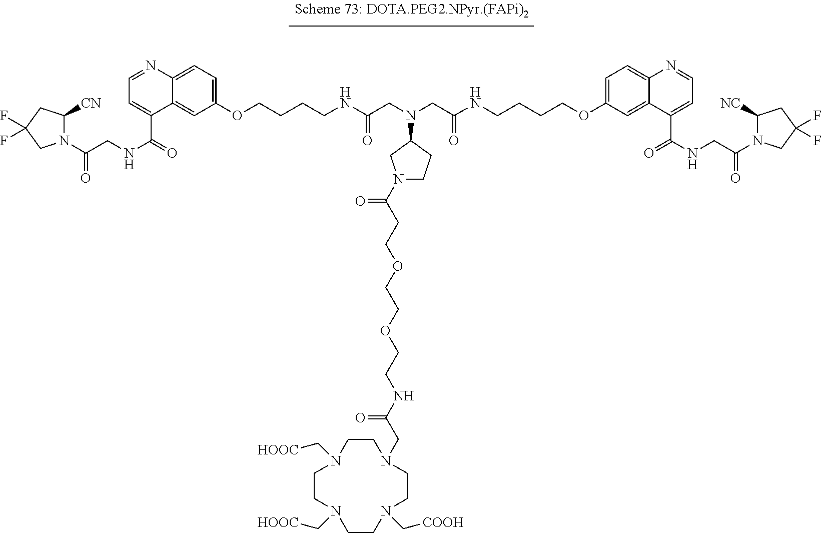 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCNC(=O)CN4CCN(CC(=O)O)CCN(CC(=O)O)CCN(CC(=O)O)CC4)C3)cc12]

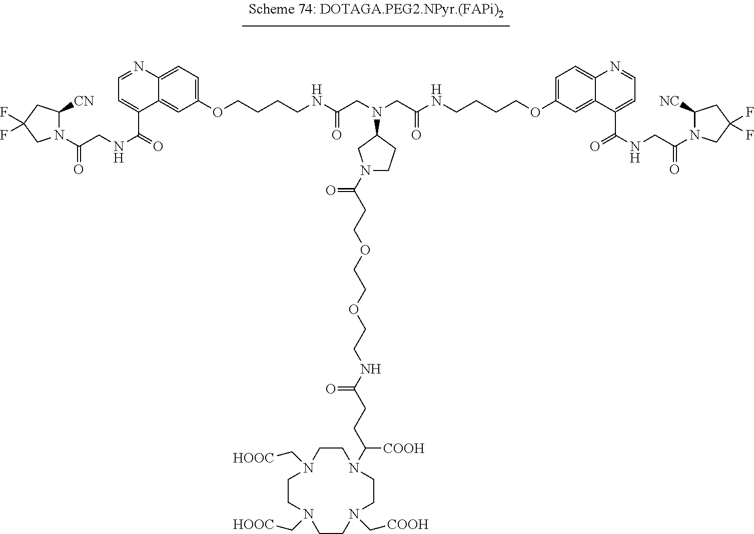 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCNC(=O)CCC(C(=O)O)N4CCN(CC(=O)O)CCN(CC(=O)O)CCN(CC(=O)O)CC4)C3)cc12]

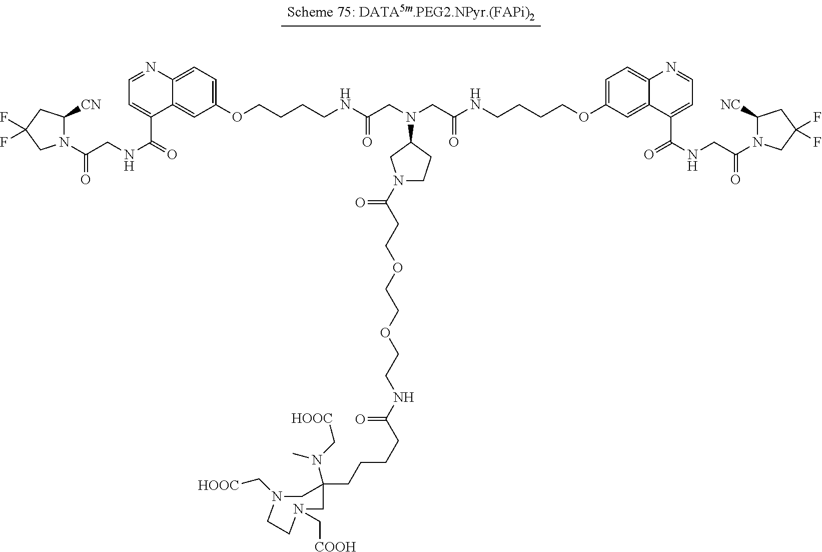 [SMILES: CN(CC(=O)O)C1(CCCCC(=O)NCCOCCOCCC(=O)N2CC[C@H](N(CC(=O)NCCCCOc3ccc4nccc(C(=O)NCC(=O)N5CC(F)(F)C[C@H]5C#N)c4c3)CC(=O)NCCCCOc3ccc4nccc(C(=O)NCC(=O)N5CC(F)(F)C[C@@H]5C#N)c4c3)C2)CN(CC(=O)O)CCN(CC(=O)O)C1]

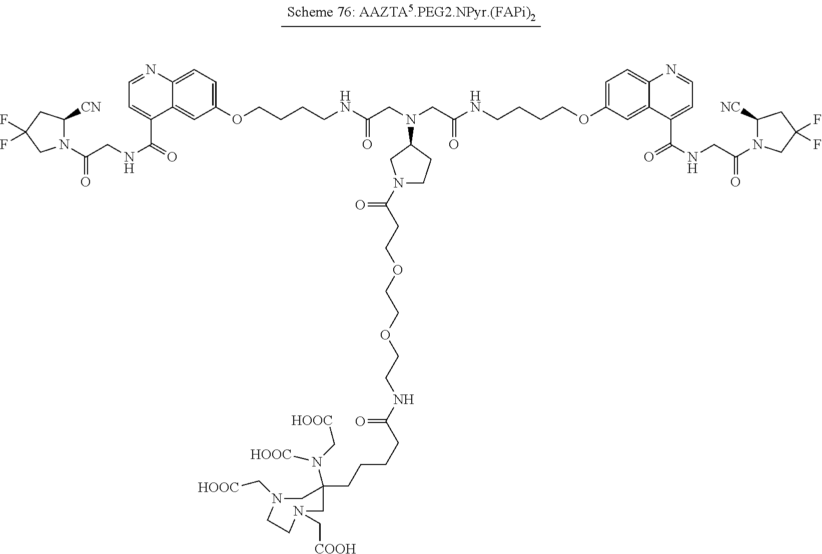 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCNC(=O)CCCCC4(N(CC(=O)O)C(=O)O)CN(CC(=O)O)CCN(CC(=O)O)C4)C3)cc12]

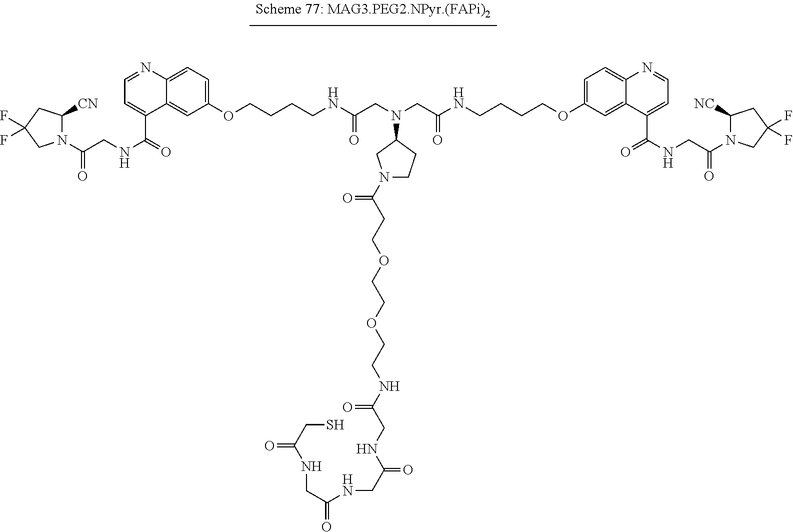 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCNC(=O)CNC(=O)CNC(=O)CNC(=O)CS)C3)cc12]

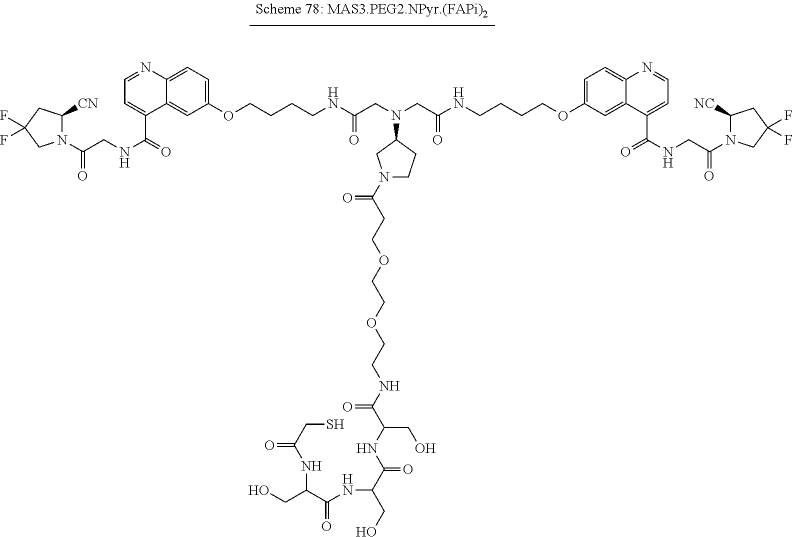 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCNC(=O)C(CO)NC(=O)C(CO)NC(=O)C(CO)NC(=O)CS)C3)cc12]

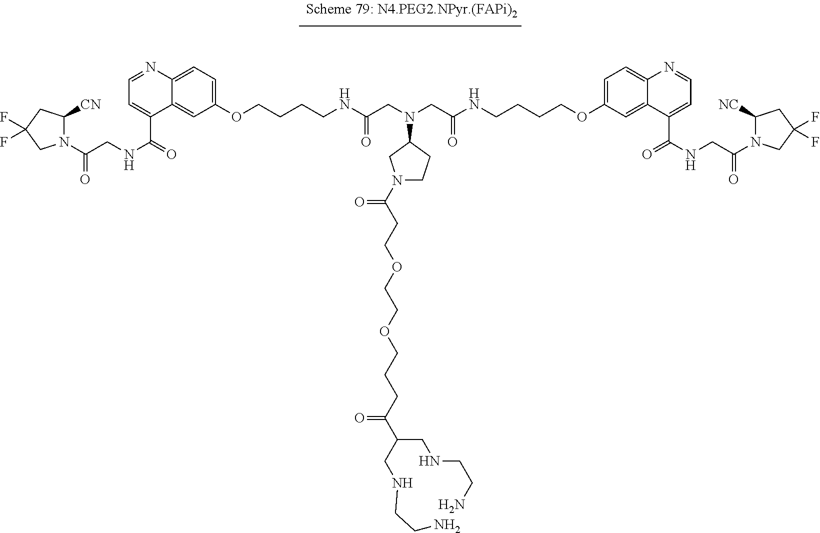 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCCC(=O)C(CNCCN)CNCCN)C3)cc12]

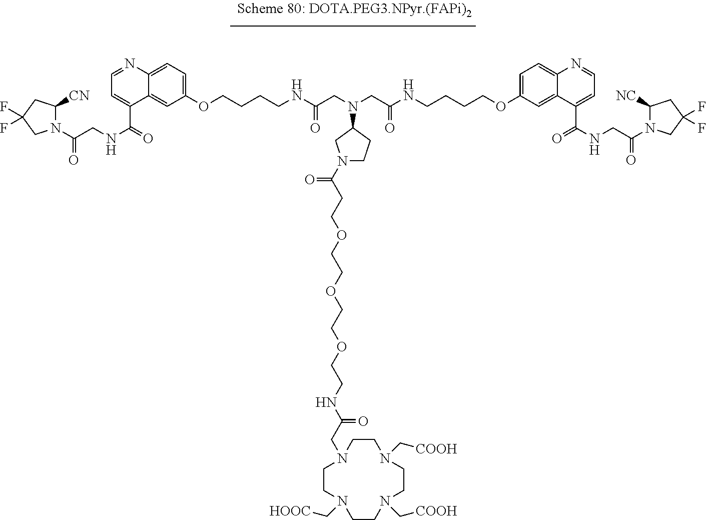 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCOCCNC(=O)CN4CCN(CC(=O)O)CCN(CC(=O)O)CCN(CC(=O)O)CC4)C3)cc12]

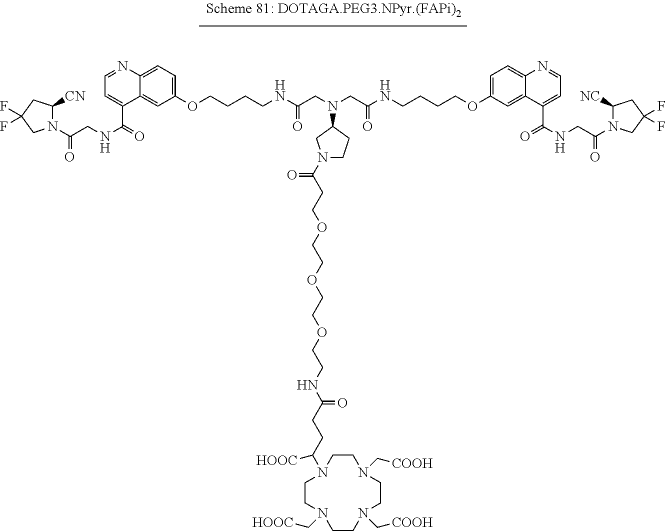 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCOCCNC(=O)CCC(C(=O)O)N4CCN(CC(=O)O)CCN(CC(=O)O)CCN(CC(=O)O)CC4)C3)cc12]

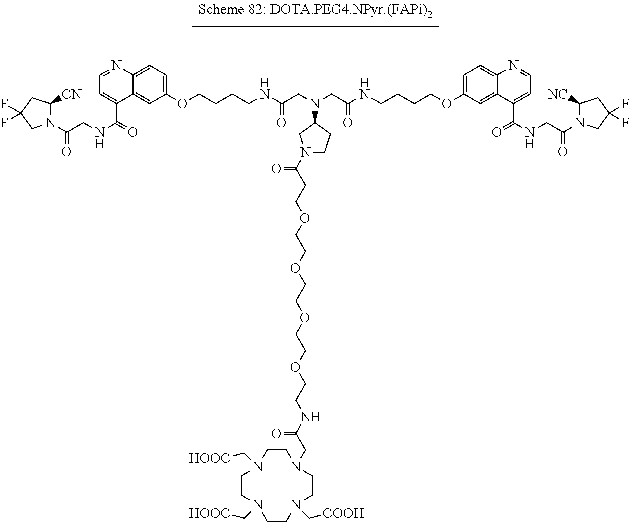 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCOCCOCCNC(=O)CN4CCN(CC(=O)O)CCN(CC(=O)O)CCN(CC(=O)O)CC4)C3)cc12]

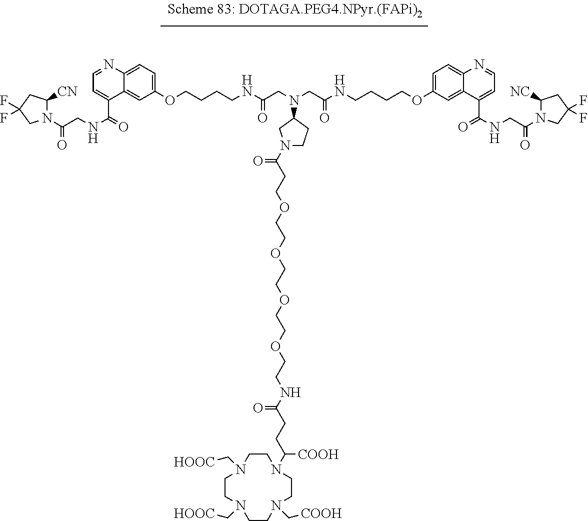 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCOCCOCCNC(=O)CCC(C(=O)O)N4CCN(CC(=O)O)CCN(CC(=O)O)CCN(CC(=O)O)CC4)C3)cc12]

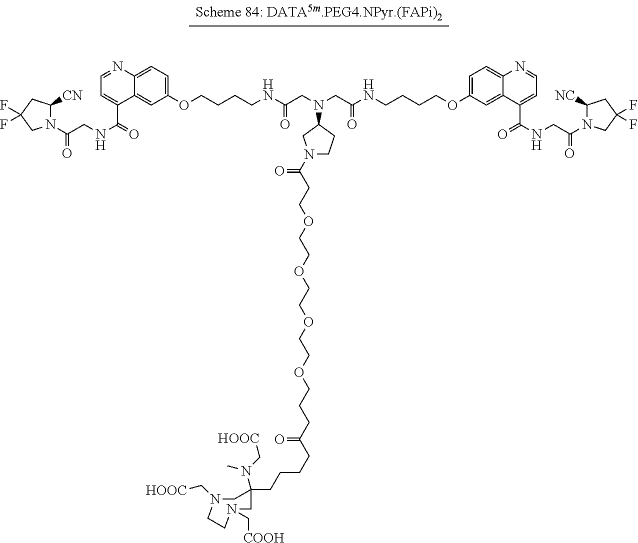 [SMILES: CN(CC(=O)O)C1(CCCCC(=O)CCCOCCOCCOCCOCCC(=O)N2CC[C@H](N(CC(=O)NCCCCOc3ccc4nccc(C(=O)NCC(=O)N5CC(F)(F)C[C@H]5C#N)c4c3)CC(=O)NCCCCOc3ccc4nccc(C(=O)NCC(=O)N5CC(F)(F)C[C@@H]5C#N)c4c3)C2)CN(CC(=O)O)CCN(CC(=O)O)C1]

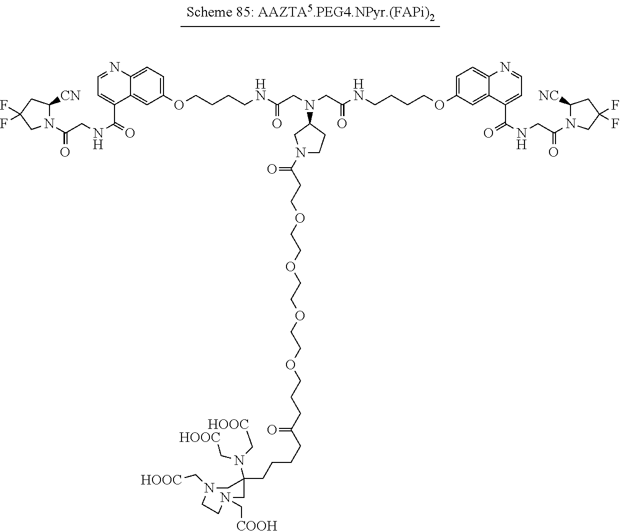 [SMILES: N#C[C@@H]1CC(F)(F)CN1C(=O)CNC(=O)c1ccnc2ccc(OCCCCNC(=O)CN(CC(=O)NCCCCOc3ccc4nccc(C(=O)NCC(=O)N5CC(F)(F)C[C@@H]5C#N)c4c3)[C@H]3CCN(C(=O)CCOCCOCCOCCOCCCC(=O)CCCCC4(N(CC(=O)O)CC(=O)O)CN(CC(=O)O)CCN(CC(=O)O)C4)C3)cc12]

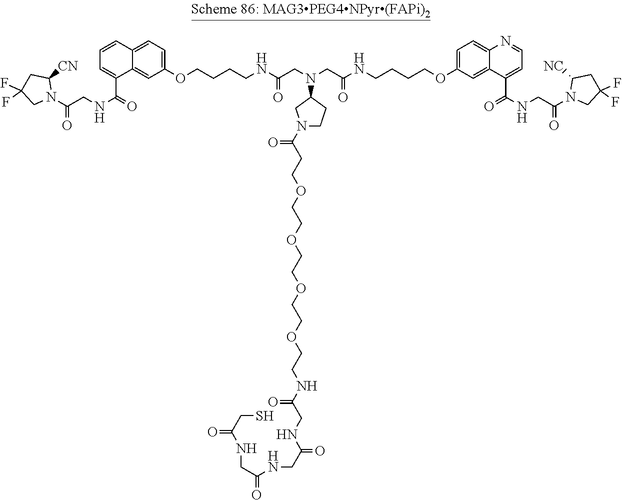 [SMILES: N#C[C@@H]1CC(F)(F)CN1C(=O)CNC(=O)c1cccc2ccc(OCCCCNC(=O)CN(CC(=O)NCCCCOc3ccc4nccc(C(=O)NCC(=O)N5CC(F)(F)C[C@H]5C#N)c4c3)[C@H]3CCN(C(=O)CCOCCOCCOCCOCCNC(=O)CNC(=O)CNC(=O)CNC(=O)CS)C3)cc12]

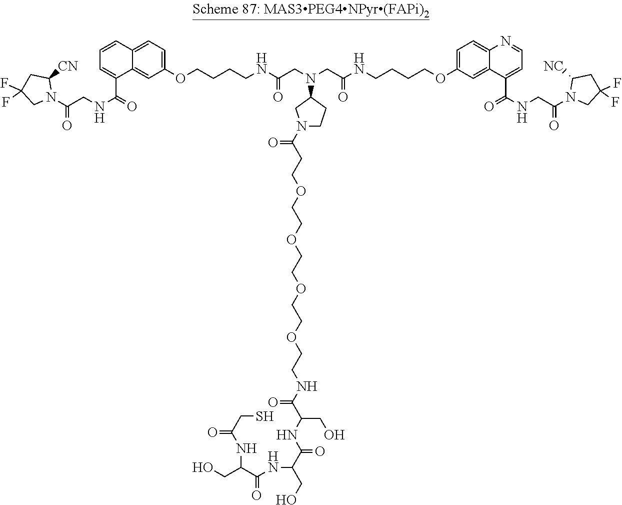 [SMILES: N#C[C@@H]1CC(F)(F)CN1C(=O)CNC(=O)c1cccc2ccc(OCCCCNC(=O)CN(CC(=O)NCCCCOc3ccc4nccc(C(=O)NCC(=O)N5CC(F)(F)C[C@H]5C#N)c4c3)[C@H]3CCN(C(=O)CCOCCOCCOCCOCCNC(=O)C(CO)NC(=O)C(CO)NC(=O)C(CO)NC(=O)CS)C3)cc12]

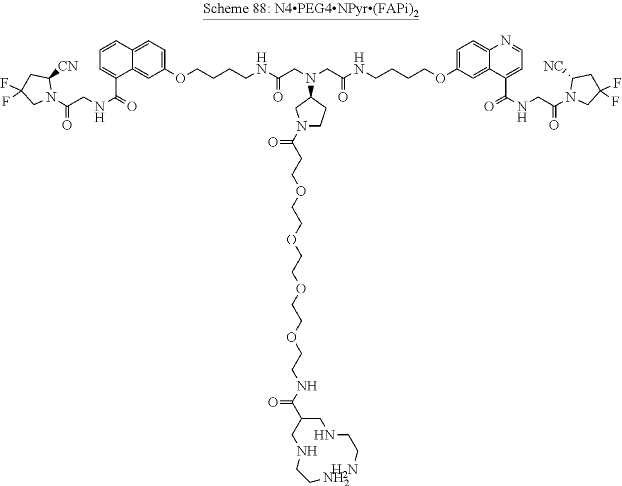 [SMILES: N#C[C@@H]1CC(F)(F)CN1C(=O)CNC(=O)c1cccc2ccc(OCCCCNC(=O)CN(CC(=O)NCCCCOc3ccc4nccc(C(=O)NCC(=O)N5CC(F)(F)C[C@H]5C#N)c4c3)[C@H]3CCN(C(=O)CCOCCOCCOCCOCCNC(=O)C(CNCCN)CNCCN)C3)cc12]

### Example 8

Examples of inventive compounds with two spacer units (S1+S2) are shown below.

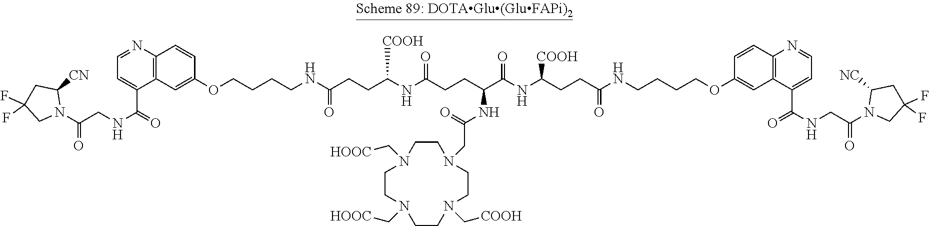 [SMILES: N#C[C@@H]1CC(F)(F)CN1C(=O)CNC(=O)c1ccnc2ccc(OCCCCNC(=O)CC[C@@H](NC(=O)CC[C@H](NC(=O)CN3CCN(CC(=O)O)CCN(CC(=O)O)CCN(CC(=O)O)CC3)C(=O)N[C@H](CCC(=O)NCCCCOc3ccc4nccc(C(=O)NCC(=O)N5CC(F)(F)C[C@H]5C#N)c4c3)C(=O)O)C(=O)O)cc12]

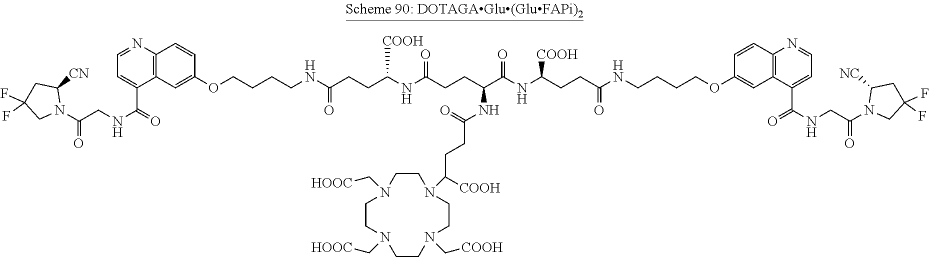 [SMILES: N#C[C@@H]1CC(F)(F)CN1C(=O)CNC(=O)c1ccnc2ccc(OCCCCNC(=O)CC[C@@H](NC(=O)CC[C@H](NC(=O)CCC(C(=O)O)N3CCN(CC(=O)O)CCN(CC(=O)O)CCN(CC(=O)O)CC3)C(=O)N[C@H](CCC(=O)NCCCCOc3ccc4nccc(C(=O)NCC(=O)N5CC(F)(F)C[C@H]5C#N)c4c3)C(=O)O)C(=O)O)cc12]

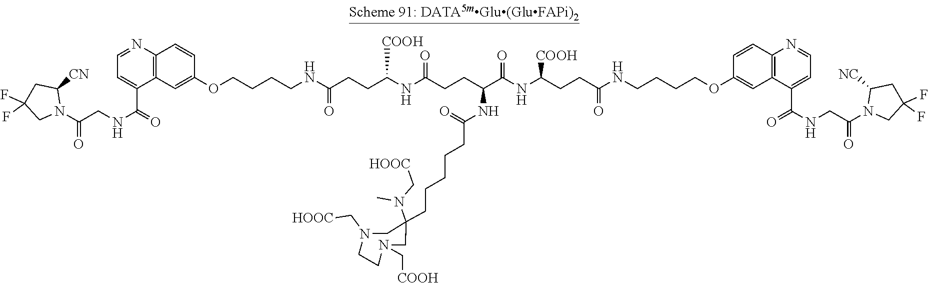 [SMILES: CN(CC(=O)O)C1(CCCCCC(=O)N[C@@H](CCC(=O)N[C@H](CCC(=O)NCCCCOc2ccc3nccc(C(=O)NCC(=O)N4CC(F)(F)C[C@H]4C#N)c3c2)C(=O)O)C(=O)N[C@H](CCC(=O)NCCCCOc2ccc3nccc(C(=O)NCC(=O)N4CC(F)(F)C[C@H]4C#N)c3c2)C(=O)O)CN(CC(=O)O)CCN(CC(=O)O)C1]

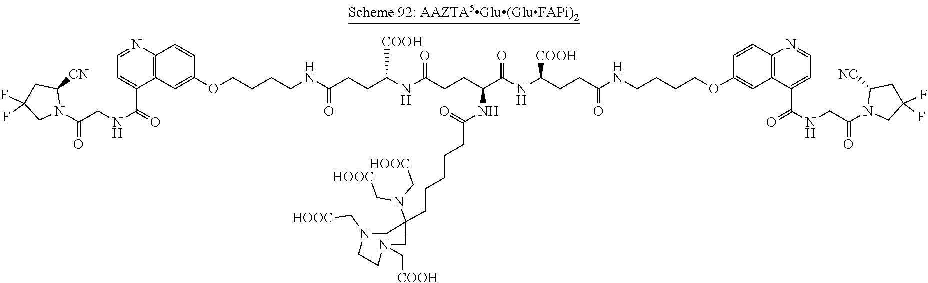 [SMILES: N#C[C@@H]1CC(F)(F)CN1C(=O)CNC(=O)c1ccnc2ccc(OCCCCNC(=O)CC[C@@H](NC(=O)CC[C@H](NC(=O)CCCCCC3(N(CC(=O)O)CC(=O)O)CN(CC(=O)O)CCN(CC(=O)O)C3)C(=O)N[C@H](CCC(=O)NCCCCOc3ccc4nccc(C(=O)NCC(=O)N5CC(F)(F)C[C@H]5C#N)c4c3)C(=O)O)C(=O)O)cc12]

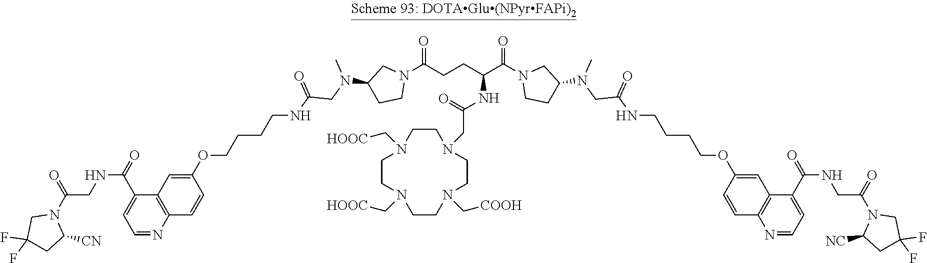 [SMILES: CN(CC(=O)NCCCCOc1ccc2nccc(C(=O)NCC(=O)N3CC(F)(F)C[C@H]3C#N)c2c1)[C@@H]1CCN(C(=O)CC[C@H](NC(=O)CN2CCN(CC(=O)O)CCN(CC(=O)O)CCN(CC(=O)O)CC2)C(=O)N2CC[C@@H](N(C)CC(=O)NCCCCOc3ccc4nccc(C(=O)NCC(=O)N5CC(F)(F)C[C@H]5C#N)c4c3)C2)C1]

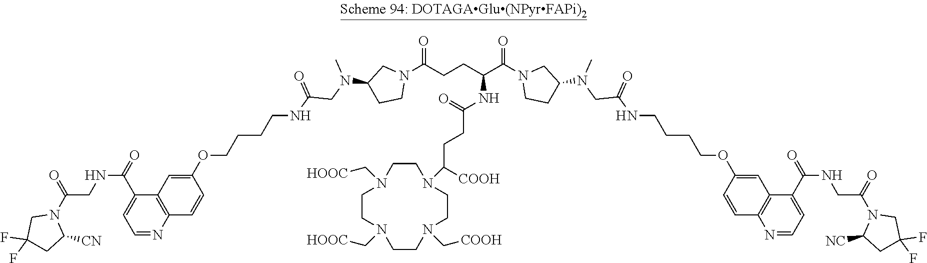 [SMILES: CN(CC(=O)NCCCCOc1ccc2nccc(C(=O)NCC(=O)N3CC(F)(F)C[C@H]3C#N)c2c1)[C@@H]1CCN(C(=O)CC[C@H](NC(=O)CCC(C(=O)O)N2CCN(CC(=O)O)CCN(CC(=O)O)CCN(CC(=O)O)CC2)C(=O)N2CC[C@@H](N(C)CC(=O)NCCCCOc3ccc4nccc(C(=O)NCC(=O)N5CC(F)(F)C[C@H]5C#N)c4c3)C2)C1]

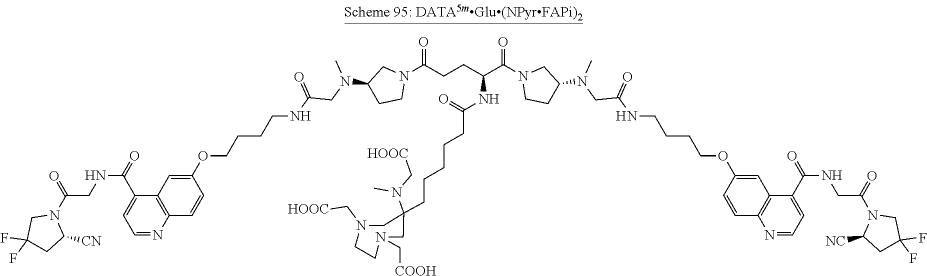 [SMILES: CN(CC(=O)NCCCCOc1ccc2nccc(C(=O)NCC(=O)N3CC(F)(F)C[C@H]3C#N)c2c1)[C@@H]1CCN(C(=O)CC[C@H](NC(=O)CCCCCC2(N(C)CC(=O)O)CN(CC(=O)O)CCN(CC(=O)O)C2)C(=O)N2CC[C@@H](N(C)CC(=O)NCCCCOc3ccc4nccc(C(=O)NCC(=O)N5CC(F)(F)C[C@H]5C#N)c4c3)C2)C1]

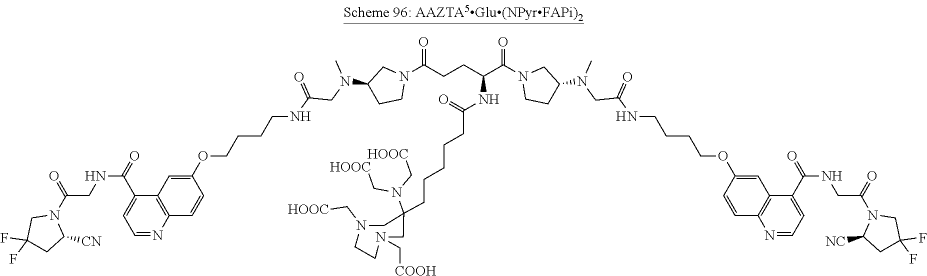 [SMILES: CN(CC(=O)NCCCCOc1ccc2nccc(C(=O)NCC(=O)N3CC(F)(F)C[C@H]3C#N)c2c1)[C@@H]1CCN(C(=O)CC[C@H](NC(=O)CCCCCC2(N(CC(=O)O)CC(=O)O)CN(CC(=O)O)CCN(CC(=O)O)C2)C(=O)N2CC[C@@H](N(C)CC(=O)NCCCCOc3ccc4nccc(C(=O)NCC(=O)N5CC(F)(F)C[C@H]5C#N)c4c3)C2)C1]

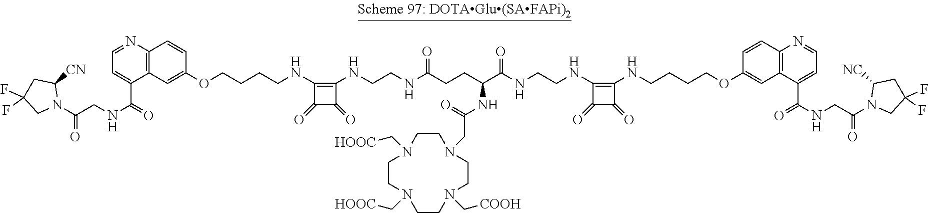 [SMILES: N#C[C@@H]1CC(F)(F)CN1C(=O)CNC(=O)c1ccnc2ccc(OCCCCNc3c(NCCNC(=O)CC[C@H](NC(=O)CN4CCN(CC(=O)O)CCN(CC(=O)O)CCN(CC(=O)O)CC4)C(=O)NCCNc4c(NCCCCOc5ccc6nccc(C(=O)NCC(=O)N7CC(F)(F)C[C@H]7C#N)c6c5)c(=O)c4=O)c(=O)c3=O)cc12]

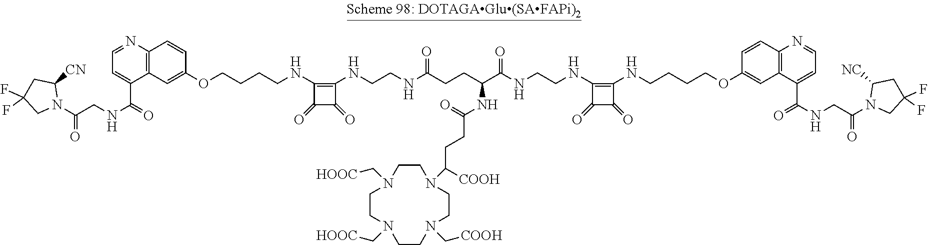 [SMILES: N#C[C@@H]1CC(F)(F)CN1C(=O)CNC(=O)c1ccnc2ccc(OCCCCNc3c(NCCNC(=O)CC[C@H](NC(=O)CCC(C(=O)O)N4CCN(CC(=O)O)CCN(CC(=O)O)CCN(CC(=O)O)CC4)C(=O)NCCNc4c(NCCCCOc5ccc6nccc(C(=O)NCC(=O)N7CC(F)(F)C[C@H]7C#N)c6c5)c(=O)c4=O)c(=O)c3=O)cc12]

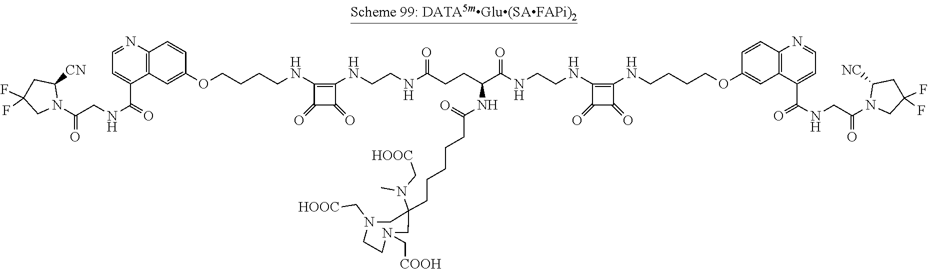 [SMILES: CN(CC(=O)O)C1(CCCCCC(=O)N[C@@H](CCC(=O)NCCNc2c(NCCCCOc3ccc4nccc(C(=O)NCC(=O)N5CC(F)(F)C[C@H]5C#N)c4c3)c(=O)c2=O)C(=O)NCCNc2c(NCCCCOc3ccc4nccc(C(=O)NCC(=O)N5CC(F)(F)C[C@H]5C#N)c4c3)c(=O)c2=O)CN(CC(=O)O)CCN(CC(=O)O)C1]

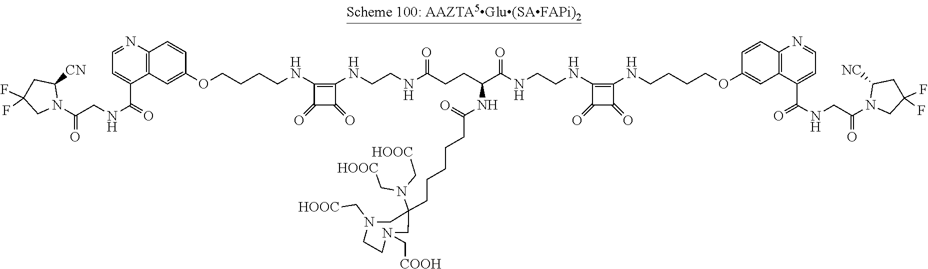 [SMILES: N#C[C@@H]1CC(F)(F)CN1C(=O)CNC(=O)c1ccnc2ccc(OCCCCNc3c(NCCNC(=O)CC[C@H](NC(=O)CCCCCC4(N(CC(=O)O)CC(=O)O)CN(CC(=O)O)CCN(CC(=O)O)C4)C(=O)NCCNc4c(NCCCCOc5ccc6nccc(C(=O)NCC(=O)N7CC(F)(F)C[C@H]7C#N)c6c5)c(=O)c4=O)c(=O)c3=O)cc12]

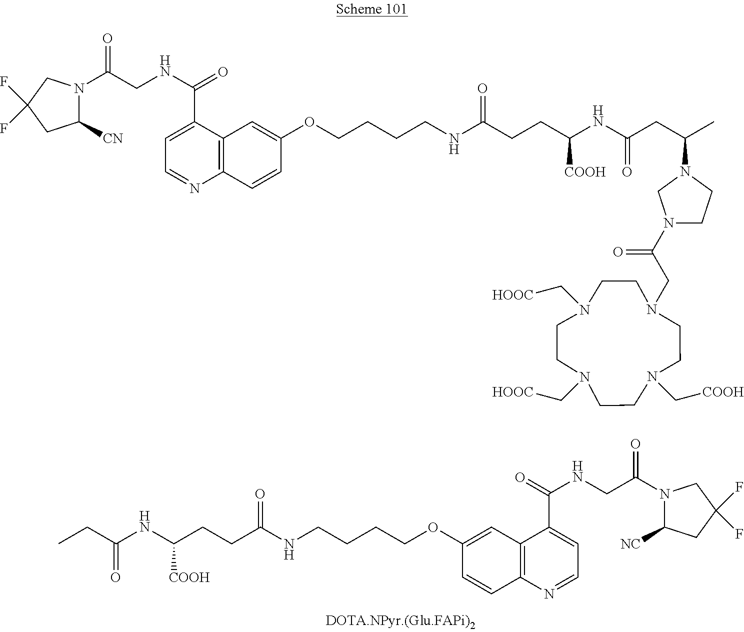

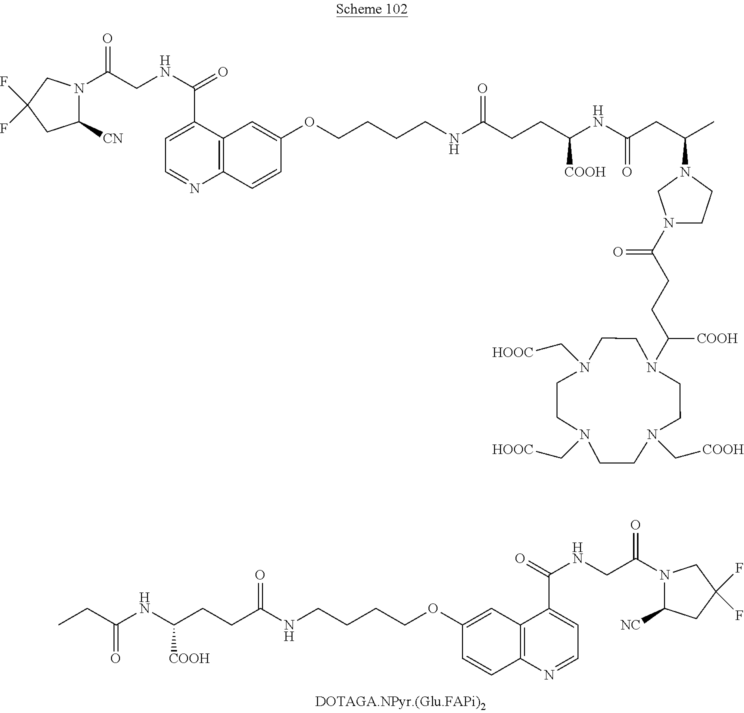

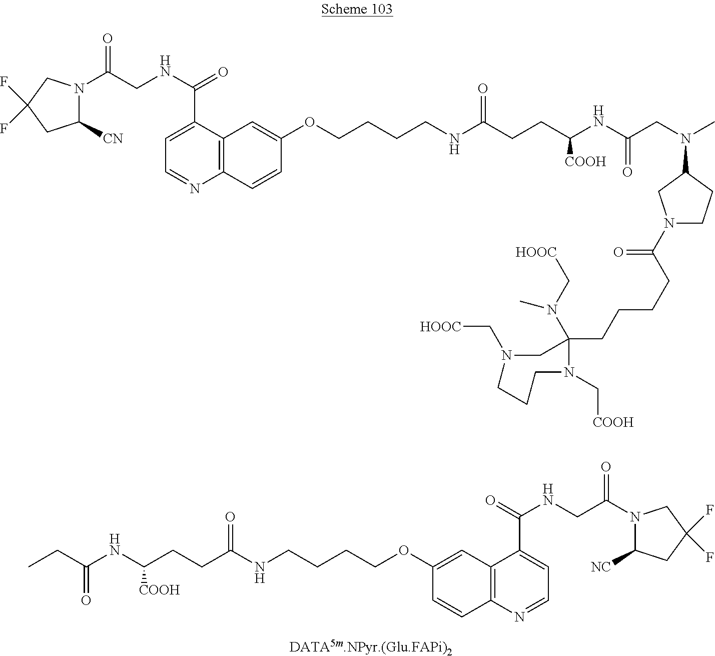

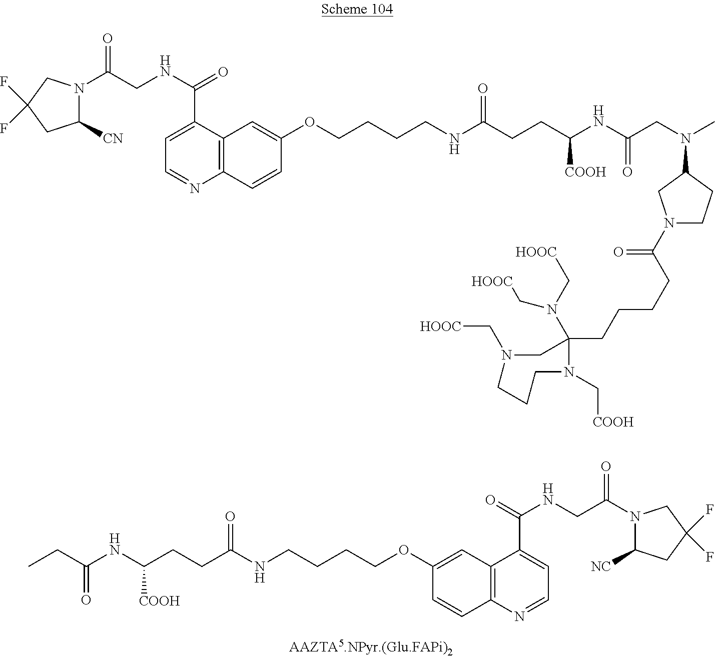

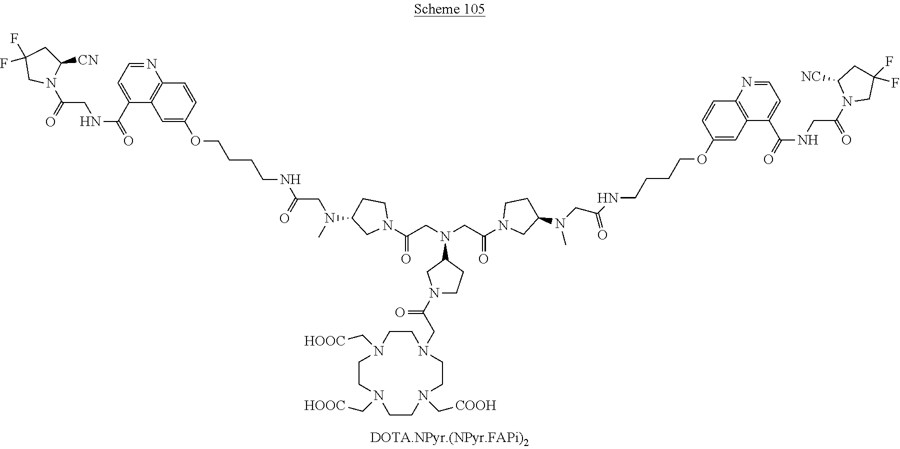

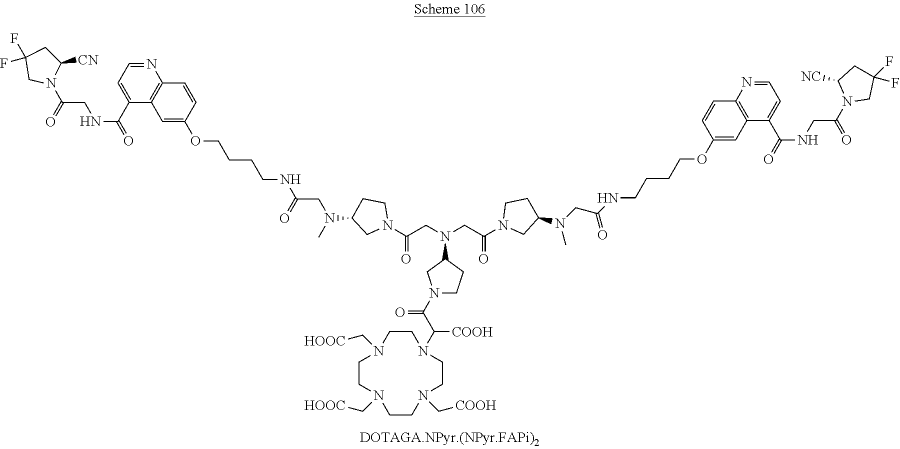

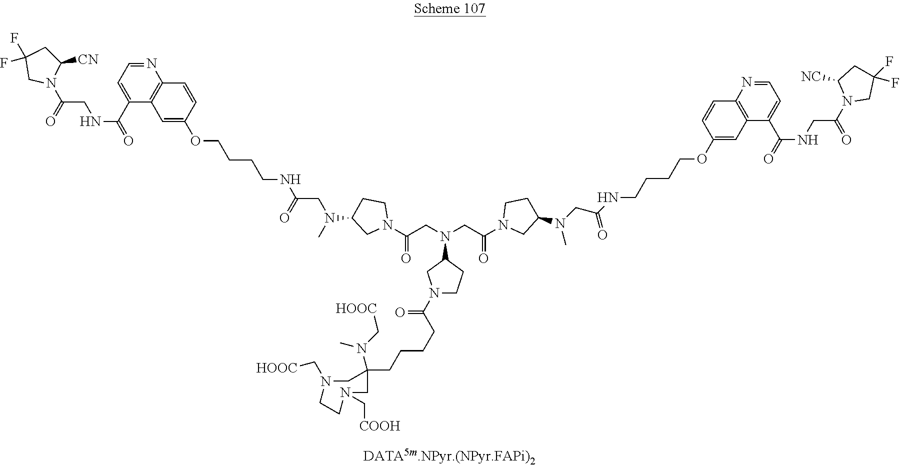

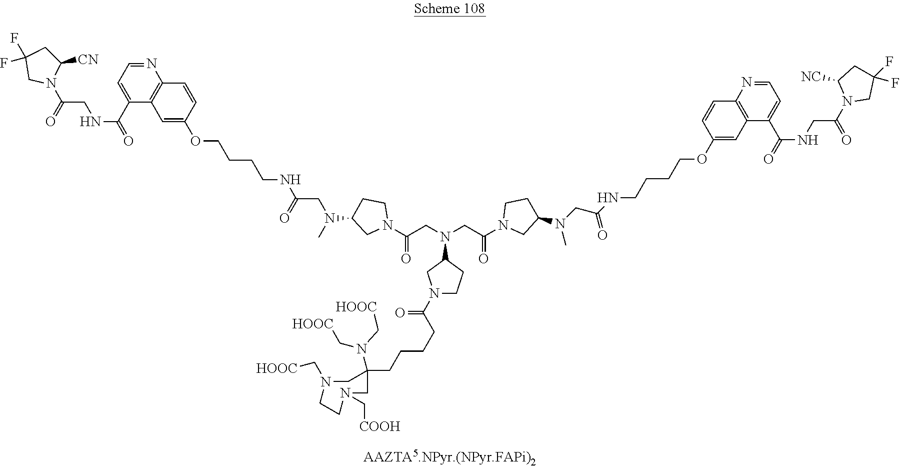

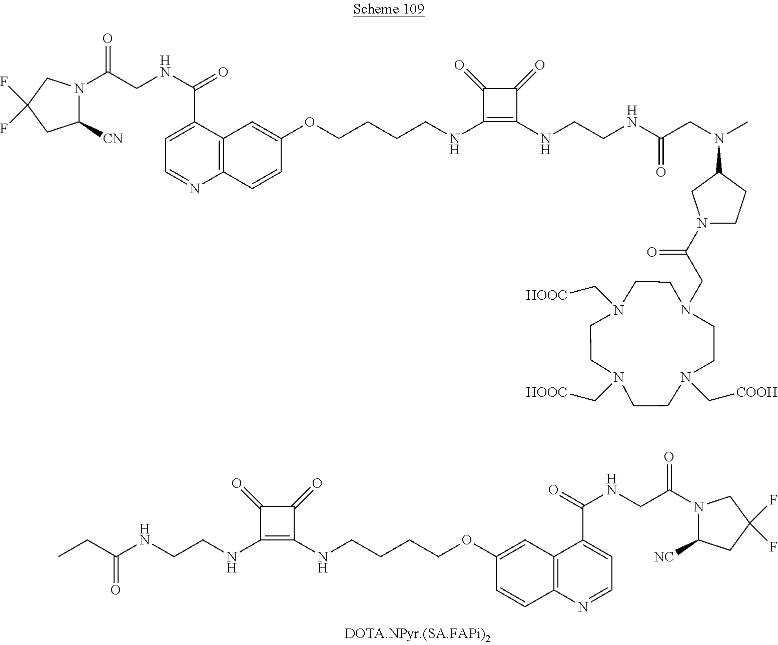

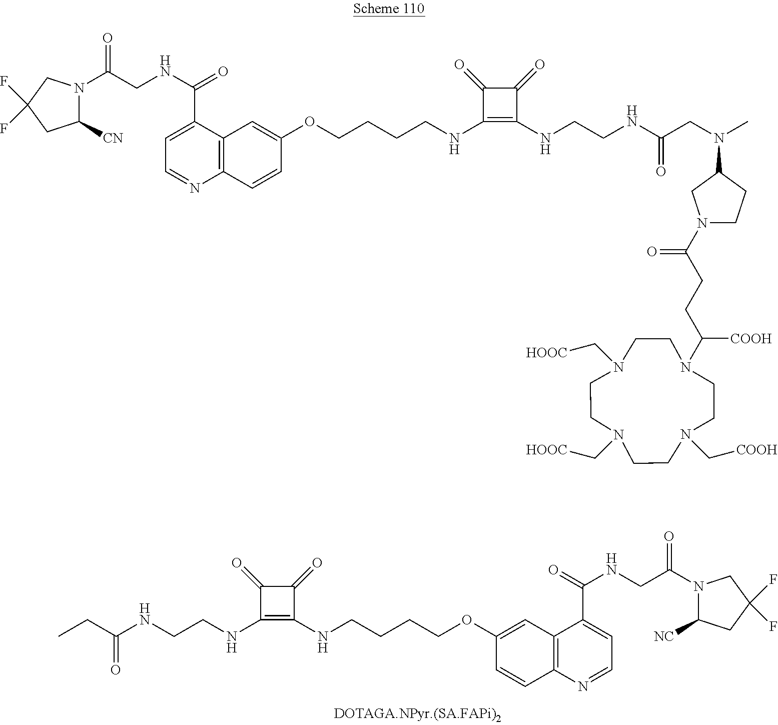

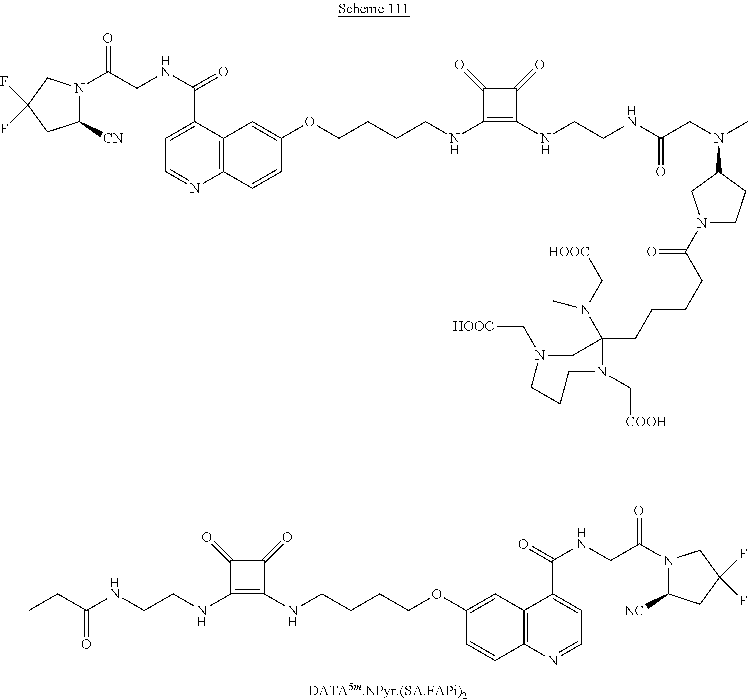

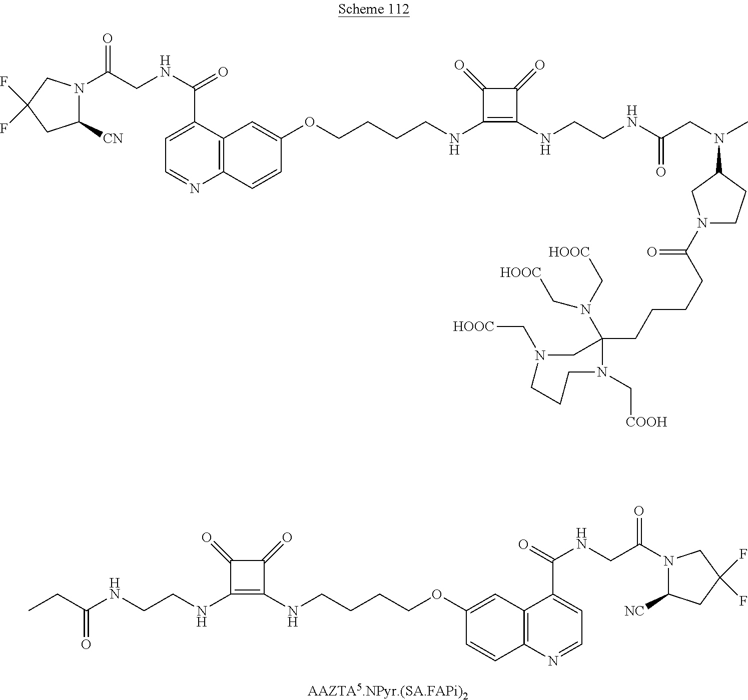

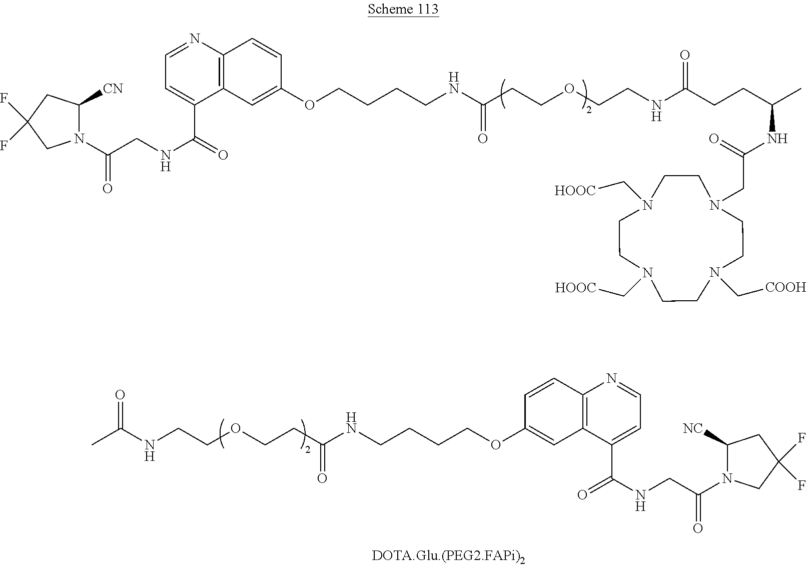

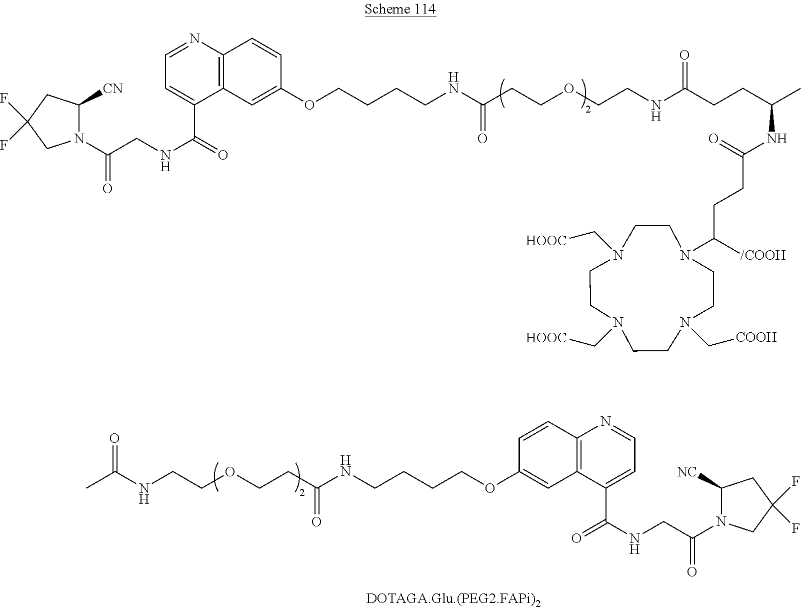

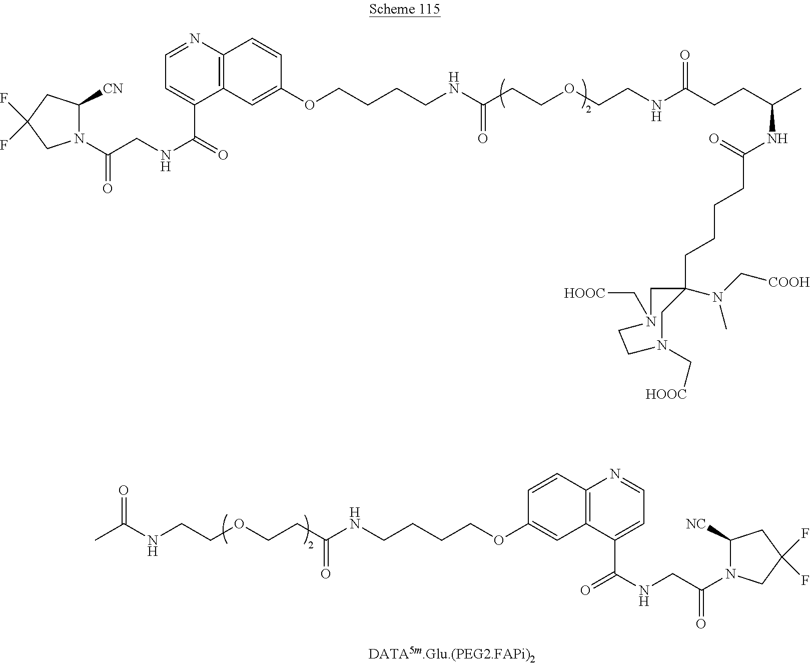

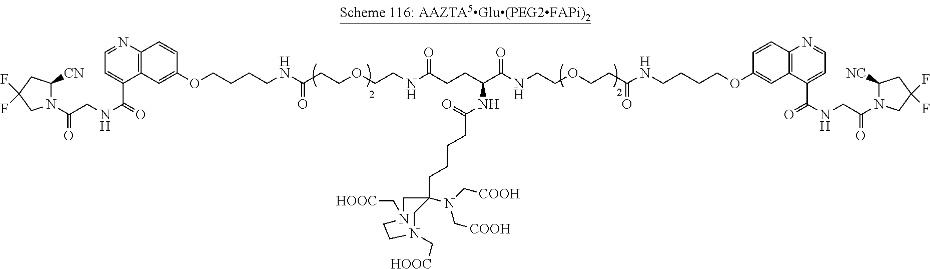

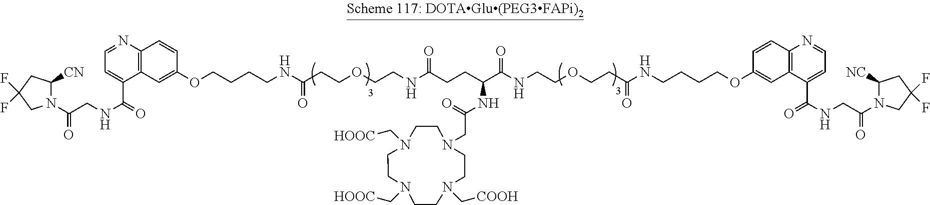

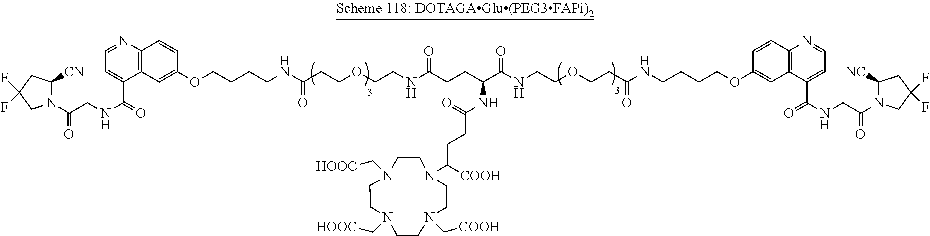

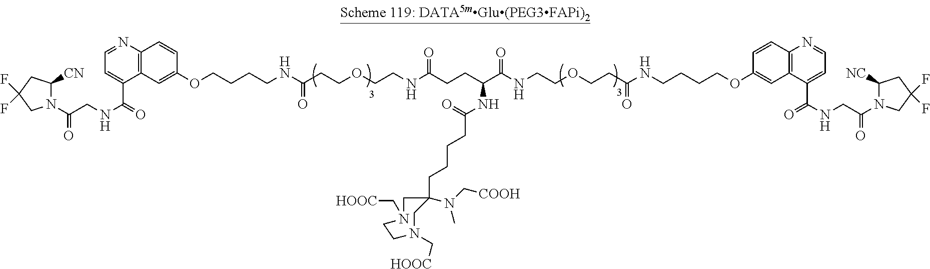

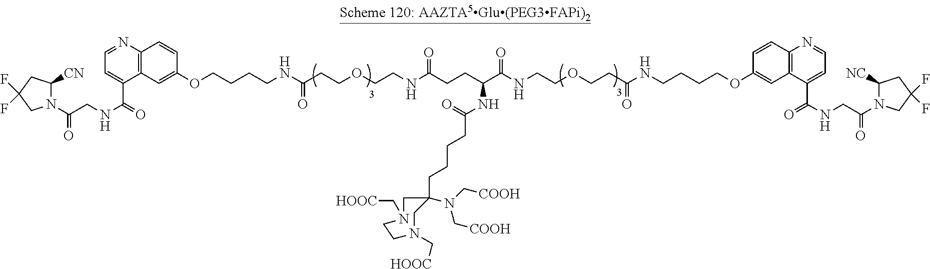

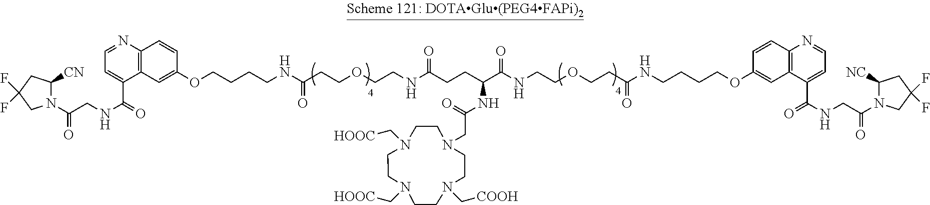

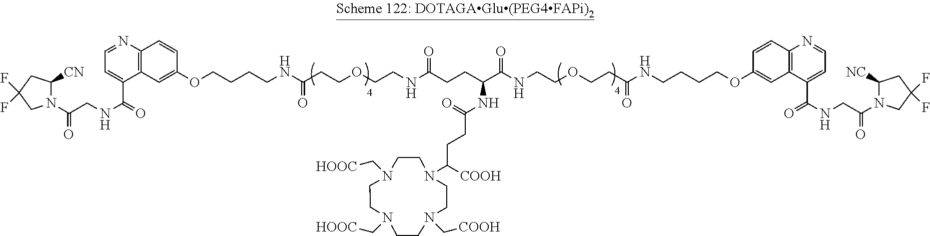

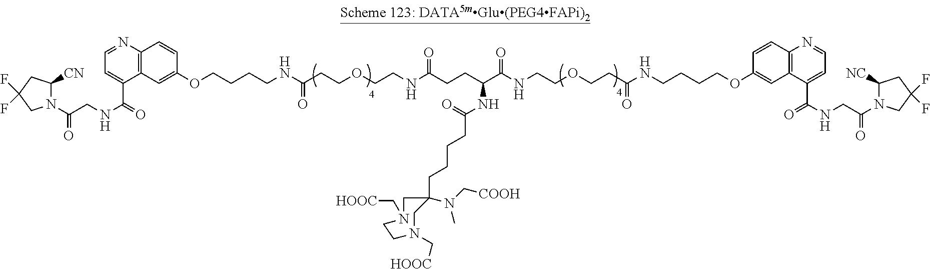

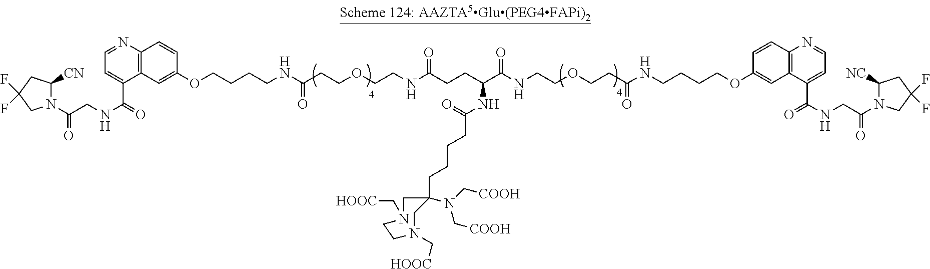

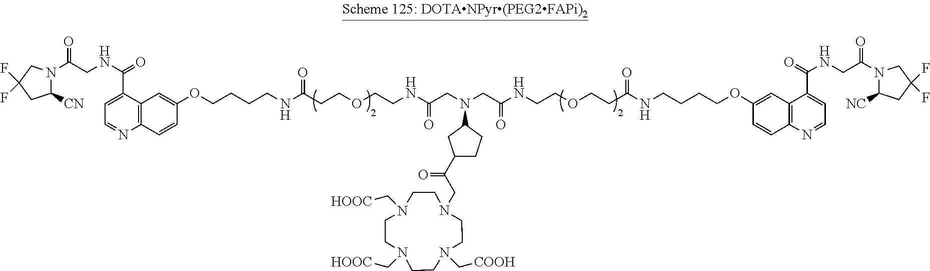

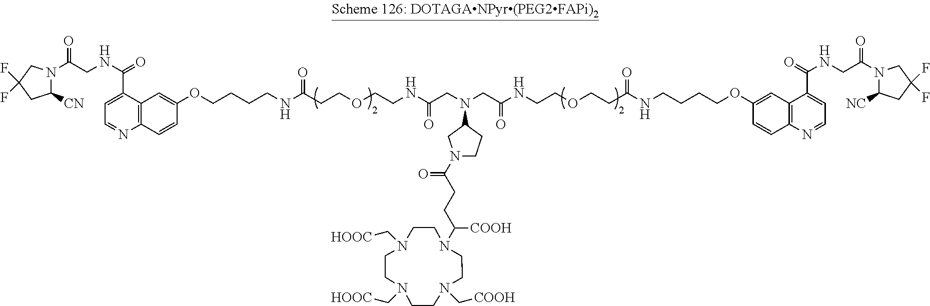

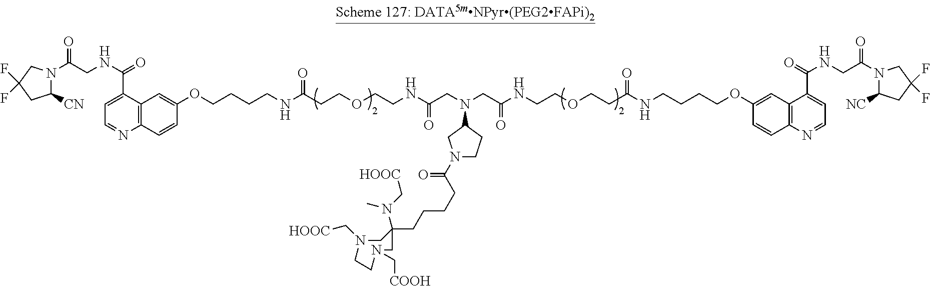

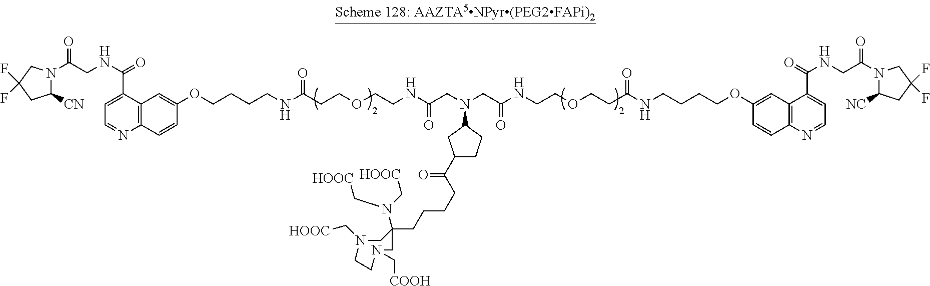

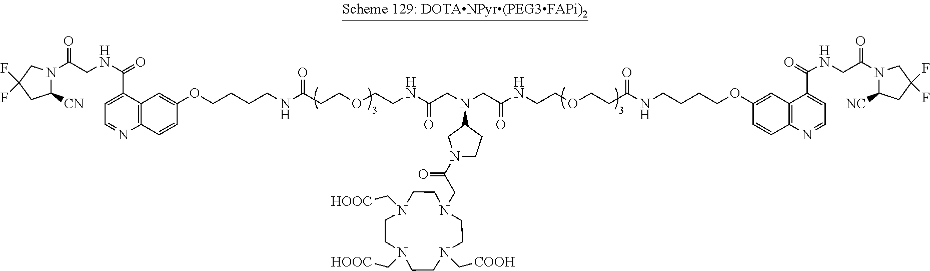

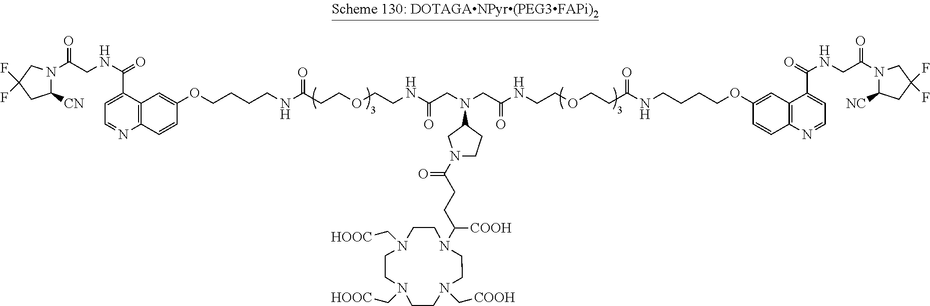

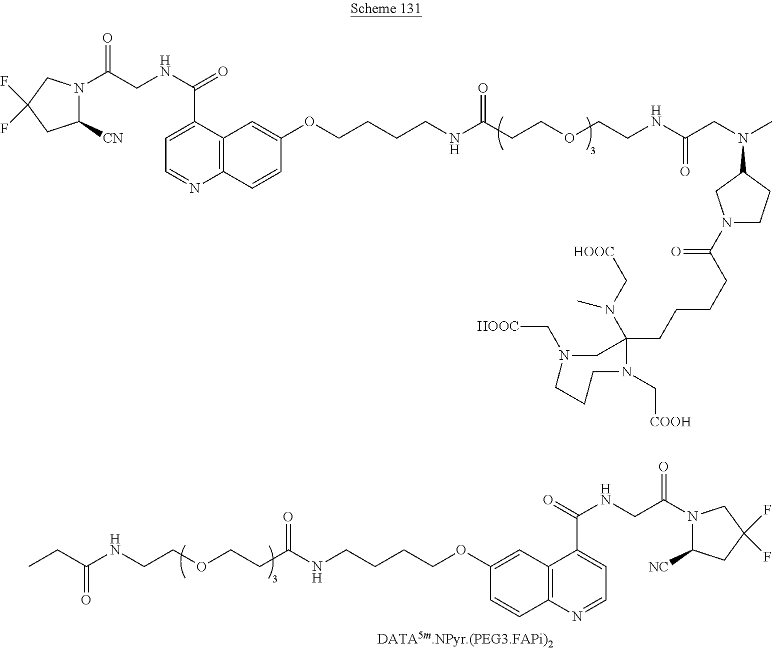

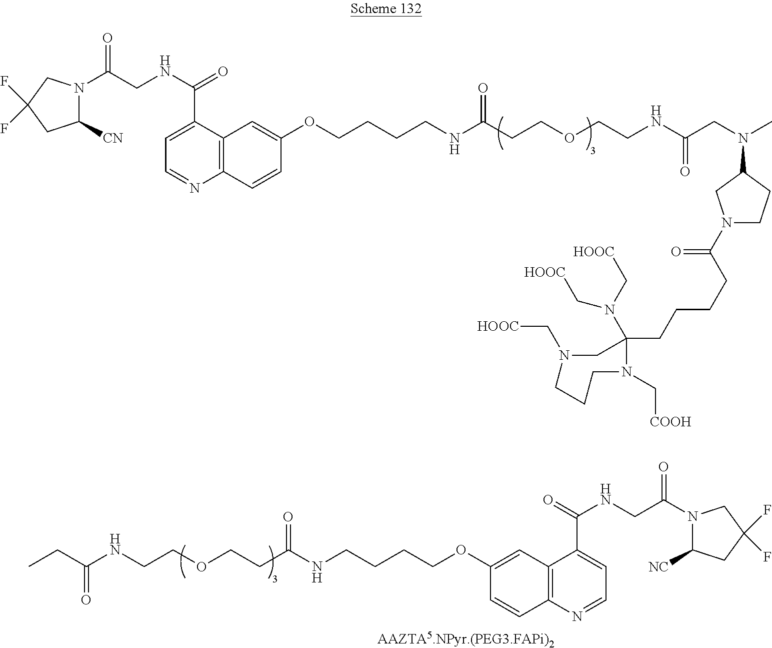

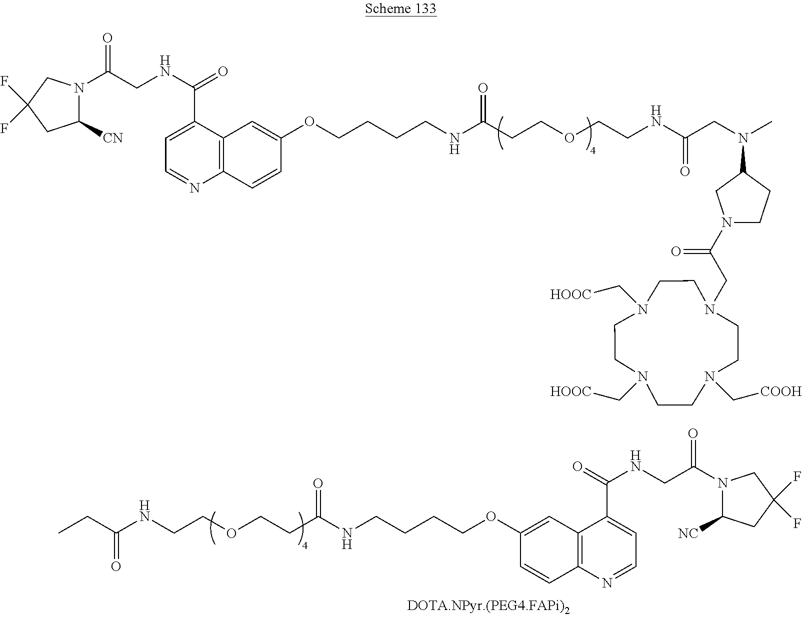

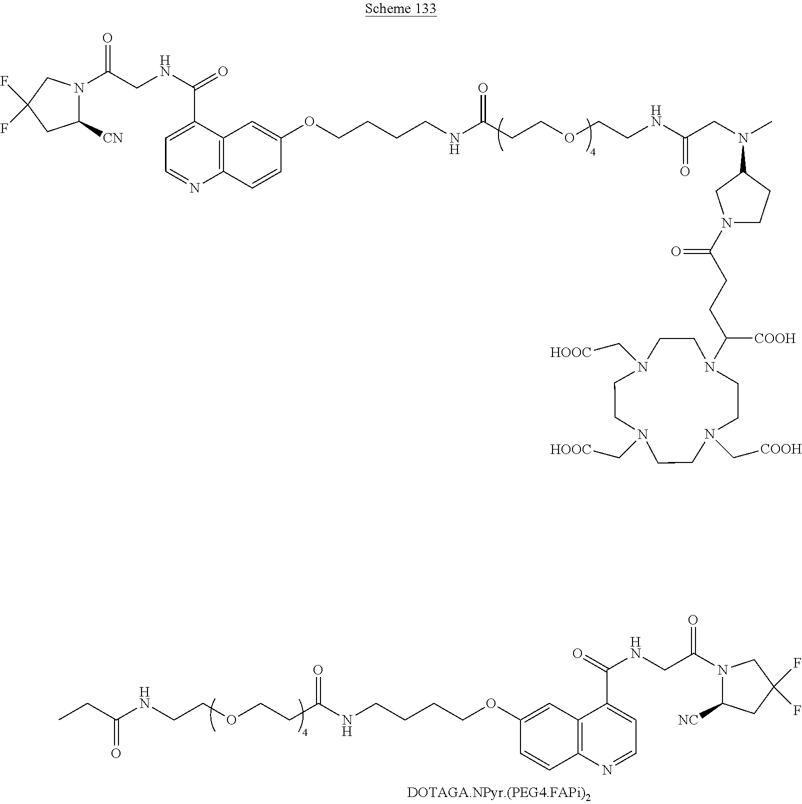

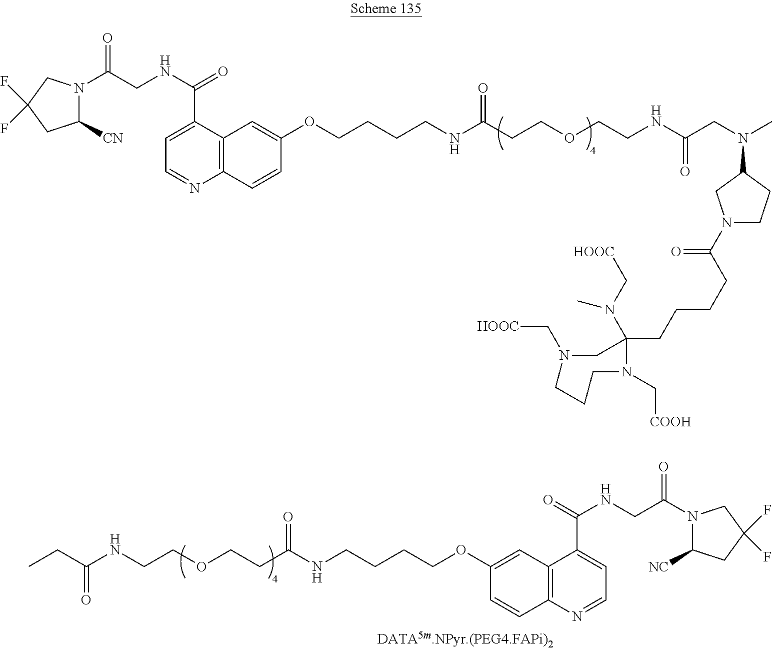

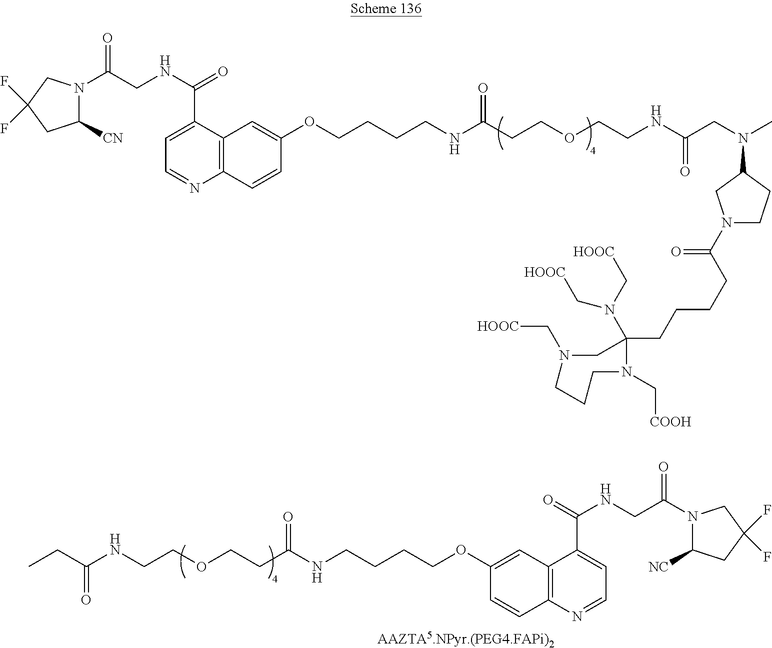

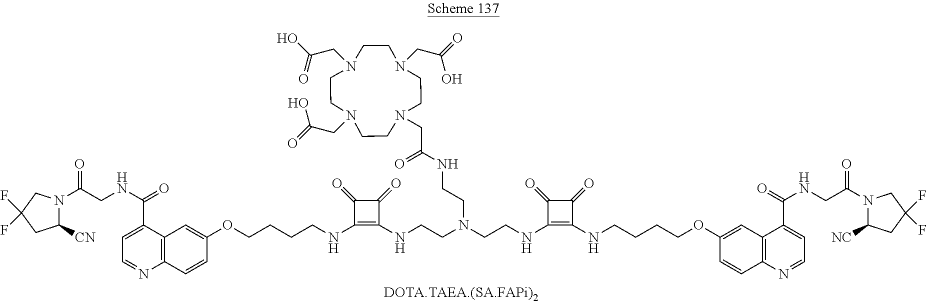 [SMILES: N#C[C@@H]1CC(F)(F)CN1C(=O)CNC(=O)c1ccnc2ccc(OCCCCNc3c(NCCN(CCNC(=O)CN4CCN(CC(=O)O)CCN(CC(=O)O)CCN(CC(=O)O)CC4)CCNc4c(NCCCCOc5ccc6nccc(C(=O)NCC(=O)N7CC(F)(F)C[C@@H]7C#N)c6c5)c(=O)c4=O)c(=O)c3=O)cc12]

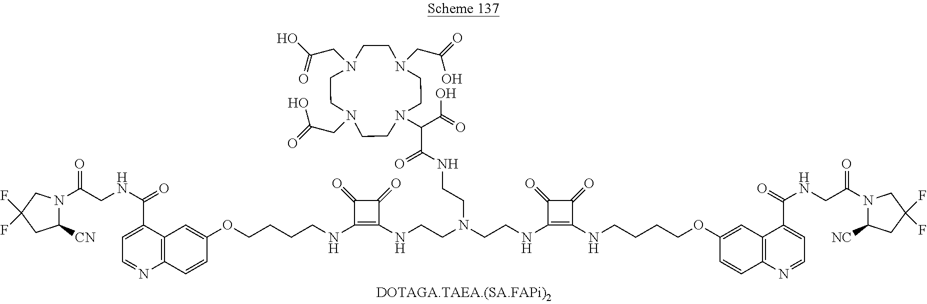 [SMILES: N#C[C@@H]1CC(F)(F)CN1C(=O)CNC(=O)c1ccnc2ccc(OCCCCNc3c(NCCN(CCNC(=O)C(C(=O)O)N4CCN(CC(=O)O)CCN(CC(=O)O)CCN(CC(=O)O)CC4)CCNc4c(NCCCCOc5ccc6nccc(C(=O)NCC(=O)N7CC(F)(F)C[C@@H]7C#N)c6c5)c(=O)c4=O)c(=O)c3=O)cc12]

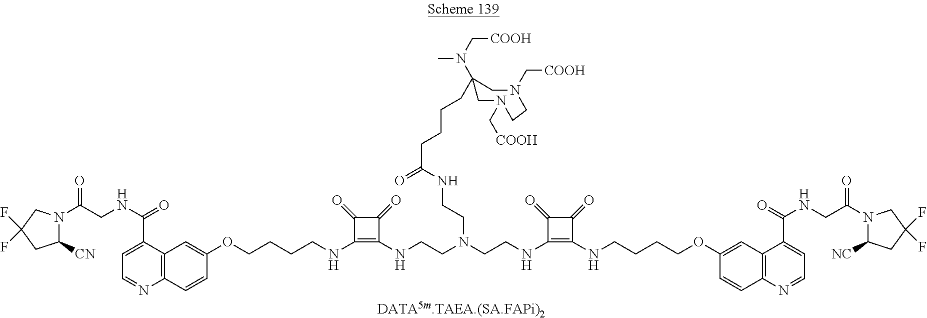 [SMILES: CN(CC(=O)O)C1(CCCCC(=O)NCCN(CCNc2c(NCCCCOc3ccc4nccc(C(=O)NCC(=O)N5CC(F)(F)C[C@H]5C#N)c4c3)c(=O)c2=O)CCNc2c(NCCCCOc3ccc4nccc(C(=O)NCC(=O)N5CC(F)(F)C[C@@H]5C#N)c4c3)c(=O)c2=O)CN(CC(=O)O)CCN(CC(=O)O)C1]

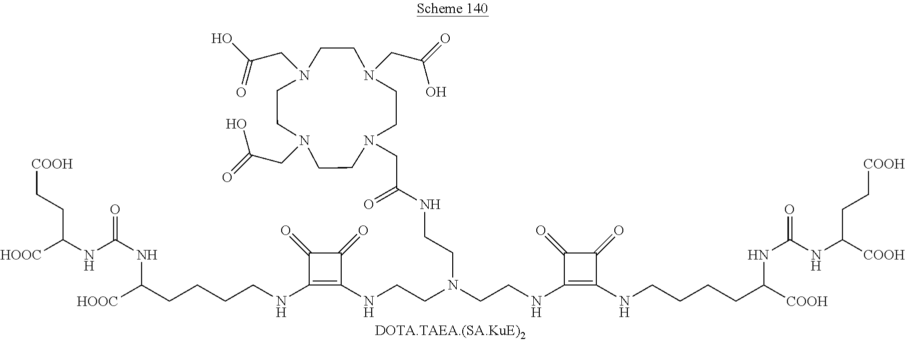 [SMILES: O=C(O)CCC(NC(=O)NC(CCCCNc1c(NCCN(CCNC(=O)CN2CCN(CC(=O)O)CCN(CC(=O)O)CCN(CC(=O)O)CC2)CCNc2c(NCCCCC(NC(=O)NC(CCC(=O)O)C(=O)O)C(=O)O)c(=O)c2=O)c(=O)c1=O)C(=O)O)C(=O)O]

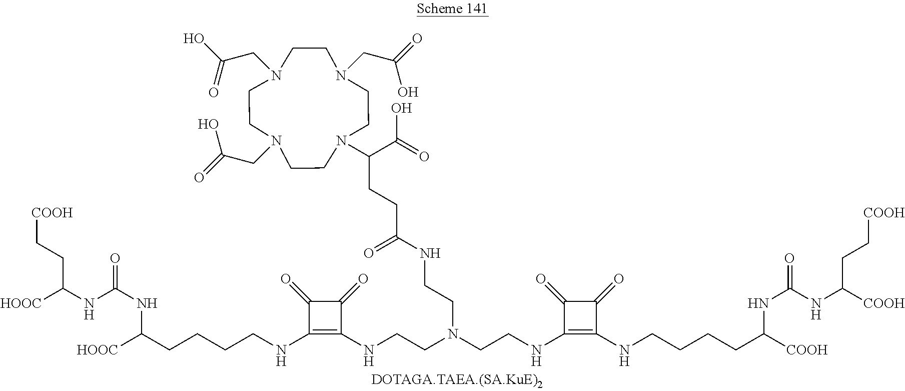 [SMILES: O=C(O)CCC(NC(=O)NC(CCCCNc1c(NCCN(CCNC(=O)CCC(C(=O)O)N2CCN(CC(=O)O)CCN(CC(=O)O)CCN(CC(=O)O)CC2)CCNc2c(NCCCCC(NC(=O)NC(CCC(=O)O)C(=O)O)C(=O)O)c(=O)c2=O)c(=O)c1=O)C(=O)O)C(=O)O]

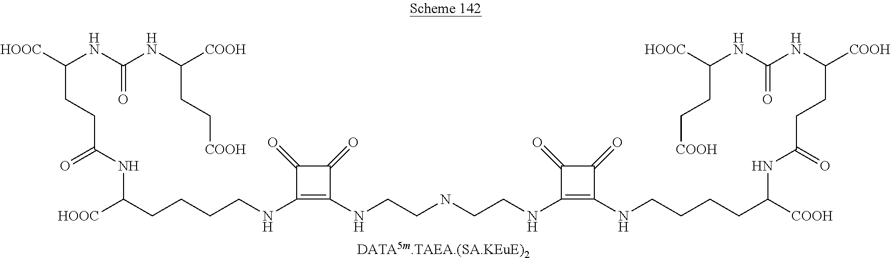 [SMILES: O=C(O)CCC(NC(=O)NC(CCC(=O)NC(CCCCNc1c(NCCNCCNc2c(NCCCCC(NC(=O)CCC(NC(=O)NC(CCC(=O)O)C(=O)O)C(=O)O)C(=O)O)c(=O)c2=O)c(=O)c1=O)C(=O)O)C(=O)O)C(=O)O]

### Example 9

Examples of inventive compounds with three spacer units (S1+S2+S3) are shown below.

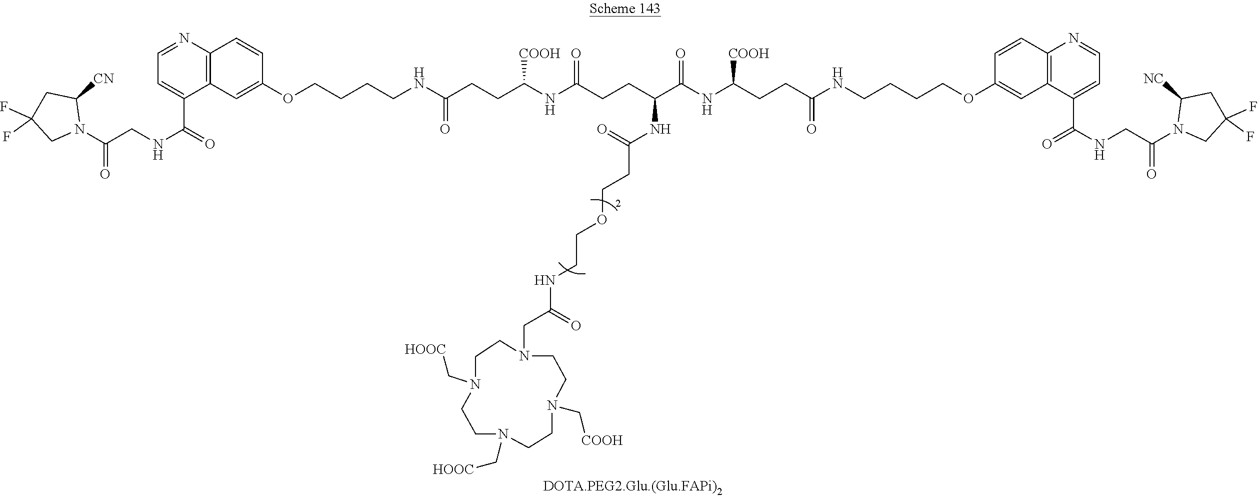

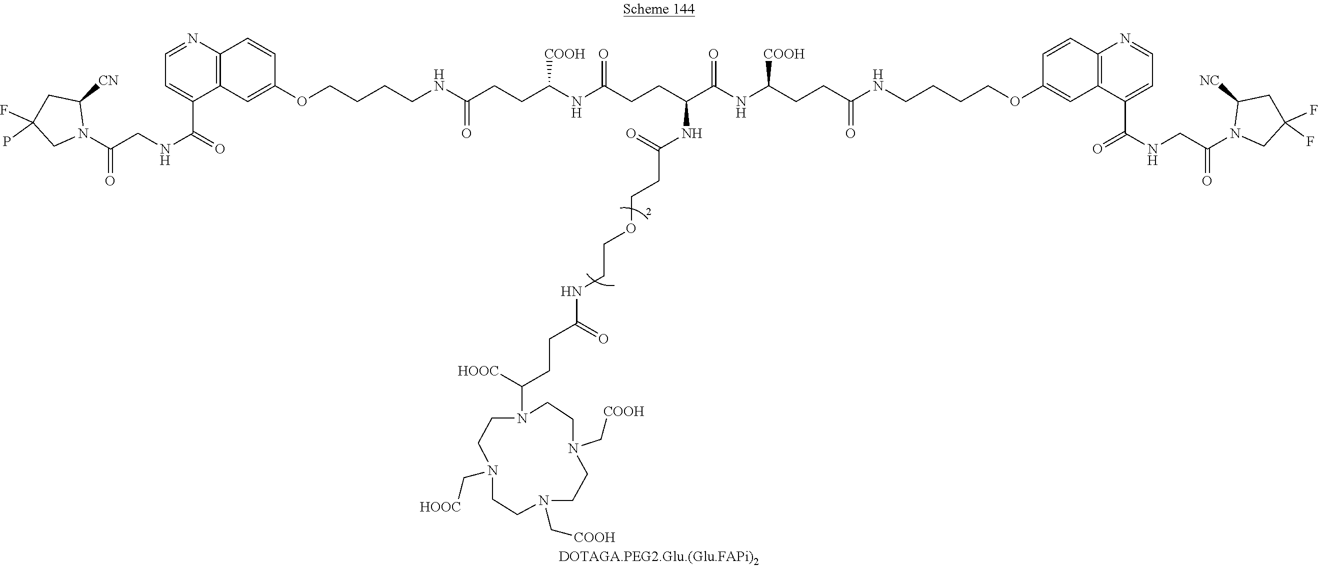

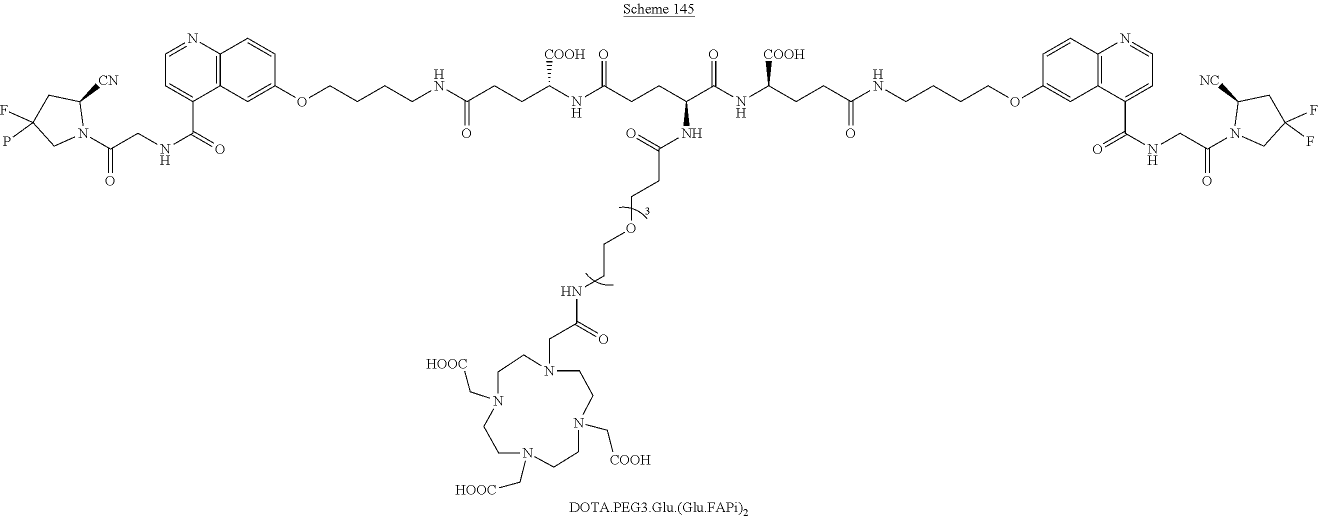

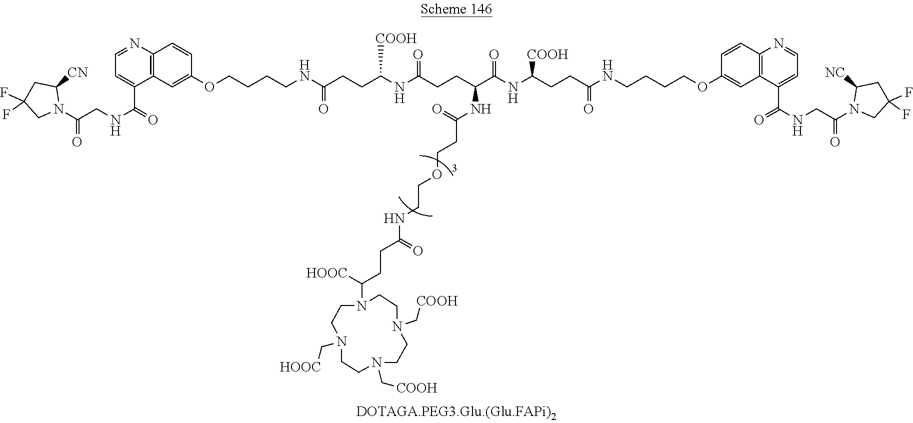

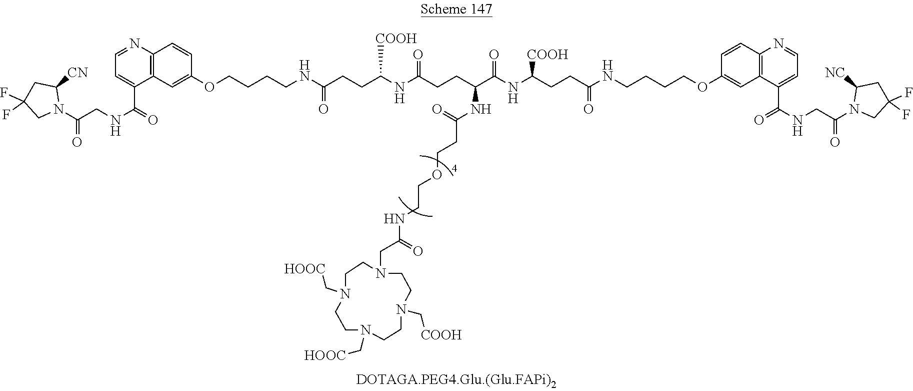

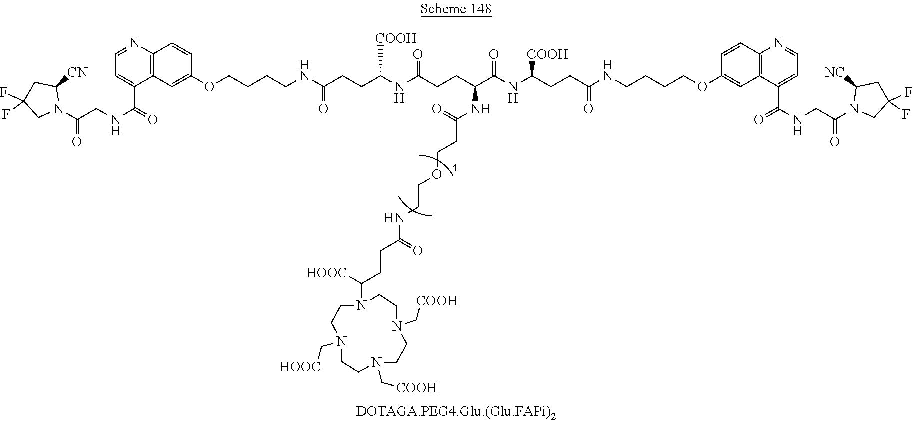

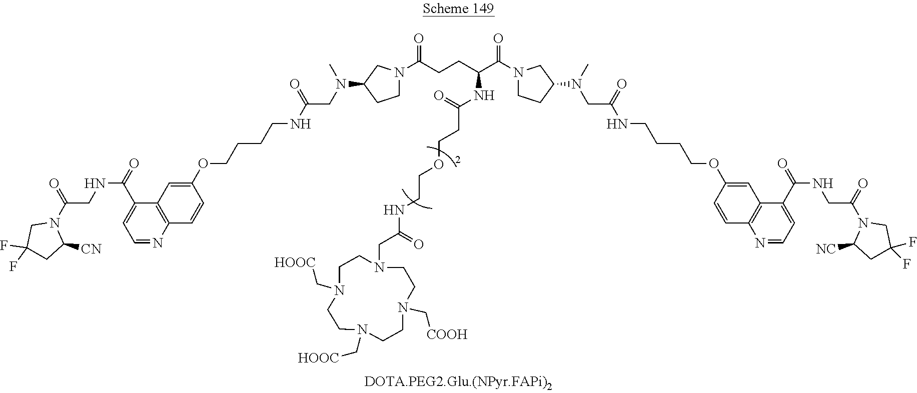

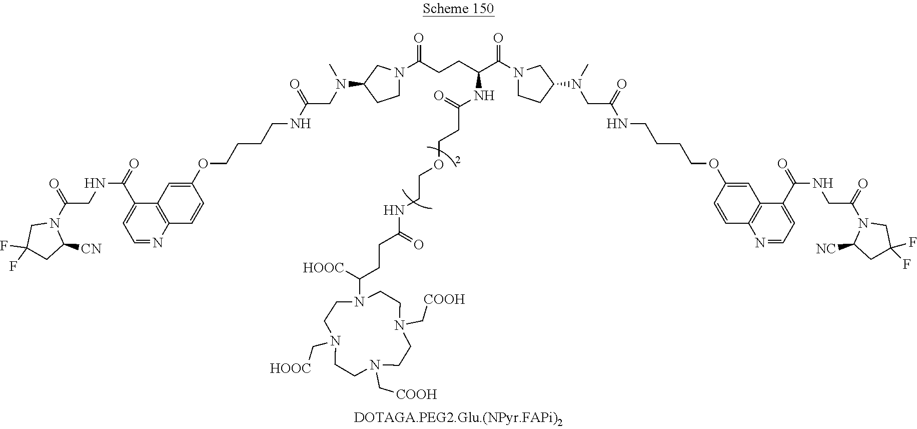

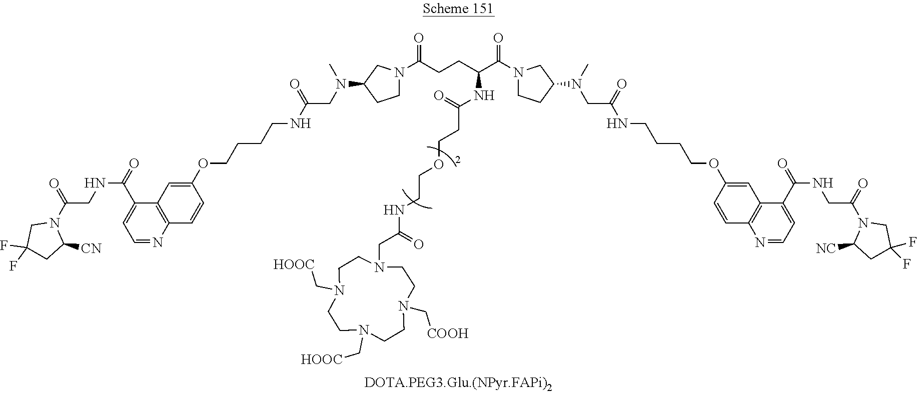

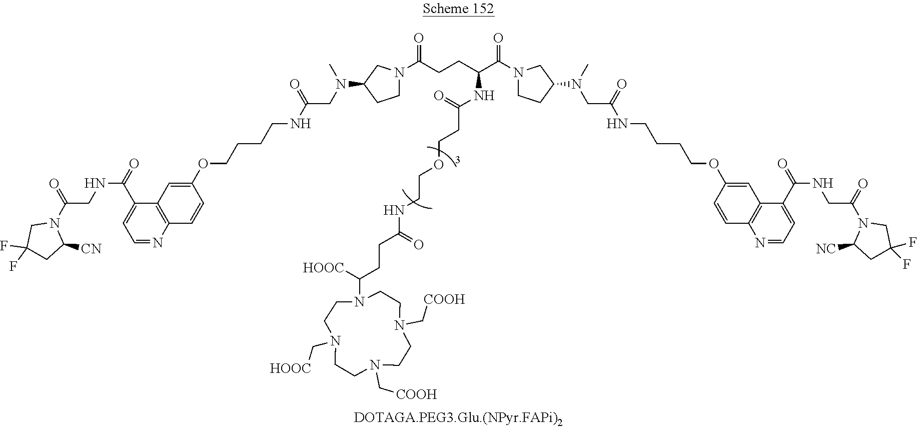

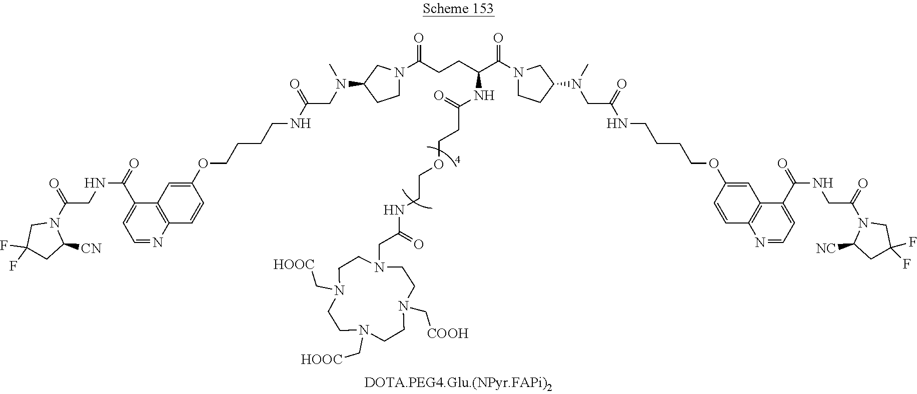

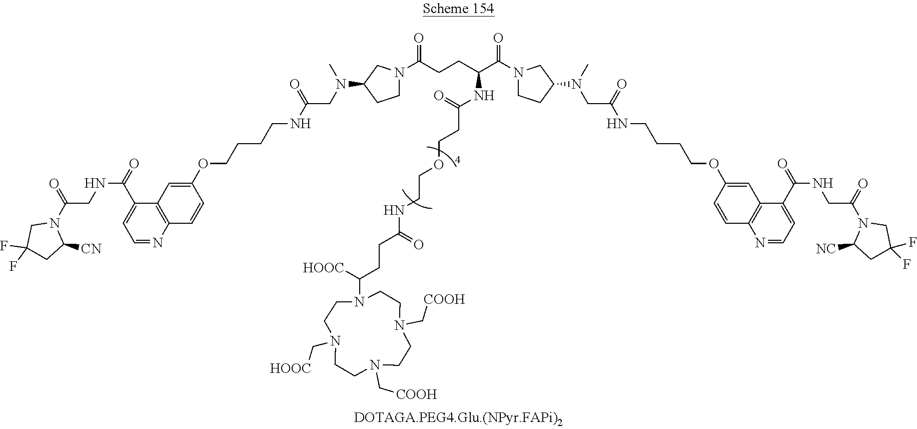

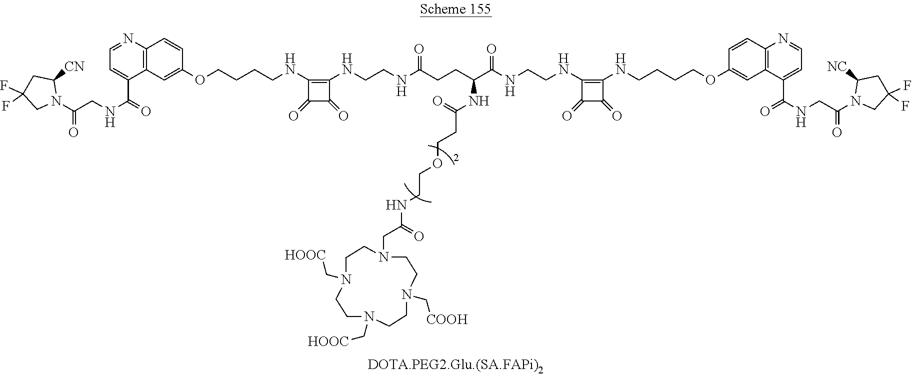

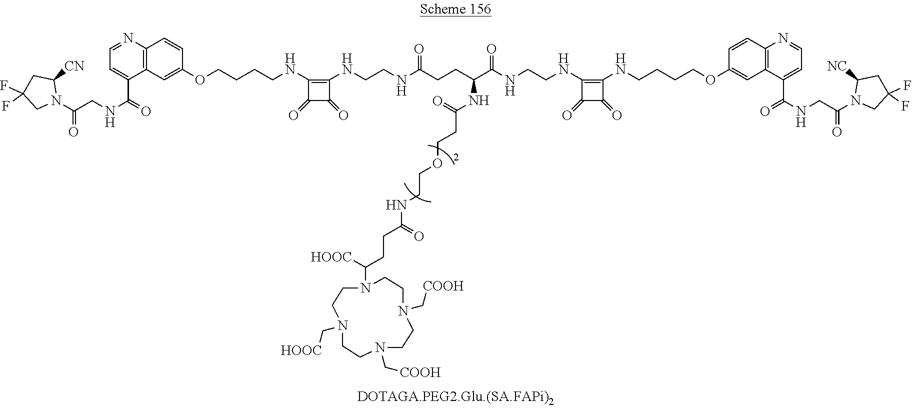

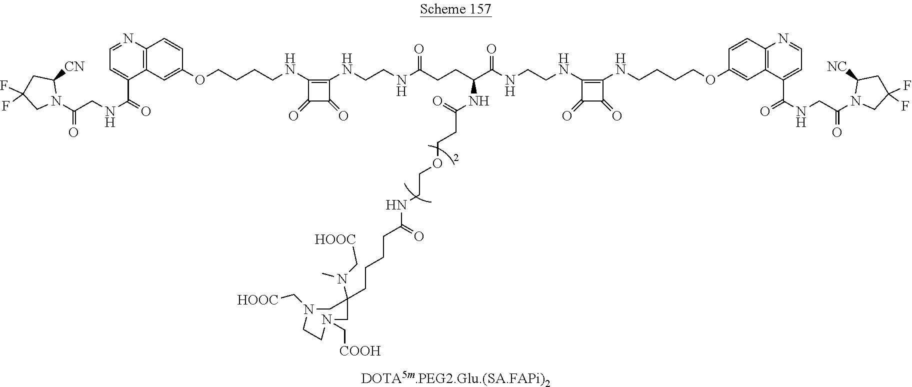

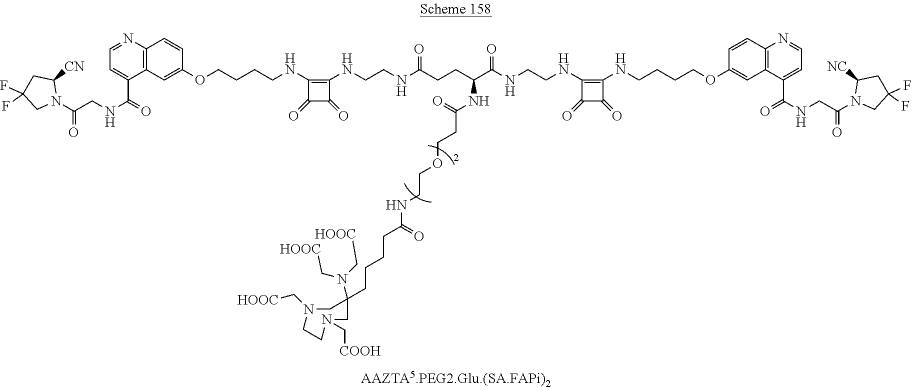

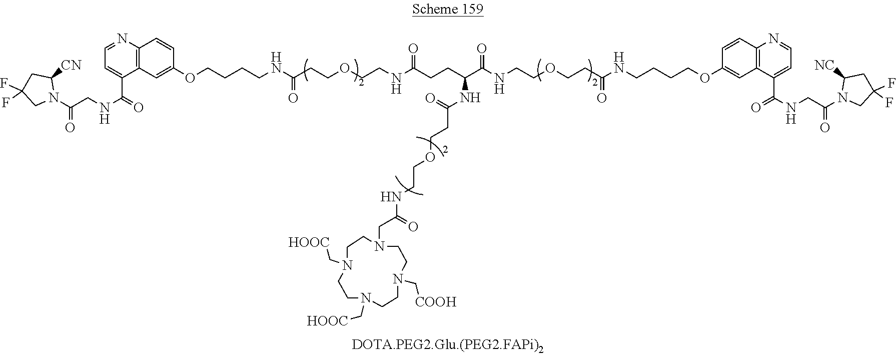

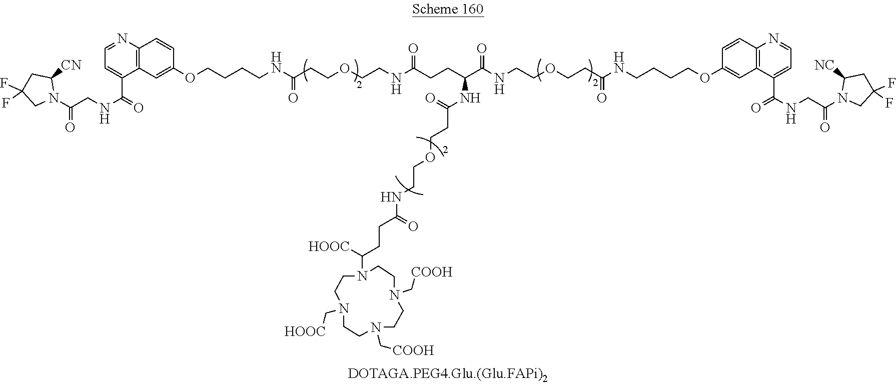

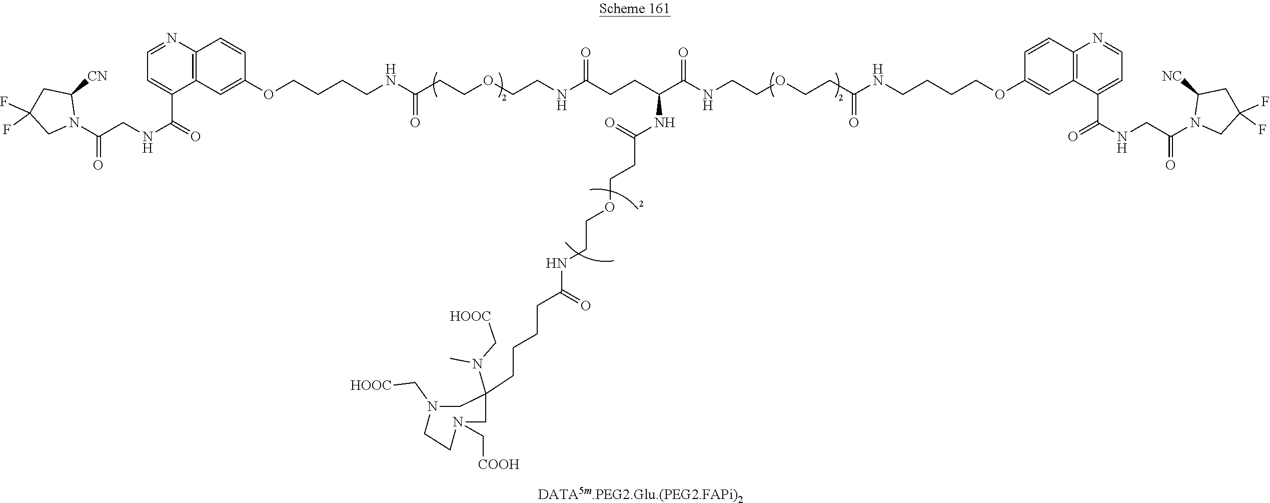

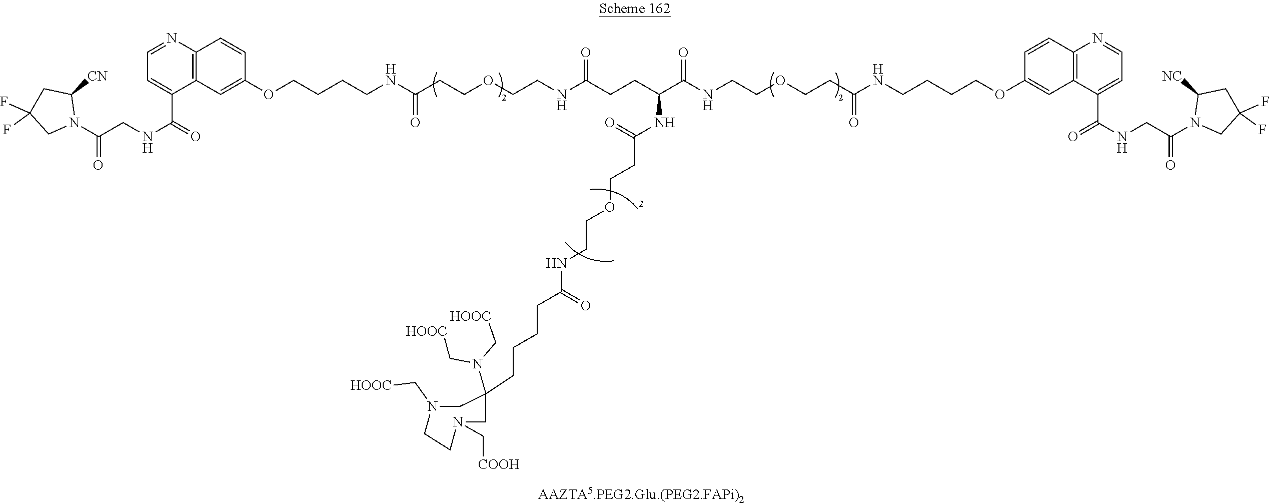

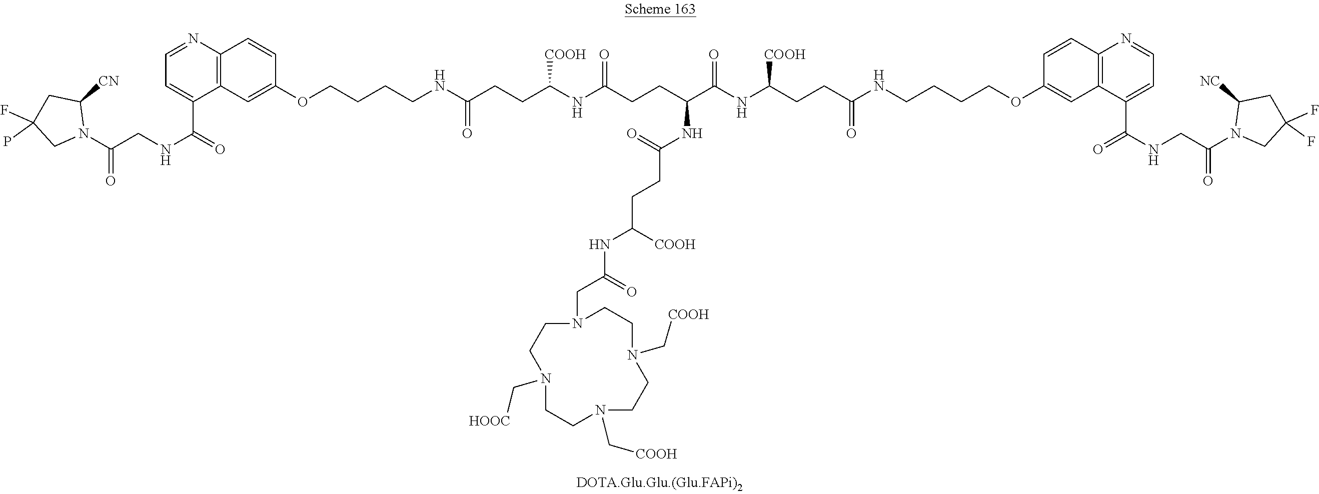

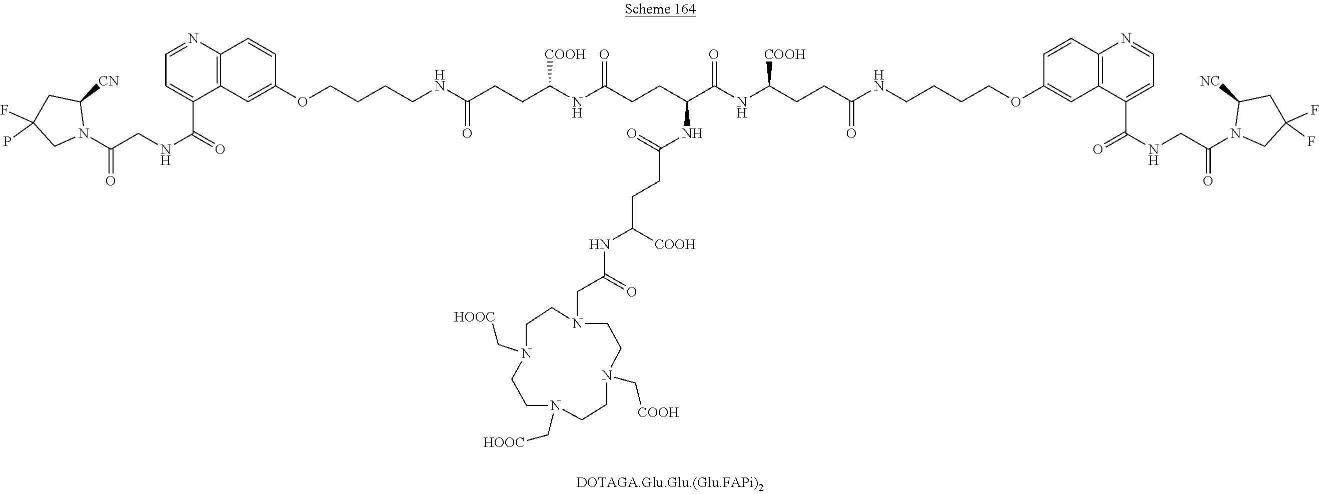

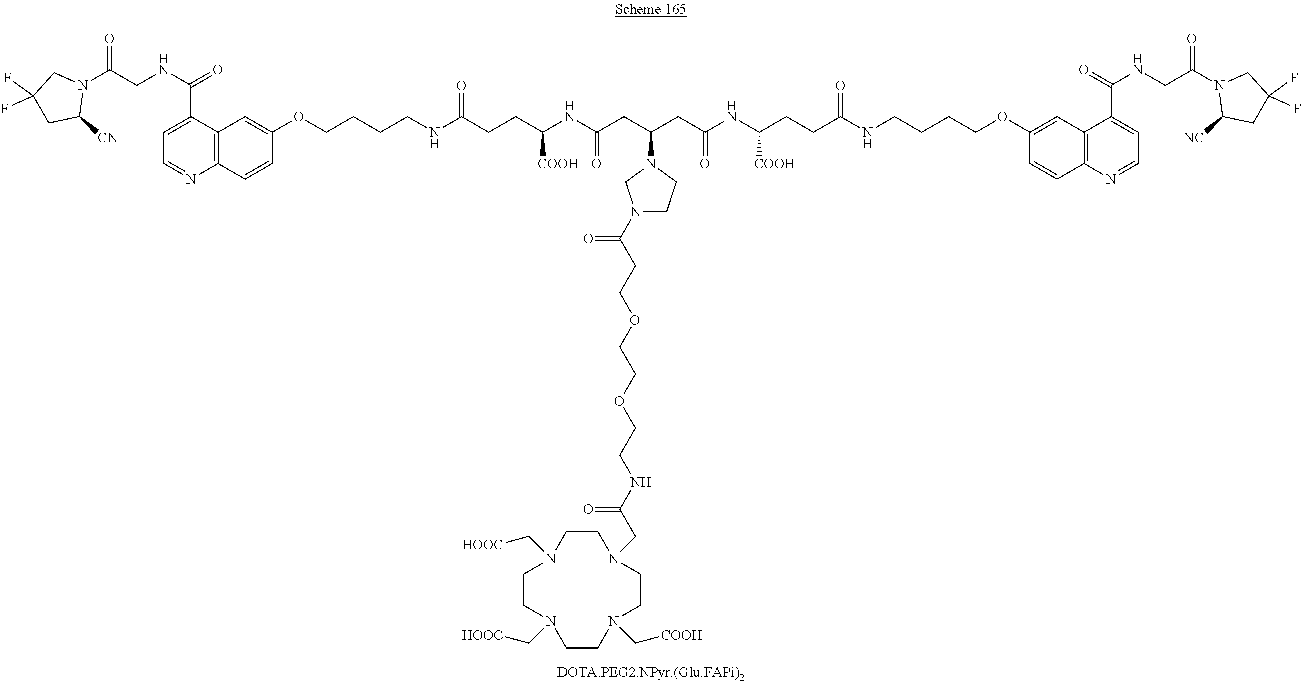 [SMILES: N#C[C@@H]1CC(F)(F)CN1C(=O)CNC(=O)c1ccnc2ccc(OCCCCNC(=O)CC[C@@H](NC(=O)C[C@H](CC(=O)N[C@H](CCC(=O)NCCCCOc3ccc4nccc(C(=O)NCC(=O)N5CC(F)(F)C[C@@H]5C#N)c4c3)C(=O)O)N3CCN(C(=O)CCOCCOCCNC(=O)CN4CCN(CC(=O)O)CCN(CC(=O)O)CCN(CC(=O)O)CC4)C3)C(=O)O)cc12]

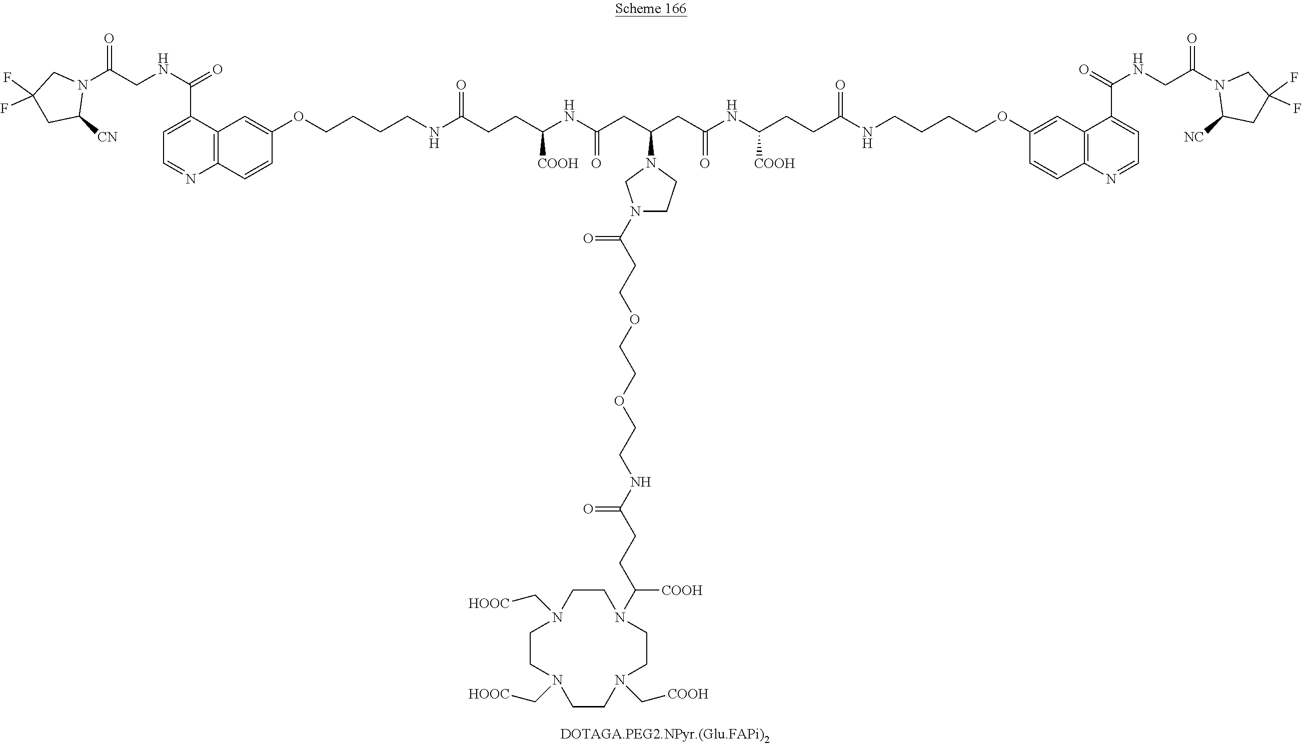 [SMILES: N#C[C@@H]1CC(F)(F)CN1C(=O)CNC(=O)c1ccnc2ccc(OCCCCNC(=O)CC[C@@H](NC(=O)C[C@H](CC(=O)N[C@H](CCC(=O)NCCCCOc3ccc4nccc(C(=O)NCC(=O)N5CC(F)(F)C[C@@H]5C#N)c4c3)C(=O)O)N3CCN(C(=O)CCOCCOCCNC(=O)CCC(C(=O)O)N4CCN(CC(=O)O)CCN(CC(=O)O)CCN(CC(=O)O)CC4)C3)C(=O)O)cc12]

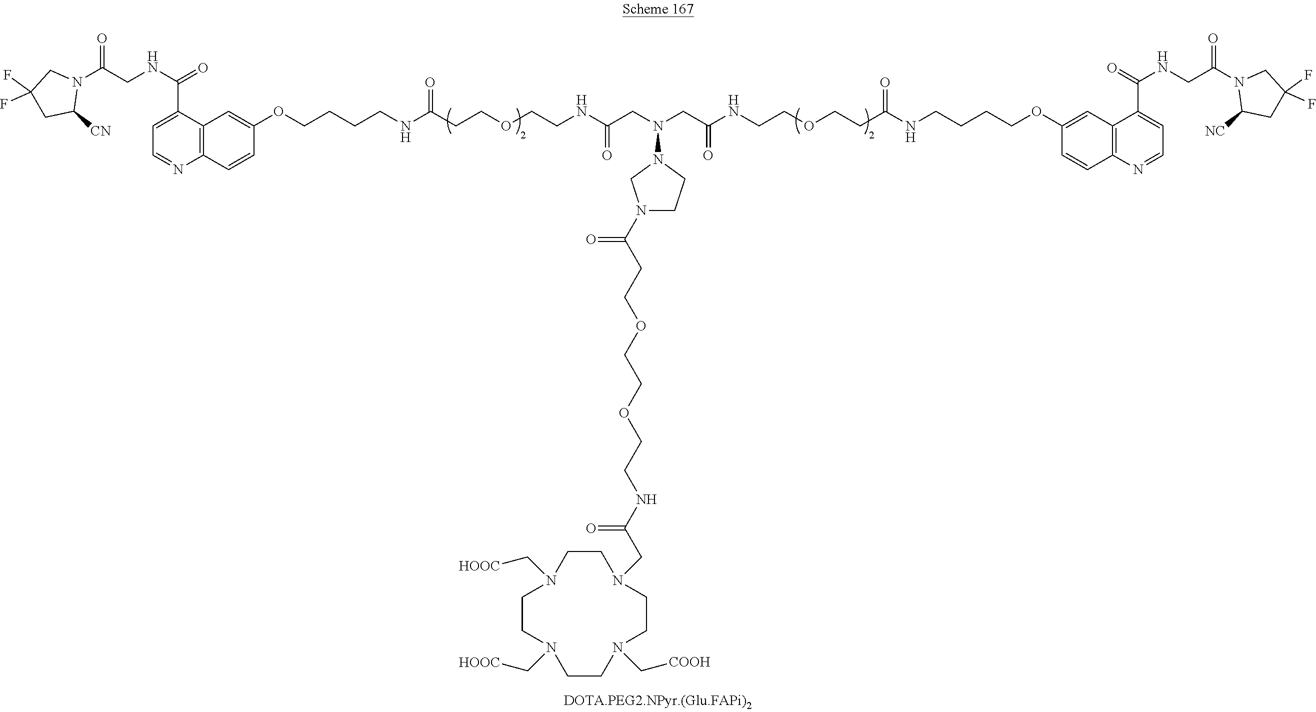

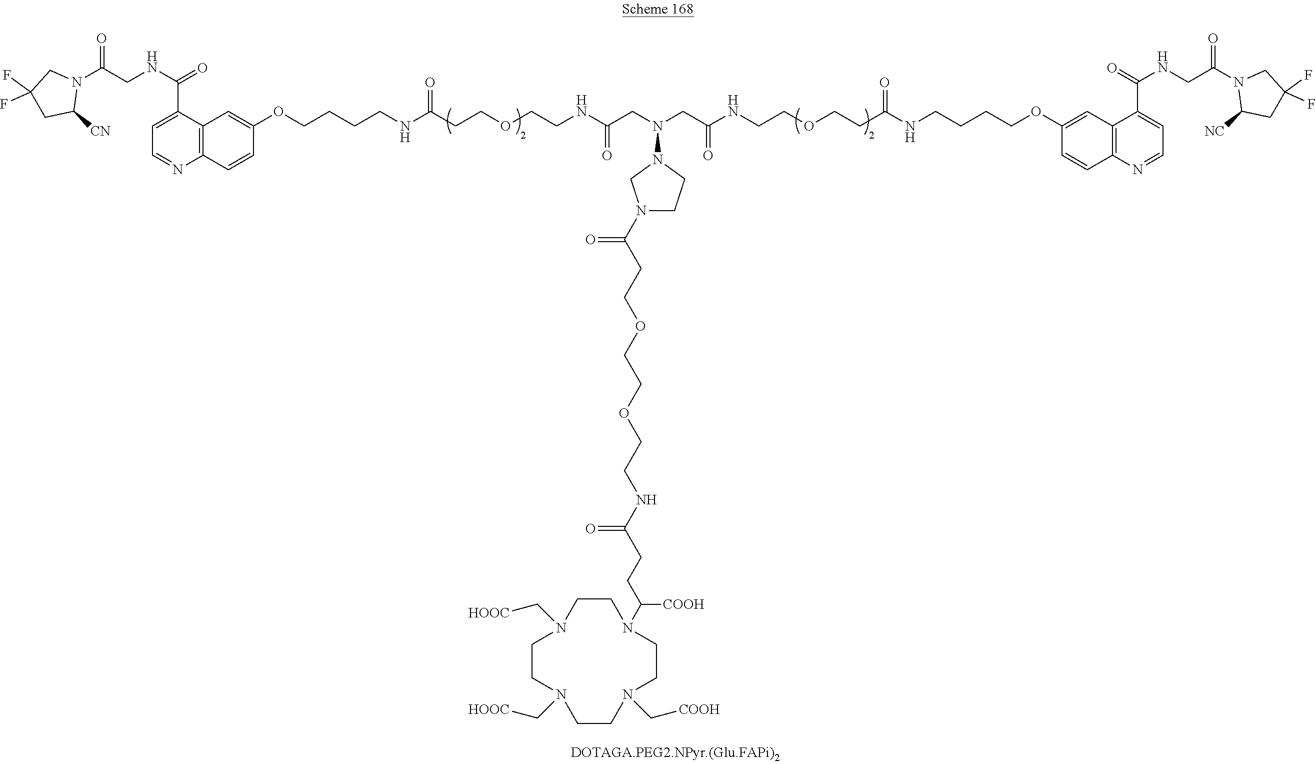

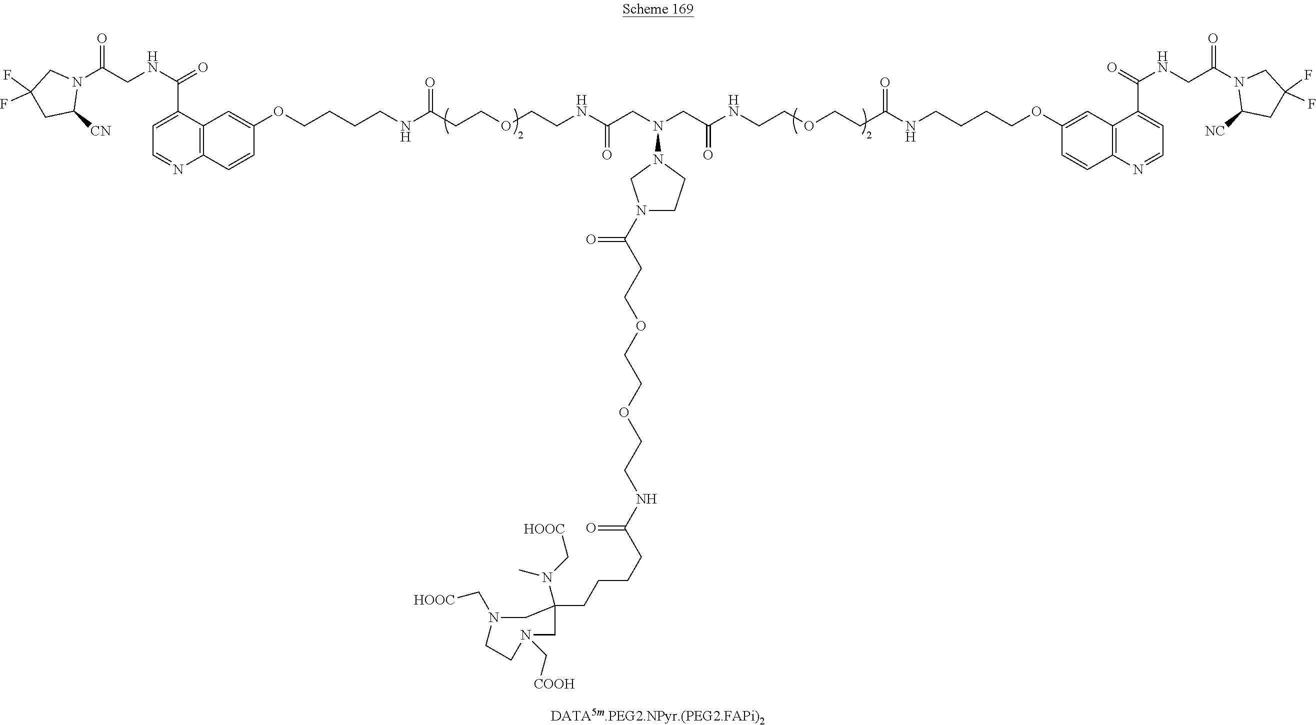

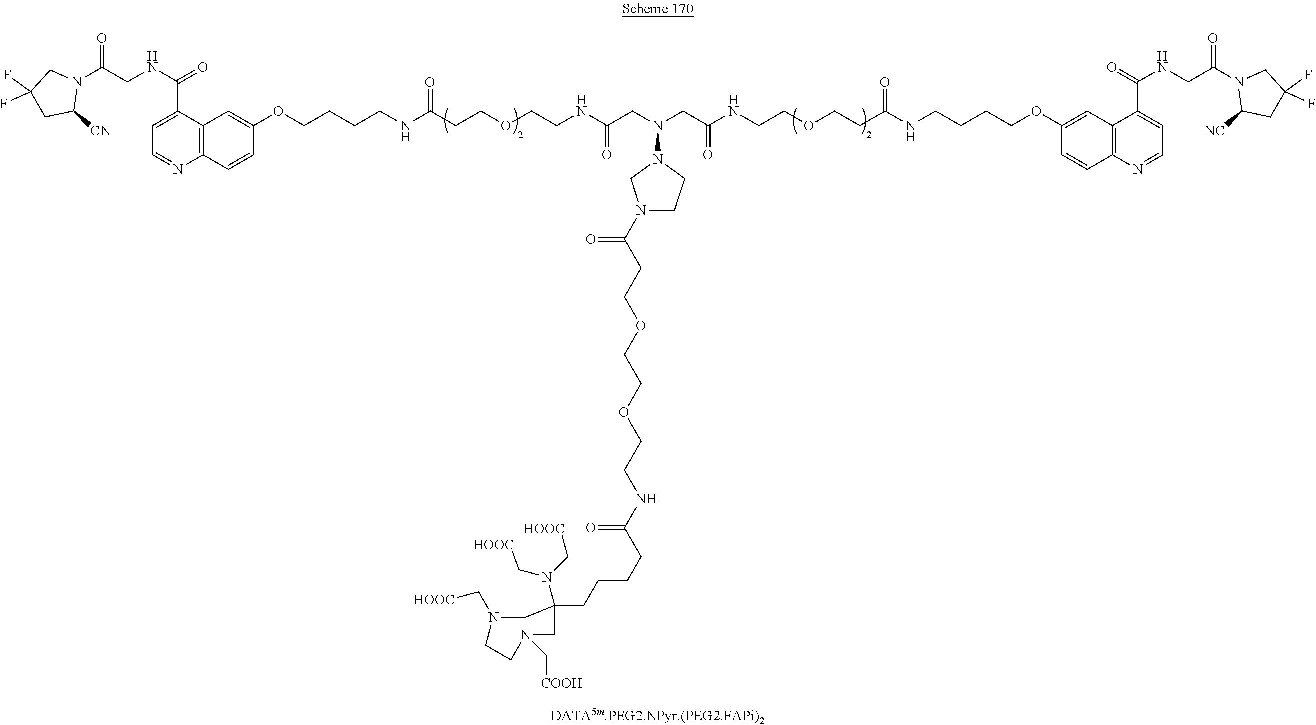

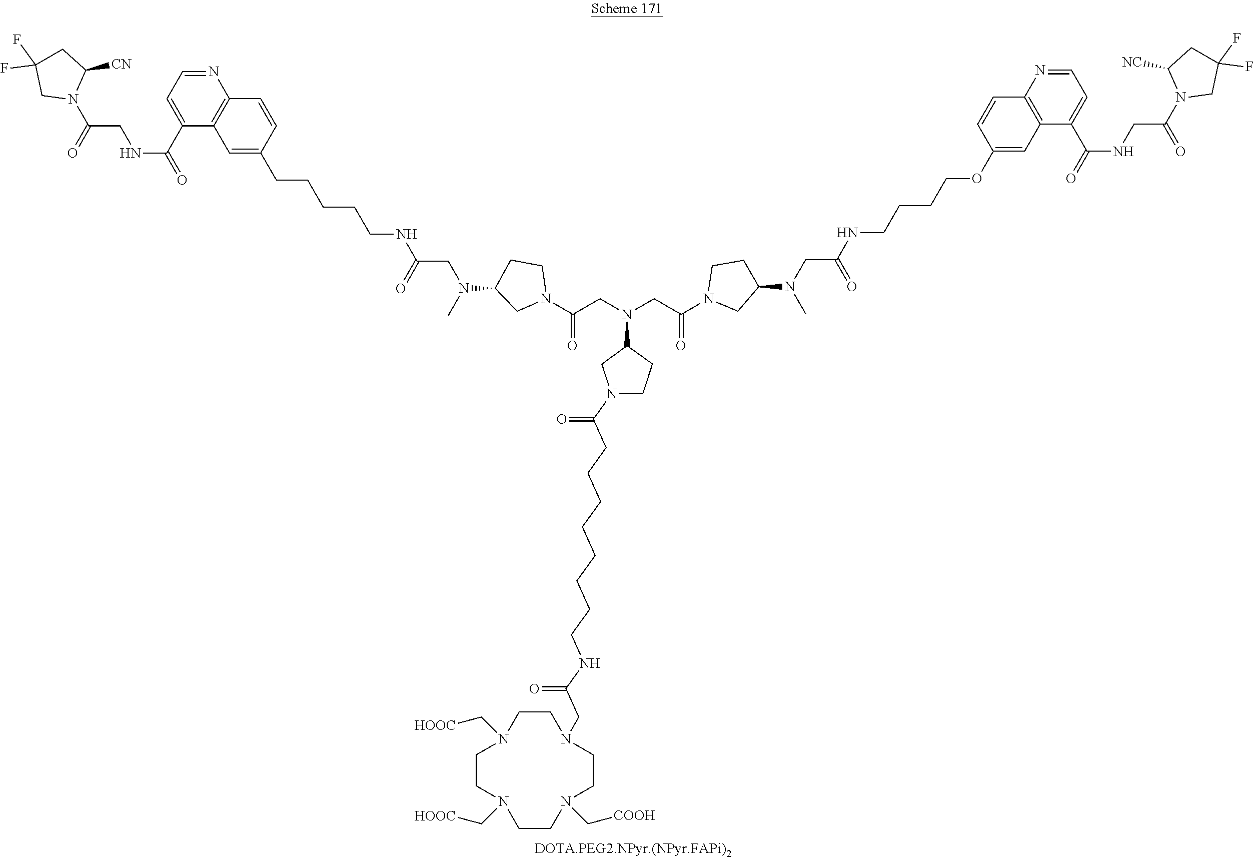

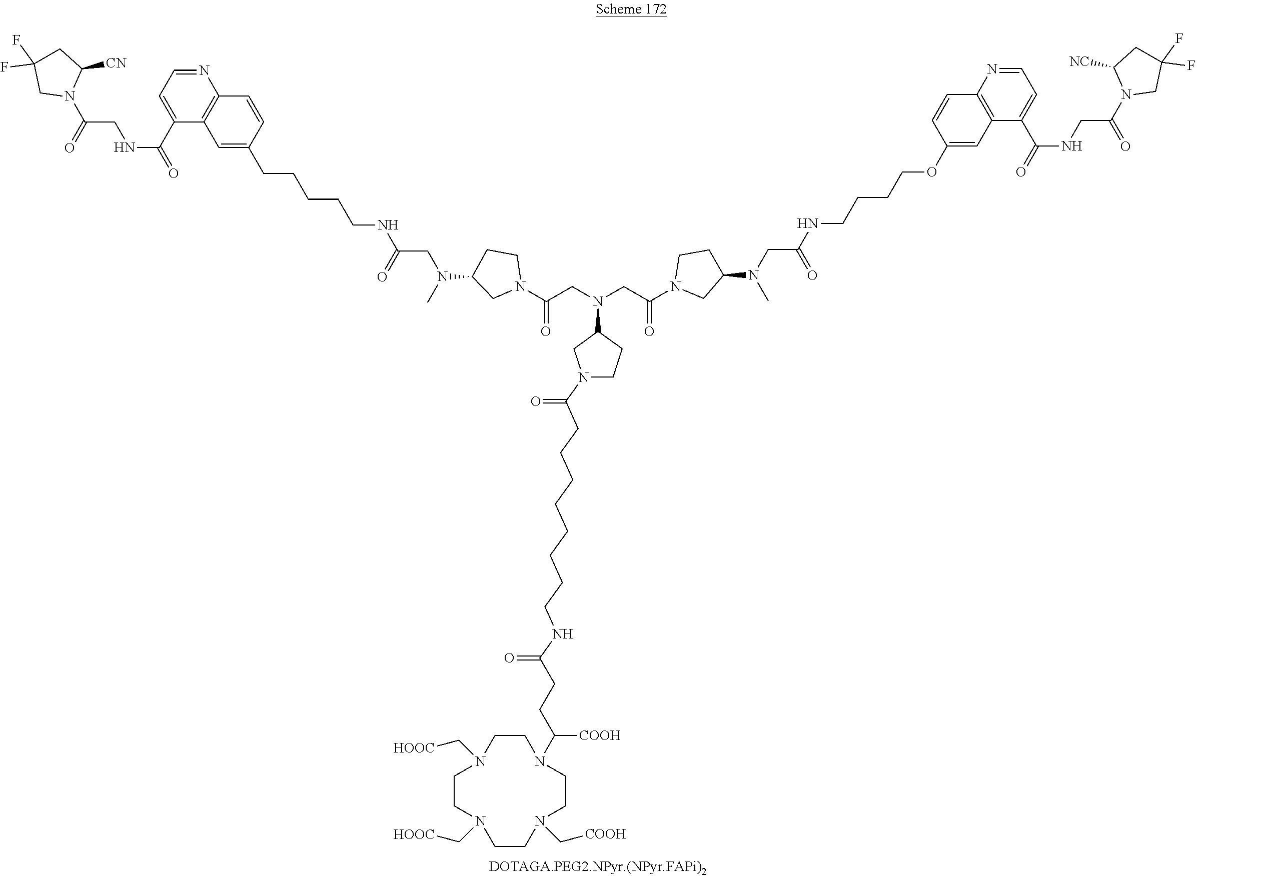

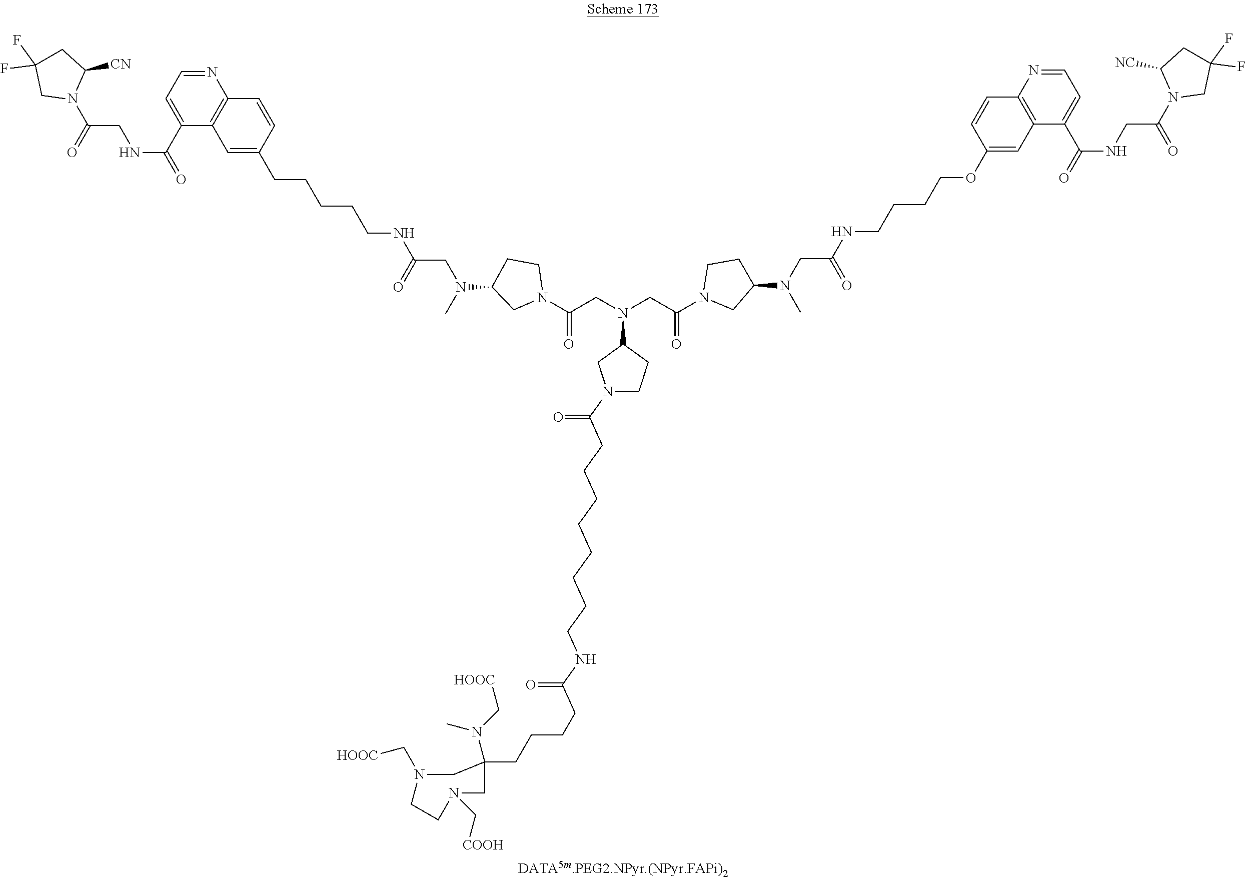

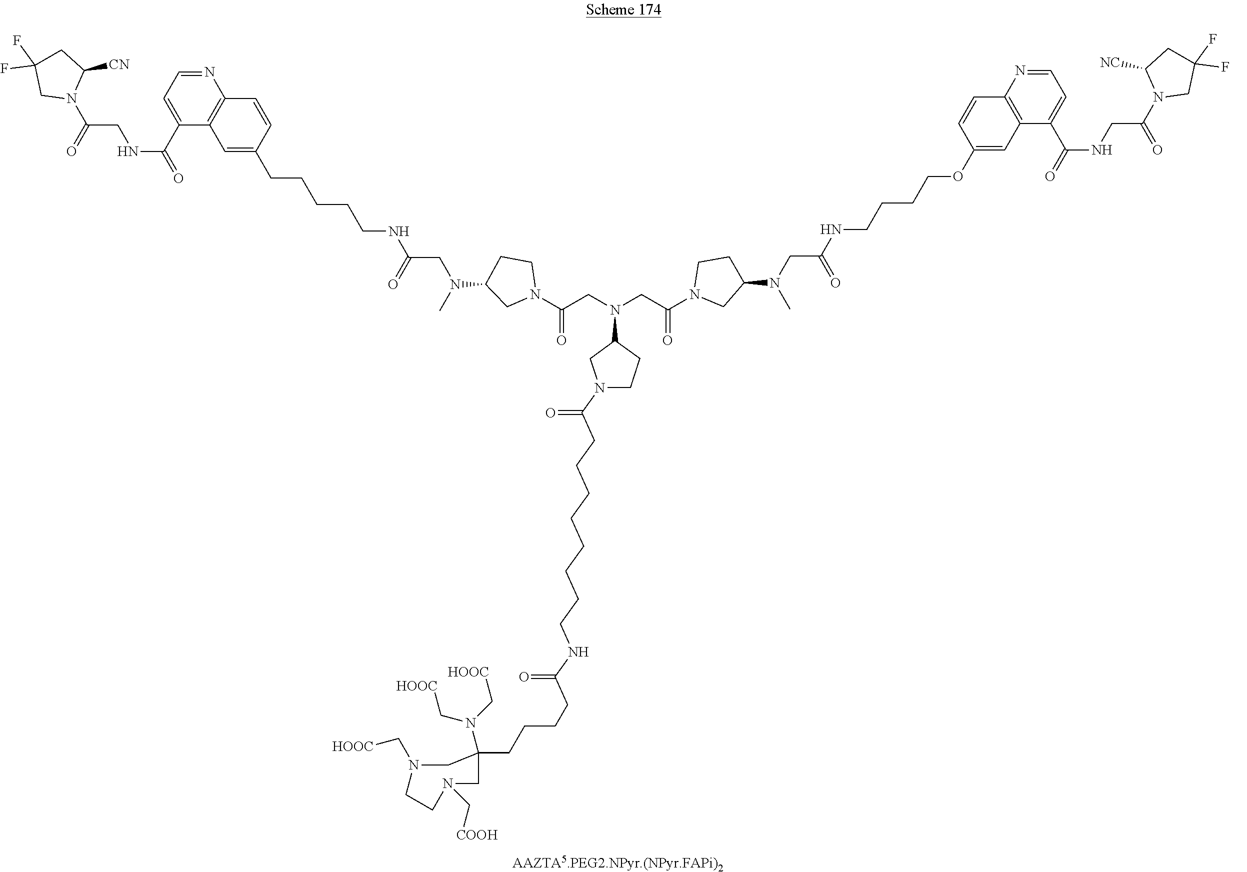

1. A dimeric labeling precursor for nuclear medical diagnosis and theranostics, having the structure 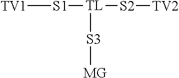
in which TV1 is a first targeting vector, TV2 is a second targeting vector, MG is a chelator or a linker for the complexation or covalent binding of a radioisotope, S1 is a first spacer, S2 is a second spacer, S3 is a third spacer and TL is a tris linker;
TV1 and TV2 are independently chosen from one of the structures [1]to [43]: 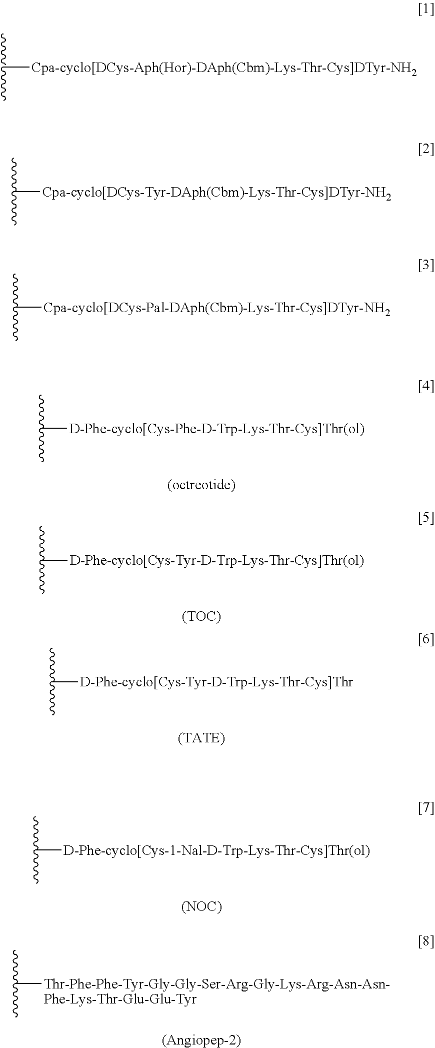 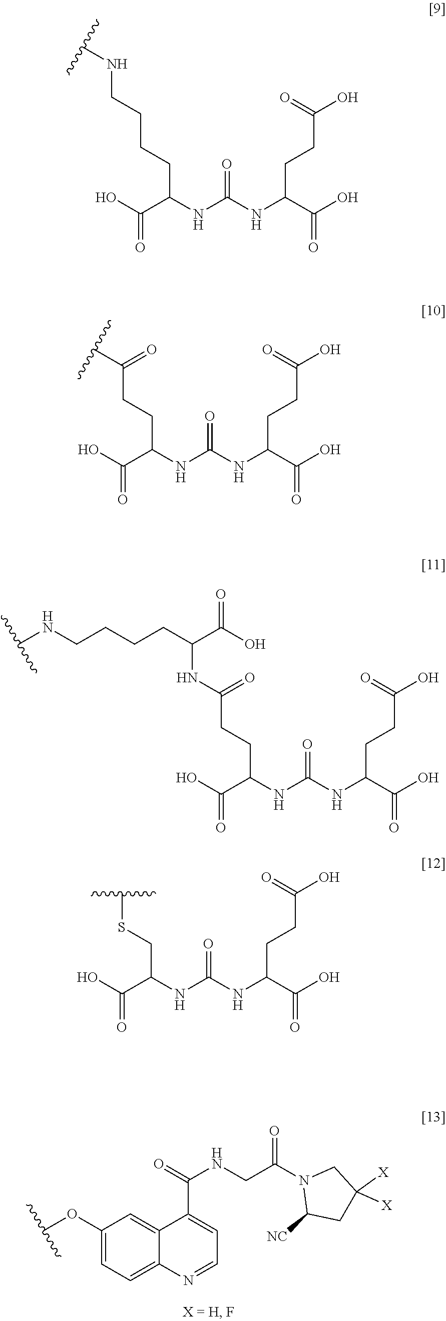 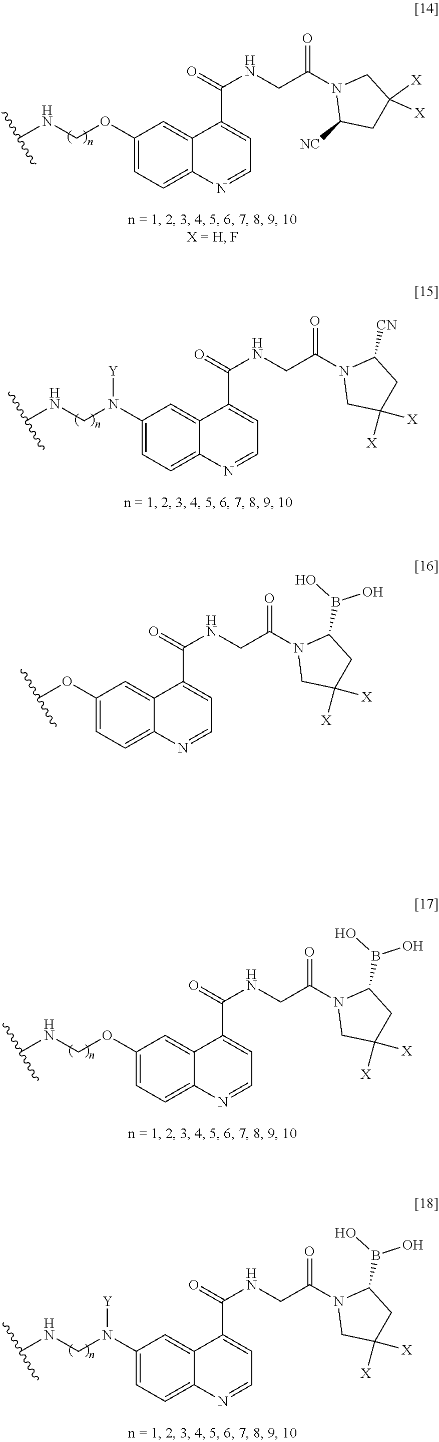 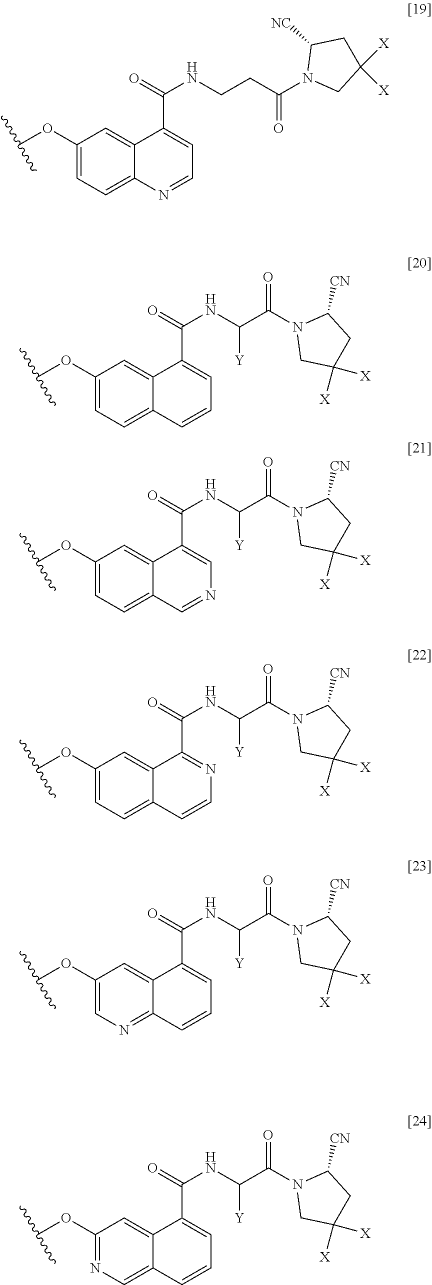 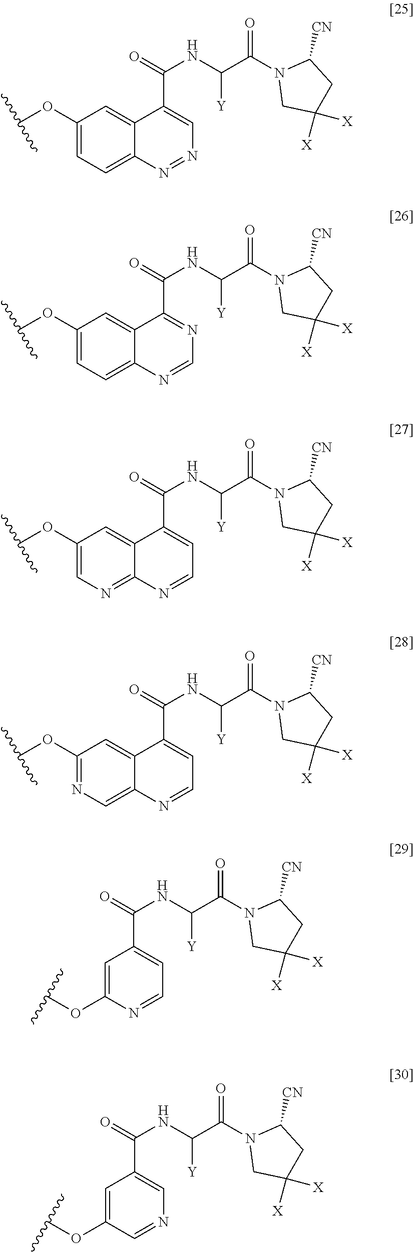 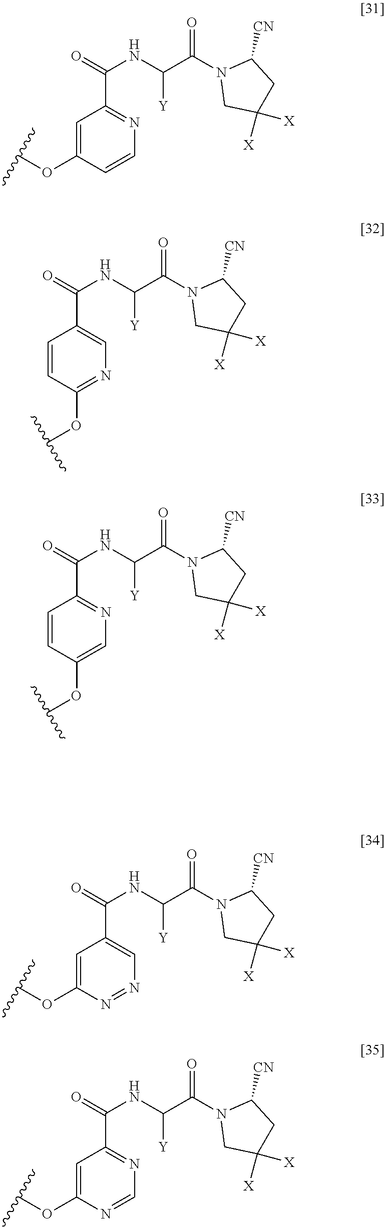 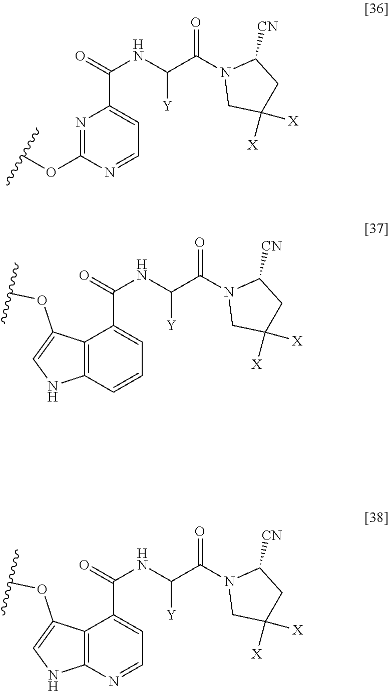 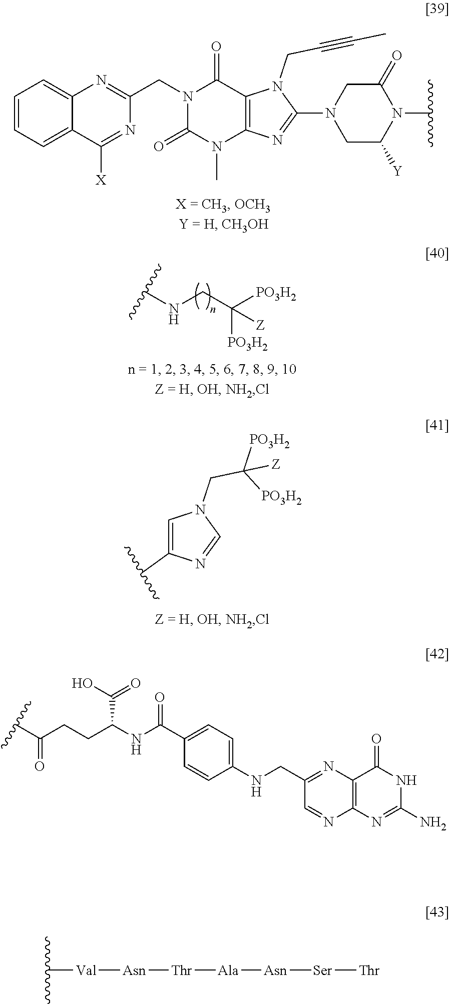
where
structures [1] to [8] and [43] denote peptides;
X═H or F;
Y═H, CH₃, CH(CH₃)₂, C(CH₃)₃ or (CH₂)ₙCH₃ with n=1, 2, 3, 4, 5, 6, 7, 8, 9 or 10;
the tris linker TL is chosen from one of structures [52] to [116]:

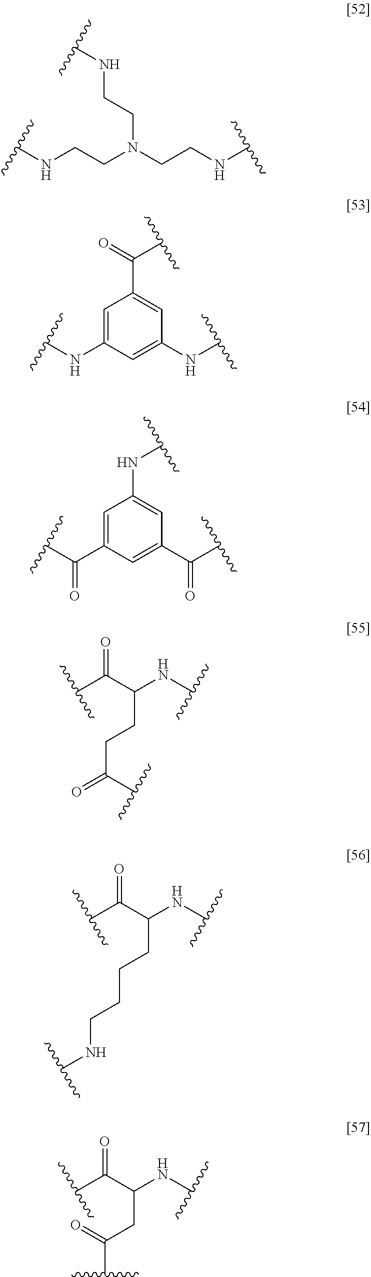 [SMILES: *NCCN(CCN*)CCN* ; *Nc1cc(N*)cc(C(*)=O)c1 ; *Nc1cc(C(*)=O)cc(C(*)=O)c1 ; *NC(CCC(*)=O)C(*)=O ; *NCCCCC(N*)C(*)=O ; *NC(CC(*)=O)C(*)=O]

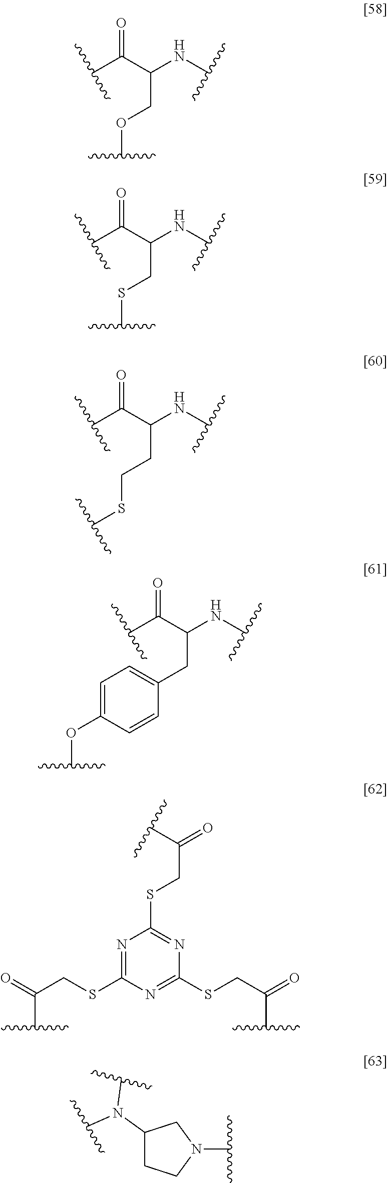

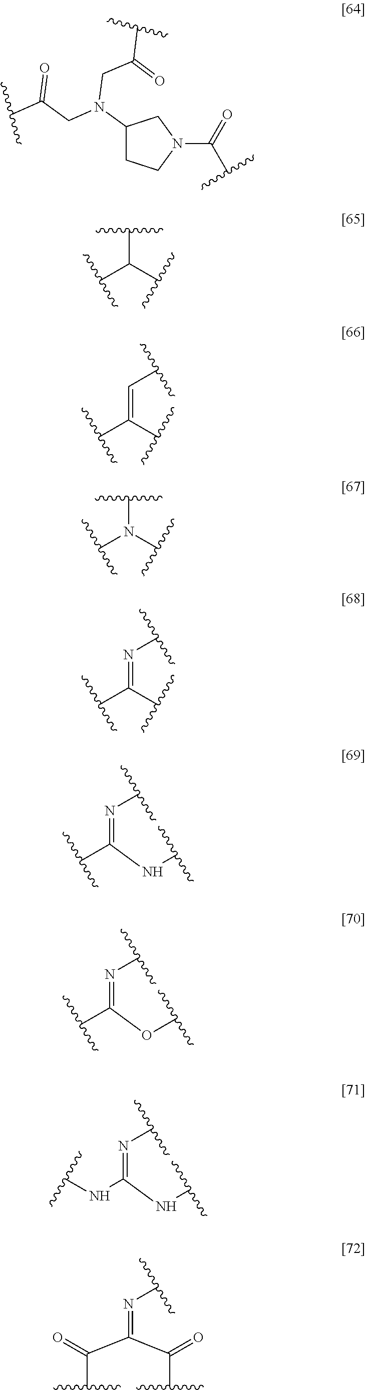

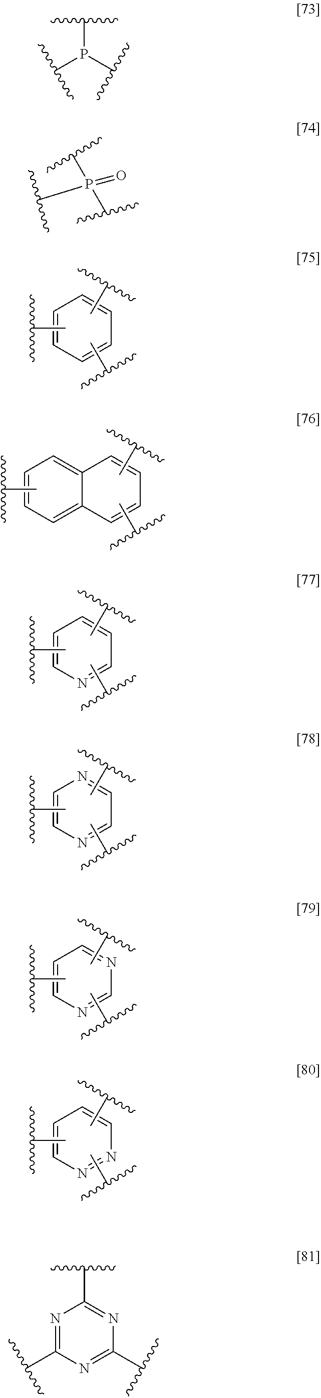

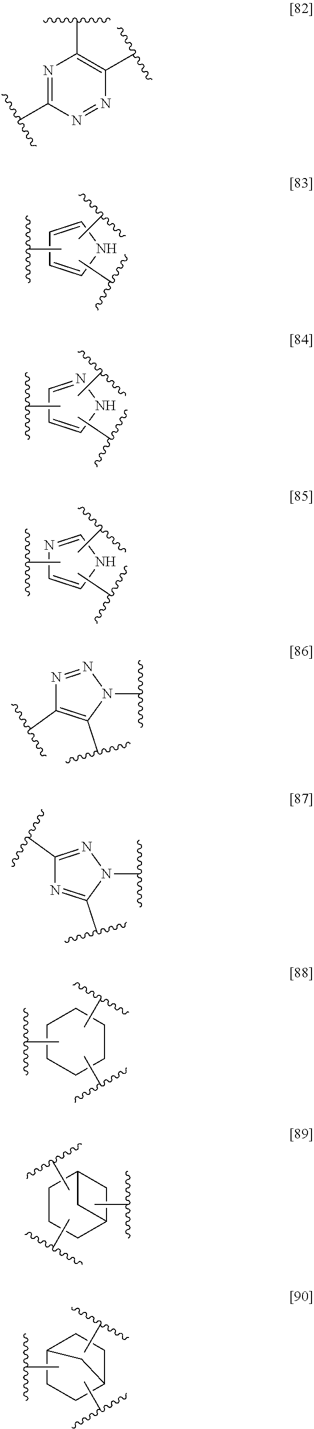

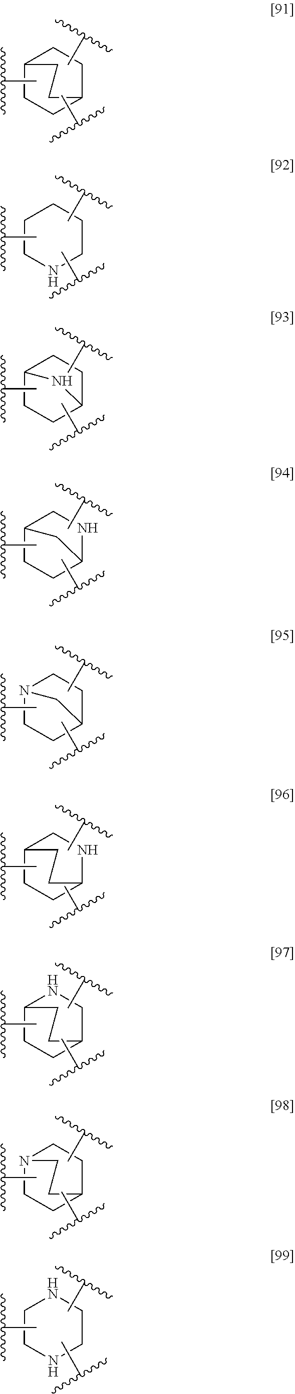

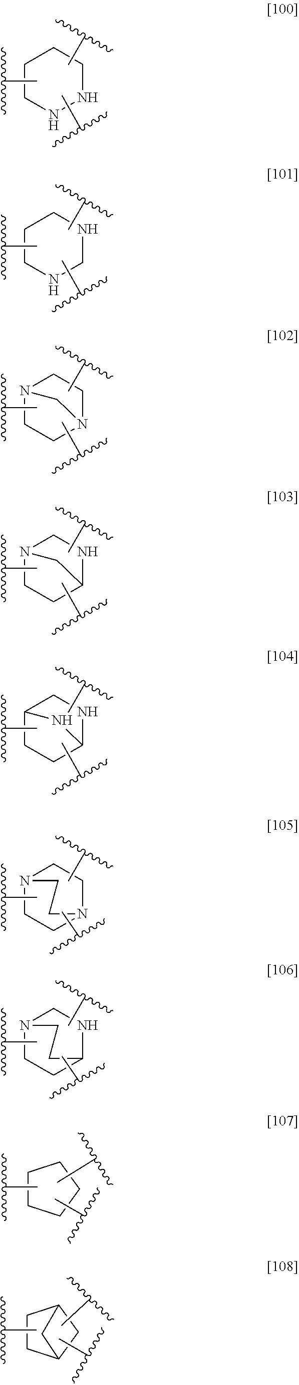

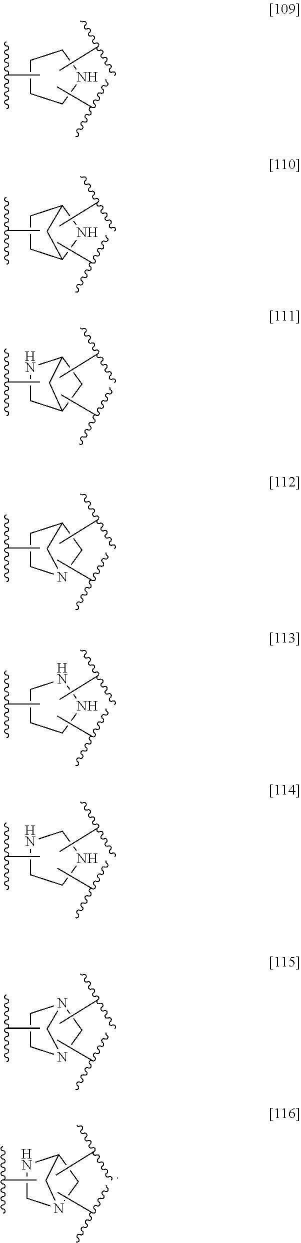

2. The labeling precursor as claimed in claim 1, wherein MG is a chelator chosen from the group comprising H₄pypa, ethylenediaminetetraacetate (EDTA), diethylenetriaminepenta(methylenephosphonic acid) (EDTMP), diethylenetriaminepentaacetate (DTPA) and derivatives thereof, nona-1,4,7-triamine triacetate (NOTA) and derivatives thereof, triazacyclononanephosphinic acid TRAP, 1,4,7-triazacyclononane-1,4-bis[methylene(hydroxymethyl)phosphinic acid]-7-[methylene(2-carboxyethyl)phosphinic acid](NOPO), dodeca-1,4,7,10-tetraaminetetraacetate (DOTA), 2-(1,4,7,10-tetraazacyclododecane-4,7,10)pentanedioic acid (DOTAGA) and other DOTA derivatives, trideca-1,4,7,10-tetraaminetetraacetate (TRITA), tetradeca-1,4,8,11-tetraaminetetraacetate (TETA) and derivatives thereof, pentadeca-1,4,7,10,13-pentaaminepentaacetate (PEPA), hexadeca-1,4,7,10,13,16-hexaaminehexaacetate (HEHA) and derivatives thereof, N,N′-bis(2-hydroxybenzyl)ethylenediamine-N,N′-diacetate (HBED) and derivatives thereof, DEDPA and derivatives thereof, deferoxamine (DFO) and derivatives thereof, trishydroxypyridinone (THP) and derivatives thereof tetraazacyclodecanephosphinic acid (TEAP) and derivatives thereof, 6-amino-6-methylperhydro-1,4-diazepane-N,N,N′,N′-tetraacetate (AAZTA) and derivatives thereof, (1-N-(4-aminobenzyl)-3,6,10,13,16,19-hexaazabicyclo[6.6.6]eicosane-1,8-diamine) (sarcophagine SAR) and derivatives thereof, 3-[(2′-aminoethyl)amino]-2-[(2″-aminoethyl)aminomethyl]propionic acid N₄ and other N₄ derivatives, 6-(4-isothiocyanatobenzyl)-3,3,9,9-tetramethyl-4,8-diazaundecane-2,10-dione dioxime (PnAO) and derivatives, mercaptoacetylglycylglycine (MAG2) and derivatives thereof, mercaptoacetylglycylglycylglycine (MAG3) and derivatives thereof, mercaptoacetylserylserylserine (MAS3) and derivatives thereof, (N-(2-mercaptoethyl)-2-[(2-mercaptoethyl)amino]acetamide) (MAMA) and derivatives thereof, ethylenedicysteine (EC) and derivatives thereof, dimercaptosuccinic acid (dmsa) and derivatives thereof, diaminodithiol (DADT), diaminodisulfide (DADS), N₂S₂ chelators and derivatives thereof, aminothiols and derivatives thereof, salts of the aforementioned chelators; hydrazinenicotinamides (HYNIC) and hydrazinenicotinamide derivatives.

3. The labeling precursor as claimed in claim 2, wherein MG is dodeca-1,4,7,10-tetraaminetetraacetate (DOTA), 1,4-bis(carboxymethyl)-6-[methyl-carboxymethylamino]-6-pentanoic acid-1,4-diazepane (DATA^{5m}) or 1,4-bis(carboxymethyl)-6-[bis(carboxymethyl)amino]-6-pentanoic acid-1,4-diazepane (AAZTA).

4. The labeling precursor as claimed in claim 1, wherein MG is chosen from

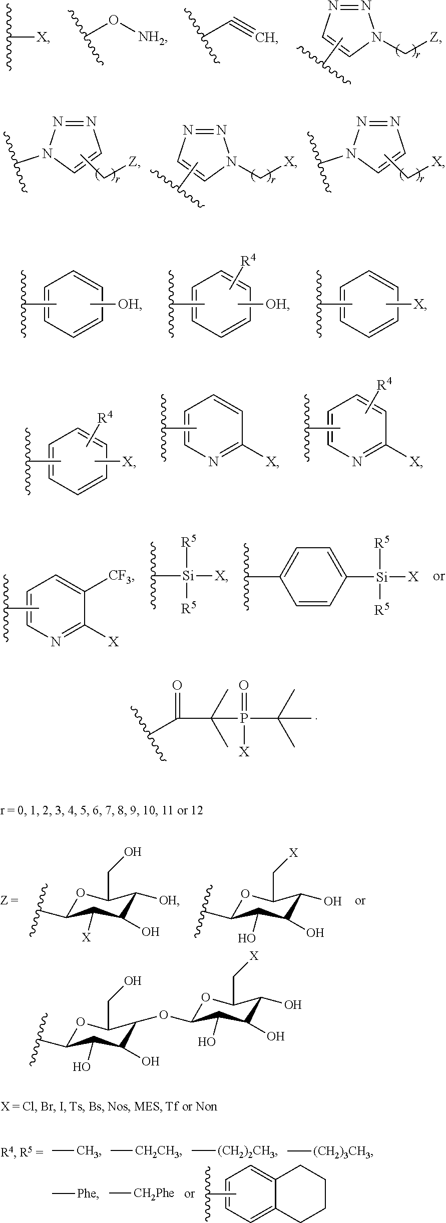

5. The labeling precursor as claimed in claim 1, wherein the spacers S1, S2, S3 independently have a structure chosen from 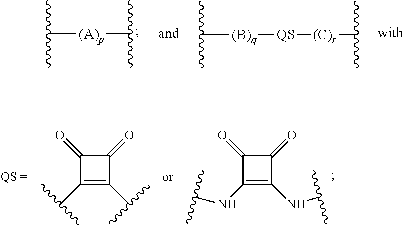
in which A, B, C are independently chosen from the group comprising amide radicals, carboxamide radicals, phosphinate radicals, alkyl radicals, triazole radicals, thiourea radicals, ethylene radicals, maleimide radicals, amino acid residues, 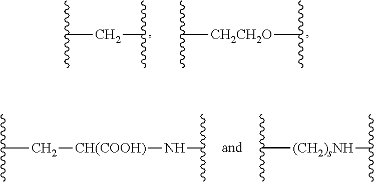
with s=1, 2, 3, 4, 5, 6, 7, 8, 9 or 10; and p, q and r are independently chosen from the set of {0, 1, 2, 3, 4, 5, 6, 7, 8, 9, 10, 11, 12, 13, 14, 15, 16, 17, 18, 19, 20}.

6. The labeling precursor as claimed in claims 1 to 4, wherein the spacers S1, S2, S3 independently have the structure

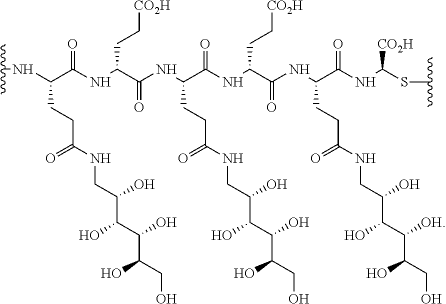 [SMILES: *N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](S*)C(=O)O]

7. The labeling precursor as claimed in claim 1, wherein the spacers S1, S2, S3 are independently chosen from a peptide group, dipeptide group or tripeptide group having the structure

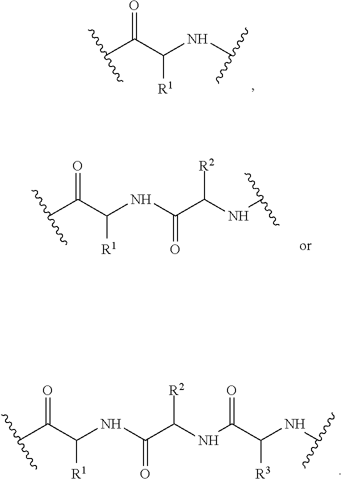

8. The labeling precursor as claimed in claim 7, wherein R¹, R², R³ are independently chosen from the group comprising —H, —CH₃, —CH(CH₃)₂, —CH₂CH(CH₃)₂, —CH(CH₃)—CH₂CH₃, —CH₂-Phe, —CH₂-Phe-OH, —CH₂SH, —(CH₂)₂—S—CH₃, —CH₂OH, —(CH)(OH)(CH₃), —(CH₂)₄NH₂, —(CH₂)₃NH(C═NH)NH₂, —CH₂COOH, —(CH₂)₂COOH, —CH₂(C═O)NH₂, —(CH₂)₂(C═O)NH₂,

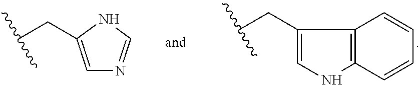 [SMILES: *Cc1cnc[nH]1 ; *Cc1c[nH]c2ccccc12]

9. The labeling precursor as claimed in claim 1, wherein TV1 is the same as TV2 (TV1=TV2).

10. The labeling precursor as claimed in claim 1, wherein TV1 and TV2 are different than one another (TV1≠TV2).

11. The labeling precursor as claimed in claim 10, wherein TV1 has one of the structures [9] to [12] and TV2 has one of the structures [13] or [14].

12. The labeling precursor as claimed in claim 10, wherein TV1 has one of the structures [9] to [12] and TV2 has one of the structures [40] or [41].

13. A radiotracer for nuclear medical diagnostics and theranostics, consisting of a labeling precursor as claimed in claim 1 and a radioisotope chosen from the group comprising ⁴⁴Sc, ⁴⁷Sc, ⁵⁵Co, ⁶²Cu, ⁶⁴Cu, ⁶⁷Cu, ⁶⁶Ga, ⁶⁷Ga, ⁶⁸Ga ⁸⁹Zr, ⁸⁶Y, ⁹⁰Y, ⁸⁹Zr, ⁹⁰Nb, ^{99m}Tc, ¹¹¹In, ¹³⁵Sm, ¹⁴⁰Pr ¹⁵⁹Gd, ¹⁴⁹Tb, ¹⁶⁰Tb, ¹⁶¹Tb, ¹⁶⁵Er, ¹⁶⁶Dy, ¹⁶⁶Ho, ¹⁷⁵Yb, ¹⁷⁷Lu, ¹⁸⁶Re, ¹⁸⁸Re, ²¹¹At, ²¹²Pb, ²¹³Bi, ²²⁵Ac, ²³²Th, ¹⁸F, ¹³¹I or ²¹¹At.

14. The labeling precursor as claimed in claim 2, wherein the NOTA derivative is 1,4,7-triazacyclononane,1-glutaric acid,4,7-acetate (NODAGA), the HBED derivative is N,N′-bis[2-hydroxy-5-carboxyethyl]benzyl)ethylenediamine-N,N′-diacetate (HBED-CC), the DEDPA derivative is 1,2-[[6-(carboxyl)pyridin-2-yl]methylamine]ethane (H₂dedpa) or 1,2-[[6-(carboxyl)pyridin-2-yl]methylamine]ethane-N,N′-diacetate (H₄octapa), the THP derivative is H₃THP-Ac or H₃THP-mal, the AAZTA derivative is 5-[(6-amino)-1,4-diazepane]pentanoic acid-N,N,N′,N′-tetraacetate (AAZTA⁵) or 5-[[6-(N-methyl)amino]-1,4-diacetate-1,4-diazepane]-pentanoic acid-N,N′,N′-triacetate (DATA^{5m}); the sarcophagine SAR derivative is 1,8-diamino-3,6,10,13,16,19-hexaazabicyclo[6.6.6]eicosane ((NH₂)₂SAR), the PnAO derivative is 3,3′-(1,4-butanediyldiamino)-bis(3-methyl-2-butanone) dioxime (BMS181321), and the MAG3 derivative is N₃S-adipat.
